(19) United States
(12) Patent Application Publication
(10) Pub. No.: US 2025/0082768 A1
(43) Pub. Date: Mar. 13, 2025

(54) CONJUGATING REAGENTS AND CONJUGATES THEREOF

(71) Applicant: CAMBRIDGE ENTERPRISE LIMITED, Cambridge, Cambridgeshire (GB)

(72) Inventors: David Robert Spring, Cambridge (GB); Stephen James Walsh, Cambridge (GB); Friederike Marie Dannheim, Cambridge (GB)

(21) Appl. No.: 18/291,380

(22) PCT Filed: Jul. 26, 2022
(86) PCT No.: PCT/EP2022/071003

§ 371 Date: Jan. 23, 2024

(30) Foreign Application Priority Data
Jul. 26, 2021 (GB) 2110726.3

(51) Int. Cl. A61K 47/68; A61P 35/00; C07K 16/28; C07K 16/32

(52) U.S. Cl. CPC A61K 47/68031; A61K 47/6849; A61K 47/6855; A61K 47/6889; A61P 35/00; C07K 16/2878; C07K 16/32

(57) ABSTRACT

The present invention relates to antibody conjugates, and methods of manufacturing the same. In particular, the present invention relates to a conjugate comprising an antibody, a linker and at least one active agent, wherein: the linker connects the at least one active agent(s) to the antibody; ii) the linker is attached to the antibody through 5 to 8 independent covalent bonds; and iii) each covalent bond between the linker and the antibody is formed from the reaction between a sulfur atom on the antibody and a functional group on the linker.

The present invention relates to antibody conjugates, and methods of manufacturing the same. More especially, the present invention relates to providing an antibody, linker and an active agent, for example a drug or labelling moiety, to produce a conjugate. Additionally, the present invention provides an improved linker for use in conjugates and methods of introducing said linker into said conjugates. More specifically, the conjugate may be an antibody drug conjugate (ADC).

The selective delivery of drugs to malignant cells without undesired toxicity towards healthy tissues is one of the primary goals in the development of modern cancer therapeutics. Antibody-drug conjugates (ADCs) are a promising class of targeted therapeutics which aim to address this need (Walsh 2021, Beck 2014, Teicher 2012). By harnessing the exquisite specificity of antibodies for cell-surface antigens overexpressed by cancer cells, ADCs enable the selective delivery of highly cytotoxic payloads to tumour sites. The utility of this therapeutic strategy has been showcased by the recent Food and Drug Administration (FDA) approval of enfortumab vedotin (Padcev™), trastuzumab deruxtecan (Enhertu™) sacituzumab govitecan (Trodelvy™), and belantamab mafodotin (Blenrep™) increasing the number of ADCs on the market to ten (Challita-Eid 2016, Modi 2020, Kaplon 2020, Syed 2020 and Hamadani (2021)).

However, current methods for the production of ADCs still suffer from various shortcomings. One common strategy for ADCs formation involves the conjugation of the payload to surface-exposed lysine residues via N-hydroxysuccinimide (NHS) ester motifs. Another approach involves conjugation to antibody cysteine residues. Human IgG1 antibodies contain four interchain disulfides, which can be reduced to reveal eight nucleophilic cysteine residues that can be conjugated to linkers containing electrophilic motifs such as maleimides. Due to the high abundance of reactive lysine (>50) and cysteine (8) residues in antibodies, modifications at these residues suffer from an inherent lack of site-selectivity and result in highly heterogeneous ADCs which vary in terms of conjugation site and the number of drug molecules attached to each antibody (drug-to-antibody ratio, DAR) (Kim 2014, Jackson 2016, Freedy 2016, Chudasama 2016). Conjugation site and DAR have been shown to have a significant effect on both the safety and efficacy of ADCs (Hamblett 2004, Shen 2012, Strop 2013). All of the currently FDA-approved ADCs are synthesised via lysine or cysteine conjugation and exist as heterogenous mixtures of conjugates with a DAR distribution of 0-8 and up to 70 different conjugation sites; this makes their pharmacokinetic profiles unnecessarily complex and difficult to predict (Kim 2014, Agarwal 2015, Godwin 2017, Bross 2001, Hedrich 2018).

Furthermore, one of the most commonly employed cysteine modification strategies—maleimide bioconjugation—has been shown to yield unstable linkages which are prone to undergo retro-Michael additions under physiological conditions (Alley 2008, Lyon 2014). This instability may lead to premature dissociation of the payload from the antibody and result in undesired toxicity in healthy tissues.

Several strategies to circumvent these issues through the incorporation of unnatural amino acids with bioorthogonal functionality or modification of antibody glycans have been reported (Fang 2014, Amant 2019, Amant 2019*, Walker 2019, Axup 2012, Zimmerman 2014). While these approaches have indeed produced ADCs with high stability, defined attachment points and precise DAR, the generation of such non-native antibody formats is generally laborious, low-yielding and may present an immunogenic risk. Therefore, the establishment of site-selective conjugation strategies using native antibodies is preferable. Disulfide bridging linkers have emerged as an attractive class of reagents that enable the synthesis of homogenous ADCs from native antibodies (Forte 2018). These linkers contain two cysteine-reactive groups that may undergo reaction with reduced interchain disulfides in an IgG molecule leading to covalent rebridging of the antibody chains. Dibromomaleimides (Behrens 2015, Smith 2010), pyridazinediones (Robinson 2017), bissulfones (Badescu 2014) and arylene-dipropiolonitrile (Koniev 2018) reagents have all been used in this context to successfully generate ADCs with demonstrated in vitro and in vivo activity. Also recently reported is the development of disulfide rebridging divinylpyrimidine (DVP) linkers (Walsh 2019, Walsh 2020). The rebridging approach yields immense improvements in homogeneity when compared to stochastic cysteine modifications and results in a defined DAR of 4 in every synthetic batch. However, a major limitation of this approach is the formation of “half-antibody” species during bioconjugation, which is the result of non-native intrachain cross-linking of the cysteine residues in the hinge region of the antibody. Half-antibody formation has been observed with all kinds of disulfide bridging linkers (Forte 2018). The loss of covalent linkages between the antibody heavy chains effected by this non-native rebridging is associated with reduced antibody stability; therefore the development of methods which abrogate half-antibody formation is highly desirable (Bahou 2019).

The present invention aims to provide a disulfide bridging linker platform which addresses the reported stability issues yet retains the advantages of a precise ratio between the active agent and the antibody, and ability to distribute the active agent. Furthermore, the present invention aims to provide a disulfide bridging linker platform which addresses the problem of half-antibody formation, and provides greater control to the number of drug molecules added to an antibody.

Accordingly, in a first aspect of the present invention, there is provided a conjugate comprising an antibody, a linker and at least one active agent, wherein:
i) the linker connects the at least one active agent(s) to the antibody;
ii) the linker is attached to the antibody through 5 to 8 independent covalent bonds; and
iii) each covalent bond between the linker and the antibody is formed from the reaction between a sulfur atom on the antibody and a functional group on the linker.

The present invention provides a linker for use in conjugates, with utility in linking antibodies and active agents, for example antibodies and cytotoxins to provide antibody-drug conjugate (ADC) molecules. The linker provides an improved method for controlling loading of active agents on the antibody. Thus, the present invention provides a linker for use in providing homogeneous ADCs with controlled drug-to-antibody ratios.

The linker also provides an improved targeted payload of the active agent, and thus may improve the activity of the conjugate where the active agent exerts a biological activity, such as cytotoxicity. Additionally or alternatively, the linker provides the conjugate with increased stability as compared to currently known linker molecules for use in conjugates. This may improve the tolerability of such conjugates.

The linker may be directly bound to five to eight thiol groups (sulfur atoms) of cysteine residues in the antibody. Thus, the linker connects to four reduced interchain disulfide bonds via one or both of the sulfur atoms associated with the four reduced interchain disulfide bonds. Accordingly, the linker serves to re-bridges between one and four reduced interchain disulfide bonds in the antibody. Thus, the linker may be connected to the antibody (e.g. human IgG1 antibodies) via 5 to 8 covalent bonds to the eight thiol groups of the cysteine residues involved in the interchain disulfide bonds. Specifically, the linker may be connected to the antibody (e.g. human IgG1 antibodies) via 5 to 8 covalent bonds to the eight thiol groups of the cysteine residues involved in the interchain disulfide bonds at the hinge region of the antibody.

Preferably, the linker is directly bound to six to eight, or more preferably seven to eight, thiol groups (sulfur atoms) of cysteine residues in the antibody.

Thus, the linker may re-bridge one to four of the reduced interchain disulfide bonds in the antibody (e.g. human IgG1 antibodies), via 5 to 8 covalent bonds to the eight thiol groups (sulfur atoms) of the cysteine residues involved in the interchain disulfide bond. Preferably, the linker is directly bound to six to eight, or more preferably seven to eight, thiol groups (sulfur atoms) of cysteine residues in the antibody. Thus, the linker preferably re-bridges two to four, or more preferably three to four, of the reduced interchain disulfide bonds in the antibody (e.g. human IgG1 antibodies), via 6 to 8 (or more preferably 7 to 8) covalent bonds to the eight thiol groups (sulfur atoms) of the cysteine residues involved in the interchain disulfide bonds. Most preferably, the linker is directly bound to eight thiol groups (sulfur atoms) of cysteine residues in the antibody. Thus, the linker re-bridges all four of the reduced interchain disulfide bonds in the antibody (e.g. human IgG1 antibodies), via 8 covalent bonds to the eight thiol groups (sulfur atoms) of the cysteine residues involved in the interchain disulfide bonds.

In a second aspect of the present invention, there is provided a conjugating reagent capable of reaction with an antibody, said conjugating reagent comprising a linker, at least one active agent and 8 functional groups capable of reaction with between 5 and 8 sulfur atoms on the antibody.

Thus, suitably, the conjugating reagent of the invention is a compound of Formula (II), shown below:

(D-L¹-FG)ₙ-L-(Z)₈ (Formula II)
wherein:
   FG′ is a functional linking moiety;
   n is an integer selected from 0 to 20;
   L and L¹ are independently linkers;
   Z is a functional group capable of reaction with a sulfur atom from a cysteine moiety of an antibody; and
   D is an active agent;
or a pharmaceutically acceptable salt, solvate or hydrate thereof.

This conjugating reagent may be reacted with an antibody to form the conjugate of the first aspect of the invention.

Preferably, the conjugating reagent comprises a linker, at least one active agent and 8 functional groups capable of reaction with between 6 and 8, more preferably 7 to 8, and most preferably 8, sulfur atoms on the antibody.

In a third aspect of the invention, there is provided an intermediate conjugate comprising an antibody, a linker and at least one functional group capable reacting with another moiety to form a functional linking moiety, wherein:
i) the linker connects the at least one functional group to the antibody;
ii) the linker is attached to the antibody through 5 to 8 independent covalent bonds; and
iii) each covalent bond between the linker and the antibody is formed from the reaction between a sulfur atom on the antibody and a functional group on the linker.

Thus, the intermediate conjugate may be reacted with at least one active agent to form the conjugate of the first aspect of the invention. This reaction between the intermediate conjugate and the active agent(s) can take place via reaction of the at least one functional group on the intermediate conjugate and another functional moiety on the active agent. As such, a functional linking moiety linking the intermediate conjugate to the active agent(s) is formed.

In a fourth aspect of the present invention, there is provided a compound capable of reaction with both an antibody and at least one active agent, said compound comprising a linker, 8 functional groups capable of reaction with between 5 and 8 sulfur atoms on the antibody and at least one functional group capable reacting with another moiety to form a functional linking moiety. The functional linking moiety serving as the means by which the at least one active agent may be attached to the compound.

Thus, suitably, the compound of the invention is a compound of Formula (I), shown below:

(FG)ₙ-L-(Z)₈ (Formula I)
wherein:
   FG is a functional group capable of reaction with another moiety to form a functional linking moiety;
   n is an integer selected from 0 to 20;
   L is a linker; and
   Z is a functional group capable of reaction with a sulfur atom from a cysteine moiety of an antibody;
or a pharmaceutically acceptable salt, solvate or hydrate thereof.

The compound of the fourth aspect is a precursor of the conjugating reagent of the second aspect of the invention. The compound of the fourth aspect is also a precursor of the intermediate conjugate of the third aspect of the invention. The conjugating reagent can be formed by reacting the compound of Formula (I) with an active agent, which may have a linker group already attached. The intermediary conjugate can be formed by reacting the compound of Formula (I) with an antibody (an antibody that has had its interchain disulfide bonds reduced).

In a fifth aspect of the present invention, there is provided a pharmaceutical composition comprising, a conjugate of the first aspect of the invention, where the active agent is a drug, and a carrier, excipient or diluent. The fifth aspect also provides a conjugate of the first aspect of the invention, where the active agent is a drug, for use in a method of treatment.

It will be understood that the pharmaceutical composition according to fifth aspect of the present invention may comprise a mixture of conjugates of the first aspect of the invention. That is, it will be understood that the pharmaceutical composition may comprise a mixture of conjugates wherein the linker is attached to the antibody through either 5, 6, 7 or 8 independent covalent bonds. It will also be understood that pharmaceutical composition may also comprise conjugates wherein the linker is attached to the antibody through 1 to 4 independent covalent bonds.

Suitably, the pharmaceutical composition comprises a mixture of conjugates wherein at least 65% of the conjugates comprise a linker which is attached to the antibody through 8 independent covalent bonds. More suitably, the pharmaceutical composition comprises a mixture of conjugates wherein at least 70% of the conjugates comprise a linker which is attached to the antibody through 8 independent covalent bonds. Even more suitably, the pharmaceutical composition comprises a mixture of conjugates wherein at least 75% of the conjugates comprise a linker which is attached to the antibody through 8 independent covalent bonds. Yet more suitably, the pharmaceutical composition comprises a mixture of conjugates wherein at least 80% of the conjugates comprise a linker which is attached to the antibody through 8 independent covalent bonds. Still more suitably, the pharmaceutical composition comprises a mixture of conjugates wherein at least 85% of the conjugates comprise a linker which is attached to the antibody through 8 independent covalent bonds. Still more suitably, the pharmaceutical composition comprises a mixture of conjugates wherein at least 90% of the conjugates comprise a linker which is attached to the antibody through 8 independent covalent bonds. Still more suitably, the pharmaceutical composition comprises a mixture of conjugates wherein at least 95% of the conjugates comprise a linker which is attached to the antibody through 8 independent covalent bonds. Most suitably, the pharmaceutical composition comprises a mixture of conjugates wherein at least 98% of the conjugates comprise a linker which is attached to the antibody through 8 independent covalent bonds.

Thus, suitably, at least 65% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. More suitably, at least 70% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. Yet more suitably, at least 75% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. Even more suitably, at least 85% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. Even more suitably, at least 90% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. Even more suitably, at least 95% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds. Most suitably, at least 98% of the conjugate present in the pharmaceutical composition is a conjugate of the first aspect of the invention wherein the linker re-bridges four of the reduced interchain disulfide bonds in the antibody (e.g. human IgG1 antibodies), via 8 covalent bonds to each of the eight thiol groups (sulfur atoms) of the cysteine residues involved in the interchain disulfide bonds.

In a sixth aspect of the present invention there is provided the use of a conjugate of the first aspect of the invention, where the active agent is a cytotoxic drug, in the manufacture of a medicament for treating a proliferative disease. The sixth aspect also provides a conjugate of the first aspect of the invention, where the active agent is a cytotoxic drug, for use in the treatment of a proliferative disease. The sixth aspect also provides a method of treating a proliferative disease comprising administering a therapeutically effective amount of a conjugate of the first aspect of the invention, where the active agent is a cytotoxic drug, to a patient in need thereof.

### Definitions

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention extends to any novel one, or any novel combination, of the features disclosed in this specification (including any accompanying claims, abstract and drawings), or to any novel one, or any novel combination, of the steps of any method or process so disclosed.

The terms “specifically binds” and “specific binding” refer to the binding of an antibody to a predetermined molecule (e.g., an antigen). Typically, the antibody binds with an affinity of at least about 1×10⁷ M⁻¹, and binds to the predetermined molecule with an affinity that is at least two-fold greater than its affinity for binding to a non-specific molecule (e.g., BSA, casein) other than the predetermined molecule or a closely-related molecule.

### Antibodies

The term “antibody” herein is used in the broadest sense and specifically covers monoclonal antibodies, polyclonal antibodies, dimers, multimers, multispecific antibodies (e.g., bispecific antibodies), multivalent antibodies and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full-length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include chimeric antibodies, humanized antibodies and human antibodies.

Examples of cell binding agents include those agents described for use in WO 2007/085930, which is incorporated herein.

Tumour-associate antigens and cognate antibodies for use in embodiments of the present invention are listed below, and are described in more detail on pages 14 to 86 of WO 2017/186894, which is incorporated herein.

### Active Agent

The active agent may be a drug, a labelling moiety or a targeting moiety. Preferably, the active agent is a drug or a labelling agent. The active agent needs to comprise a functional group that can be linked to the linker. Such a group may include, for example, an amino group, an imine group or a hydroxy group. In situations where there exists more than one active agent, it is to be understood that the two or more active agents may either be the same or different. For example, in situations where there exist two or more active agents, the two or more active agents may independently be selected from a drug, a labelling moiety or a targeting moiety. It will also be understood that in situations where there exist more than one drug, a labelling moiety or a targeting moiety, each drug, labelling moiety and targeting moiety may be the same or different.

### Drug

The drug may be a cytotoxic payload or a therapeutic compound, peptide or polypeptide. In particular, the drug is preferably a cytotoxin.

Preferably the cytotoxin is a biologically active cytotoxic material. The cytotoxin may be selected from the group comprising auristatins, maytansinoids, tubulysins, calicheamicins, duocarmycins, pyrrolobenzodiazepines (in particular pyrrolobenzodiazepine dimers), camptothecin analogues and doxorubicin.

However, additionally or alternatively, the cytotoxin could also be selected from other known cytotoxins including ricin subunits and other peptide based cytotoxic materials, although such materials are less commonly utilised in the field of the art.

### Labelling Moiety

The labelling moiety may be a fluorophore. Suitable fluorophores include fluorescein isothiocyanate (FITC), phycoerythrin (PE), allophycocyanin (APC), Indocicarbocyanine (Cy5), Indocarbocyanine (Cy3), as well as those known by the trade names Alexa Fluor (such as 350, 405, 488, 532, 546, 568, 594, 647, 680, 700, 750) and DyLight (such as 405, 488, 550, 650, 680, 755, 800).

The labelling moiety may also be a biotin tag, derived from biotin.

The labelling moiety may also be a radioisotope or a radioisotope containing moiety. Suitably, the labelling moiety is a positron emission tomography (PET) tracer. Suitable PET tracers include, for example, [18F] Fludeoxyglucose (18F) (FDG)-glucose analogue, [11C]acetate, [11C]methionine, [11C] choline, copper ₆₄Cu dotatate, [18F] EF5, [18F] fluciclovine, [18F]fluorocholine, [18F]fluoroethyl-L-tyrosine, [18F]fluoromisonidazole, [18F]fluorothymidine F-18, [₆₄Cu] Cu-ETS2, [68Ga] DOTA-pseudopeptides, [68Ga] DOTA-TATE and [68Ga] prostate-specific membrane antigen (PSMA).

### Targeting Moiety

The targeting moiety is a moiety that helps to direct to the conjugate to a particular location within the body. It will be appreciated that any suitable targeting moiety may be used. Suitably, the targeting moiety is a carbohydrate, such as alpha-D-galacto-hexopyranosyl-(1->3)-2-acetamido-2-deoxy-D-galacto-hexopyranose (Gal-alpha 1,3-GalNAc).

### Conjugates of the Invention

According to one aspect of the invention there is provided a conjugate comprising an antibody, a linker and at least one active agent, wherein:
i) the linker connects the at least one active agent(s) to the antibody;
ii) the linker is attached to the antibody through 5 to 8 independent covalent bonds; and
iii) each covalent bond between the linker and the antibody is formed from the reaction between a sulfur atom on antibody and a functional group on the linker.

Suitably, each covalent bond between the linker and the antibody is formed from the reaction between a sulfur atom on the antibody and an electrophilic functional group on the linker.

In certain embodiments, the conjugate comprises between one and four re-bridging moieties of Formula (III), shown below:

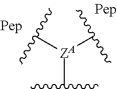

wherein:
   Z^{A} is a functional linking group;
   Pep indicates the position where the moiety is attached to the antibody, either directly or indirectly, such that the re-bridging moiety re-bridges a reduced interchain disulfide bond in the antibody; and

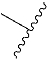

indicates the position where the moiety is attached to linker.
   Suitably, the conjugate comprises between two and four re-bridging moieties of Formula (III). More suitably, the conjugate comprises between three and four re-bridging moieties of Formula (III). Most suitably, the conjugate comprises four re-bridging moieties of Formula (III).

It will be understood that the functional linking group Z^{A} can be any functional group that is capable of linking the antibody to the linker. The functional linking group Z^{A} is thus a functional grouping that is capable of forming a covalent attachment to sulfur atoms present on the antibody. Suitably, the sulfur atoms are sulfur atoms from cysteine residues on the antibody, and more suitably, sulfur atoms from cysteine residues from reduced interchain disulfide bonds of the antibody.

Functional linking groups capable of linking the antibody to the linker in this way are well known in the art. Thus, the skilled person will be able to select suitable functional linking groups to use with the invention. Examples of suitable functional linking groups, Z^{A}, include, for instance, those described in, for example, Xu 2021, Stieger 2021, Walsh 2019, Walsh 2020, Badescu 2014, Koniev 2018, Robinson 2017, Behrens 2015 and WO2019/011078.

In certain embodiments, Z^{A} is selected from one of the functional linking groups shown below, and thus the conjugate comprises between one and four (preferably between two and four, more preferably between three and four, and most preferably four) re-bridging moieties independently selected from one of the following groups:

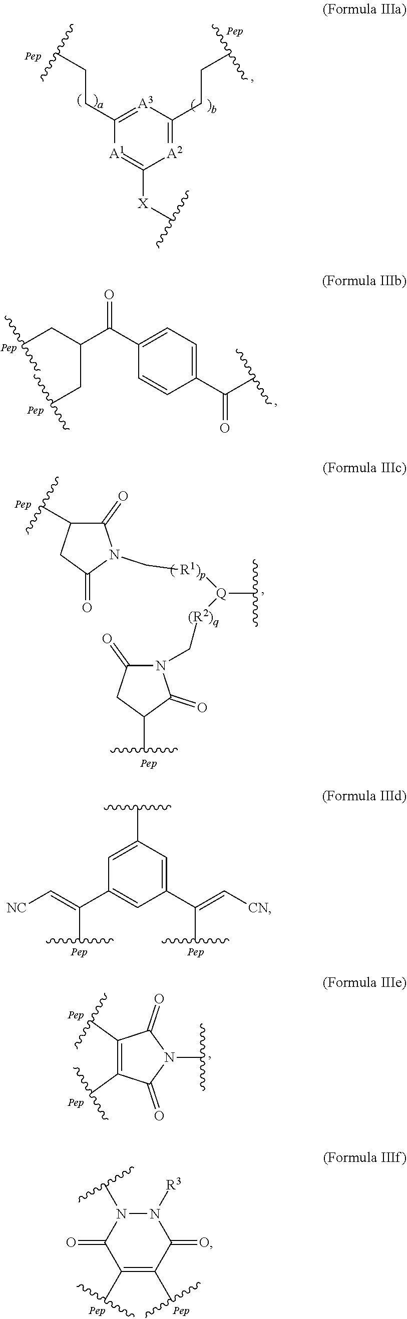

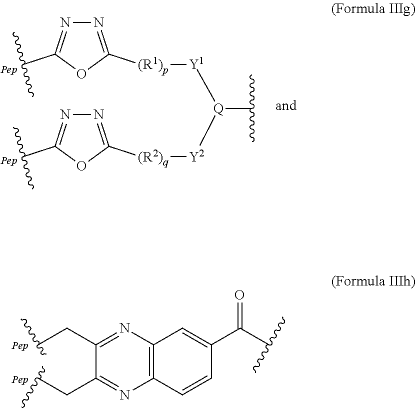

wherein:
   one or two of A¹, A² and A³ are N and the other one or two of A¹, A² and A³ are CH, or all three of A¹, A² and A³ are N or C;
   a and b are integers selected from 0 or 1;
   X is selected from N, NR^{N}, O and S, wherein R^{N} is H or C₁₋₂alkyl;
   R¹ and R² are independently selected from C₁-C₆ alkylene and C₁-C₆ alkylene containing O in the backbone;
   R³ is selected from hydrogen or a C₁-C₄ alkyl;
   Y¹ and Y² are independently absent or selected from O, NR⁴, C(═O),
   C(═O)NR⁴ or NR⁵C(═O);
   p and q are independently integers selected from 0 or 1;
   Q is CR⁶, N or aryl;
   R⁴, R⁵ and R⁶ are independently selected from hydrogen and C₁-C₄alkyl;
   Pep indicates the position where the moiety is linked to the antibody, either directly or indirectly; and

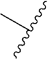

indicates the position where the moiety is attached to linker.

In certain embodiments, two of A¹, A² and A³ are N (e.g. A¹ and A² are N) and the other of A¹, A² and A³ is CH (e.g. A³ is CH). Suitably, in this embodiment, integers a and b are 1.

In other embodiments, all three of A¹, A² and A³ are N. Suitably, in this embodiment, integers a and b are 1.

In some embodiments, one of A¹, A² and A³ is N (e.g. A³ is N) and the other two of A¹, A² and A³ are CH (e.g. A¹ and A² are CH). Suitably, in this embodiment, integers a and b are 0.

Suitably, X is NH or O. Most suitably, X is NH.

In certain embodiments, R¹ and R² are independently selected from C₁-C₆ alkylene. Suitably, R¹ and R² are independently selected from C₁-C₄ alkylene.

In some embodiments, R³ is a C₁-C₄ alkyl (e.g. a methyl group).

In some embodiments, Y¹ and Y² are independently absent or selected from O, NR⁴ and C(═O). Suitably, Y¹ and Y² are independently absent.

Suitably, Q is CR⁶ or N. In some embodiments, Q is CR⁶. In other embodiments, Q is N.

In some embodiments, R⁴, R⁵ and R⁶ are independently selected from hydrogen and methyl. Suitably, R⁴, R⁵ and R⁶ are each hydrogen.

In certain embodiments, the conjugate comprises between one and four (preferably between two and four, more preferably between three and four, and most preferably four) re-bridging moieties independently selected from the following groups:

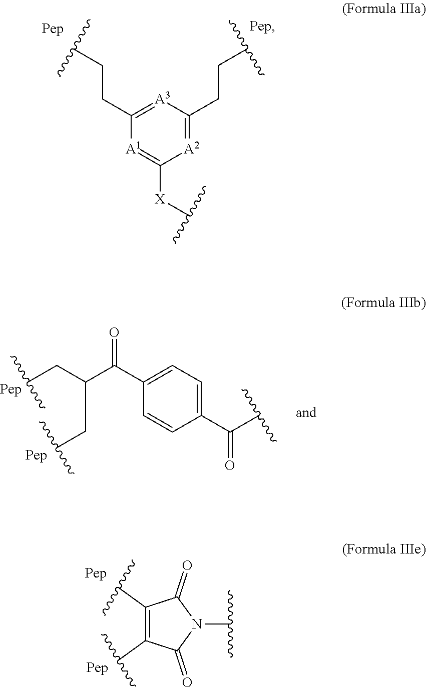

wherein each of A¹, A², A³, X and Pep are as defined herein.

In other embodiments, the conjugate comprises between one and four (preferably between two and four, more preferably between three and four, and most preferably four) re-bridging moieties of general formula (IIIa), shown below:

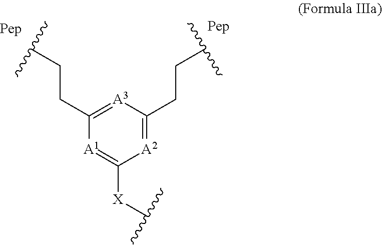

wherein each of A¹, A², A³, X, Pep and

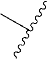

are as defined herein.

In some embodiments, A¹ and A² are N.

In other embodiments, A¹ and A³ are N.

In some embodiments, X is selected from NR^{N}, O and S, where R^{N} is H or C₁₋₂ alkyl. In these embodiments, only a single active agent can be attached is to X via a linker.

In some of these embodiments, X is NR^{N}. X may be NH, NCH₃ or NCH₂CH₃.

In others of these embodiments, X is O.

In others of these embodiments, X is S.

In some embodiments, X is N. In these embodiments, X can be attached to the linker via two covalently bonds, as shown below:

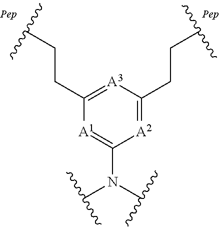

wherein each of A¹, A², A³, Pep and

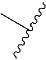

are as defined herein.

### Linker (L)

The linker is a group (functional grouping) that joins the antibody to the active agent(s). The linker attaches to the antibody via 5 to 8 (suitably via 6 to 8, more suitably via 7 to 8, and most suitably via 8) covalent bonds formed between sulfur atoms on the antibody and functional groups (e.g. electrophilic functional groups) on the linker. The sulfur atoms on the antibody may be sulfur atoms on cysteine residues on the antibody, such as sulfur atoms from the cysteine residues on the antibody that are involved in the interchain disulfide linkages of the antibody (i.e. sulfur atoms from cysteine residues made available following the reduction of antibody's interchain disulfide linkages).

The linker may therefore attach to the antibody via the functional linking groups (Z^{A}). The linker may therefore connect the functional linking groups (Z^{A}) to the at least one active agent. The linker can connect to the at least one active agent(s) via a functional linking moiety (FG′) as defined hereinbelow.

It will be understood that the linker may be any group (functional grouping) that is capable of connecting the at least one active agent to the antibody.

In certain embodiments, the linker is such that at least two of the re-bridging moieties (i.e. the groups of Formula (III)) are separated by between 10 and 60 atoms. Suitably, the linker is such that at least two of the re-bridging moieties (i.e. the groups of Formula (III)) are separated by between 10 and 50 atoms. More suitably, the linker is such that at least two of the re-bridging moieties (i.e. the groups of Formula (III)) are separated by between 10 and 40 atoms. Even more suitably, the linker is such that at least two of the re-bridging moieties (i.e. the groups of Formula (III)) are separated by between 12 and 40 atoms. Still more suitably, the linker is such that at least two of the re-bridging moieties (i.e. the groups of Formula (III)) are separated by between 15 and 40 atoms. Most suitably, the linker is such that at least two of the re-bridging moieties (i.e. the groups of Formula (III)) are separated by between 16 and 38 atoms.

In some embodiments, the linker comprises one or more groups selected from an alkylenediamine moiety, a polyethylene glycol moiety, an amino acid residue, an arylene-containing moiety, a heteroarylene-containing moiety, a heterocyclyl-containing moiety, a cycloalkyl-containing moiety and combinations thereof. Suitably, the linker comprises between 1 and 20 of the above-mentioned groups. More suitably, the linker comprises between 1 and 15 of the above-mentioned groups. Even more suitably, the linker comprises between 1 and 10 of the above-mentioned groups.

Suitably, the linker comprises one or more groups selected from an alkylenediamine moiety, a polyethylene glycol moiety, an amino acid residue and combinations thereof.

The alkylenediamine moiety will be understood to be a functional grouping that contains or is derived from an alkylenediamine moiety, such as, an ethylenediamine.

The polyethylene glycol moiety will be understood to be a functional grouping that comprises one or more alkylene glycol moieties, such as, one or more ethylene glycol moieties. In certain embodiments, the linker comprises between 1 and 10 alkylene glycol moieties (e.g. ethylene glycol moieties).

The amino acid residues will be understood to constitute both natural and unnatural amino acid residues. Suitably, the amino acid residue will be a natural amino acid residue. The amino acid residue may also comprise two or more amino acids, such as, for example, dipeptide and tripeptide moieties. Suitable amino acid residues include Phe, Lys, Val, Ala, Cit, Leu, Ile, Arg, and Trp residues.

The arylene-containing moiety will be understood to be a functional grouping that comprises one or more arylene groups. Suitably, the arylene will be a 6-membered arylene such as phenylene. In certain embodiments, the arylene-containing moiety is a functional grouping that includes a 1,3,5-benzyl group.

The heteroarylene-containing moiety will be understood to be a functional grouping that comprises one or more heteroarylene groups. Suitably, the heteroarylene will be a functional grouping that includes a 6-membered heteroaryl such as triazine. In certain embodiments, the arylene-containing moiety is a functional grouping that includes a 1,3,5 triazine group.

The heterocyclyl-containing moiety will be understood to be a functional grouping that comprises one or more heterocycle groups. Suitably, the heterocycle is a 6-membered heterocycle such as triazinane. In certain embodiments, the heterocyclyl-containing moiety is a functional grouping that includes a 1,3,5 triazinane group.

The cycloalkyl-containing moiety will be understood to be a functional grouping that comprises one or more cycloalkyl groups. Suitably, the cycloalkyl is a 6-membered cycloalkyl such as cyclohexane. In certain embodiments, the cycloalkyl-containing moiety is a functional grouping that includes a 1,3,5 cyclohexane.

### Preferred Conjugates

In certain embodiments, the conjugate is a compound of Formula (IIA), shown below:

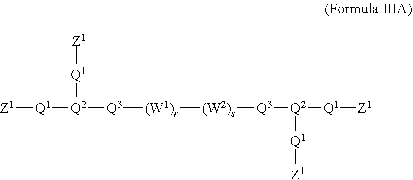

wherein:
   each Z¹ is a re-bridging moiety of Formula (III) as defined hereinabove that is attached to an antibody at the positions shown in Formula (III);
   each Q¹ is independently a bond or a group of Formula IVa:

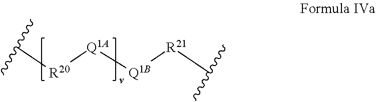

wherein:
   Q^{1A} is absent or selected from a C(═O), OC(═O), NR⁷C(═O),
   C(═O)NR⁷, C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₁₀ alkylene
   and C₁-C₁₀ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently absent or a group of Formula IVb:

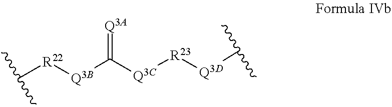

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₁₀alkylene and C₁-C₁₀alkylene containing O in the backbone;
   Q^{3D} is absent or selected from C(═O), OC(═O), NR¹²C(═O) and C(═O)NR¹²;
W¹ is a group of Formula IVc or Formula IVe:

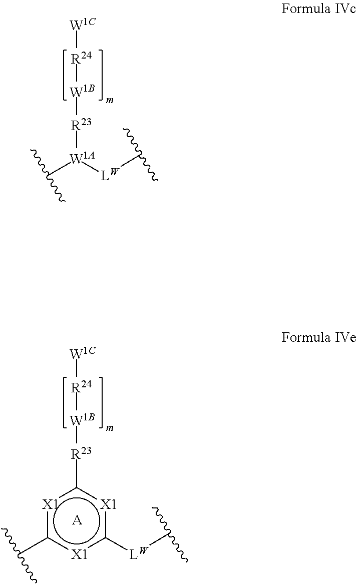

wherein:
   W^{1A} is selected from N and CR¹⁴, wherein R¹⁴ is a selected from hydrogen and C₁-C₄alkyl;
   L^{W} is absent or selected from a C₁-C₆alkylene and a C₁-C₆alkylene containing amido, O or N in the backbone;
   Ring A is a 6-membered aryl, a 6-membered heteroaryl, a 6-membered heterocyclyl or a 6-membered cycloalkyl, wherein each X₁ is independently selected from CH or N;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₁₂alkylene and C₁-C₁₂alkylene containing O in the backbone;
   m is an integer selected from 1 or 2;
   W^{1C} is selected from Formula W^{C1} or Formula W^{C2}:

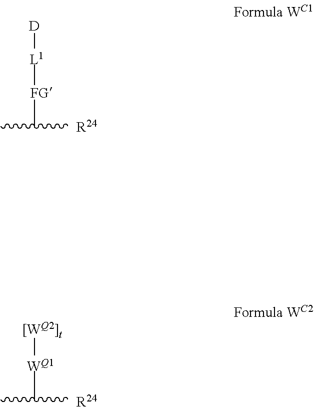

wherein:
   W^{Q1} is a group of Formula W^{C3}:

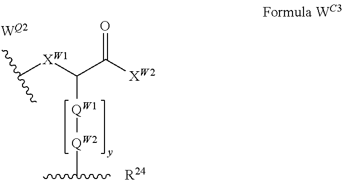

wherein:
Q^{W1} is absent or selected from a C₁-C₁₂alkylene and a
C₁-C₁₂alkylene containing O or N in the backbone; and
Q^{W2} is selected from —C(═O)—, —OC(═O)—, —C(═O)O—, —
NHC(═O)—, and —C(═O)NH—;
y is an integer selected from 0 or 1;
X^{W1} is selected from O or NH;
X^{W2} is selected from hydrogen, C₁-C₄alkyl, OR^{x1} and
NR^{x1}R^{x2}, wherein R^{x1} and R^{x2} are independently selected from hydrogen and C₁-C₄alkyl;

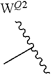

indicates the position where X^{W1} is attached to a group of formula W^{Q2};

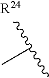

indicates the position where W^{Q1} is attached to a group of formula R²⁴
W^{Q2} is a group of Formula W^{C4}:

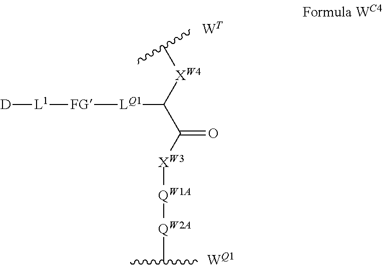

wherein:
Q^{W1A} is absent or selected from a C₁-C₁₂alkylene and a C₁-C₁₂alkylene containing O or N in the backbone; and
Q^{W2A} is selected from —C(═O)—, —OC(═O)—, —C(═O)O—, —NHC(═O)—, and —C(═O)NH—; L^{Q1} is a linker comprising one or more groups selected from a C₁-C₂₀alkylene, an alkylenediamine moiety, a (poly)ethylene glycol moiety, an amino acid residue and combinations thereof;
X^{W3} is selected from O or NH;

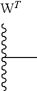

indicates the position where X^{W4} is attached to another group of formula W^{Q2} via the Q^{W2A} substituent group, or
indicates the position where X^{W4} is attached to one of the following groups: a hydrogen or —C(═O)R^{X3}, wherein R^{x3} is selected from hydrogen and C₁-C₄alkyl;

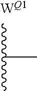

indicates the position where Q^{W2A} is attached to the group of formula W^{Q1} via the X^{W1} group; X^{W4} is selected from O or NH:
L^{Q1} is a linker comprising one or more groups selected from a C₁-C₂₀alkylene, an alkylenediamine moiety, a (poly)ethylene glycol moiety, an amino acid residue and combinations thereof;
t is an integer selected from 1 to 8;
FG′ is a functional linking moiety;
L¹ is a linker; and
D is an active agent;
W² is a group of Formula IVd:

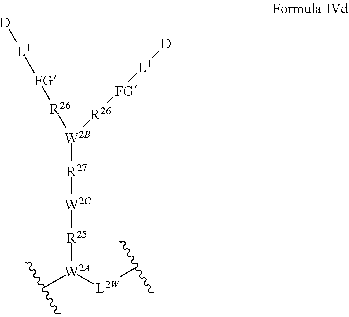

wherein:
   W^{2A} is selected from N and CR¹⁷;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —
   OC(═O)O—, —NR¹⁷C(═O)—, —C(═O)NR¹⁷—, —NR¹⁷C(═O)NR¹⁸—, —C(═NR¹⁷)—and —C(═S)—;
   L^{2W} is absent or selected from a C₁-C₆alkylene and a C₁-C₆alkylene containing amido, O or N in the backbone;
   R¹⁷ and R¹⁸ are independently selected from hydrogen and C₁-C₄alkyl;
   R²⁵, R²⁶ and R²⁷ are independently selected from a bond, C₁-C₁₂alkylene and C₁-C₁₂alkylene containing O in the backbone; and
   FG′, L¹ and D are as defined above;
r is an integer selected from 0 to 12;
s is an integer selected from 0 to 6; and
   wherein r and s are selected such that the total number of active agents per conjugate is between 1 and 12.

It will be understood that when integer v is 2 the two

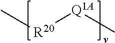

groups may be the same or different.

It will also be understood that when integer m is 2 the two W^{B} and two R²⁴ groups may be the same or different. Suitably, the two W^{B} and two R²⁴ groups are the same.

Suitably, m is 1.

Suitably, Ring A is selected from a phenyl, 1,3,5-triazine or 1,3,5 triazinane.

Suitably, W¹ is a group of Formula IVc.

In certain embodiments, W¹ is a group of Formula IVc and W^{1C} is a group of Formula W^{C1}.

In other embodiments, W¹ is a group of Formula IVc and W^{1C} is a group of Formula W^{C2}.

In certain embodiments, L^{W} is absent or selected from a C₁-C₆alkylene and a C₁-C₆alkylene containing one or more of an O or an N in the backbone.

When W¹ is a group of Formula IVc and W^{1C} is a group of Formula W^{C2}, suitably t is an integer selected from 1 to 6, most suitably t is an integer selected from 1 to 4.

Thus, it will be understood that the linker defined hereinabove in connection with the first aspect of the invention may take the form of Formula L^{X1} or Formula L^{X2} shown below:

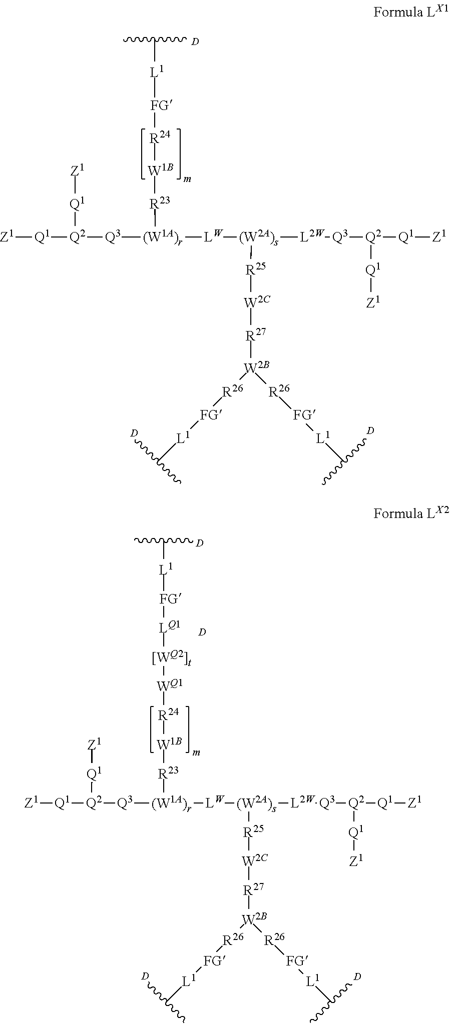

wherein each of Z¹, Q¹, Q², Q³, W^{1A} W^{1B}, W^{1C}, W^{2A}, W^{2B}, W^{2C}, W^{Q1}, W^{Q2}, R²³, R²⁴, R²⁵, R²⁶, R²⁷, FG′, L¹, L^{W}, L^{2W}, L^{Q1}, m, t, r and s are as defined herein; and

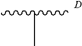

indicates the position where the linker is attached to the active agent(s).

Thus, in certain embodiments, there is provided a conjugate comprising an antibody, a linker and at least one active agent, wherein:
i) the linker connects the at least one active agent(s) to the antibody;
ii) the linker is attached to the antibody through 5 to 8 independent covalent bonds; and
iii) each covalent bond between the linker and the antibody is formed from the reaction between a sulfur atom on the antibody and a functional group on the linker; and
iv) the linker is a linker of Formula L^{X1} or Formula L^{X2}.

Suitably, each Z¹ of Formula L^{X1} and Formula L^{X2} is a re-bridging moiety independently selected from a group consisting of Formula IIIa, Formula IIIb, Formula IIIc, Formula IIId, Formula IIIe, Formula IIIf and Formula IIIg, as defined hereinabove. More suitably, each Z¹ is a re-bridging moiety independently selected from the group consisting of Formula IIIa, Formula IIIb and Formula IIIe, as defined hereinabove. Most suitably, each Z¹ is a re-bridging moiety of Formula IIIa, as defined hereinabove.

It will also be understood that when conjugate is attached to the antibody through 5 to 7 independent covalent bonds, the Z^{A} functional linking group of each re-bridging moiety of Formula III) may be attached to the antibody, either directly or indirectly, via one or both of the

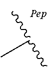

bonds shown hereinabove.

Suitably, r and s are selected such that the total number of active agents per conjugate is between 1 and 10, more suitably between 1 and 8, and most suitably between 1 and 4.

Suitably, r and s are selected such that there are 1, 2, 3, 4, 5, 6, 7 or 8 active agents per conjugate.

In some embodiments Q¹ is a bond.

In other embodiments Q¹ is a group of Formula IVa:

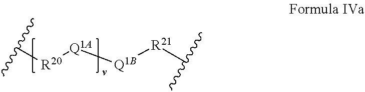

wherein:
   Q^{1A} is absent or selected from C(═O), OC(═O), NR⁷C(═O), C(═O)NR⁷, C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from C₁-C₈ alkylene and C₁-C₈ alkylene containing O in the backbone; and
   v is an integer selected from 0, 1 or 2.

Suitably, Q¹ is a group of Formula IVa wherein:
Q^{1A} is selected from C(═O), OC(═O), NR⁷C(═O), C(═O)NR⁷ and C(═NR⁸);
Q^{1B} is selected from N and CR⁹CR¹⁰;
R⁷, R⁸, R⁹ and R¹⁰ are independently selected from hydrogen and C₁-C₄alkyl;
R²⁰ and R²¹ are independently selected from C₁-C₈ alkylene and C₁-C₈ alkylene containing O in the backbone; and
v is an integer selected from 0, 1 or 2.

More suitably, Q¹ is a group of Formula IVa wherein:
Q^{1A} is selected from C(═O), NR⁷C(═O) and C(═O)NR⁷;
Q^{1B} is selected from N and CR⁹CR¹⁰;
R⁷, R⁹ and R¹⁰ are independently selected from hydrogen and C₁-C₄alkyl;
R²⁰ and R²¹ are independently selected from C₁-C₆ alkylene; and
v is 1 or 2, preferably 1.

In some embodiments, Q² is N. In other embodiments, Q² is CR¹¹, wherein R¹¹ is selected from hydrogen and methyl.

In some embodiment, Q³ is a group of Formula IVb:

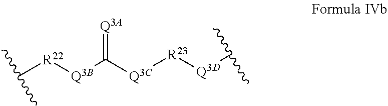

wherein:
   Q^{3A} is selected from NR¹² and O;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond a C₁-C₈ alkylene and a C₁-C₈ alkylene containing O in the backbone;
   Q^{3D} is absent or selected from C(═O), NR¹²C(═O) and C(═O)NR¹²;

In some embodiments, Q³ is a group of Formula IVb¹:

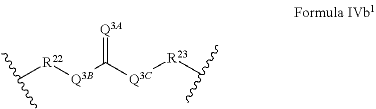

wherein:
   Q^{3A} is selected from NR¹² and O;
   Q^{3B} and Q^{3C} are independently selected from a bond, O and NR¹³;
   R¹² and R¹³ are independently selected from hydrogen and C₁-C₄alkyl; and
   R²² and R²³ are independently a C₁-C₆alkylene and a C₁-C₈ alkylene containing O in the backbone.

In some embodiments, Q³ is a group of Formula IVb¹, wherein:
Q^{3A} is O;
Q^{3B} is a bond;
Q^{3C} is NR¹³;
R¹³ is selected from hydrogen and C₁-C₄alkyl; and
R²² and R²³ are independently a C₁-C₆alkylene.

In some embodiments, W¹ is a group of Formula IVc, wherein:
W^{1A} is selected from N and CR¹⁴, wherein R¹⁴ is a selected from hydrogen and C₁-C₄alkyl;
L^{W} is absent or selected from a C₁-C₆alkylene optionally containing an O in the backbone;
W^{1B} is a group selected from —OC(═O)—, —C(═O)O—, —NR¹⁵C(═O)—, —C(═O)NR¹⁵— and —NR¹⁵C(═O)NR¹⁶—;
R¹⁵ and R¹⁶ are independently selected from hydrogen and C₁-C₄alkyl;
R²³ and R²⁴ are independently selected from a bond and a C₁-C₆alkylene;
m is an integer selected from 1 or 2 (suitably m is 1);
W^{1C} is a group of Formula W^{C}₁;
FG′ is a functional linking moiety;
L¹ is a linker; and
D is an active agent.

In other embodiments, W¹ is a group of Formula IVc, wherein:
W^{1A} is N;
L^{W} is absent or a C₁-C₆alkylene;
W^{1B} is a group selected from —NR¹⁵C(═O)— and —C(═O)NR¹⁵—;
R¹⁵ is selected from hydrogen and C₁-C₄alkyl;
R²³ and R²⁴ are independently selected from a bond and a C₁-C₆alkylene;
m is an integer selected from 1 or 2 (suitably m is 1);
W^{1C} is a group of Formula W^{C}₁;
FG′ is a functional linking moiety;
L¹ is a linker; and
D is an active agent.

In other embodiments, W¹ is a group of Formula IVc, wherein:
W^{1A} is selected from N and CR¹⁴, wherein R¹⁴ is a selected from hydrogen and C₁-C₄alkyl;
L^{W} is absent or selected from a C₁-C₆alkylene optionally containing O in the backbone;
W^{1B} is a group selected from —OC(═O)—, —C(═O)O—, —NR¹⁵C(═O)—, —C(═O)NR¹⁵— and —NR¹⁵C(═O)NR¹⁶—;
R¹⁵ and R¹⁶ are independently selected from hydrogen and C₁-C₄alkyl;
R²³ and R²⁴ are independently selected from a bond and a C₁-C₈alkylene and a C₁-C₈ alkylene containing O in the backbone;
m is an integer selected from 1 or 2 (suitably m is 1);
W^{1C} is a group of Formula W^{C2};
W^{Q1} is a group of Formula W^{C3}:

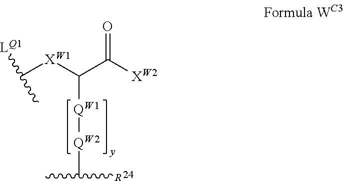

wherein:
   X^{W1} is NH;
   X^{W2} is NR^{x1}R^{x2}, wherein R^{x1} and R^{x2} are independently selected from hydrogen and C₁-C₄alkyl;
   y is an integer selected from 0 or 1;
   Q^{W1} is absent or selected from C₁-C₆alkylene and C₁-C₈alkylene containing O or N in the backbone;
   Q^{W2} is selected from —NHC(═O)— and —C(═O)NH—;
W^{Q2} is a group of Formula W^{C4}:

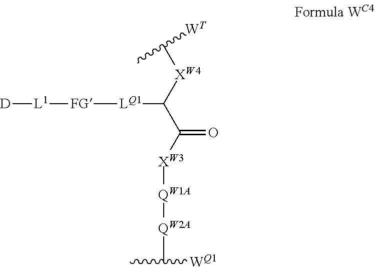

wherein:
   X^{W4} is NH;
   X^{W3} is NH;

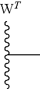

indicates the position where X^{W4} is attached to another group of formula W^{Q2} via the Q^{W2A} substituent group, or

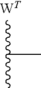

indicates the position where X^{W4} is attached to a —C(═O)R^{X3} group, wherein R^{x3} is selected from hydrogen and C₁-C₄alkyl;
Q^{W1A} is absent or selected from a C₁-C₈alkylene and a
C₁-C₆alkylene containing O or N in the backbone;
Q^{W2A} is —C(═O)—; and
y is an integer selected from 0 or 1;
L^{Q1} is a linker comprising one or more groups selected from an alkylenediamine moiety, a (poly)ethylene glycol moiety, an amino acid residue and combinations thereof;
t is an integer selected from 1 to 4;
FG′ is a functional linking moiety;
L¹ is a linker; and
D is an active agent.

In further embodiments, W¹ is a group of Formula IVc, wherein:
W^{1A} is selected from N and CR¹⁴, wherein R¹⁴ is a selected from hydrogen and C₁-C₄alkyl;
L^{W} is absent or selected from a C₁-C₆alkylene optionally containing O in the backbone;
W^{1B} is a group selected from —OC(═O)—, —C(═O)O—, —NR¹⁵C(═O)—, —C(═O)NR¹⁵— and —NR¹⁵C(═O)NR¹⁶—;
R¹⁵ and R¹⁶ are independently selected from hydrogen and C₁-C₄alkyl;
R²³ and R²⁴ are independently selected from a bond, a C₁-C₈alkylene and a C₁-C₈ alkylene containing O in the backbone;
m is an integer selected from 1 or 2 (suitably m is 1);
W^{1C} is a group of Formula W^{C2};
W^{Q1} is a group of Formula W^{C3A}:

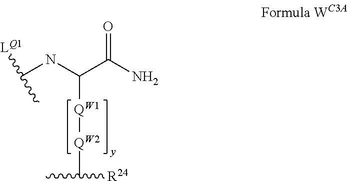

wherein:
   Q^{W1} is absent or a C₁-C₆alkylene;
   Q^{W2} is selected from —NHC(═O)— and —C(═O)NH—; and
   y is an integer selected from 0 or 1;
W^{Q2} is a group of Formula W^{C4A}:

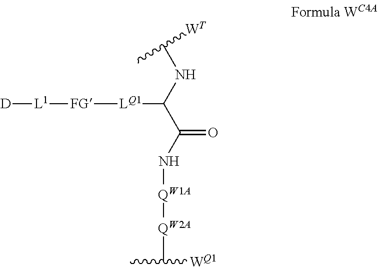

wherein:
   Q^{W1A} is a C₁-C₆alkylene optionally containing an O in the backbone;
   Q^{W2A} is —C(═O)—;

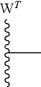

indicates the position where the NH is attached to another group of formula W^{Q2} via the Q^{W2} substituent group, or

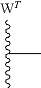

indicates the position where the NH is attached to a —C(═O)CH₃;
L^{Q1} is a group of Formula LQ₁;

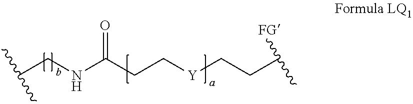

wherein:
   Y is O or NH;
   a is an integer selected from 1 to 6 (suitably a is an integer selected from 1 to 4); and
   b is an integer selected from 1 to 6 (suitably, b is an integer selected from 1 to 4, most suitably b is 4);
t is an integer selected from 1 to 4;
FG′ is a functional linking moiety;
L¹ is a linker; and
D is an active agent.

When W^{1C} is a group of Formula W^{C2}, suitably L^{Q1} is a group of Formula LQ₂;

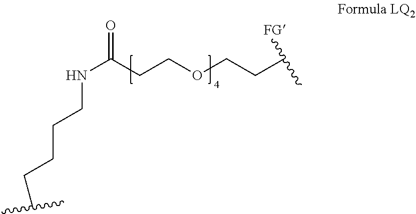

In some embodiments, W² is a group of Formula IVd, wherein:
W^{2A} is selected from N;
L^{2W} is absent or selected from a C₁-C₆alkylene optionally containing O or N in the backbone;
W^{2B} is a group selected from N and CR¹⁸;
W^{2C} is a group selected from —NR¹⁷C(═O)— or —C(═O)NR¹⁷—;
R¹⁷ is selected from hydrogen and C₁-C₄alkyl;
R¹⁸ is selected from hydrogen and C₁-C₄alkyl;
R²⁵, R²⁶ and R²⁷ are independently selected from a bond and a C₁-C₆alkylene; and
FG′, L¹ and D are as defined herein.

In other embodiments, W² is a group of Formula IVd, wherein:
W^{2A} is selected from N;
L^{2W} is absent or a C₁-C₆alkylene;
W^{2B} is N;
W^{2C} is —C(═O)NH—;
R²⁵, R²⁶ and R²⁷ are independently selected from a bond and a C₁-C₆alkylene; and
FG′, L¹ and D are as defined herein.

In certain embodiments, FG′ is a bond or a functional group formed from the reaction between: i) a ketone or aldehyde and an alkoxyamine (e.g. hydroxylamine) or hydrazine; ii) an azide and an alkyne; iii) a amine and an acyl halide or carboxylic acid; iv) electron-rich dienophile (e.g. a 1,3-nitrone alkene) and an electron-poor diene (e.g. tetrazine); and iii) a strained alkene or alkyne (e.g. norbornene or cyclooctyne) and a tetrazine. Suitably, FG′ is a bond or a functional group formed from the reaction between an azide and an alkyne or an amine and an acyl halide or carboxylic acid. Most suitably, FG′ is a functional group formed from the reaction between an azide and an alkyne.

Thus, in some embodiments FG′ is a functional group selected from a bond,

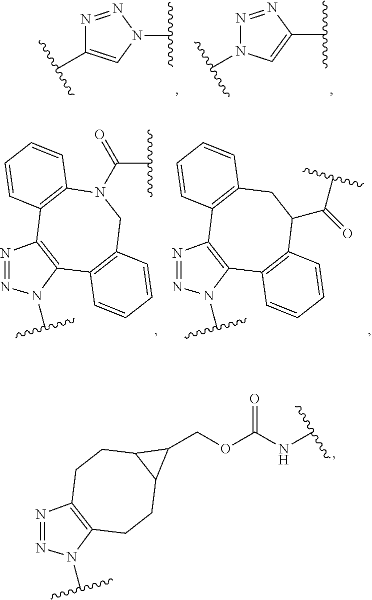

### —NH(═O)C— and —C(═O)NH—.

In other embodiments FG′ is a functional group selected from a bond,

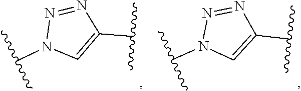

### —NH(═O)C— and —C(═O)NH—.

In some embodiments, FG′ is or

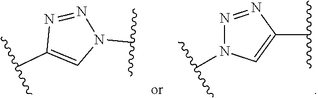 [SMILES: *c1cn(*)nn1 ; *c1cn(*)nn1]

Suitably, FG′ is

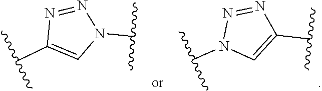 [SMILES: *c1cn(*)nn1 ; *c1cn(*)nn1]

Most suitably, FG′ is

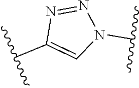

wherein N is bound to L¹.

### Linker L¹

Linker L¹ is a linker that attaches the functional linking moiety, FG′, to the active agent(s), D.

It will be appreciated that any suitable linker may be used connect the functional linking moiety, FG′, to the active agent, D.

In certain embodiments, the linker L¹ comprises one or more groups that are susceptible to enzymatic cleavage (e.g. proteolytic or peptidase cleavage, sulfatase cleavage or galactosidase cleavage). Thus, in some embodiments, the linker L¹ comprises one or more amino-acid residues, dipeptide residues or tripeptide residues, one or more aryl sulfates, one or more aryl galactosides or combinations thereof. Suitably, the linker L¹ comprises one or more amino-acid residues, dipeptide residues or tripeptide residues, wherein the amino-acid residues, dipeptide residues or tripeptide residues are as defined hereinbelow.

In certain embodiments, the linker L¹ comprises one or more groups that are susceptible to chemical cleavage (e.g. a disulfide or a hydrazone).

In some embodiments, the linker L¹ comprises one or more groups selected from a C₁-C₂₀alkylene, an alkylenediamine moiety, a (poly)ethylene glycol moiety, an amino acid residue and combinations thereof.

In certain embodiments, L¹ is selected from a bond, a C₁-C₁₀alkylene, a C₁-C₁₀alkylene containing O in the backbone and a group of formula Va:

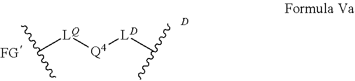

wherein:
   L^{Q} is a selected from a bond, a C₁-C₁₀alkylene and a C₁-C₁₀alkylene containing O or NH in the backbone;
   Q⁴ is a single bond, or

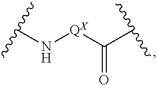

where Q^{X} is such that Q⁴ is an amino-acid residue, a dipeptide residue or a tripeptide residue; and
L^{D} is a group for attachment to the active agent.

Suitably, L^{Q} is a selected from a bond, a C₁-C₁₀alkylene and a C₁-C₁₀alkylene containing O in the backbone.

In some embodiments, L^{Q} is a selected from a bond. In other embodiments, L^{Q} is a C₁-C₁₀alkylene or a C₁-C₁₀alkylene containing O in the backbone. Suitably, L^{Q} is a C₁-C₁₀alkylene containing O in the backbone.

In certain embodiments, L¹ is selected from a bond, a C₁-C₁₀alkylene, a C₁-C₁₀alkylene containing O in the backbone and a group of formula Va¹:

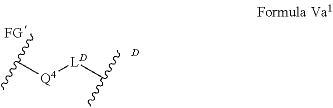

wherein:
   Q⁴ is a single bond, or

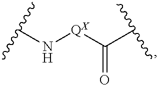

where Q^{X} is such that Q⁴ is an amino-acid residue, a dipeptide residue or a tripeptide residue; and
L^{D} is a group for attachment to the active agent.

In certain embodiments, L¹ is selected from a C₁-C₁₀alkylene and a C₁-C₁₀alkylene containing O in the backbone.

In other embodiments, L¹ is a group of Formula Va or Formula Va¹.

In some embodiments, Q⁴ is a single bond.

In other embodiments, Q⁴ is

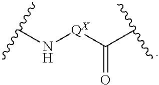

### Q^{X}

In one embodiment, Q^{x} is an amino acid residue. The amino acid may be a natural amino acid or a non-natural amino acid.

In one embodiment, Q^{X} is selected from: Phe, Lys, Val, Ala, Cit, Leu, IIe, Arg, and Trp, where Cit is citrulline.

In one embodiment, Q^{x} comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q^{x} is selected from:
^{NH}-Phe-Lys-^{C═O},
^{NH} Val-Ala-^{C═O},
^{NH} Val-Lys-^{C═O},
^{NH} Ala-Lys-^{C═O},
^{NH}-Val-Cit-^{C═O},
^{NH}-Phe Cit-^{C═O},
^{NH}-Leu-Cit-^{C═O},
^{NH}-IIe-Cit-^{C═O},
^{NH}-Phe-Arg-^{C═O}, and
^{NH}-Trp-Cit-^{C═O};
   where Cit is citrulline.

Preferably, Q^{x} is selected from:
^{NH}-Phe-Lys-^{C═O},
^{NH}-Val-Ala-^{C═O},
^{NH}-Val-Lys-^{C═O},
^{NH}-Ala-Lys-^{C═O}, and
^{NH}-Val-Cit-^{C═O}.

Most preferably, Q^{x} is selected from ^{NH}-Phe-Lys-^{C═O}, ^{NH}-Val-Cit-^{C═O} or ^{N}H-Val-Ala-^{C═O}.

Other dipeptide combinations of interest include:
^{NH}-Gly-Gly-^{C═O},
^{NH}-Pro-Pro-^{C═O}, and
^{NH}-Val-Glu-^{C═O}.

Other dipeptide combinations may be used, including those described by Dubowchik et al., Bioconjugate Chemistry, 2002, 13,855-869, which is incorporated herein by reference.

In some embodiments, Q^{x} is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin.

In one embodiment, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

In some embodiments, L^{D} is selected from:
(a) a single bond;
(b) —C(═O)—;
(c) —NH—; and

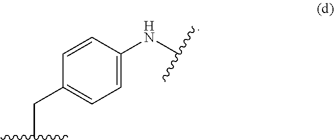 [SMILES: *Cc1ccc(N*)cc1]

In some of these embodiments, L^{D} is a single bond. If Q⁴ is also a single bond, then FG′ is attached directly to the active agent.

In other of these embodiments, L^{D} is —C(═O)— or —NH—.

In other of these embodiments, L^{D} is

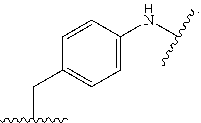 [SMILES: *Cc1ccc(N*)cc1]

This group can act as a self-immolative group in conjunction with a cleavable linking group.

### Certain Further Embodiments

In some embodiments, the conjugate is a compound of Formula (IIIA), shown below:

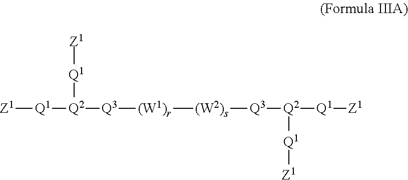

wherein:
   each Z¹ is a re-bridging moiety of Formula (IIIa) as defined hereinabove that is attached to an antibody at the positions shown in Formula (IIIa);
   each Q¹ is a group of Formula IVa:

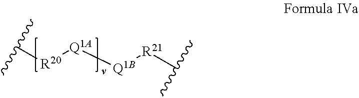

wherein:
   Q^{1A} is selected from C(═O), NR⁷C(═O) and C(═O)NR⁷;
   Q^{1B} is selected from N and CR⁹CR¹⁰;
   R⁷, R⁹ and R¹⁰ are independently selected from hydrogen and C₁-C₄alkyl;
   R²⁰ and R²¹ are independently selected from C₁-C₈ alkylene and a C₁-C₈ alkylene containing O in the backbone; and
   v is 1 or 2, preferably 1;
each Q² is N;
each Q³ is a group of Formula IVb:

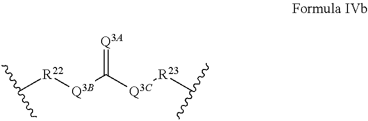

wherein:
   Q^{3A} is O;
   Q^{3B} is a bond
   Q^{3C} is NR¹³;
   R¹³ is selected from hydrogen and C₁-C₄alkyl; and
   R²² and R²³ are independently a C₁-C₆alkylene;
W¹ is a group of Formula IVc₁:

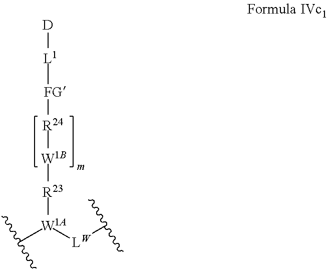

wherein:
   W^{1A} is N;
   L^{W} is absent or a C₁-C₄alkylene;
   W^{1B} is a group selected from —NR¹⁵C(═O)— and —C(═O)NR¹⁵—;
   R¹⁵ and R¹⁶ are independently selected from hydrogen and C₁-C₄alkyl;
   R²³ and R²⁴ are independently selected from a bond, a C₁-C₈alkylene and a C₁-C₈ alkylene containing O in the backbone; and
   m is an integer selected from 1 or 2 (suitably m is 1);
W² is a group of Formula IVd:

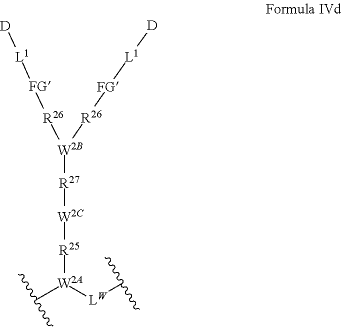

wherein:
   W^{2A} is selected from N;
   L^{2W} is absent or a C₁-C₄alkylene
   W^{2B} is N;
   W^{2c} is C(═O)NH;
   R²⁵, R²⁶ and R²⁷ are independently selected from a bond and a C₁-C₈alkylene and a C₁-C₈alkylene containing O in the backbone;
FG′ is

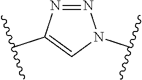

wherein N is bound to L¹;
L¹ is selected from a bond, C₁-C₁₀alkylene, a C₁-C₁₀alkylene containing O in the backbone and a group of Formula Va¹:

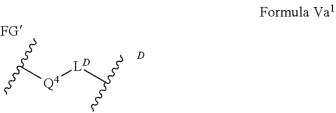

wherein:
   Q⁴ is a single bond, or

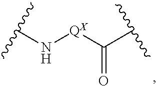

where Q^{x} is such that Q⁴ is an amino-acid residue, a dipeptide residue or a tripeptide residue; and
L^{D} is a group for attachment to the active agent;
D is an active agent, as defined herein; and
wherein r and s are selected such that the total number of active agents per conjugate is between 1 and 8.

In other embodiments, the conjugate is a compound of Formula (IIIA), shown below:

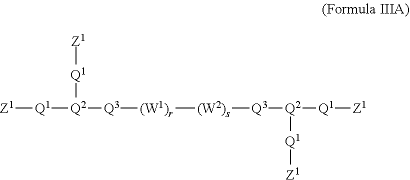

wherein:
   each Z¹ is a re-bridging moiety of Formula (IIIa) as defined hereinabove that is attached to an antibody at the positions shown in Formula (IIIa);
   each Q¹ is a group of Formula IVa:

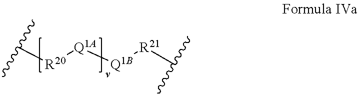

wherein:
   Q^{1A} is selected from C(═O), NR⁷C(═O) and C(═O)NR⁷;
   Q^{1B} is selected from N and CR⁹CR¹⁰;
   R⁷, R⁹ and R¹⁰ are independently selected from hydrogen and C₁-C₄alkyl;
   R²⁰ and R²¹ are independently selected from C₁-C₈ alkylene and a C₁-C₈ alkylene containing O in the backbone; and
   v is 1 or 2, preferably 1;
each Q² is N:
each Q³ is a group of Formula IVb:

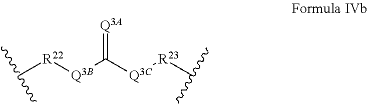

wherein:
   Q^{3A} is
   Q^{3B} is a bond
   Q^{3C} is NR¹³;
   R¹³ is selected from hydrogen and C₁-C₄alkyl; and
   R²² and R²³ are independently a C₁-C₆alkylene and a C₁-C₈ alkylene containing O in the backbone;
W¹ is a group of Formula IVc:

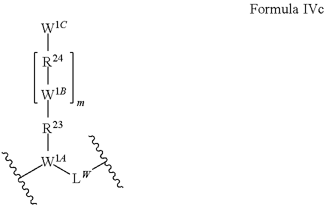

wherein:
   W^{1A} is N;
   L^{W} is absent or a C₁-C₄alkylene;
   W^{1B} is a group selected from —NR¹⁵C(═O)— and —C(═O)NR¹⁵—;
   R¹⁵ and R¹⁶ are independently selected from hydrogen and C₁-C₄alkyl;
   R²³ and R²⁴ are independently selected from a bond, a C₁-C₈alkylene and a C₁-C₈ alkylene containing O in the backbone; and
   m is an integer selected from 1 or 2 (suitably m is 1);
   W^{1C} is a group of Formula W^{2C};
   W^{Q1} is a group of Formula W^{C3A}:

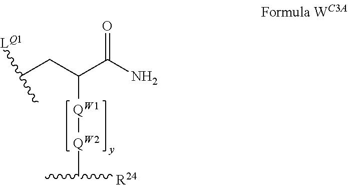

wherein:
Q^{W1} is absent or a C₁-C₆alkylene;
Q^{W2} is selected from —NHC(═O)— and —C(═O)NH—;
y is an integer selected from 0 or 1;
W^{Q2} is a group of Formula W^{C4A}:

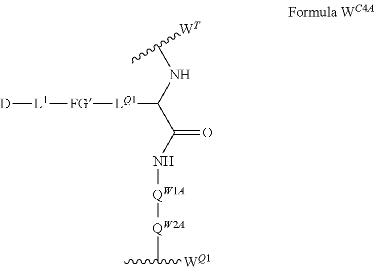

wherein:
   Q^{W1A} is a C₁-C₆alkylene optionally containing an O in the backbone;
   Q^{W2A} is —C(═O)—;

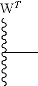

indicates the position where X^{W1} is attached to another W^{Q2} group via the Q^{W2A} substituent group, or

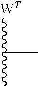

indicates the position where X^{W4} is attached to a —C(═O)CH₃;
L^{Q1} is a group of Formula LQ₁;

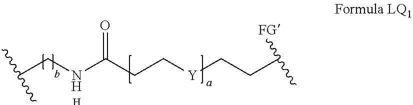

wherein:
Y is O or NH;
a is an integer selected from 1 to 6;
b is an integer selected from 1 to 6;
t is an integer selected from 1 to 4;
FG′ is

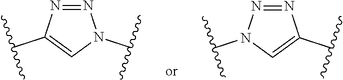

wherein N is bound to L¹
L¹ is selected from a bond, C₁-C₁₀alkylene, a C₁-C₁₀alkylene containing O in the backbone and a group of Formula Va¹:

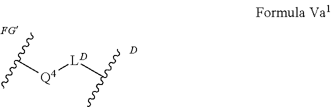

wherein:
   Q⁴ is a single bond, or

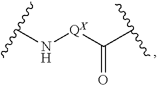

where Q^{X} is such that Q⁴ is an amino-acid residue, a dipeptide residue or a tripeptide residue; and
L^{D} is a group for attachment to the active agent;
D is an active agent, as defined herein; and
wherein r and s are selected such that the total number of active agents per conjugate is between 1 and 8.

In certain embodiments, the conjugate is selected from one of the following:

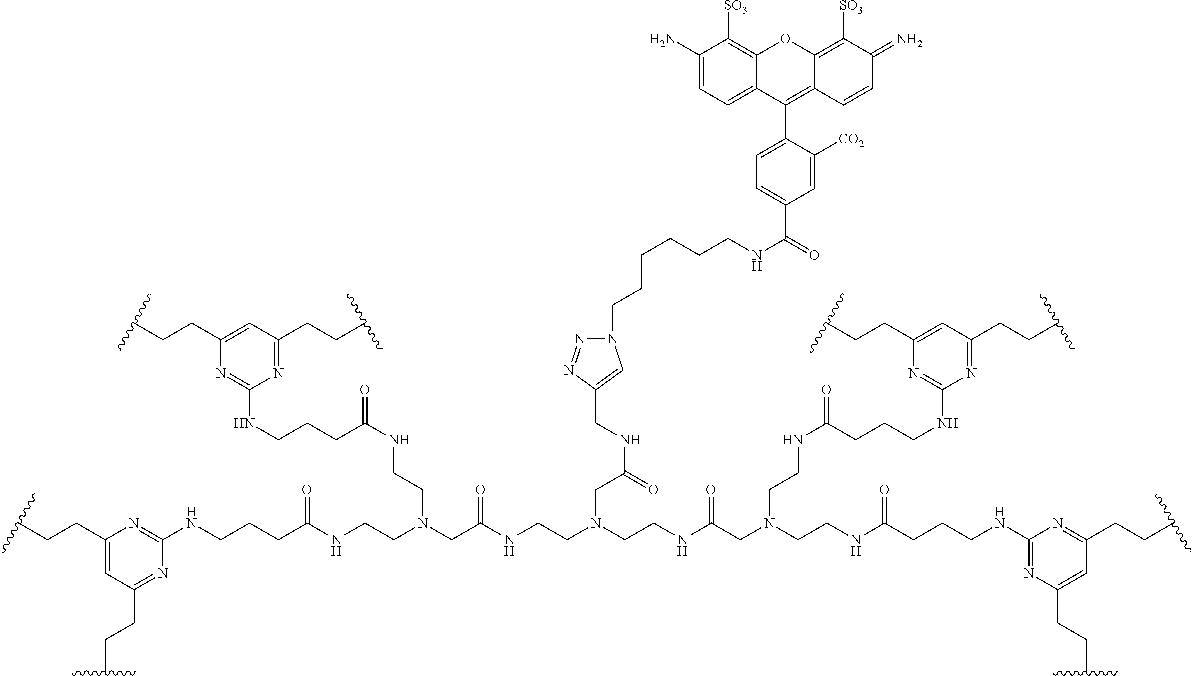

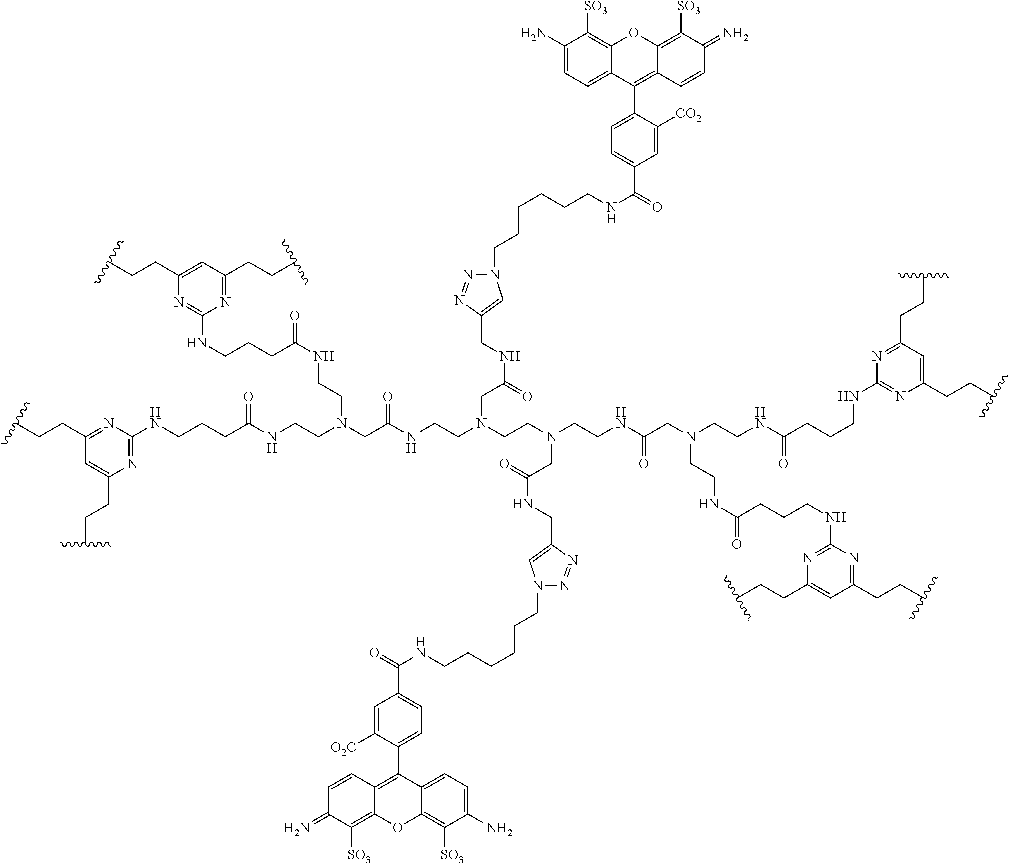

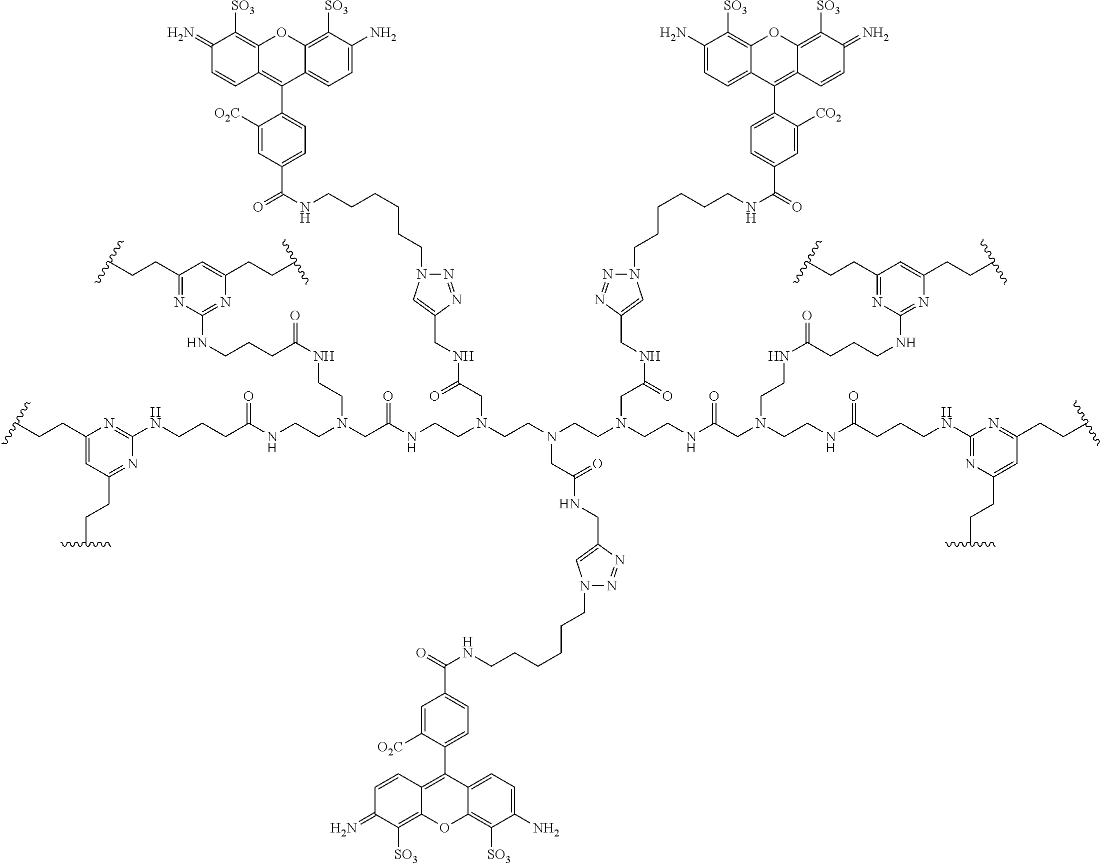

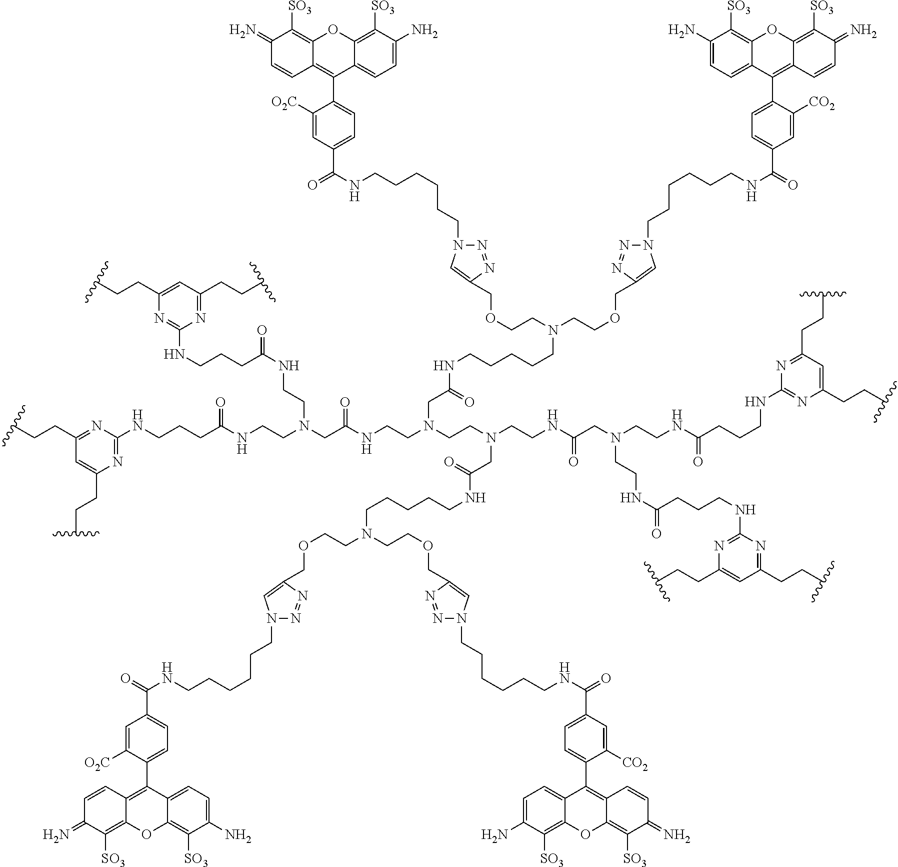

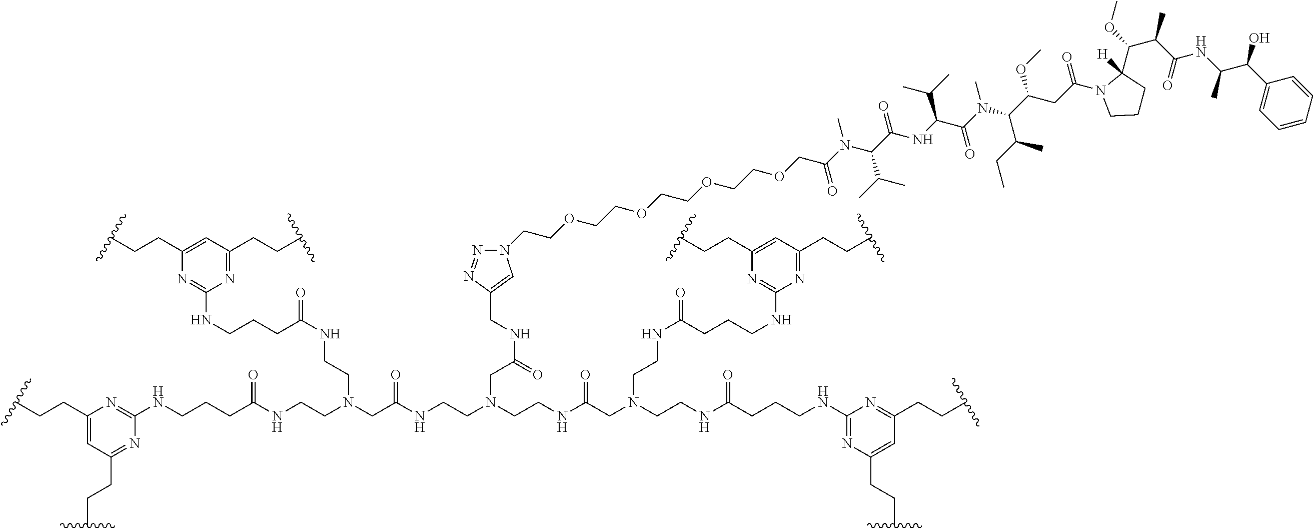 [SMILES: *CCc1cc(CC*)nc(NCCCC(=O)NCCN(CCNC(=O)CCCNc2nc(CC*)cc(CC*)n2)CC(=O)NCCN(CCNC(=O)CN(CCNC(=O)CCCNc2nc(CC*)cc(CC*)n2)CCNC(=O)CCCNc2nc(CC*)cc(CC*)n2)CC(=O)NCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)n1]

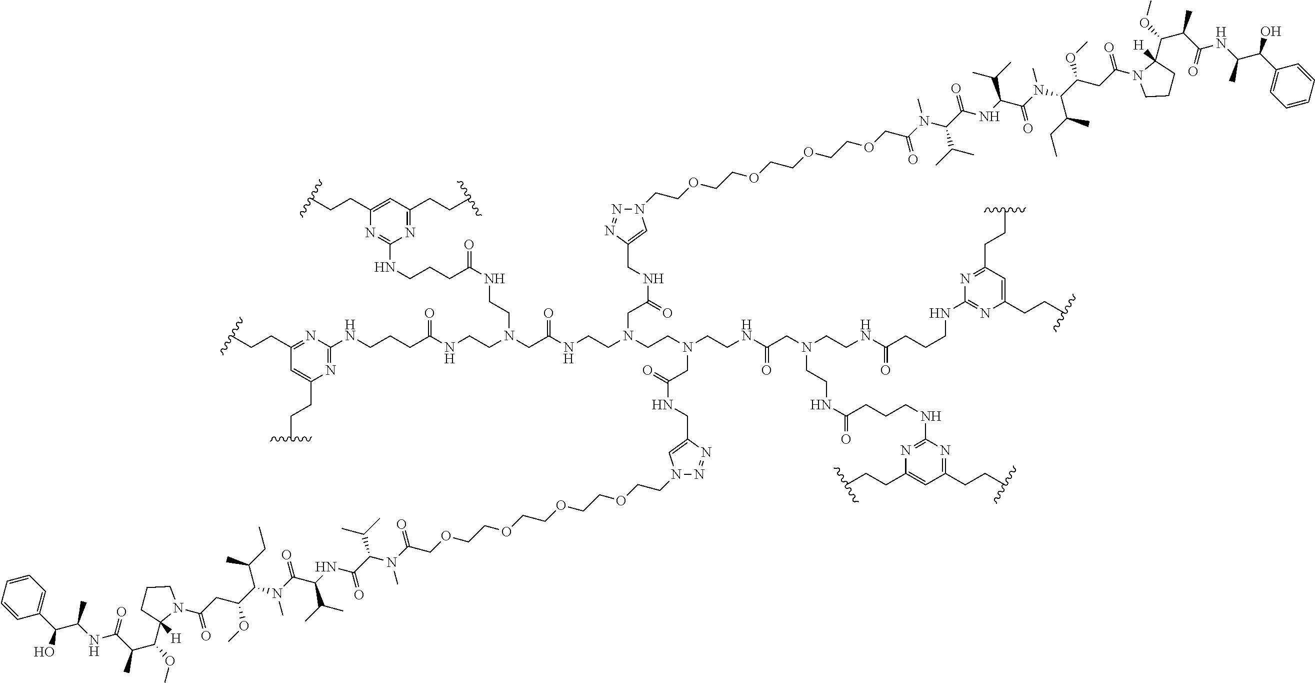 [SMILES: *CCc1cc(CC*)nc(NCCCC(=O)NCCN(CCNC(=O)CCCNc2nc(CC*)cc(CC*)n2)CC(=O)NCCN(CCN(CCNC(=O)CN(CCNC(=O)CCCNc2nc(CC*)cc(CC*)n2)CCNC(=O)CCCNc2nc(CC*)cc(CC*)n2)CC(=O)NCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)CC(=O)NCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)n1]

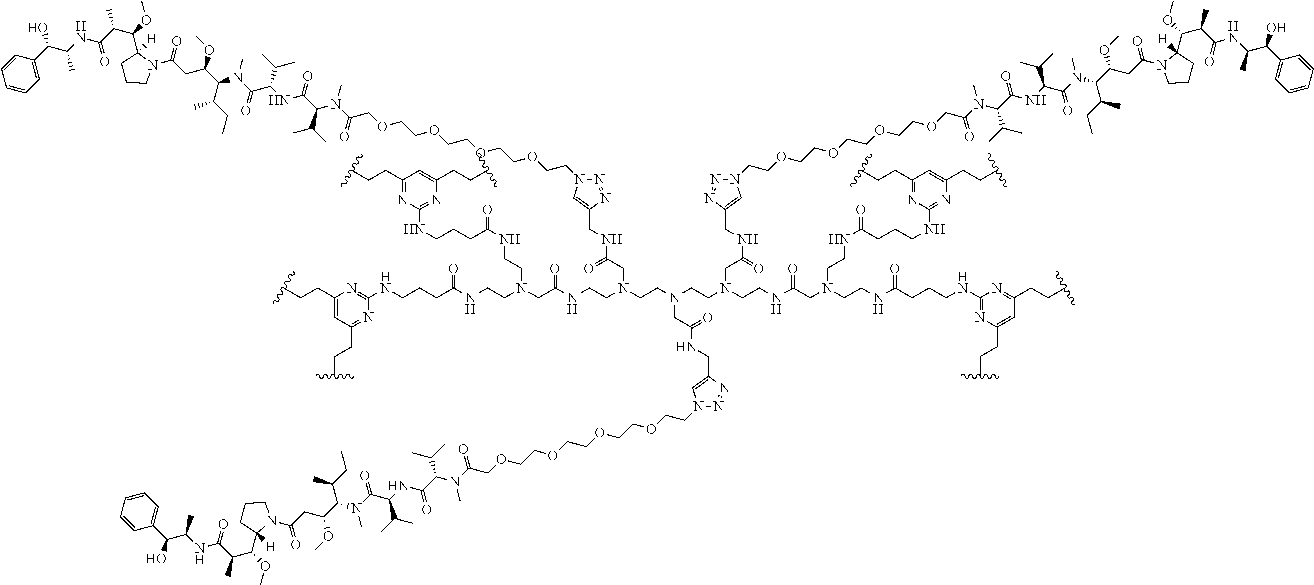

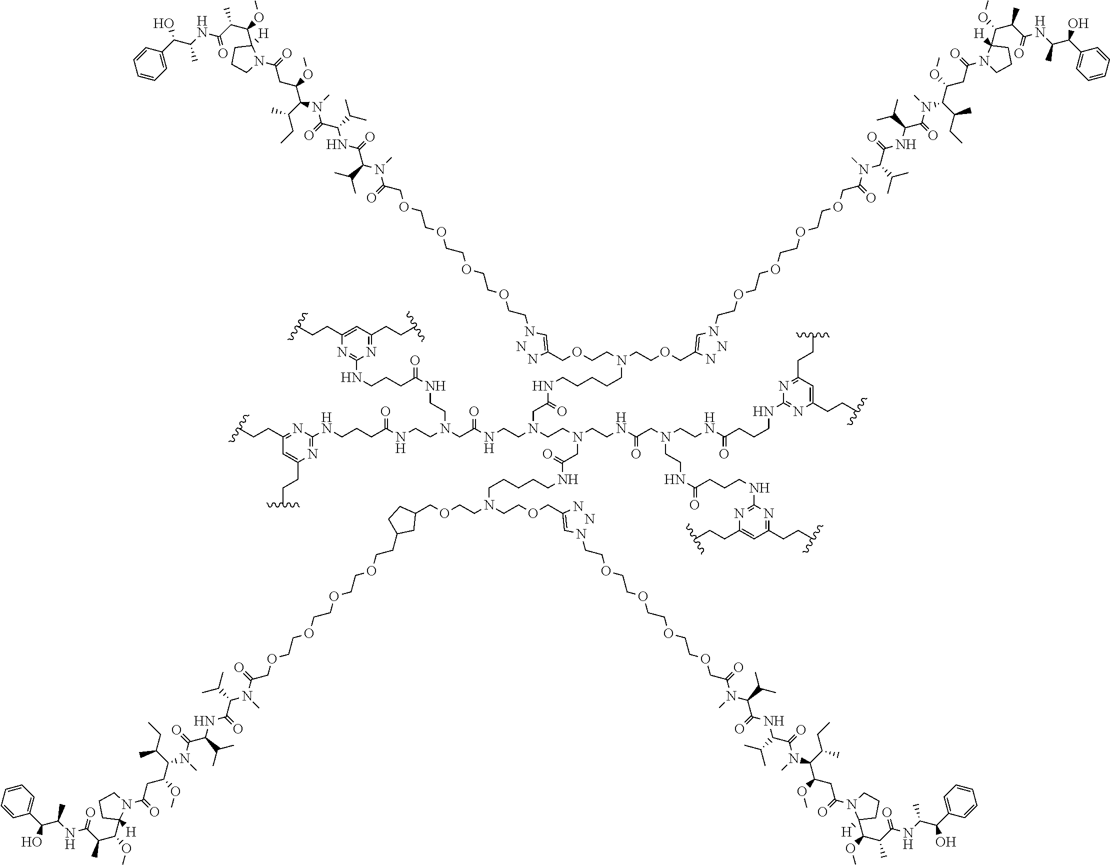

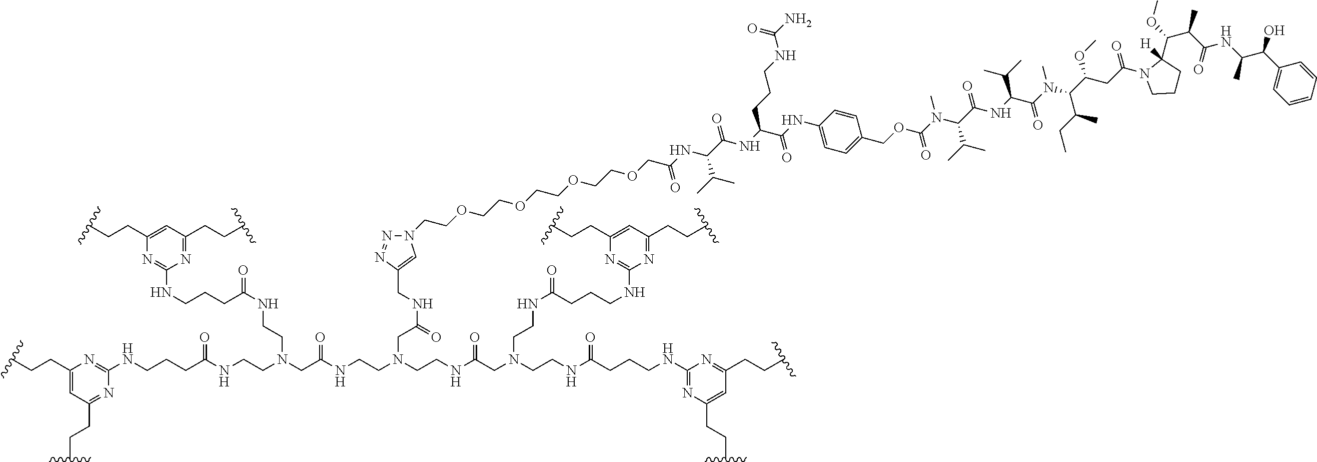 [SMILES: *CCc1cc(CC*)nc(NCCCC(=O)NCCN(CCNC(=O)CCCNc2nc(CC*)cc(CC*)n2)CC(=O)NCCN(CCNC(=O)CN(CCNC(=O)CCCNc2nc(CC*)cc(CC*)n2)CCNC(=O)CCCNc2nc(CC*)cc(CC*)n2)CC(=O)NCc2cn(CCOCCOCCOCCOCC(=O)N[C@H](C(=O)N[C@@H](CCCNC(N)=O)C(=O)Nc3ccc(COC(=O)N(C)[C@H](C(=O)N[C@H](C(=O)N(C)[C@@H]([C@@H](C)CC)[C@@H](CC(=O)N4CCC[C@H]4[C@H](OC)[C@@H](C)C(=O)N[C@H](C)[C@@H](O)c4ccccc4)OC)C(C)C)C(C)C)cc3)C(C)C)nn2)n1]

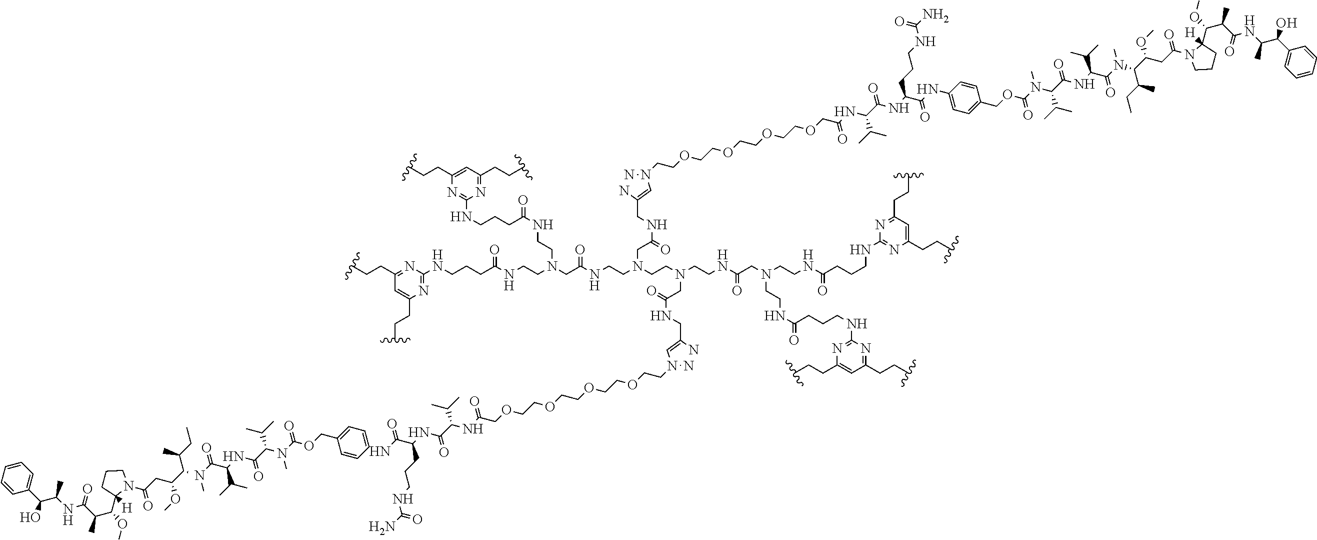

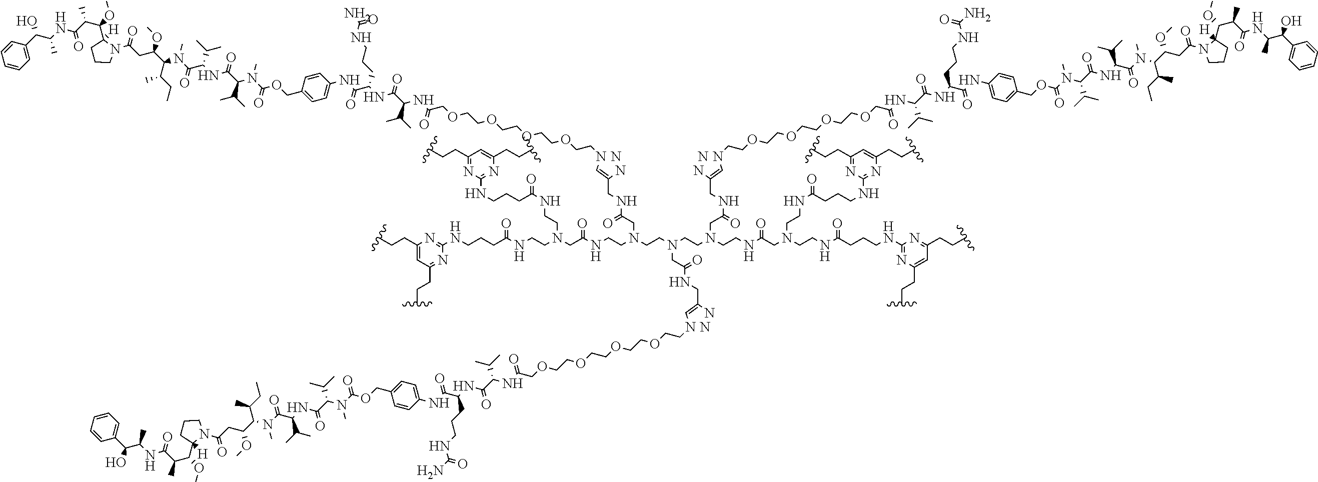

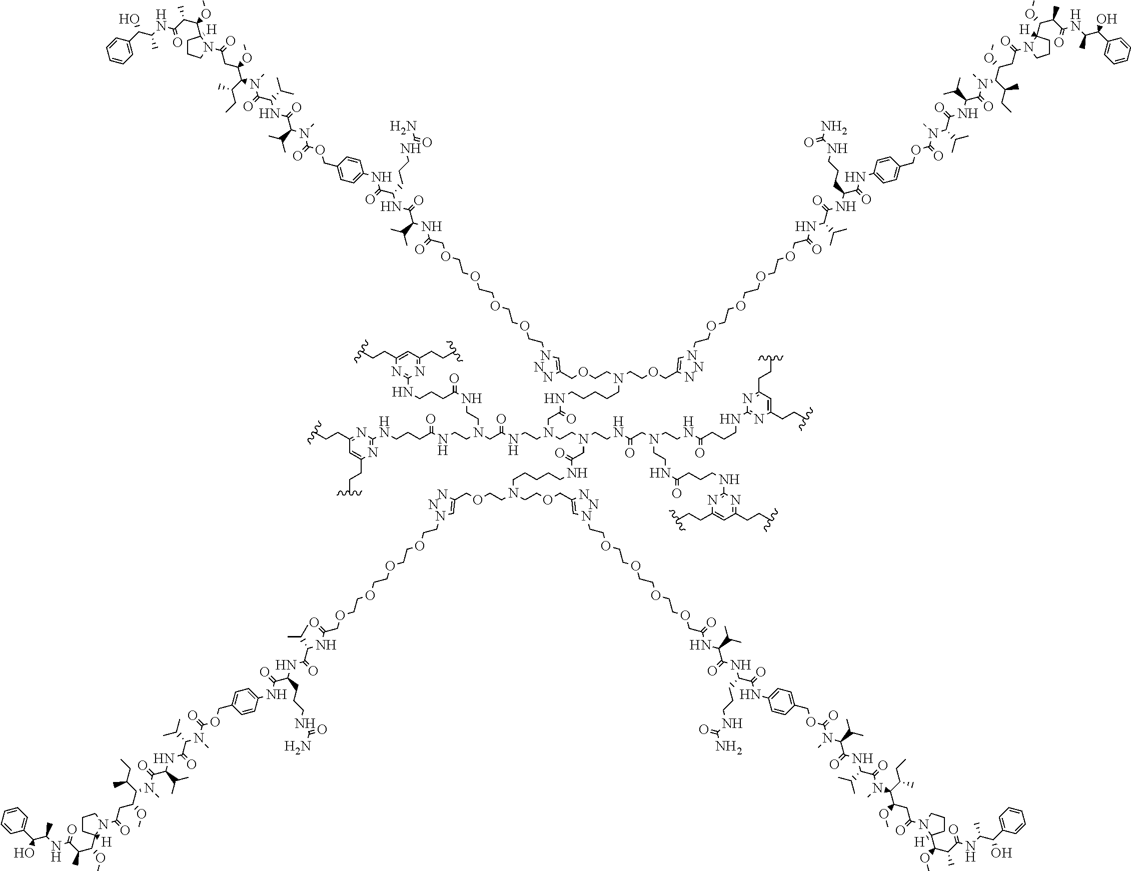

wherein:

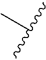

indicates the position where the antibody is attached; and
the antibody is preferably trastuzumab or brentuximab, most preferably trastuzumab.

In some embodiments, the conjugate is selected from one of the following:

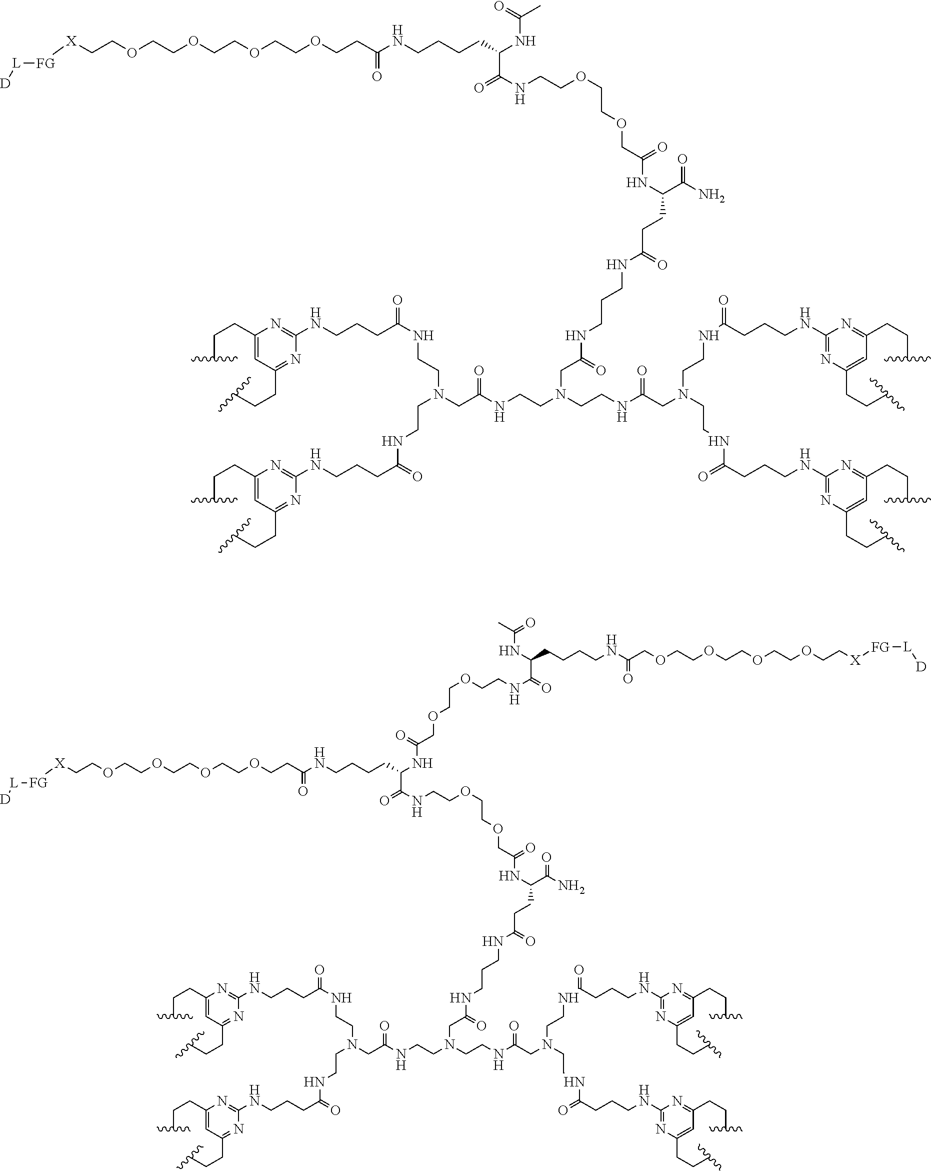

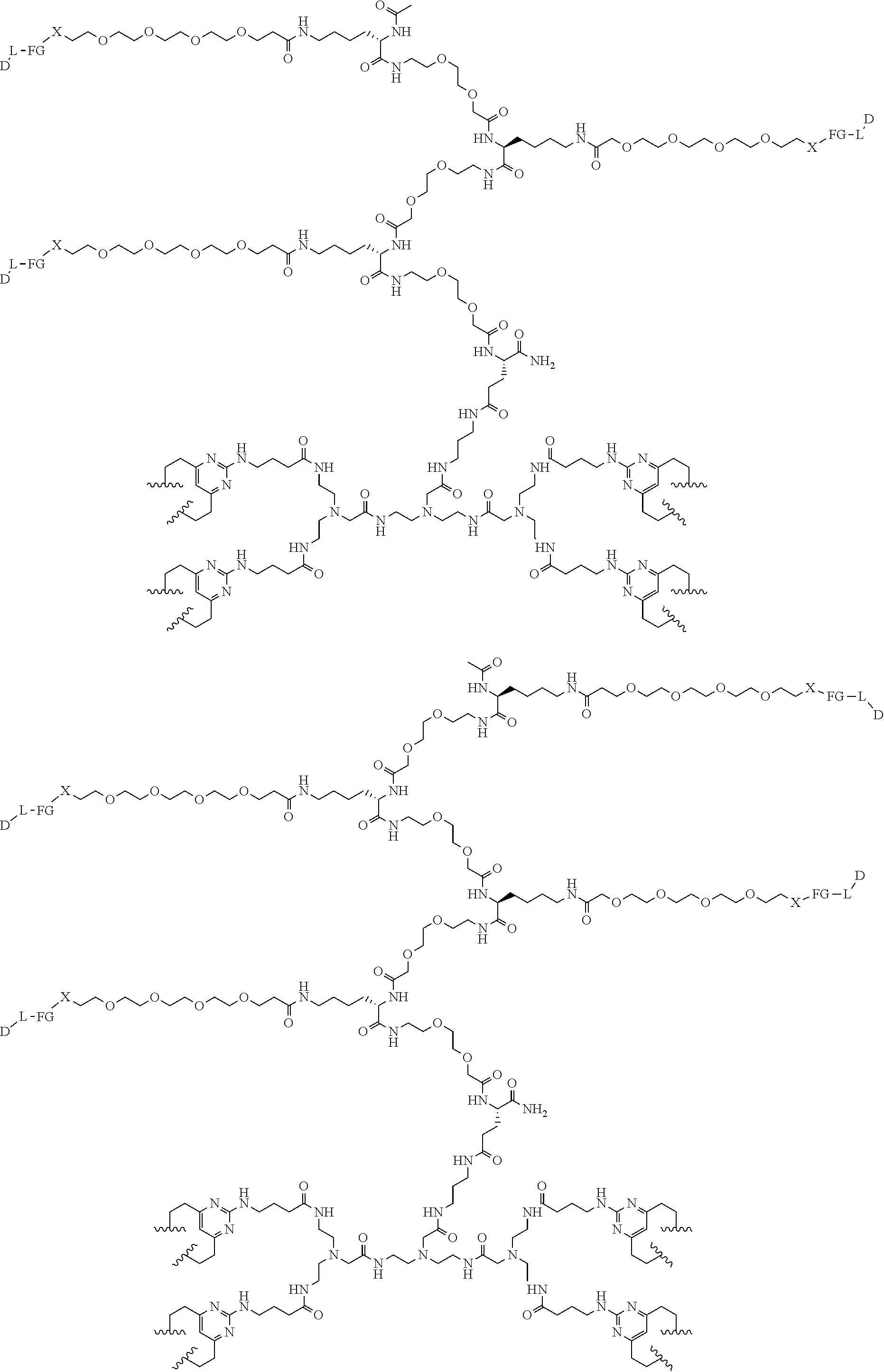

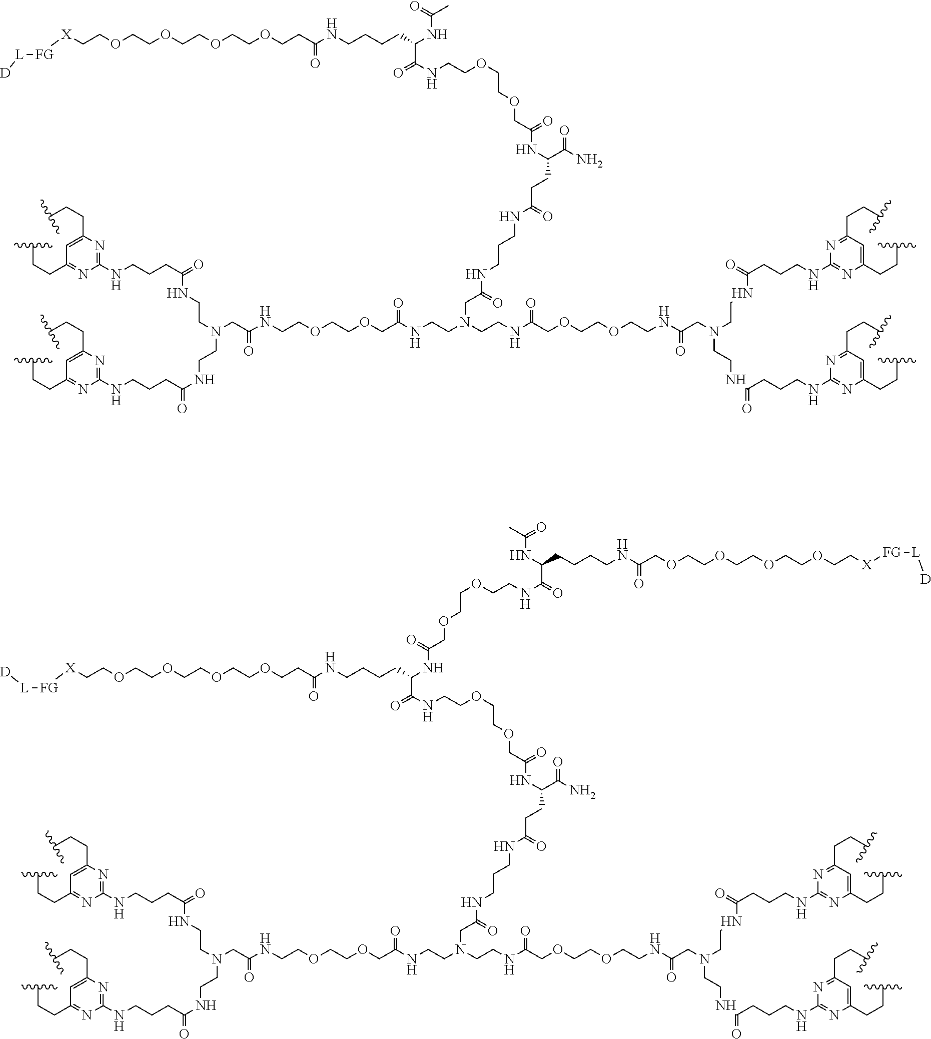

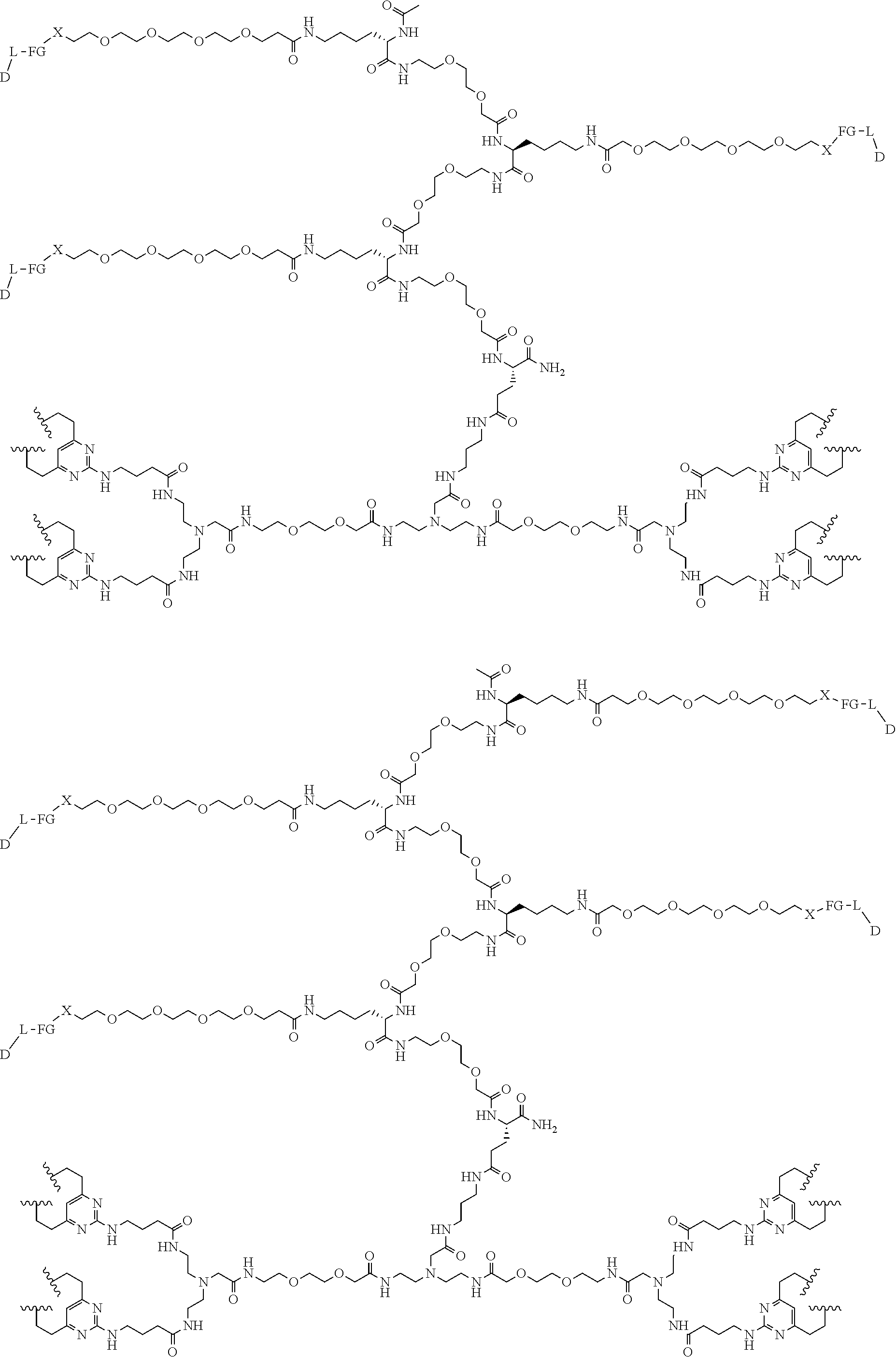

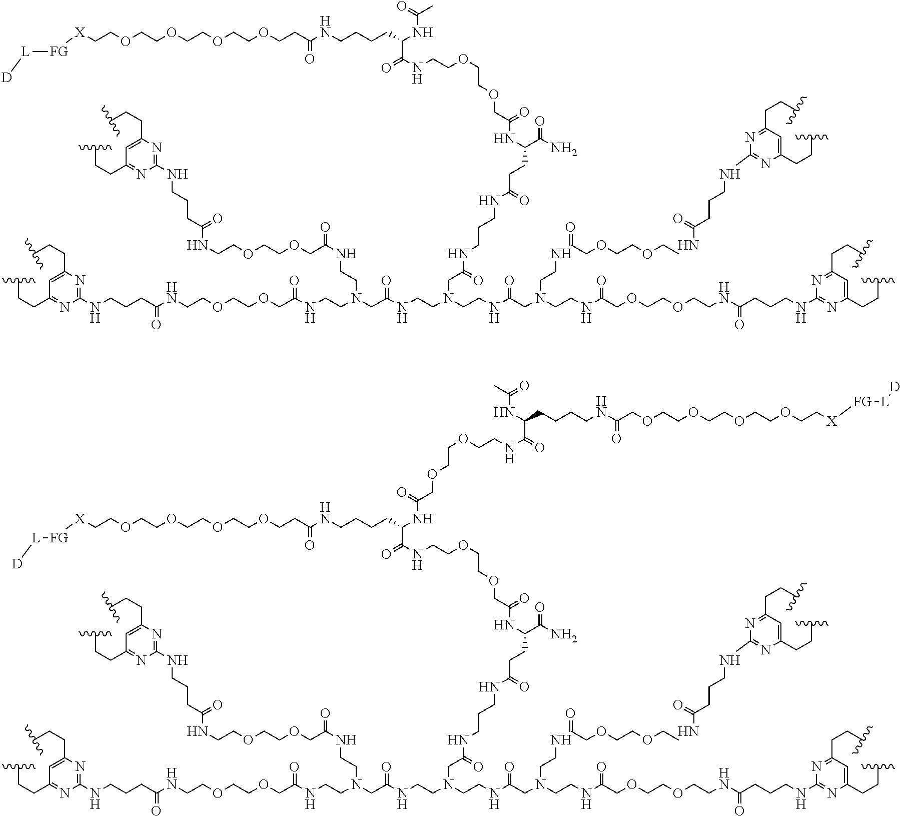

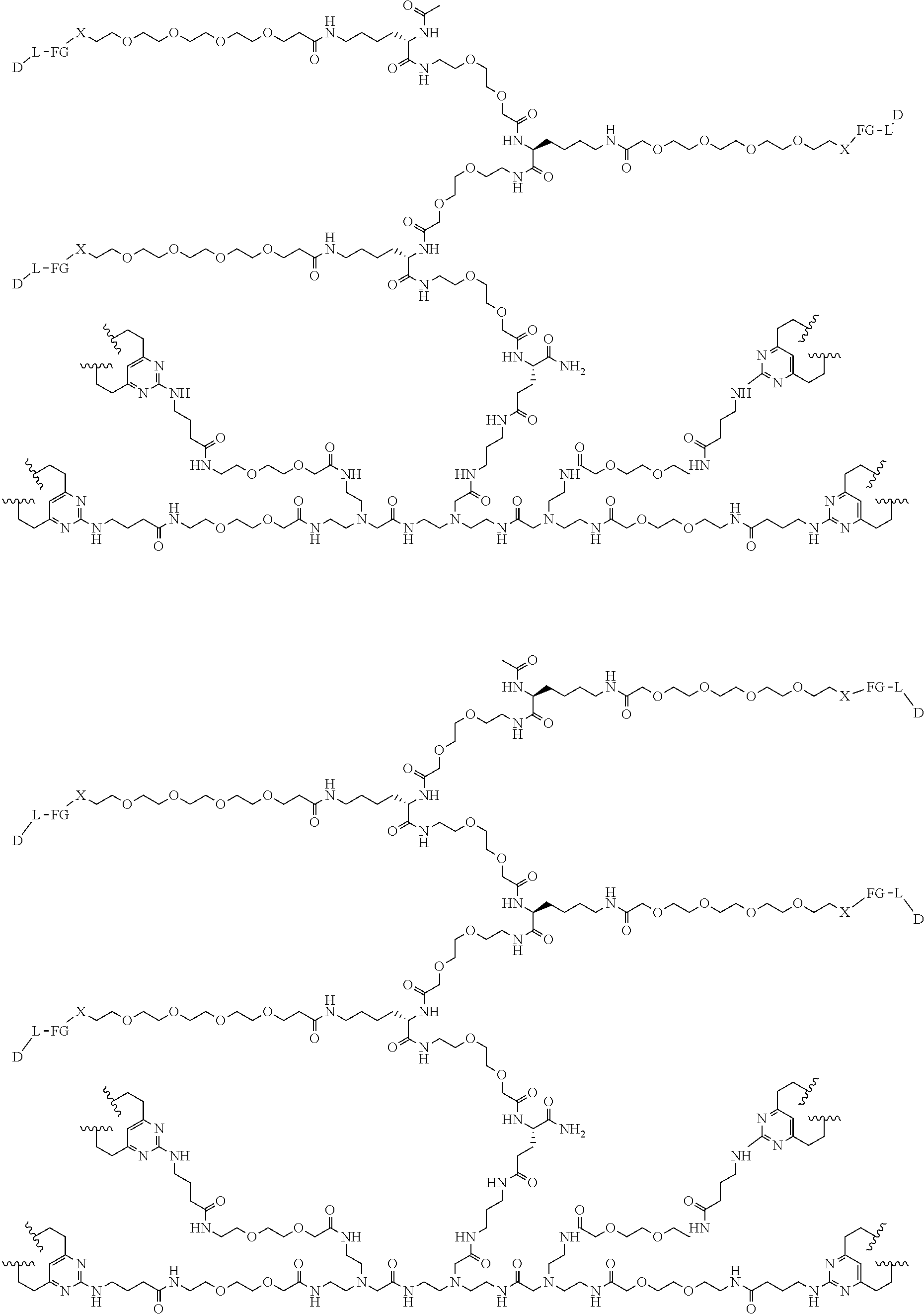

wherein:

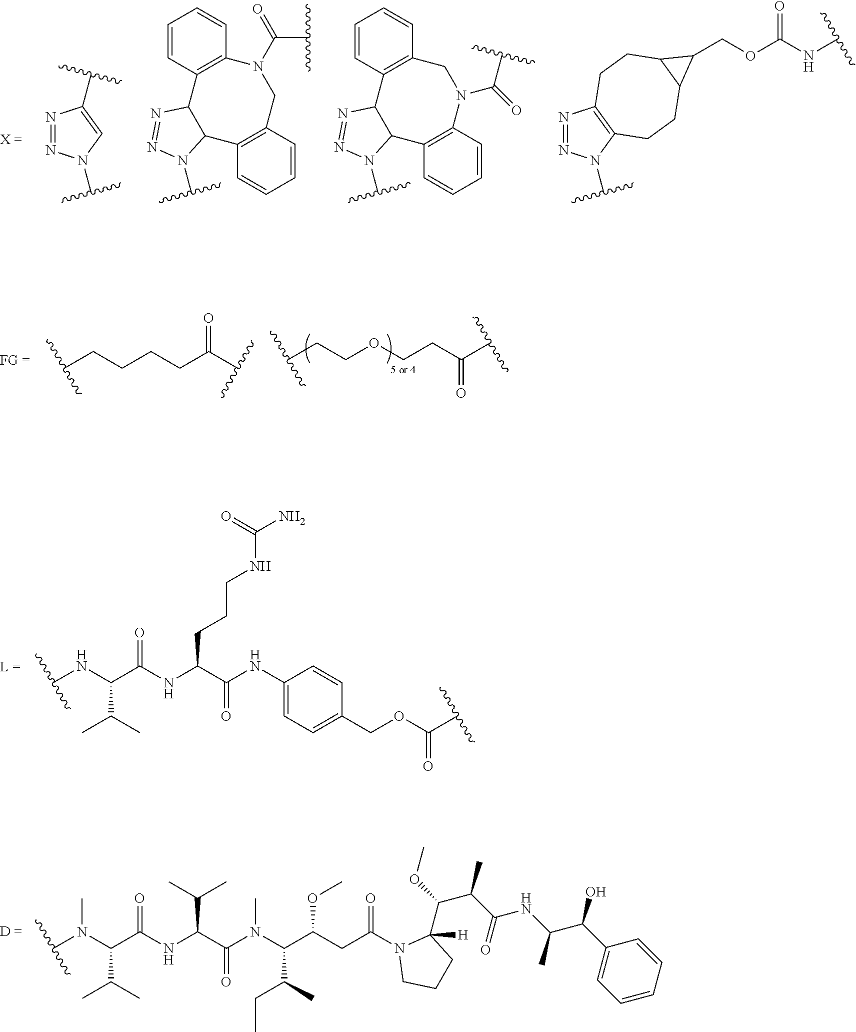

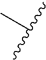

indicates the position where the antibody is attached; and
the antibody is preferably trastuzumab or brentuximab, most preferably trastuzumab.

In some embodiments, the conjugate is selected from one of the following:

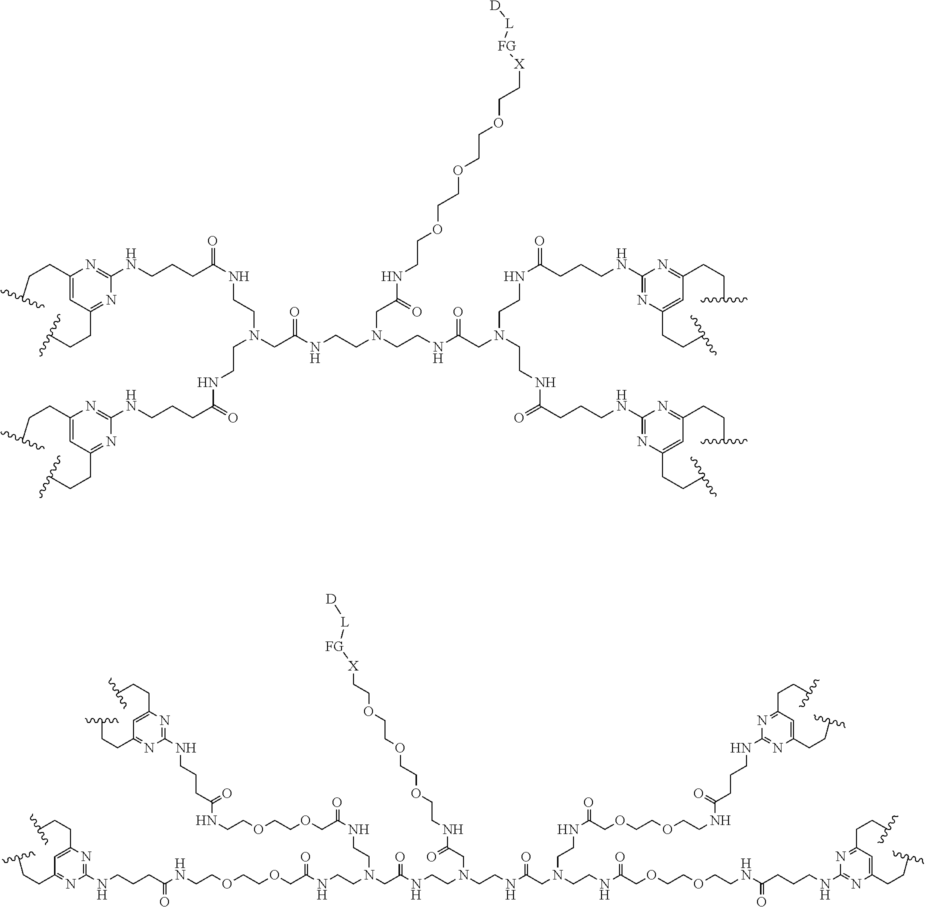

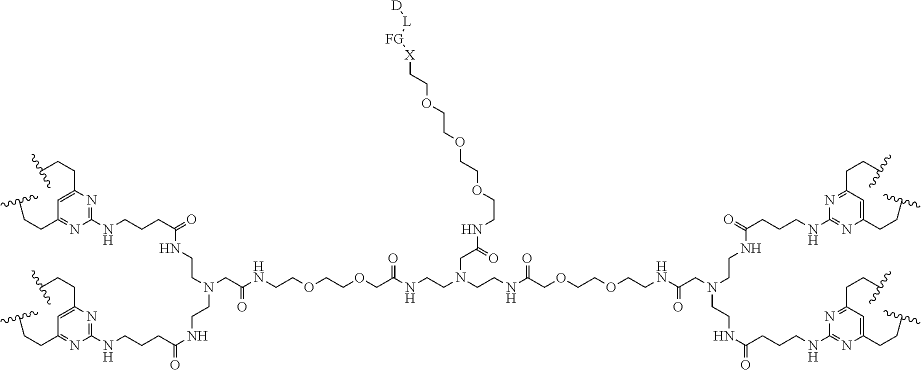

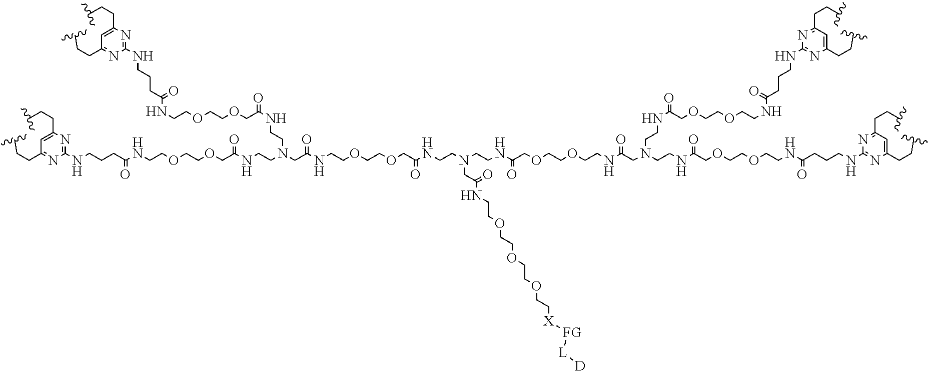

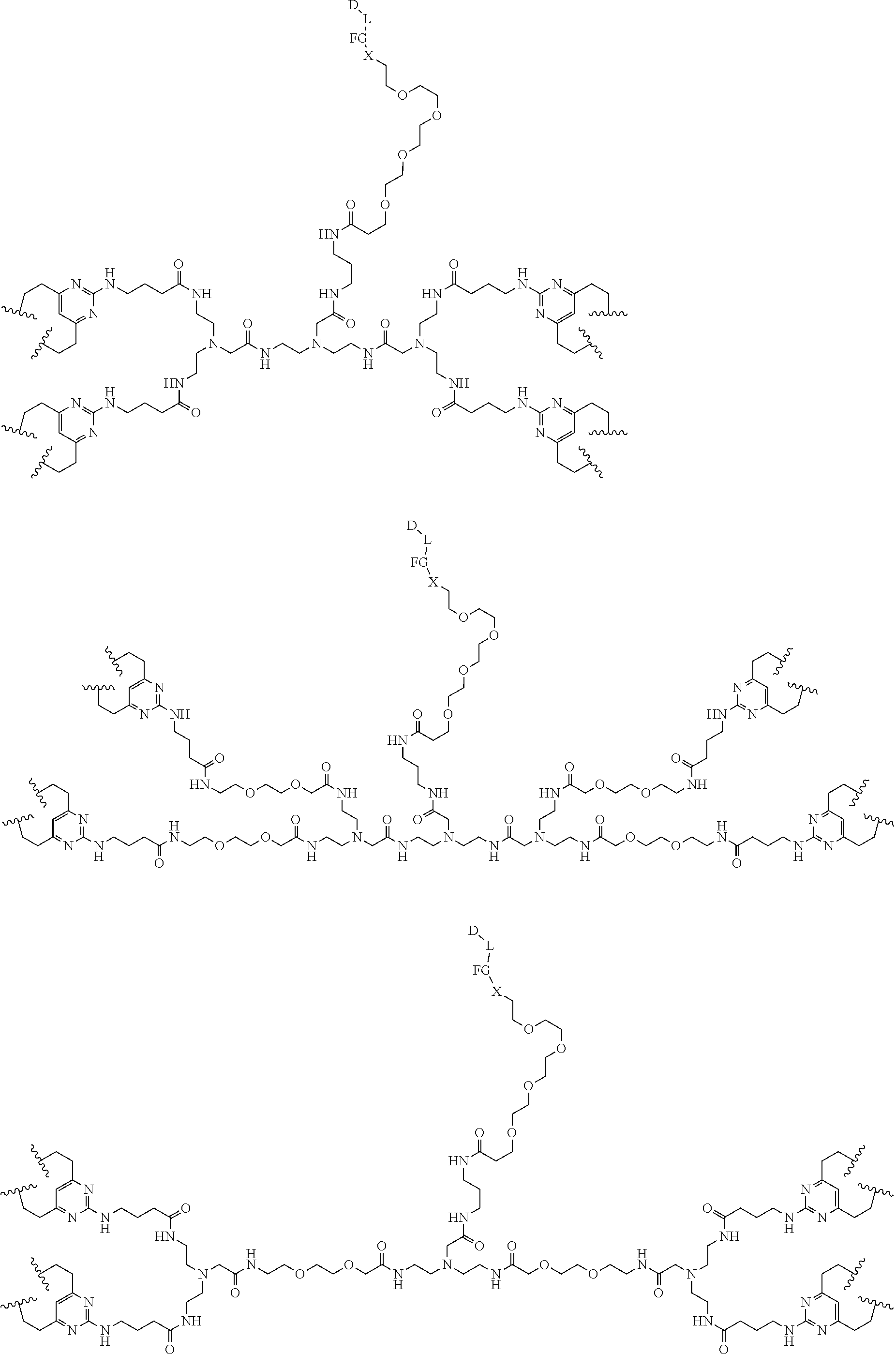

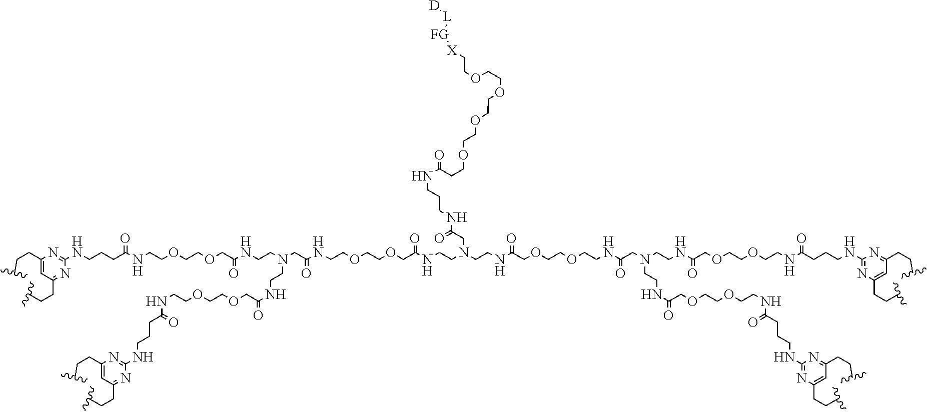

wherein:

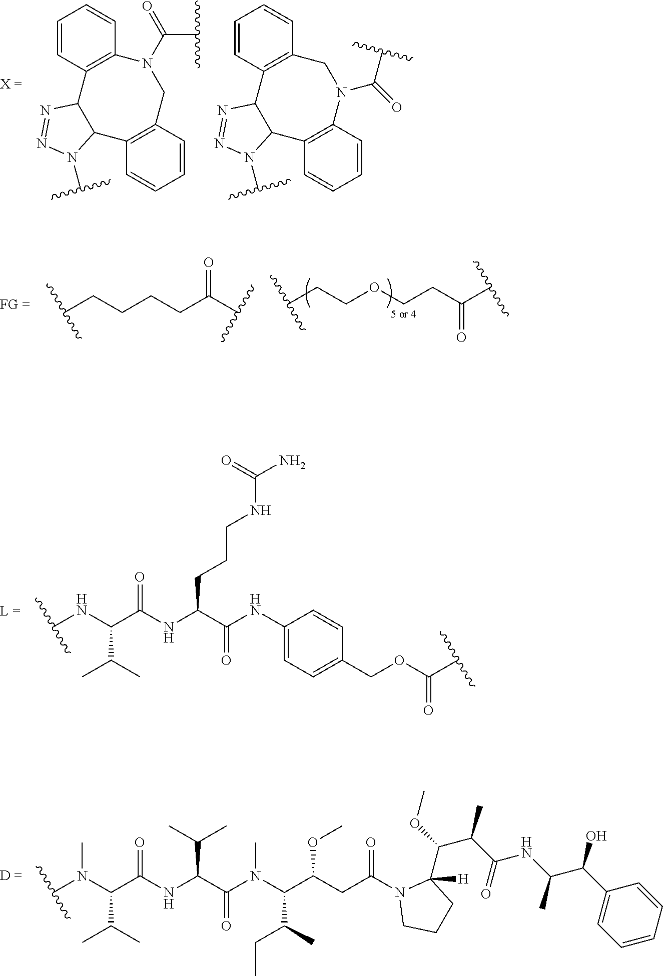

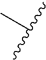

indicates the position where the antibody is attached; and
the antibody is preferably trastuzumab or brentuximab, most preferably trastuzumab.

In some embodiments, the conjugate is selected from ADC 1 to ADC 20 described below.

### Conjugating Reagents of the Invention

According to another aspect, the present invention provides a conjugating reagent of Formula (II), shown below:

(D-L¹-FG′)ₙ-L-(Z)₃ (Formula II)
wherein:
   FG′ is a functional linking moiety;
   n is an integer selected from 1 to 20;
   L and L¹ are independently linkers;
   Z is a functional group capable of reaction with a sulfur atom of an antibody; and D is an active agent;
      or a pharmaceutically acceptable salt, solvate or hydrate thereof.

Suitably, Z is an electrophilic functional group capable of reaction with a sulfur atom of an antibody.

Suitably, n is an integer selected from 1 to 12, more suitably from 1 to 10, even more suitably, from 1 to 8, and most suitably from 1 to 4.

In certain embodiments, Z is a Michael Acceptor (e.g. alkene or alkyne typically with an electron withdrawing group) that is capable of reaction with a sulfur atom of an antibody via a conjugate addition (Michael reaction). Conjugate addition reactions and the Michael reaction are well known in the field of chemistry. Thus, a skilled person will be able to select suitable Michael Acceptors for use in the conjugating reagents of the invention.

In certain embodiments, Z is a functional group that reacts with the sulfur atoms on the antibody selected from an alkene, an alkyne, a vinylarene (e.g. divinylpyrimidine), a maleimide, a halo-maleimide, a sulfone, an arylene-propiolonitrile, a pyridazinedione, a halo-(hetro)arene, a benzyl halide, a group comprising a β-unsaturated ketone, a group capable of an SNAr reaction or a group capable of alkylating sulfur.

In certain embodiments, Z is a functional group that reacts with the sulfur atoms on the antibody selected from an alkene, an alkyne, a vinylarene (e.g. divinylpyrimidine), a maleimide, a halo-maleimide, a sulfone, an arylene-propiolonitrile, a pyridazinedione or a group comprising a β-unsaturated ketone.

In certain embodiments, the conjugating reagent is of Formula (IIa), shown below:

(D-L¹-FG′)ₙ-L-(Z²)₄ (Formula IIa)
wherein:
   each Z² is a re-bridging linking group comprising two functional groups (Z) capable of reaction with a sulfur atom on an antibody; and
   D, L, L¹, FG′ and n are each as defined herein.

Preferred and suitable substituent groups for each of D, L, L¹, FG′ and n will be understood to be analogous to the preferred and suitable substituent groups for each of D, L, L¹, FG′ and n for the conjugates of the invention described hereinabove.

In certain embodiments, each Z² is a re-bridging linking group independently selected from one of the following groups:

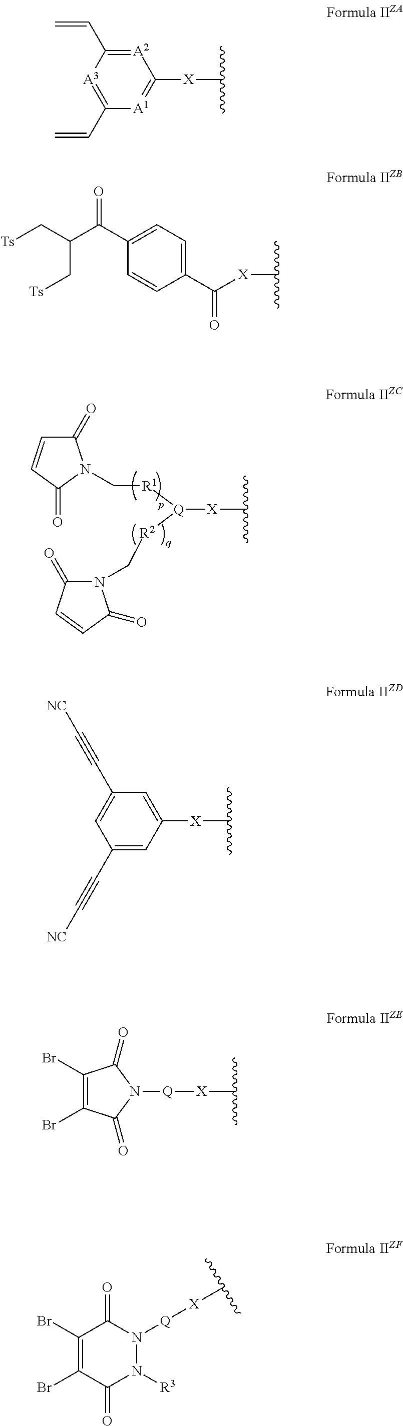

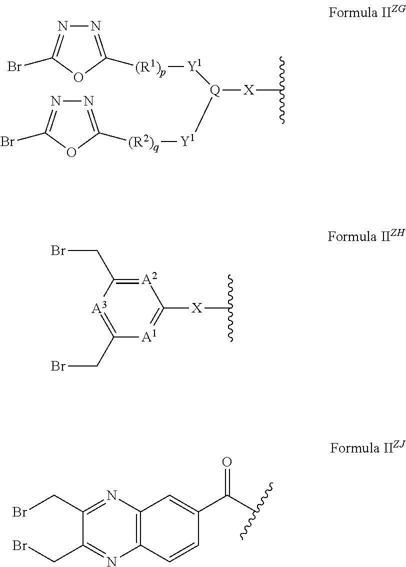

wherein each of A¹, A², A³, X, R¹, R², R³, Q, Y¹, Y², p and q are as defined hereinabove;
Ts is tosylate; and

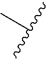

indicates the position where the moiety is attached to linker, L.

In certain embodiments, each Z² is a re-bridging linking group independently selected from a group of Formula II^{ZA}, Formula II^{ZB}, Formula II^{ZE} and Formula II^{ZH}. Suitably, each Z² is a re-bridging linking group independently selected from a group of Formula II^{ZA}, Formula II^{ZB} and Formula II^{ZE}. More suitably, each Z² is a re-bridging linking group of Formula II^{ZA}.

In certain embodiments, there is provided a conjugating reagent selected from one of the following:

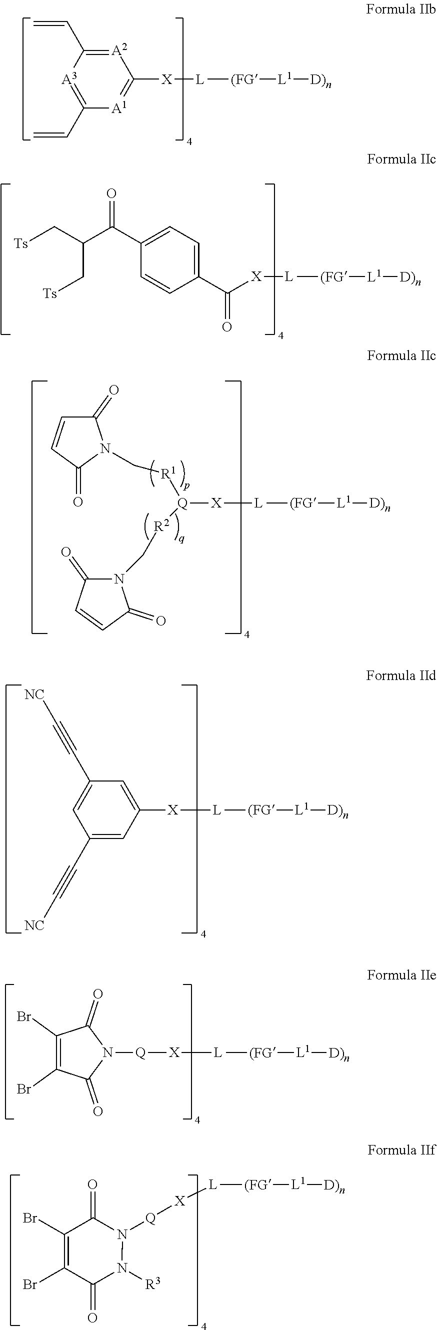

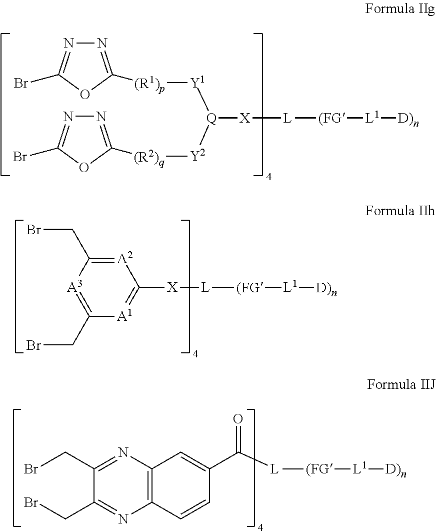

wherein each of D, L, L¹, FG′, A¹, A², A³, X, R¹, R², R³, Q, Y¹, Y², p, q and n are as defined hereinabove.

In other embodiments, the conjugating reagent is of Formula (IIB), shown below:

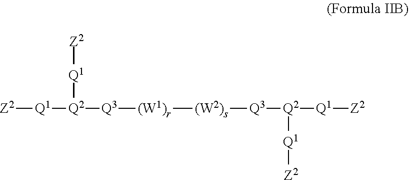

wherein each of Z², Q¹, Q², Q³, W¹, W², r and s are as defined hereinabove.

Preferred and suitable substituent groups for each of D, Q¹, Q², Q³, W¹, W², r and s will be understood to be analogous to the preferred and suitable substituent groups for each of D, Q¹, Q², Q³, W¹, W², r and s for the conjugates of the invention described hereinabove. Preferred and suitable Z² groups will be understood to those described hereinabove.

### Certain Further Embodiments

In certain embodiments, the conjugating reagent is a compound of Formula (IIB), shown below:

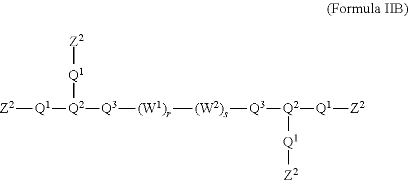

wherein:
   each Z² is a re-bridging moiety of Formula (II^{ZA}) as defined hereinabove;
   each Q¹ is a group of Formula IVa:

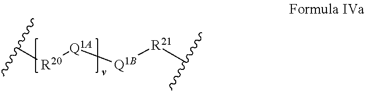

wherein:
   Q^{1A} is selected from C(═O), NR⁷C(═O) and C(═O)NR⁷;
   Q^{1B} is selected from N and CR⁹CR¹⁰;
   R⁷, R⁹ and R¹⁰ are independently selected from hydrogen and C₁-C₄alkyl;
   R²⁰ and R²¹ are independently selected from a C₁-C₈ alkylene and a C₁-C₈ alkylene containing O in the backbone; and
   v is 1 or 2, preferably 1;
each Q² is N;
each Q³ is a group of Formula IVb:

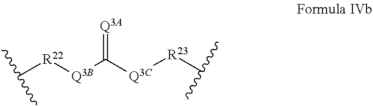

wherein:
   Q^{3A} is O;
   Q^{3B} is a bond;
   Q^{3C} is NR¹³;
   R¹³ is selected from hydrogen and C₁-C₄alkyl; and
   R²² and R²³ are independently a C₁-C₈alkylene or a C₁-C₈alkylene containing O in the backbone;
W¹ is a group of Formula IVc₁:

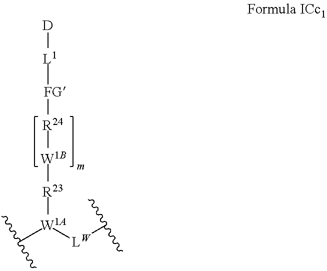

wherein:
   W^{1A} is N;
   L^{W} is absent or a C₁-C₄alkylene;
   W^{1B} is a group selected from —NR¹⁵C(═O)— and —C(═O)NR¹⁵—;
   R¹⁵ and R¹⁶ are independently selected from hydrogen and C₁-C₄alkyl;
   R²³ and R²⁴ are independently selected from a bond, a C₁-C₈alkylene and a C₁-C₈ alkylene containing O in the backbone; and
   m is an integer selected from 1 or 2 (suitably m is 1);
W² is a group of Formula IVd:

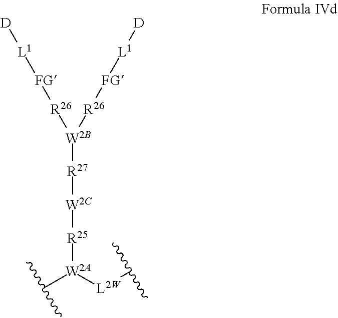

wherein:
   W^{2A} is selected from N;
   L^{2W} is absent or a C₁-C₄alkylene;
   W^{2B} is N;
   W^{2C} is —NHC(═O)— or —C(═O)NH—;
   R²⁵, R²⁶ and R²⁷ are independently selected from a bond and a C₁-C₆alkylene;
FG′ is

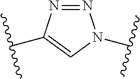

wherein N is bound to L¹;
L¹ is selected from a bond, a C₁-C₁₀alkylene, a C₁-C₁₀alkylene containing O in the backbone and a group of Formula Va¹:

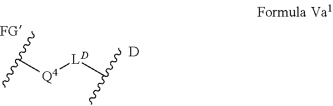

wherein:

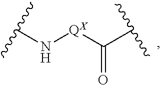

Q⁴ is a single bond, or, where Q^{X} is such that Q⁴ is an amino-acid residue, a dipeptide residue or a tripeptide residue; and
L^{D} is a group for attachment to the active agent;
D is an active agent, as defined herein; and
wherein r and s are selected such that the total number of active agents per conjugate is between 1 and 8.

In certain embodiments, the conjugating reagent is a compound of Formula (IIB), shown below:

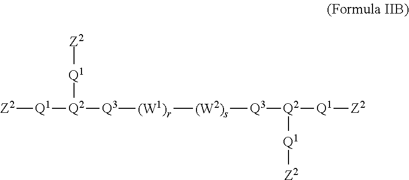

wherein:
   each Z² is a re-bridging moiety of Formula (II^{ZA}) as defined hereinabove;
   each Q¹ is a group of Formula IVa:

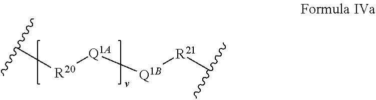

wherein:
   Q^{1A} is selected from C(═O), NR⁷C(═O) and C(═O)NR⁷;
   Q^{1B} is selected from N and CR⁹CR¹⁰;
   R⁷, R⁹ and R¹⁰ are independently selected from hydrogen and C₁-C₄alkyl;
   R²⁰ and R²¹ are independently selected from a C₁-C₈ alkylene and a C₁-C₈alkylene containing O in the backbone; and
   v is 1 or 2, preferably 1;
each Q² is N;
each Q³ is a group of Formula IVb:

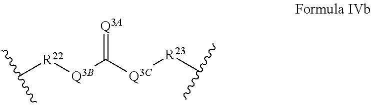

wherein:
   Q^{3A} is O;
   Q^{3B} is a bond
   Q^{3C} is NR¹³;
   R¹³ is selected from hydrogen and C₁-C₄alkyl; and
   R²² and R²³ are independently a C₁-C₈alkylene or a C₁-C₈ alkylene containing O in the backbone;
W¹ is a group of Formula IVc:

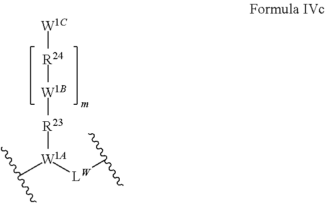

wherein:
   W^{1A} is N;
   L^{W} is absent or a C₁-C₄alkylene;
   W^{1B} is a group selected from —NR¹⁵C(═O)— and —C(═O)NR¹⁵—;
   R¹⁵ and R¹⁶ are independently selected from hydrogen and C₁-C₄alkyl; and
   R²³ and R²⁴ are independently selected from a bond, a C₁-C₈alkylene and a C₁-C₈ alkylene containing O in the backbone; and
   m is an integer selected from 1 or 2 (suitably m is 1);
   W^{Q1} is a group of Formula W^{C3A}:

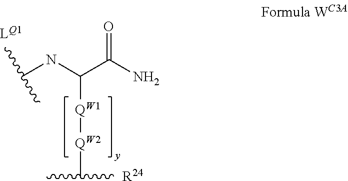

wherein:
Q^{W1} is absent or a C₁-C₆alkylene;
Q^{W2} is selected from —NHC(═O)— and —C(═O)NH—;
y is an integer selected from 0 or 1;
W^{Q2} is a group of Formula W^{C4A}:

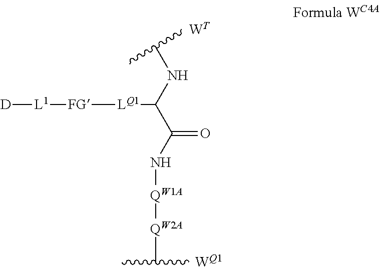

wherein:
   Q^{W1A} is a C₁-C₆alkylene optionally containing O in the backbone;
   Q^{W2A} is —C(═O)—;

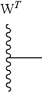

indicates the position where the NH is attached to another group of formula W^{Q2} via the Q^{W2} substituent group, or

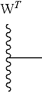

indicates the position where the NH is attached to a —C(═O)CH₃;
L^{Q1} is a group of Formula LQ₁;

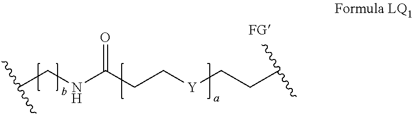

wherein:
Y is O or NH;
a is an integer selected from 1 to 6;
b is an integer selected from 1 to 6;
t is an integer selected from 1 to 4;

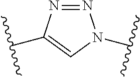 [SMILES: *c1cn(*)nn1]

FG′ is wherein N is bound to L¹;
L¹ is selected from a bond, C₁-C₁₀alkylene, a C₁-C₁₀alkylene containing O in the backbone and a group of formula Va¹:

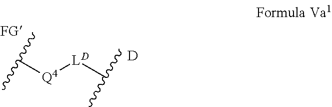

wherein:
   Q⁴ is a single bond, or

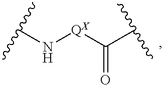

where Q^{X} is such that Q⁴ is an amino-acid residue, a dipeptide residue or a tripeptide residue; and
L^{D} is a group for attachment to the active agent;
D is an active agent, as defined herein; and
wherein r and s are selected such that the total number of active agents per conjugate is between 1 and 8.

In other embodiments, the conjugating reagent is selected from one of the following:

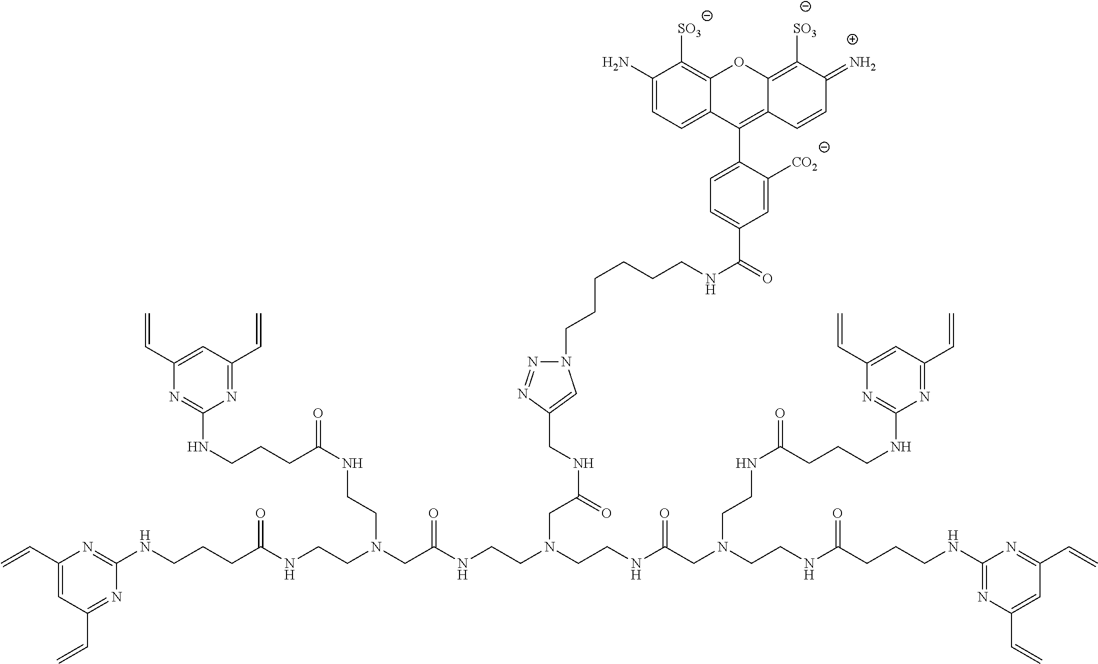

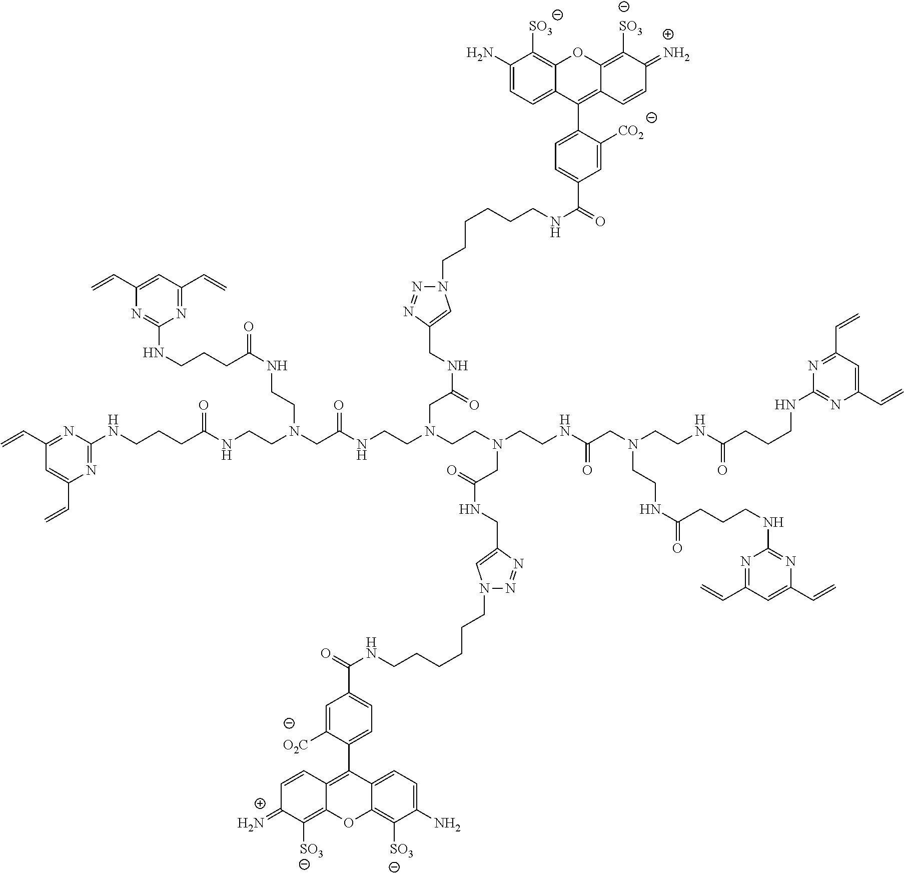

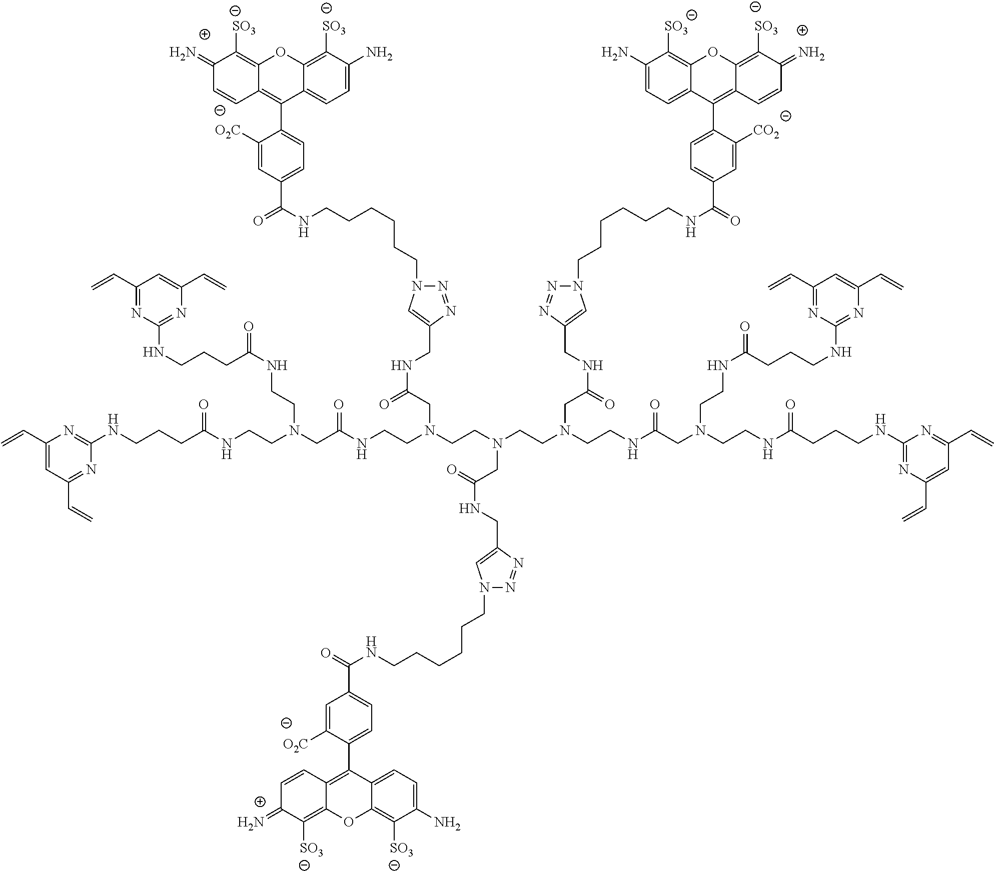

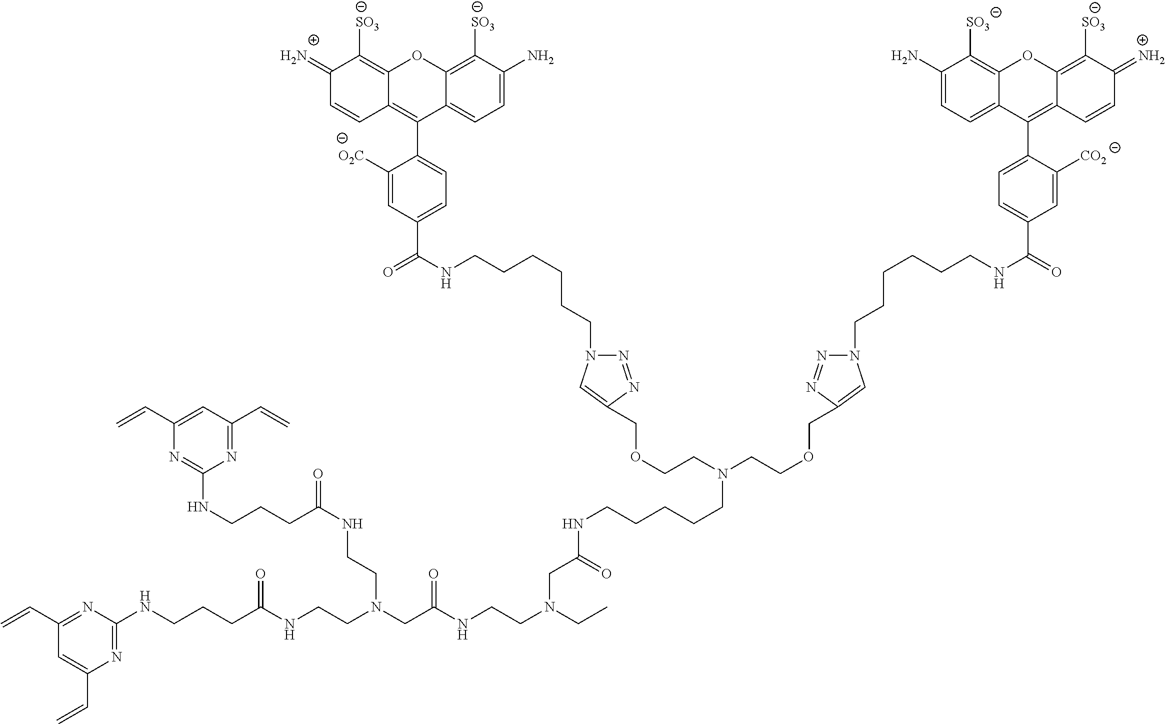

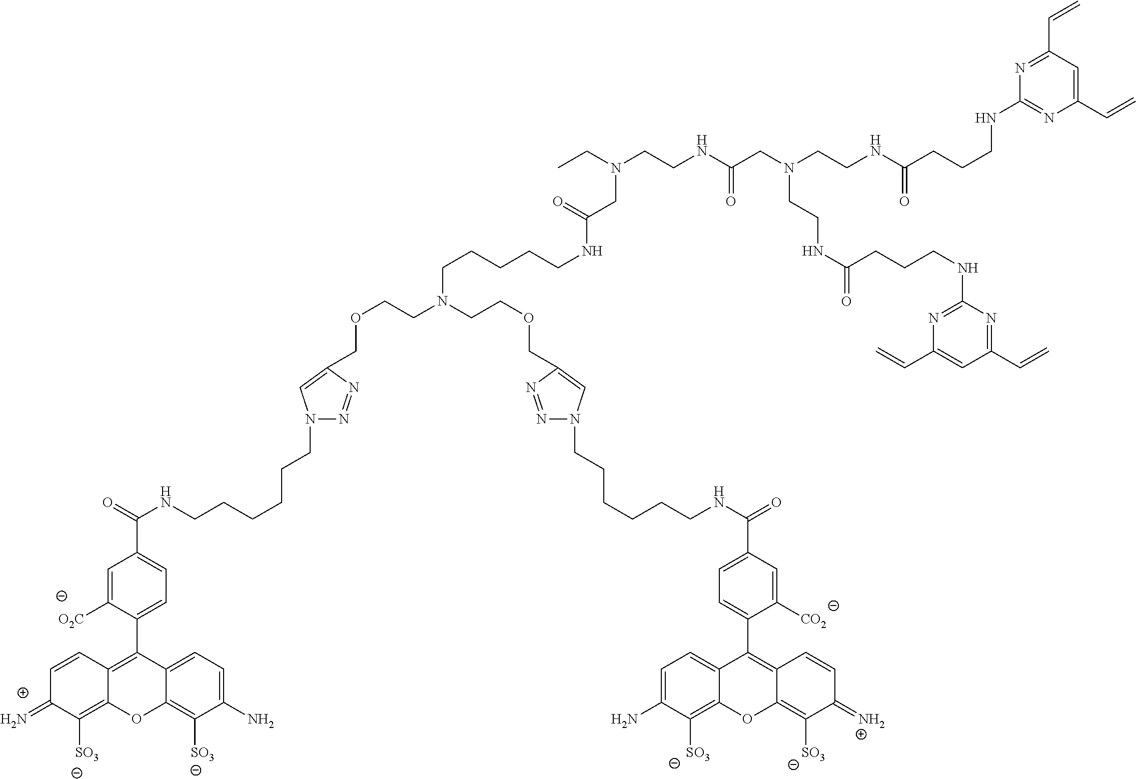

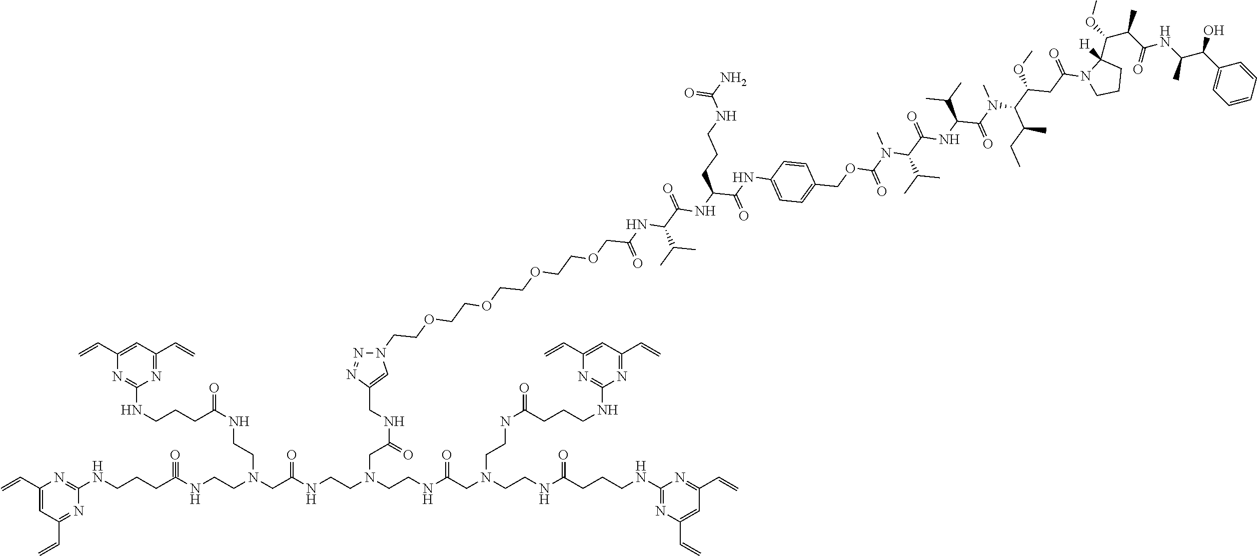

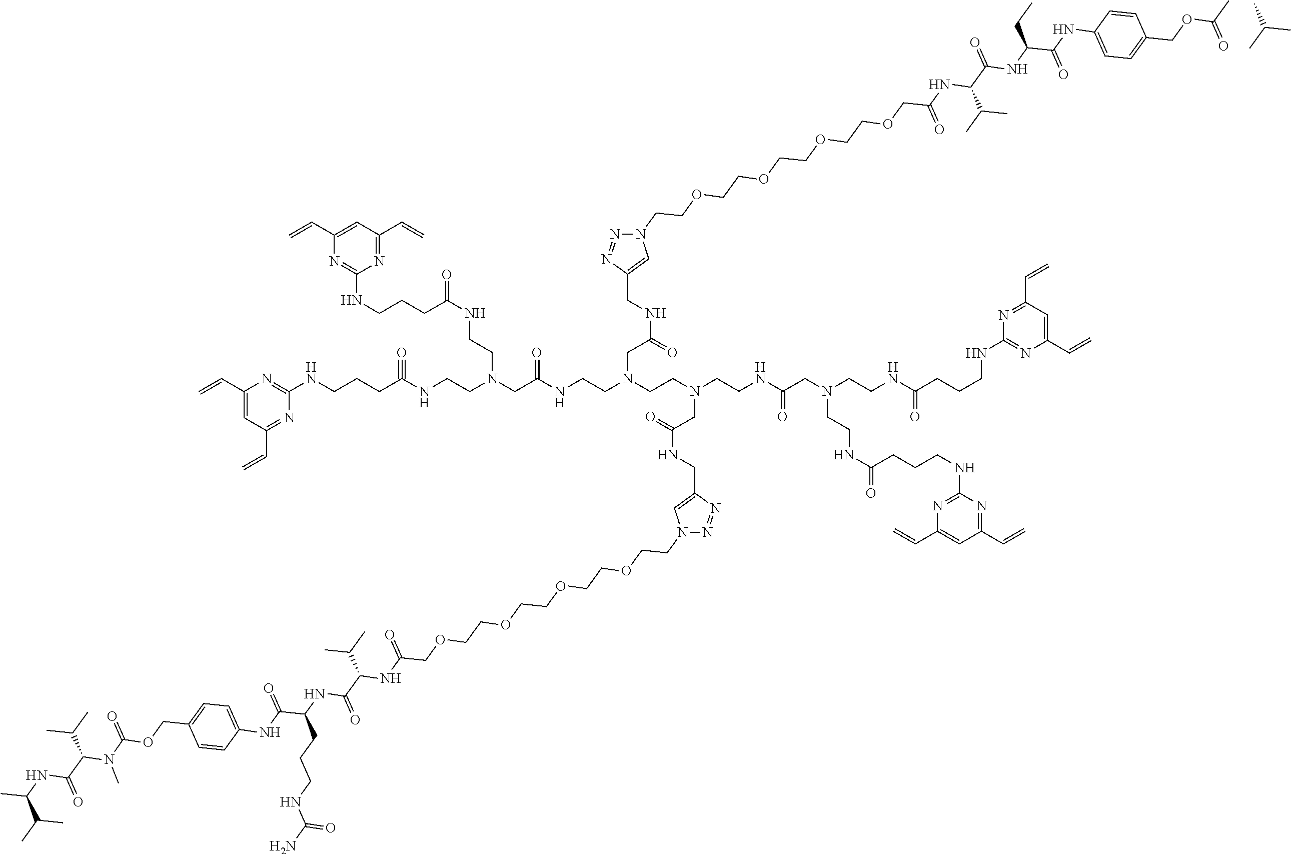

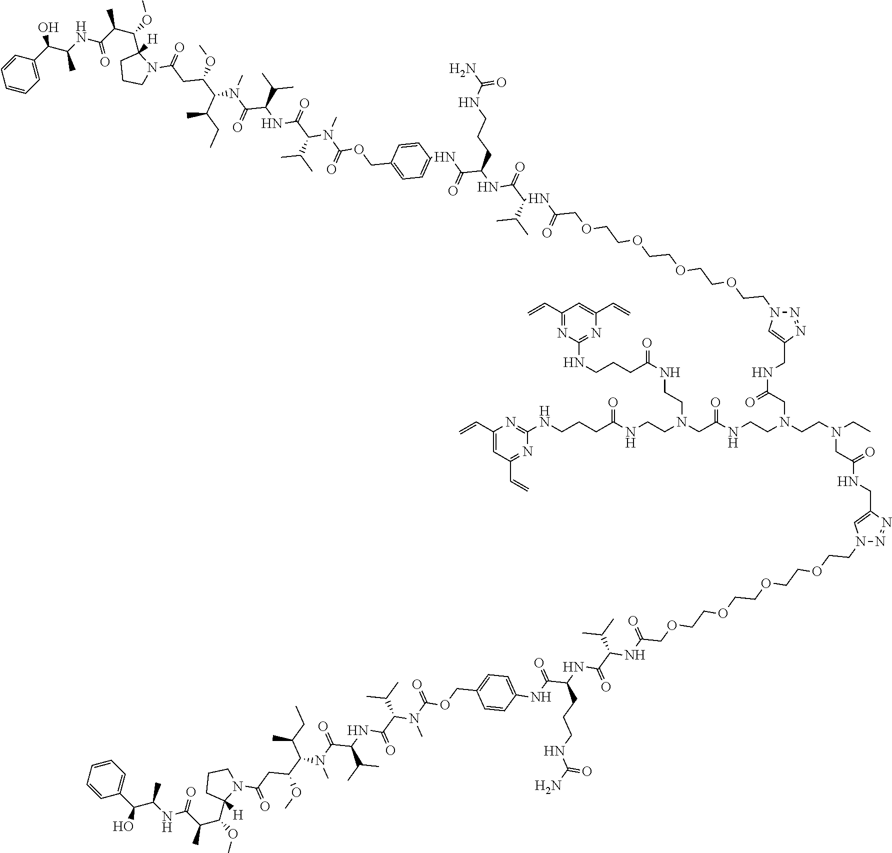

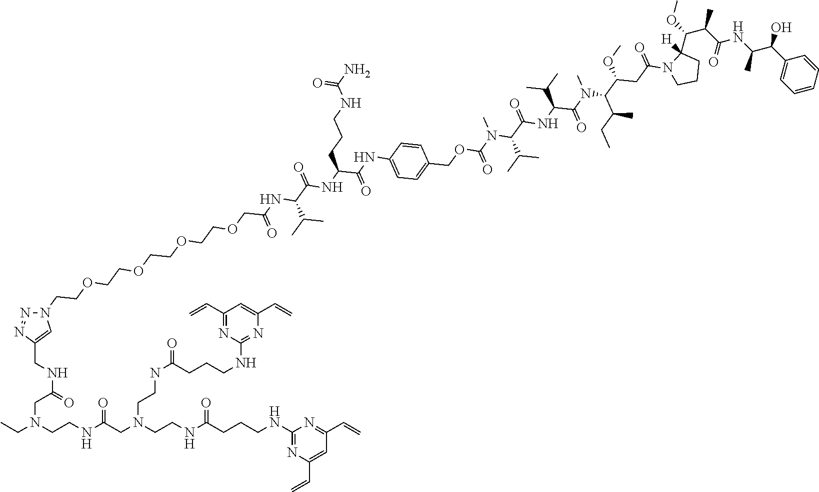

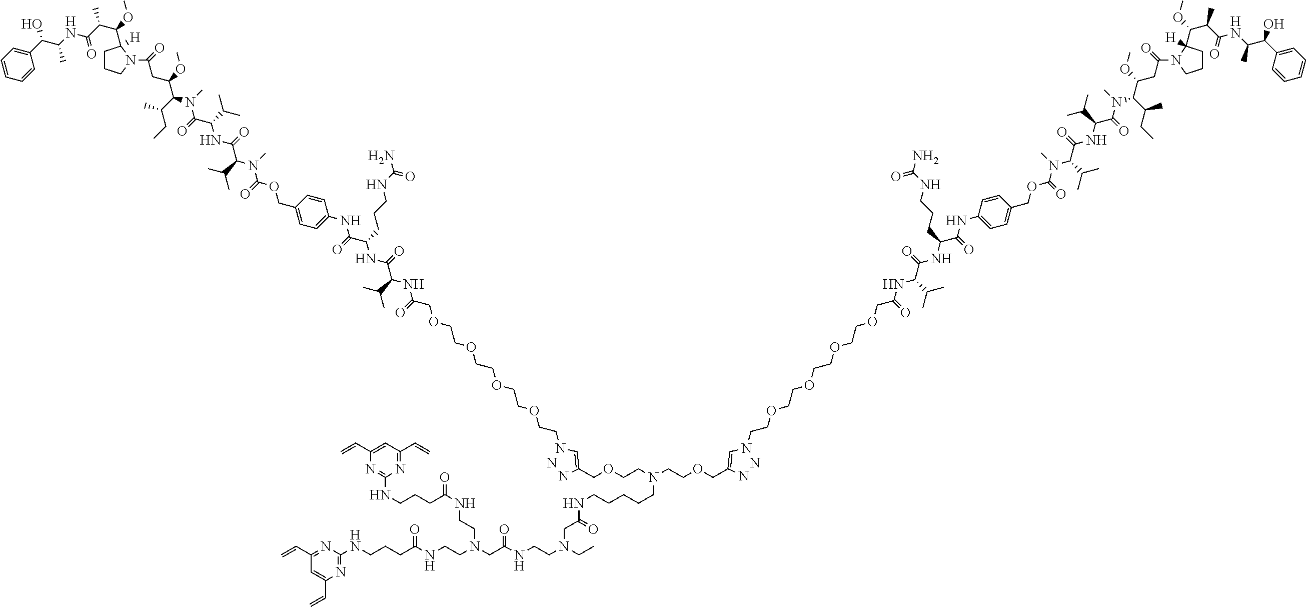

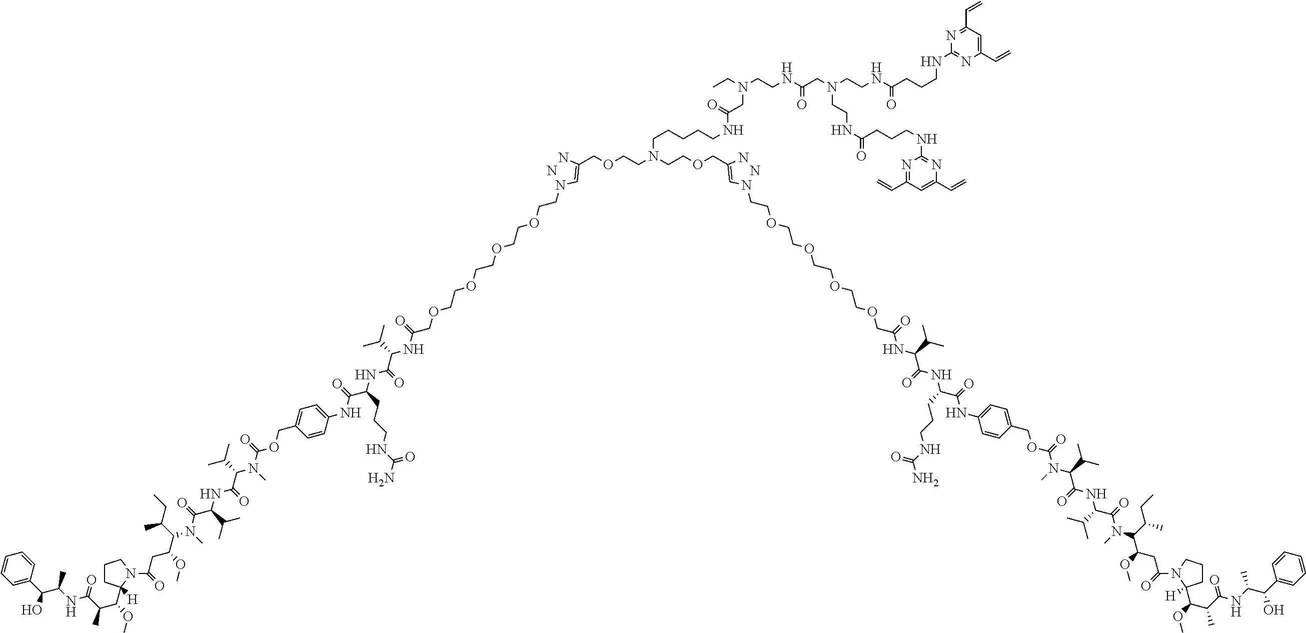

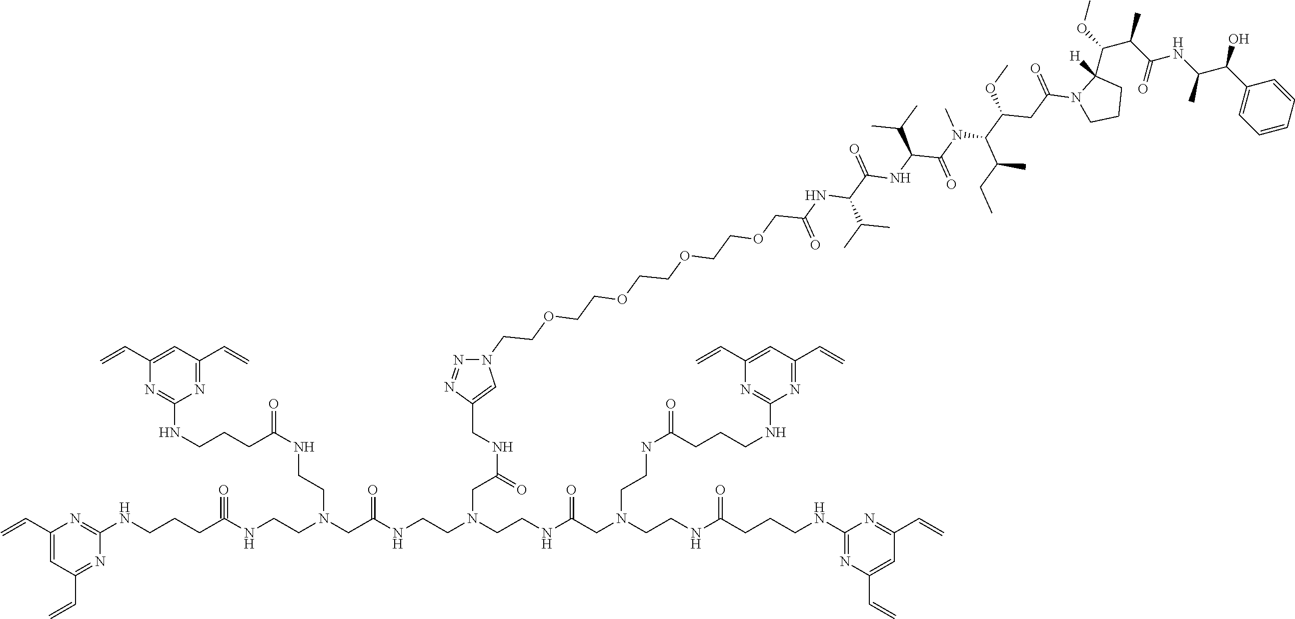

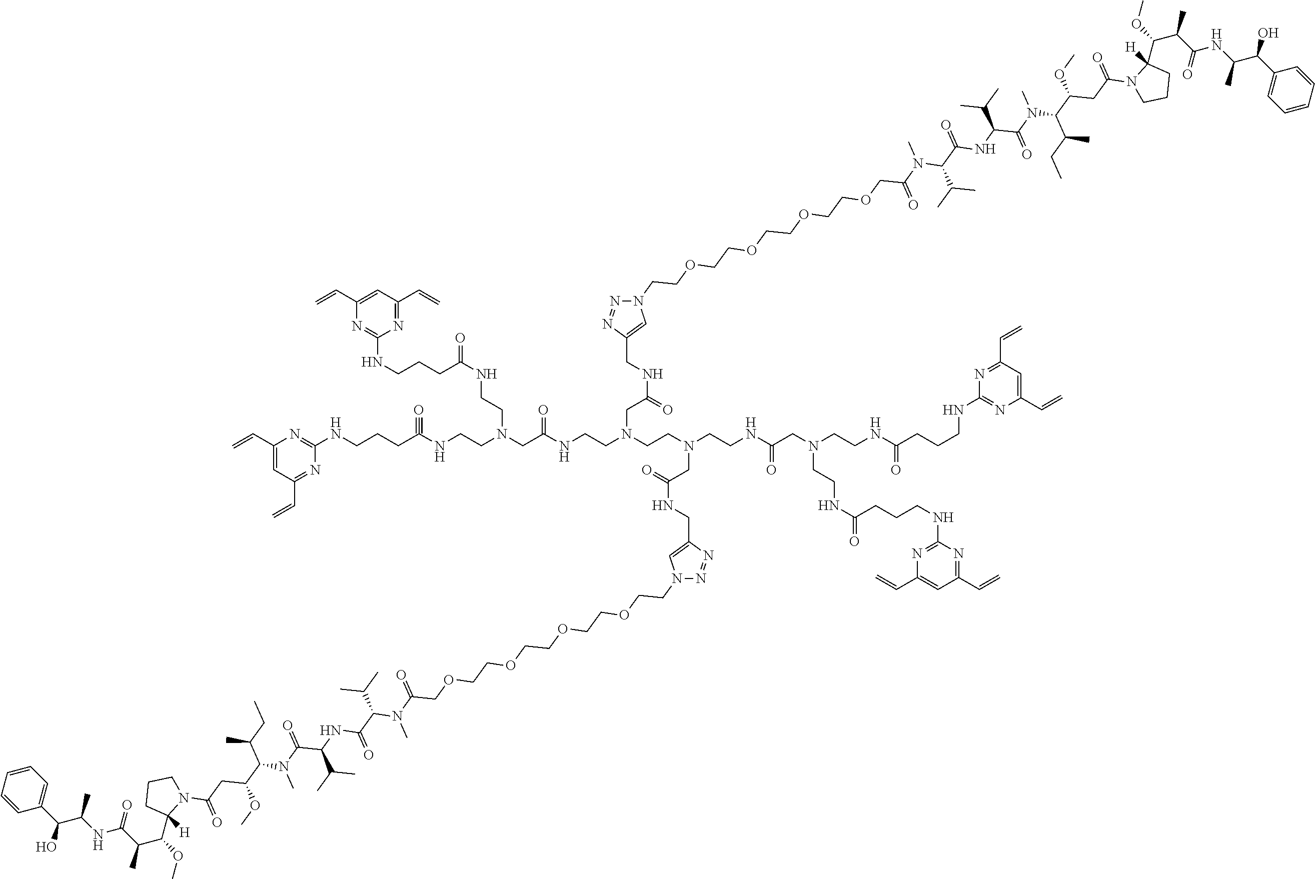

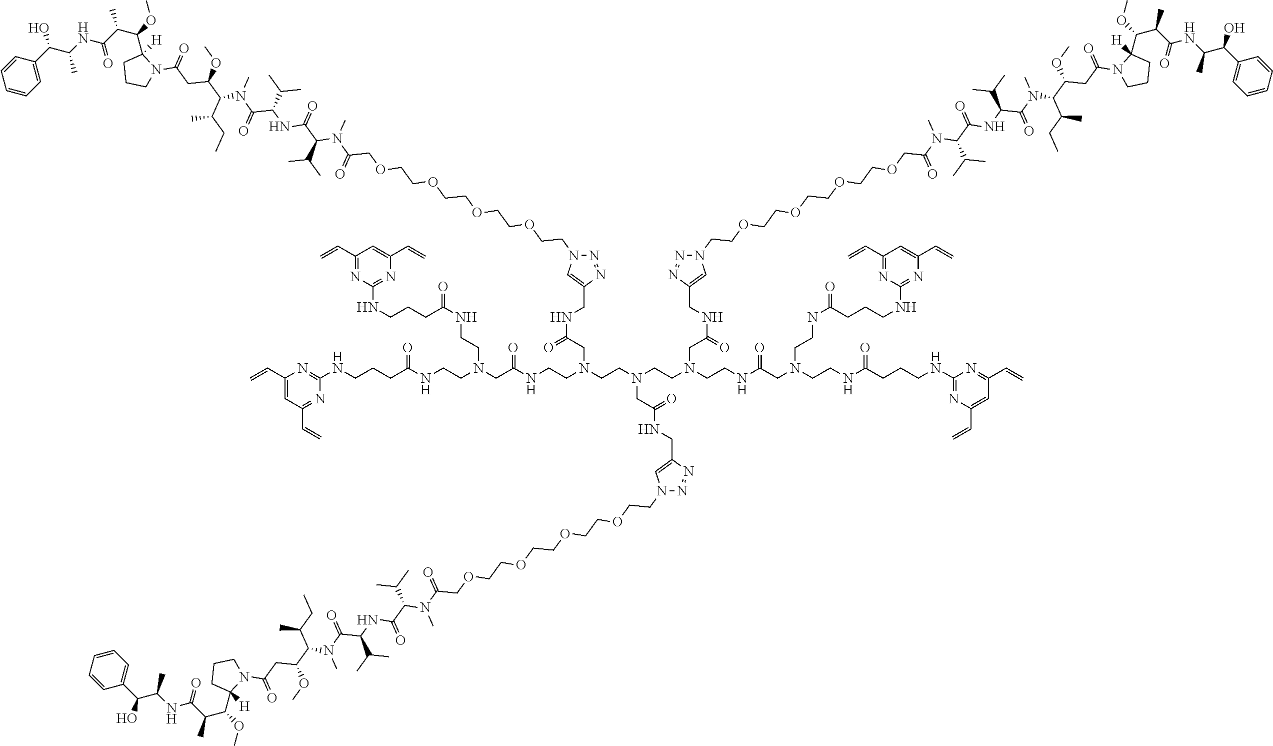

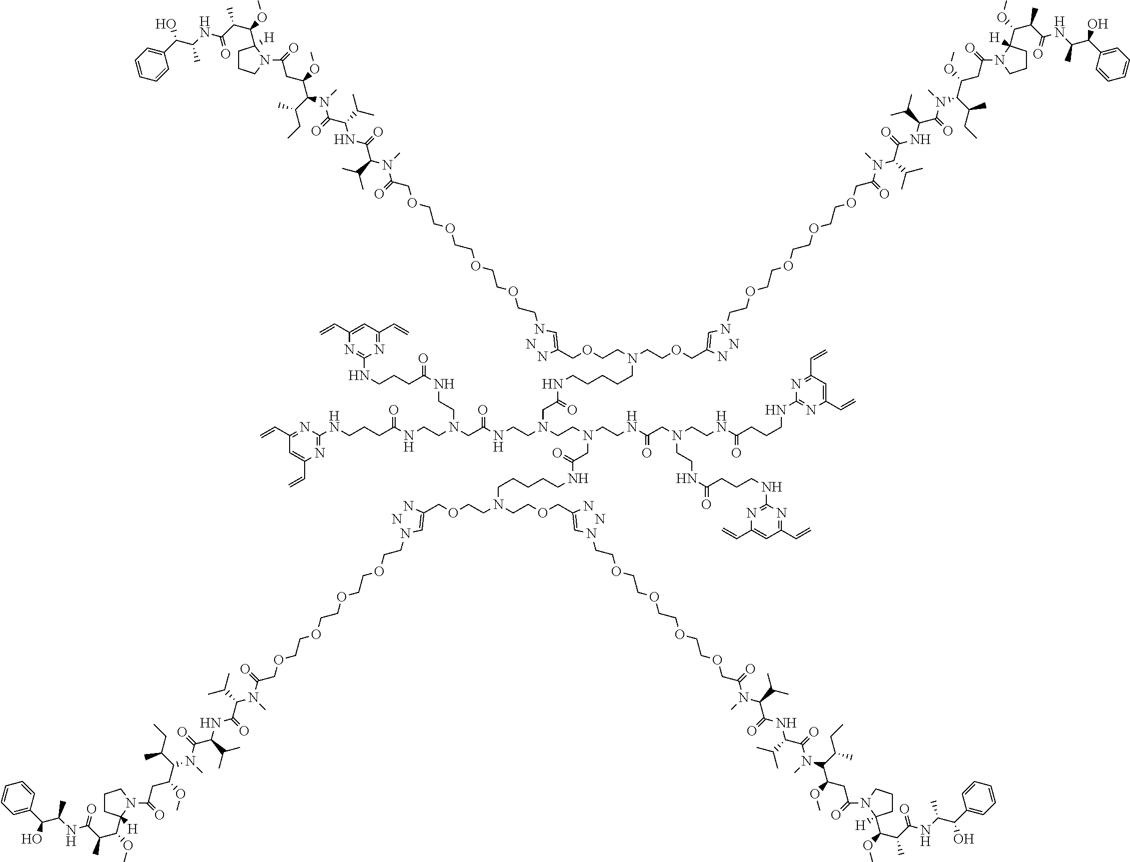

In some embodiments, the conjugating reagent is selected from one of the following:

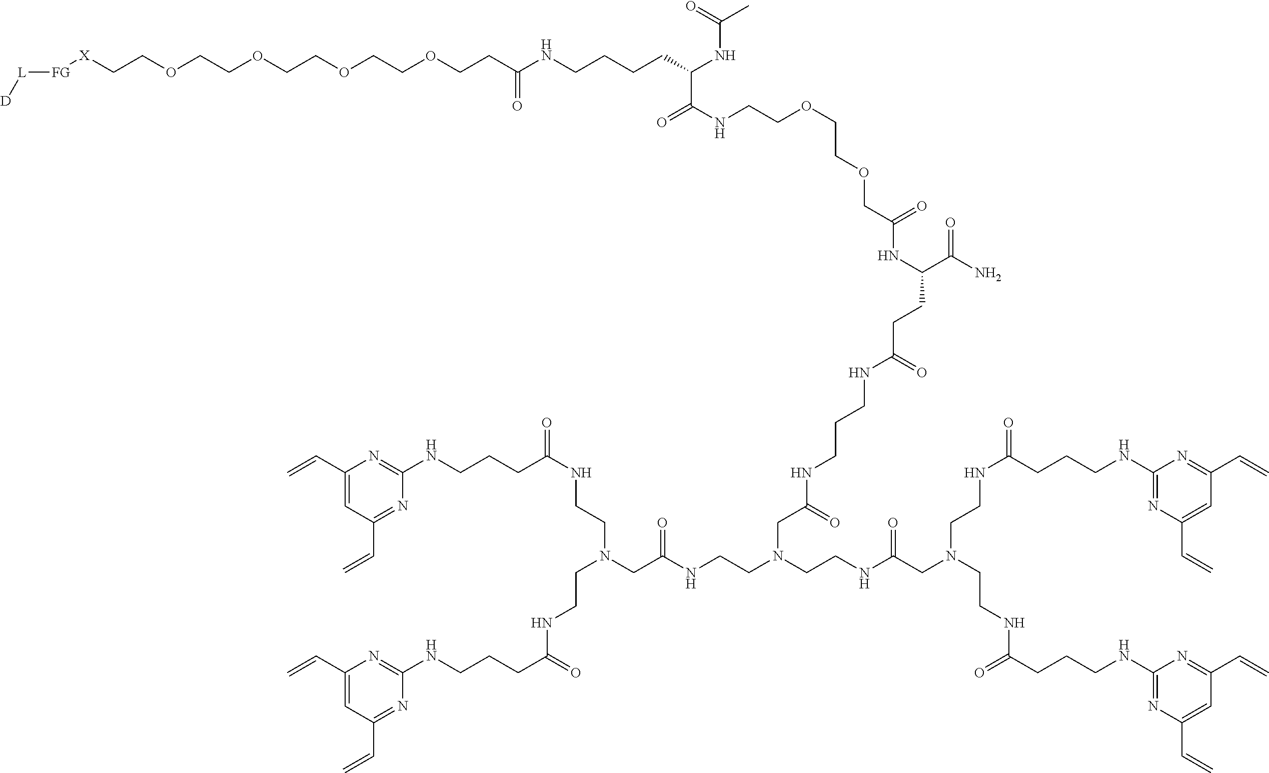

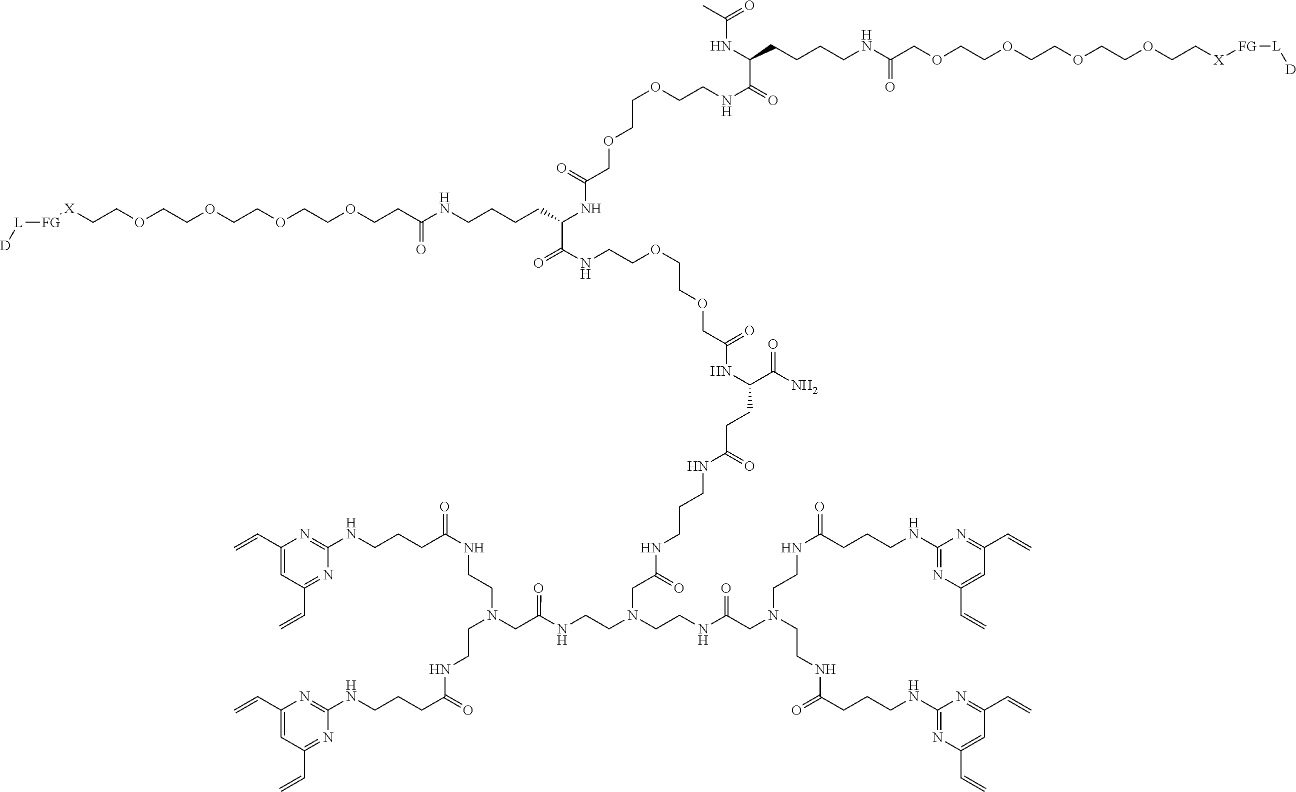

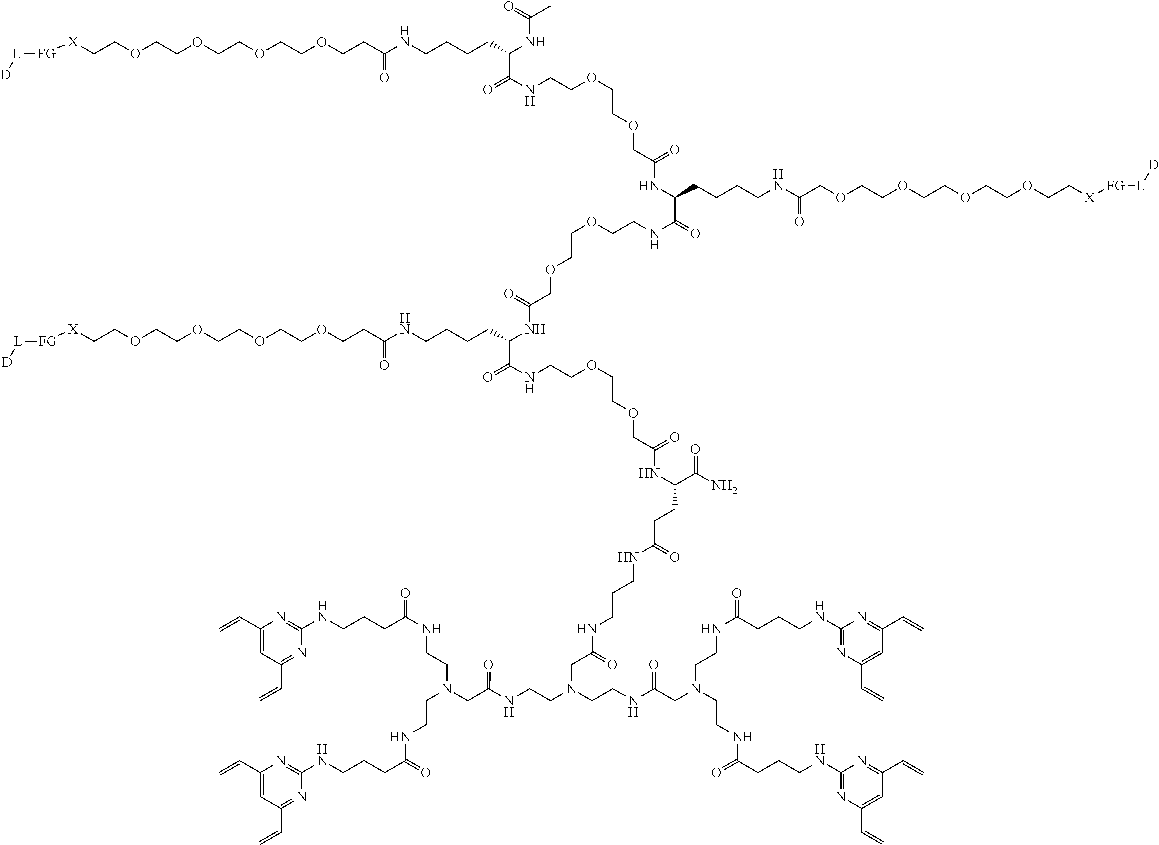

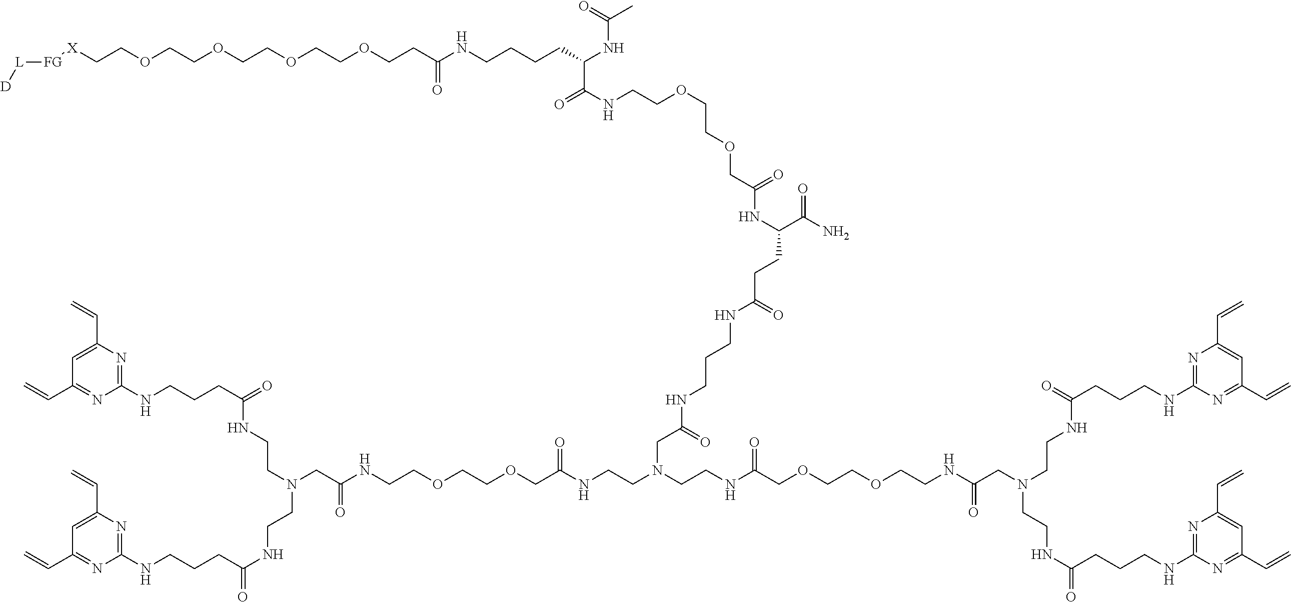

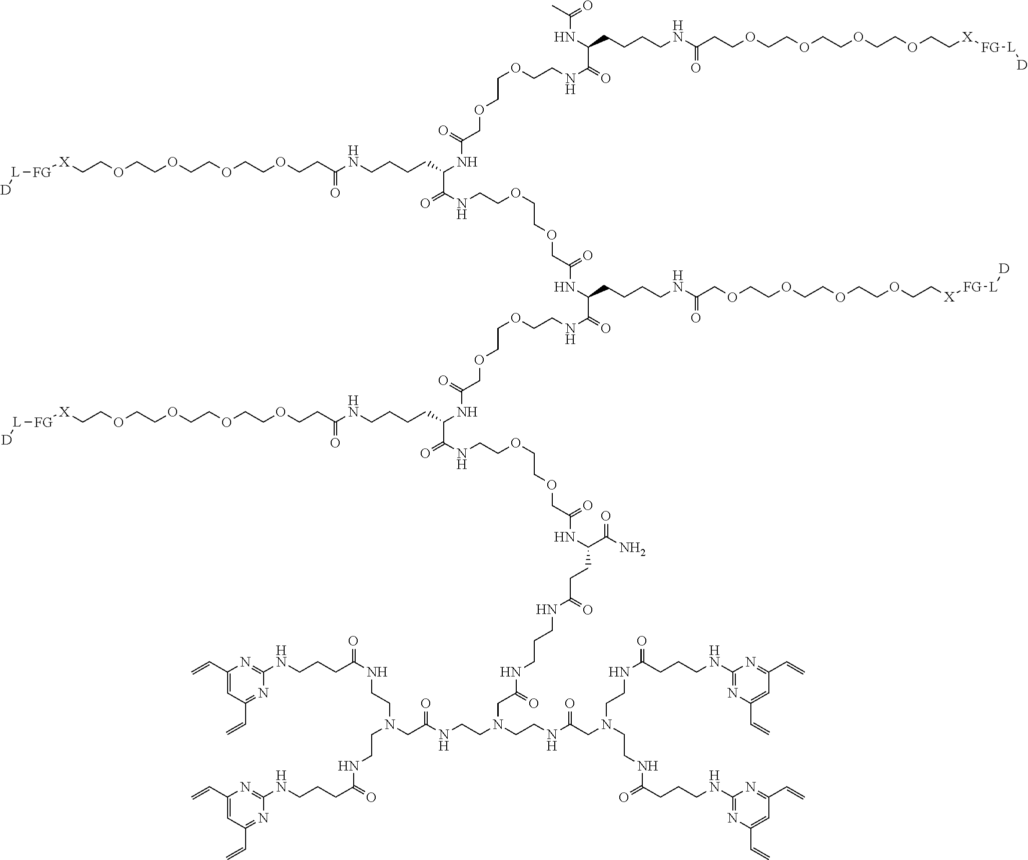

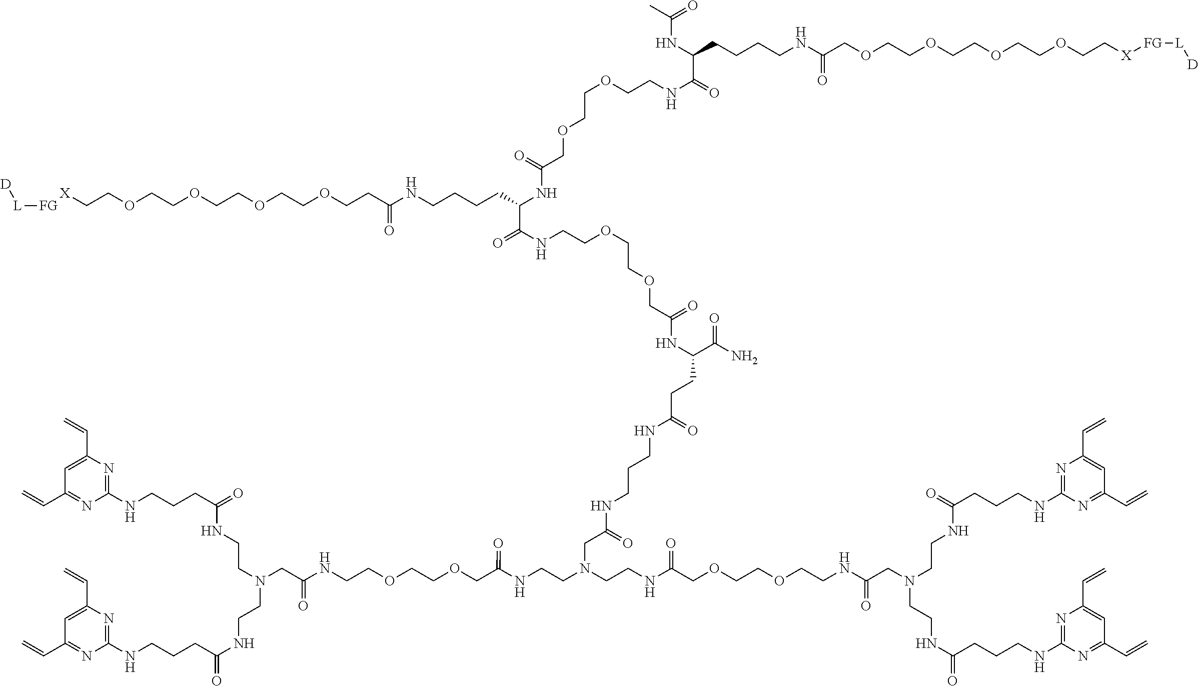

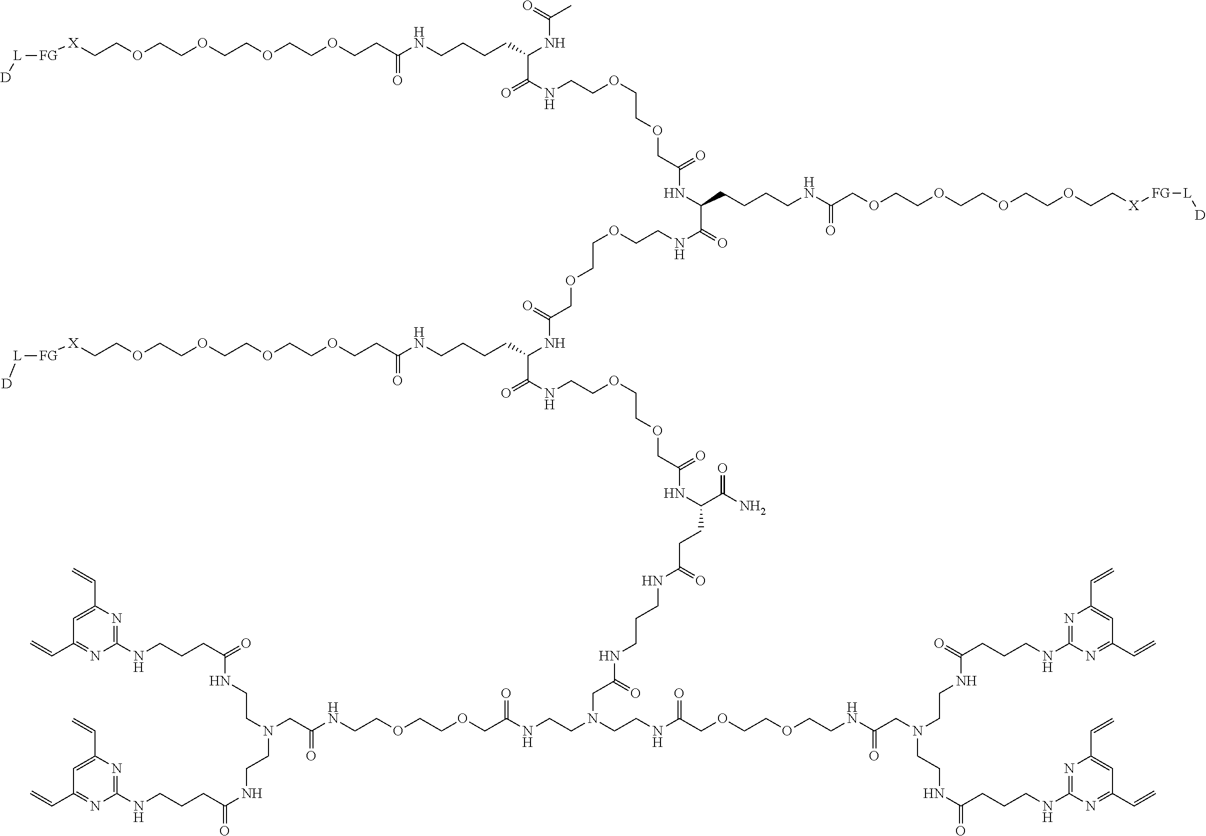

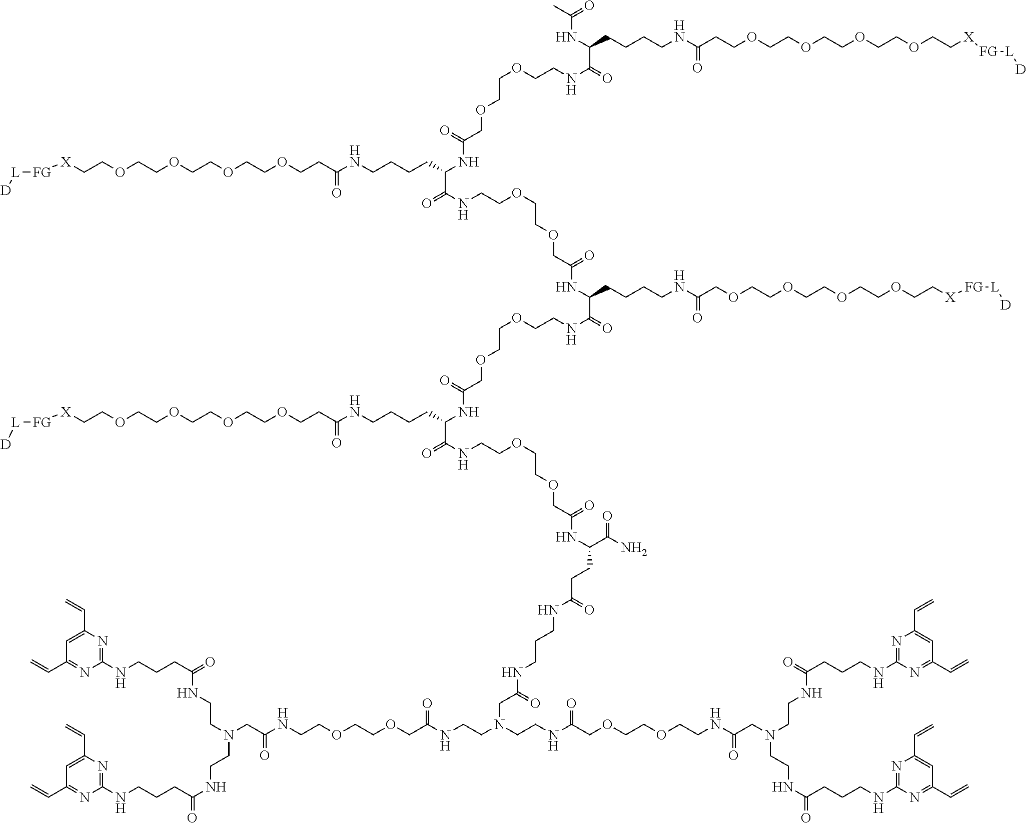

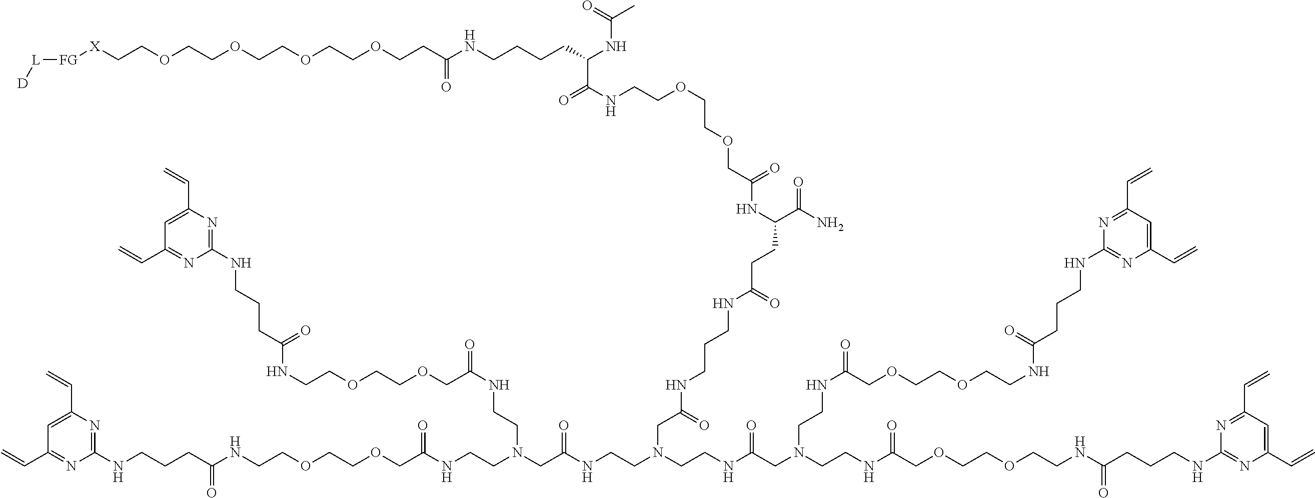

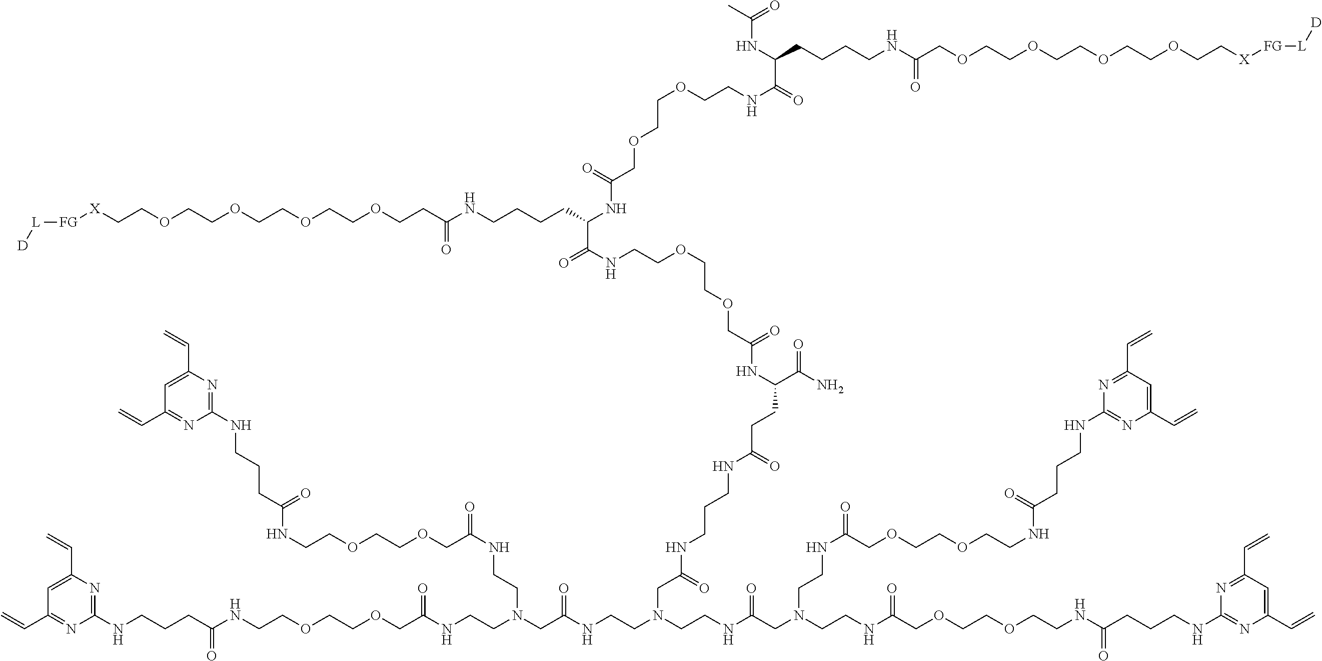

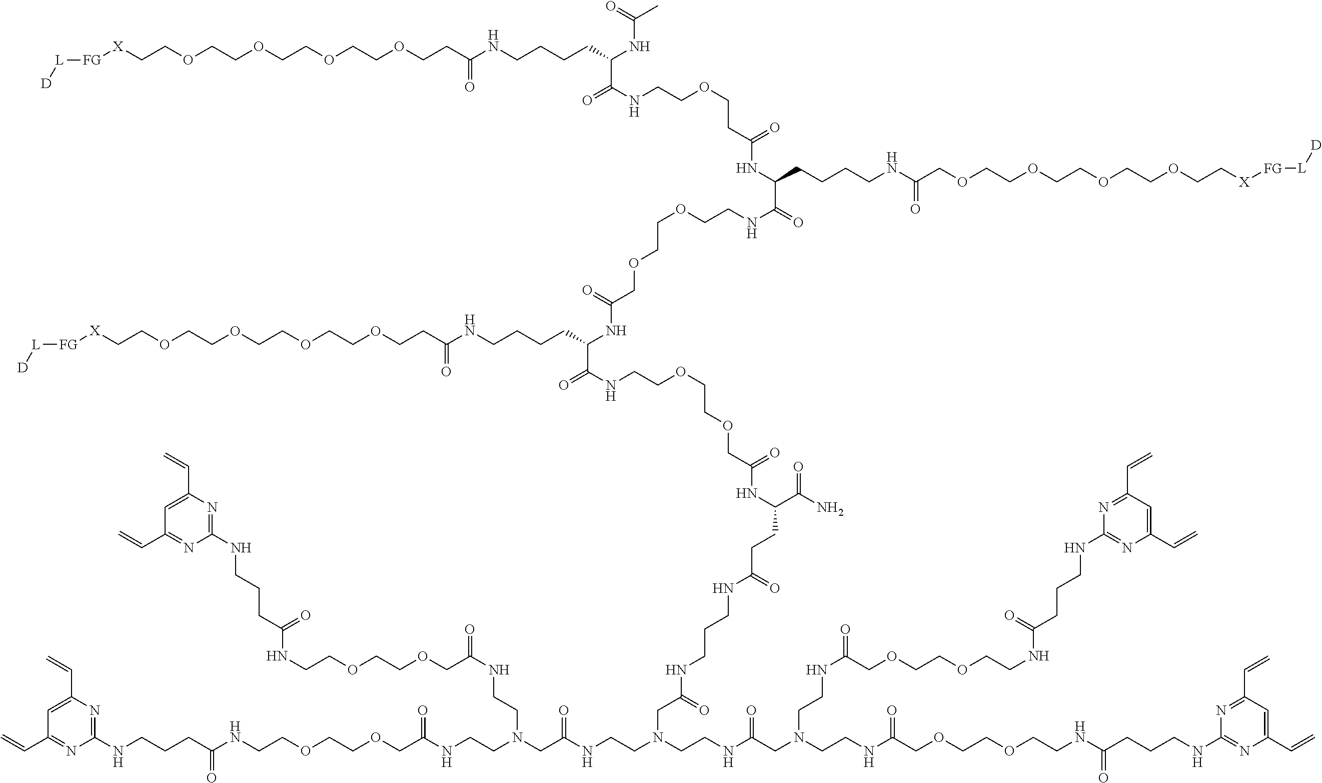

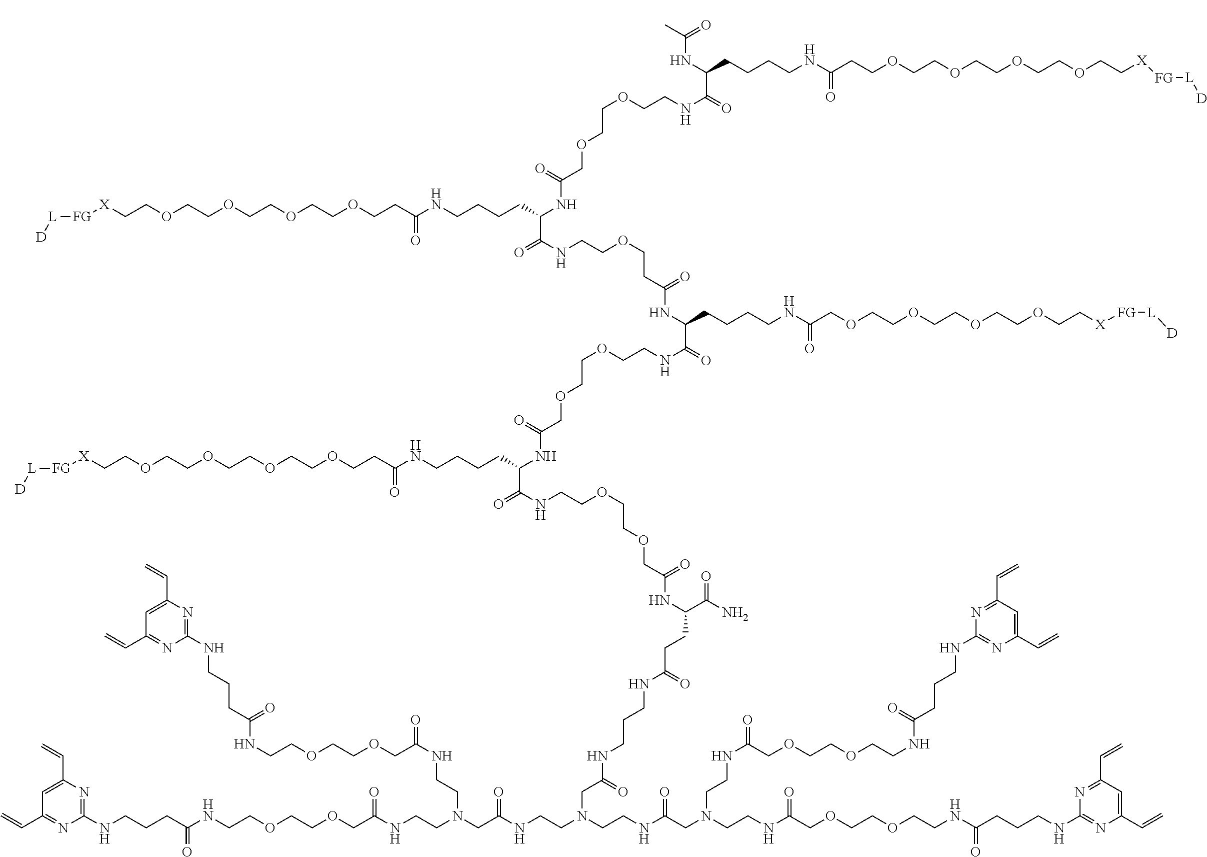

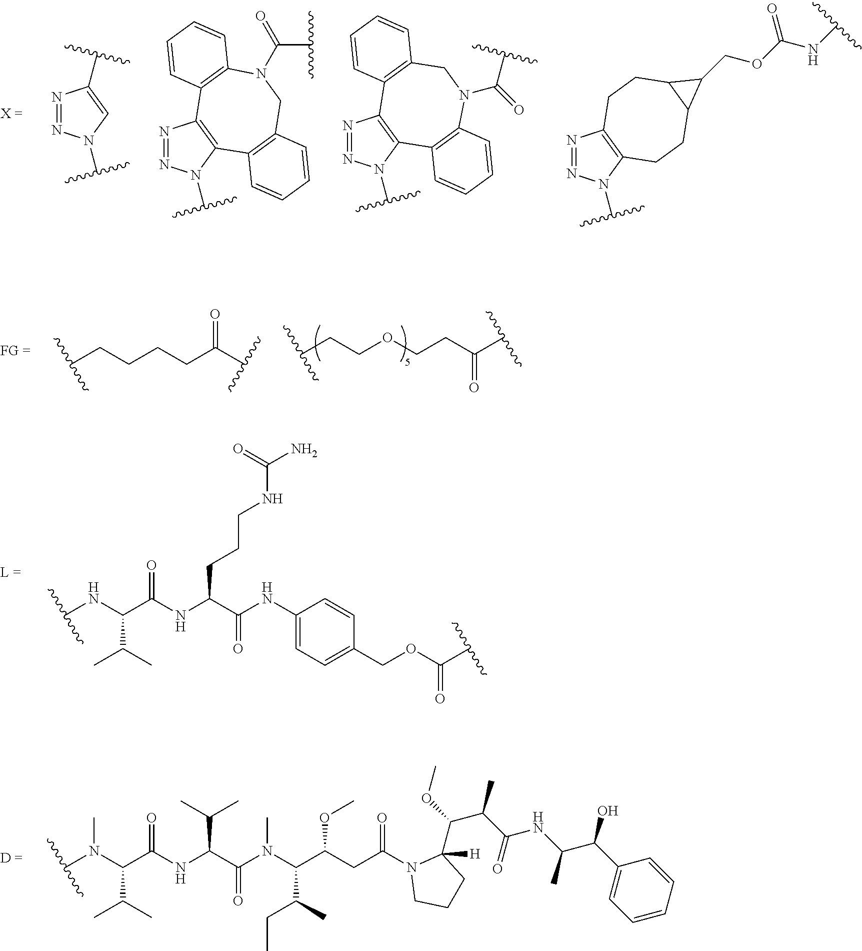

### Compounds of the Invention

According to another aspect of the invention, there is provided compound of Formula (I), shown below:

(FG)ₙ-L-(Z)₈ (Formula I)
wherein:
   FG is a functional group capable reacting with another moiety to form a functional linking moiety;
   n is an integer selected from 0 to 20;
   L is a linker; and
   Z is a functional group capable of reaction with a sulfur atom from a cysteine moiety of an antibody;
      or a pharmaceutically acceptable salt, solvate or hydrate thereof

Preferred and suitable substituent groups for each of FG, n, L and Z will be understood to be analogous to the preferred and suitable substituent groups for each of FG, n, L and Z for the conjugates and conjugating reagents of the invention described hereinabove.

Thus, in certain embodiments, FG is a functional group selected from an alkene, an alkyne, an azide, a hydroxyl, an amine, a carboxylic acid, an aldehyde, an acyl halide, a tetrazine, an alkoxyamine (e.g. hydroxylamine), a hydrazine, an electron-rich dienophile (e.g. a 1,3-nitrone alkene) and an electron-poor diene (e.g. tetrazine), nitrone, an isocyanate and an isothiocyanate.

Suitably, FG is a functional group selected from an alkene, an alkyne, an azide, a hydroxyl, an amine, a carboxylic acid, an aldehyde, an acyl halide, a tetrazine, an alkoxyamine (e.g. hydroxylamine), and a hydrazine. More suitably, FG is a functional group selected from an alkyne, an azide, hydroxyl, amine, carboxylic acid, aldehyde and acyl halide. Most suitably, FG is a functional group selected from an alkyne and an azide.

In certain embodiments, the compound is of Formula (Ia), shown below:

(FG)ₙ-L-(Z²)₄ (Formula Ia)

wherein Z², L, FG and n are each as defined hereinabove.

In some embodiments, the compound is selected from one of the following groups:

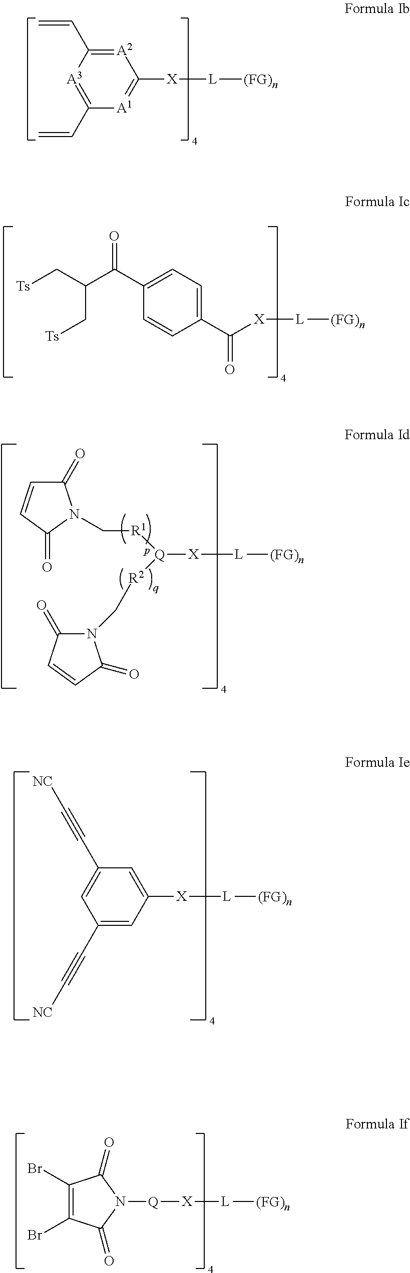

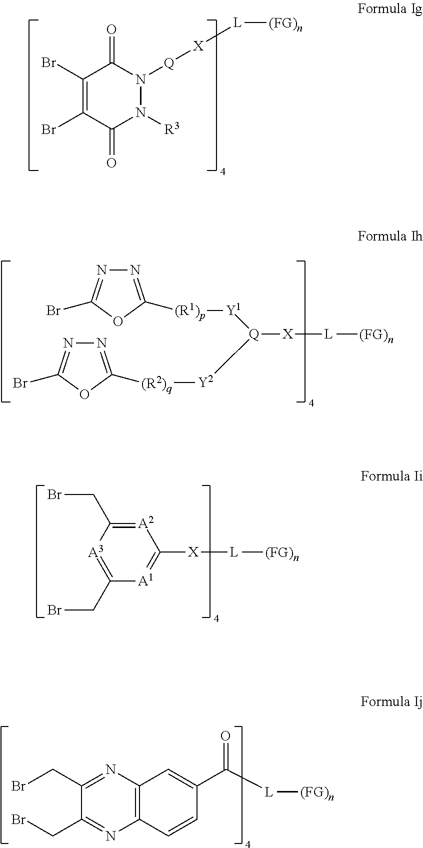

wherein each of A¹, A², A³, X, L, FG, n, Q, Y¹, Y², R¹, R², R³, q and p are as defined hereinabove.
In certain embodiments, the compound is a compound of Formula Ib, Formula Ib, Formula If or Formula Ij. Suitably, the compound is a compound of Formula Ib, Formula Ib, Formula If or Formula Ij. More suitably, the compound is a compound of Formula Ib.

In some embodiments, the compound is of Formula (Ic), shown below

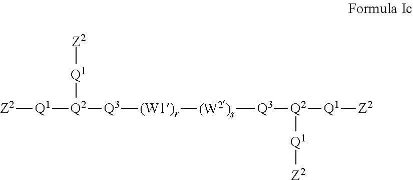

wherein:
Z², Q¹, Q², Q³, r and s are as defined herein;
W^{1′} is a group of Formula IVc or IVc₂:

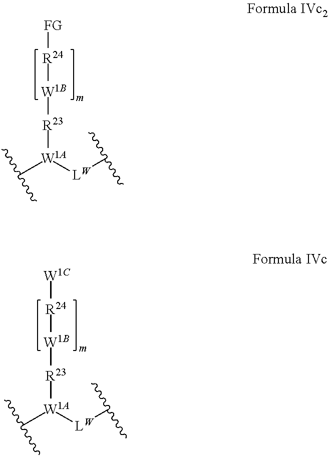

wherein:
   W^{1A} is selected from N and CR¹⁴, wherein R¹⁴ is a selected from hydrogen and C₁-C₄alkyl;
   L^{W} is absent or selected from C₁-C₆alkylene and C₁-C₆alkylene containing amino, O or N in the backbone;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₈alkylene containing O in the backbone; and
   m is an integer selected from 1 or 2 (suitably m is 1);
   W^{1C} is a group of Formula W^{C2};
   W^{Q1} is a group of Formula W^{C3A}:

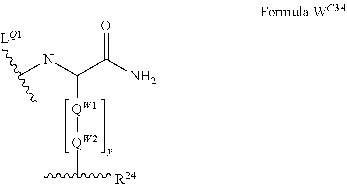

wherein:
Q^{W1} is absent or a C₁-C₆alkylene;
Q^{W2} is selected from —NHC(═O)— and —C(═O)NH—;
y is an integer selected from 0 or 1;
W^{Q2} is a group of Formula W^{C4A}:

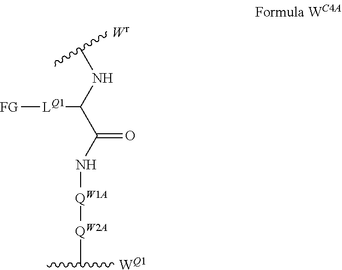

wherein:
   Q^{W1A} is a C₁-C₆alkylene optionally containing an O in the backbone;
   Q^{W2A} is —C(═O)—;

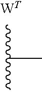

indicates the position where the NH is attached to another group of formula W^{Q2} via the Q^{W2} substituent group, or

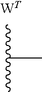

indicates the position where the NH is attached to a —C(═O)CH₃;
L^{Q1} is a group of Formula LQ₁;

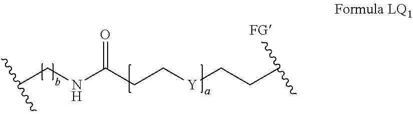

wherein:
Y is O or NH;
a is an integer selected from 1 to 6;
b is an integer selected from 1 to 6;
t is an integer selected from 1 to 4; and
FG is as defined herein; and
W^{2′} is a group of Formula IVd:

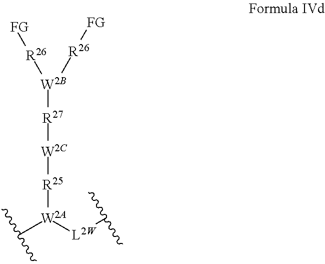

wherein:
   W^{2A} is selected from N and CR¹⁷, wherein R¹⁷ is a selected from hydrogen and C₁-C₄alkyl;
   L^{2W} is absent or selected from C₁-C₆alkylene and C₁-C₆alkylene containing amino, O or N in the backbone;
   W^{2B} is a group selected from N, CR¹⁸, —C(═O)N— and —C(═O)CR¹—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   R¹⁷ and R¹⁸ are independently selected from hydrogen and C₁-C₄alkyl;
   R²⁵, R²⁶ and R²⁷ are independently selected from a bond, C₁-C₈alkylene and C₁-C₆alkylene containing O in the backbone; and
   FG is as defined herein.

In other embodiments, the compound is of Formula (Ic), shown below:

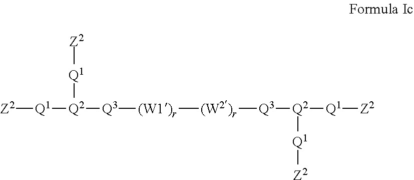

wherein:
Z², Q¹, Q², Q³, r and s are as defined herein;
W^{1′} is a group of Formula IVc₂:

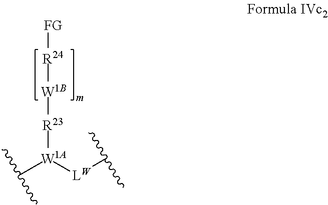

wherein:
   W^{1A} is selected from N and CR¹⁴, wherein R¹⁴ is a selected from hydrogen and C₁-C₄alkyl;
   L^{W} is absent or selected from a C₁-C₆alkylene and a C₁-C₆alkylene containing O or N in the backbone;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₈alkylene containing O in the backbone;
   m is an integer selected from 1 or 2 (suitably m is 1); and
   FG is as defined herein;
W^{2′} is a group of Formula IVd:

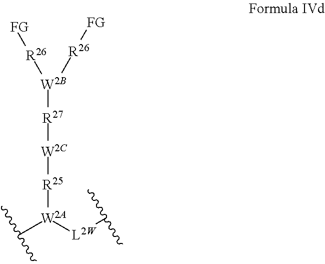

wherein:
   W^{2A} is selected from N and CR¹⁷, wherein R¹⁷ is a selected from hydrogen and C₁-C₄alkyl;
   L^{2W} is absent or selected from a C₁-C₆alkylene and a C₁-C₆alkylene containing O or N in the backbone;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   R¹⁷ and R¹⁸ are independently selected from hydrogen and C₁-C₄alkyl;
   R²⁵, R²⁶ and R²⁷ are independently selected from a bond, C₁-C₈alkylene and C₁-C₈alkylene containing O in the backbone; and
   FG is as defined herein.

In other embodiments, the compound is selected from one of the following:

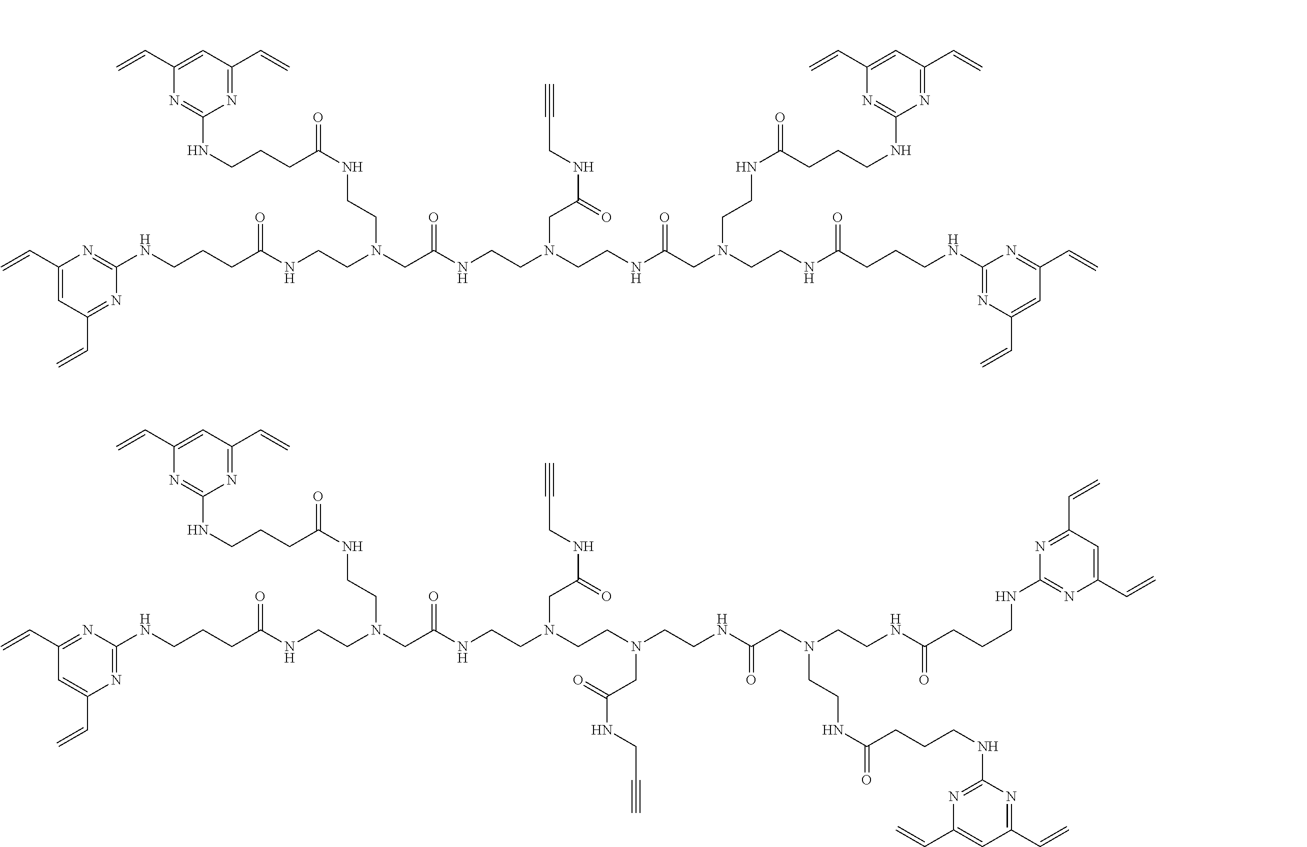

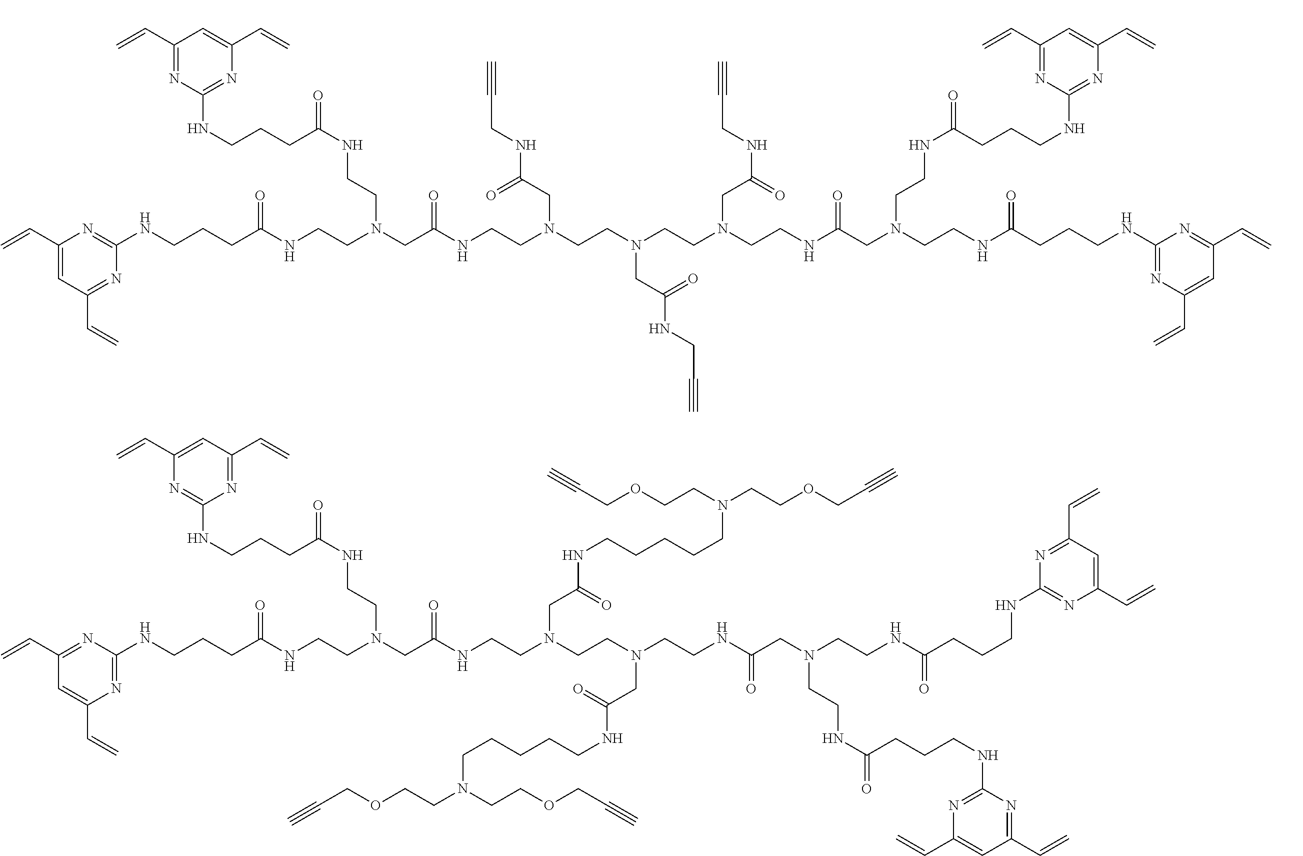

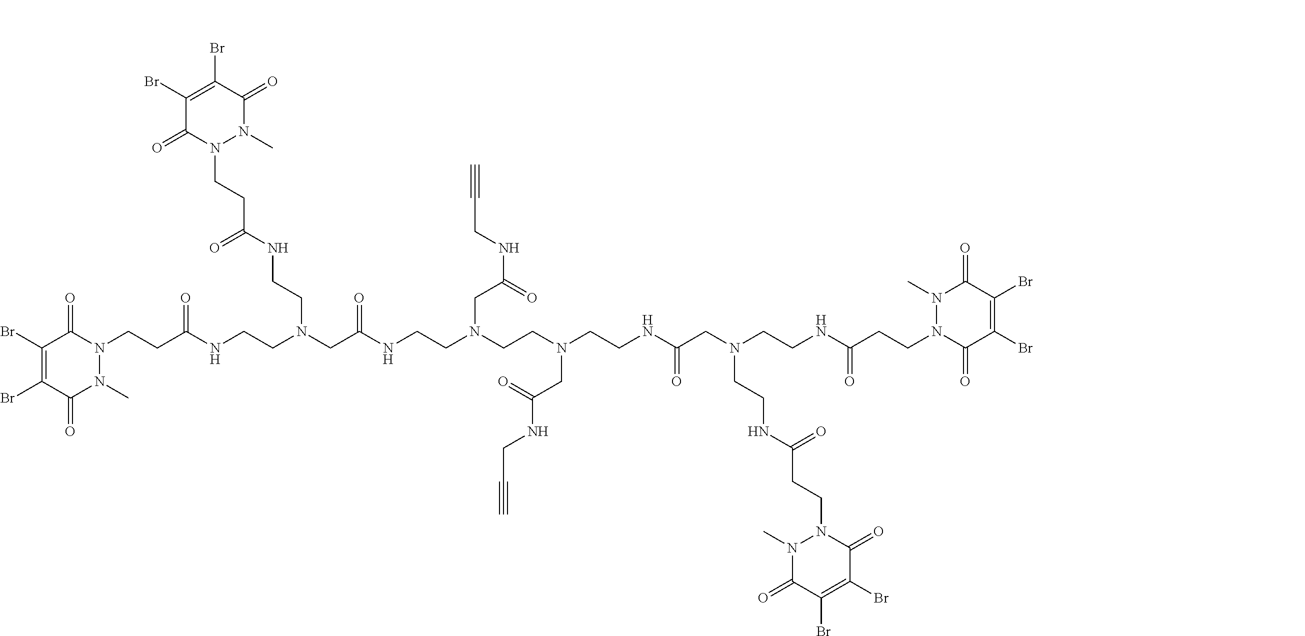 [SMILES: C#CCNC(=O)CN(CCNC(=O)CN(CCNC(=O)CCn1c(=O)c(Br)c(Br)c(=O)n1C)CCNC(=O)CCn1c(=O)c(Br)c(Br)c(=O)n1C)CCN(CCNC(=O)CN(CCNC(=O)CCn1c(=O)c(Br)c(Br)c(=O)n1C)CCNC(=O)CCn1c(=O)c(Br)c(Br)c(=O)n1C)CC(=O)NCC#C]

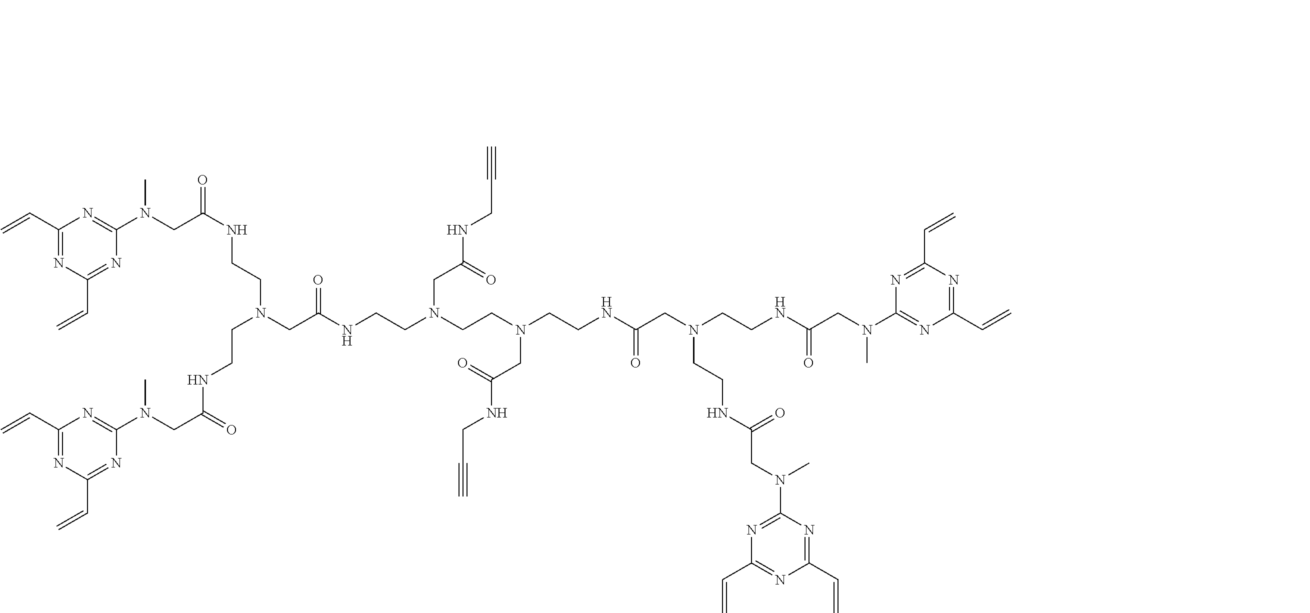 [SMILES: C#CCNC(=O)CN(CCNC(=O)CN(CCNC(=O)CN(C)c1nc(C=C)nc(C=C)n1)CCNC(=O)CN(C)c1nc(C=C)nc(C=C)n1)CCN(CCNC(=O)CN(CCNC(=O)CN(C)c1nc(C=C)nc(C=C)n1)CCNC(=O)CN(C)c1nc(C=C)nc(C=C)n1)CC(=O)NCC#C]

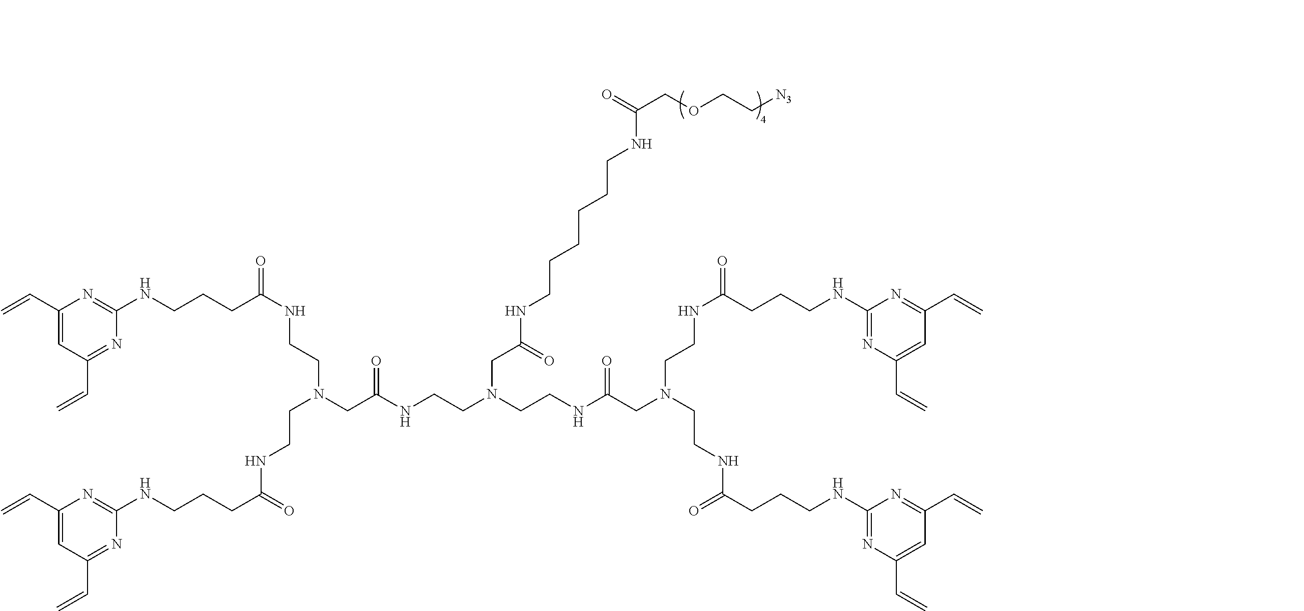

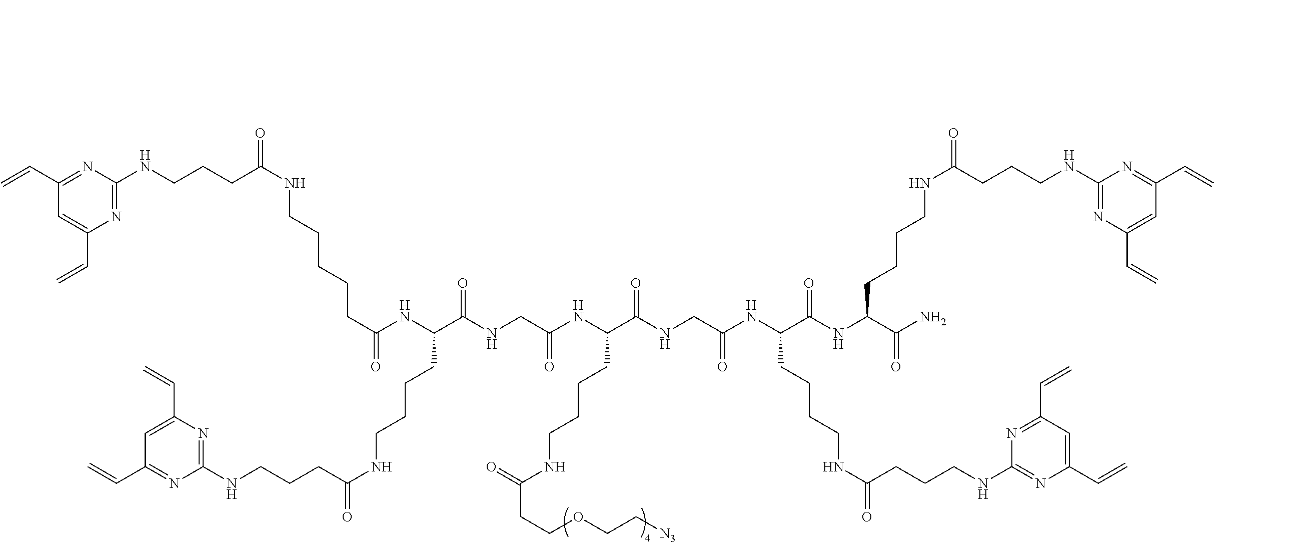

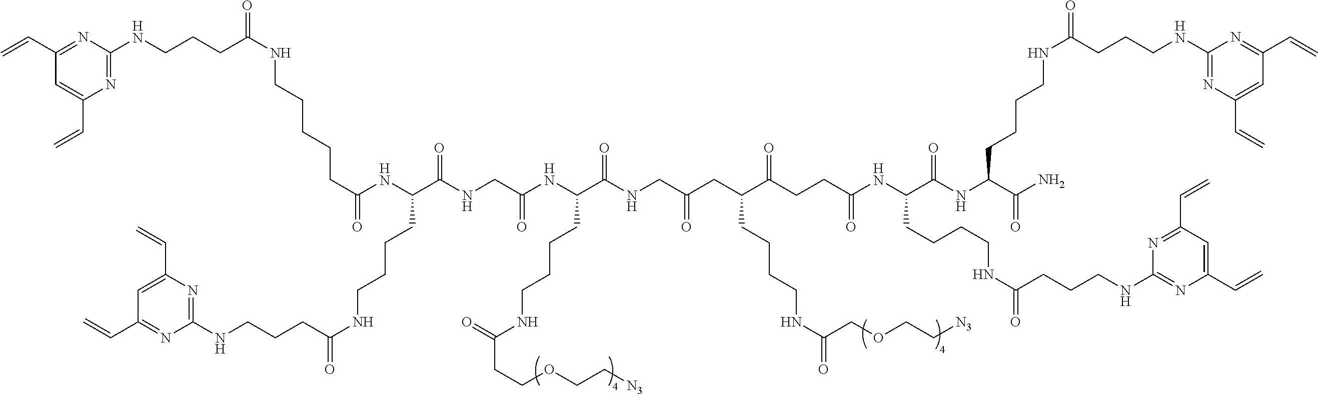

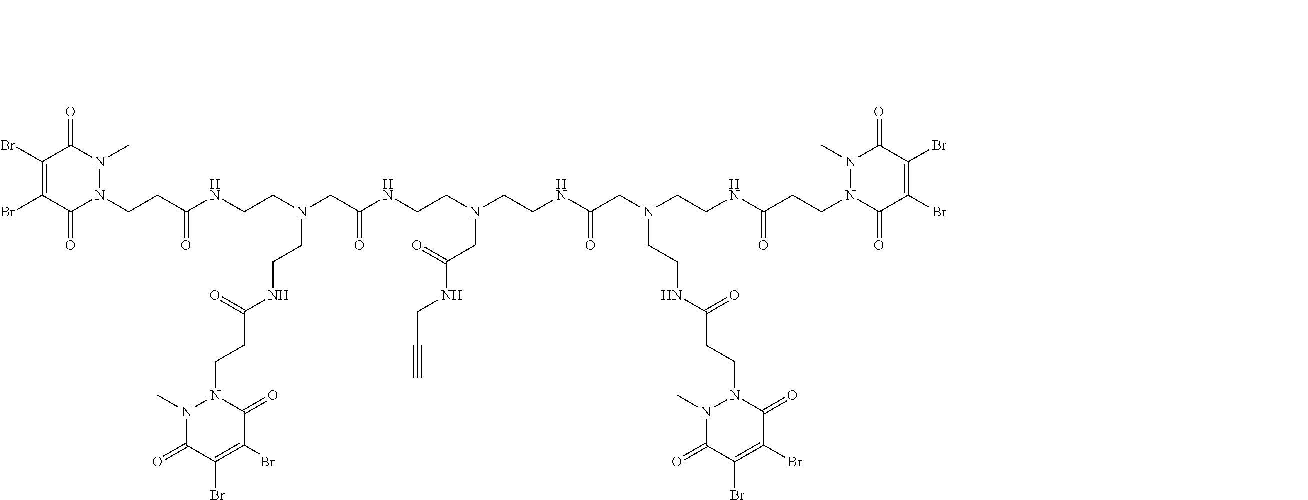 [SMILES: C#CCNC(=O)CN(CCNC(=O)CN(CCNC(=O)CCn1c(=O)c(Br)c(Br)c(=O)n1C)CCNC(=O)CCn1c(=O)c(Br)c(Br)c(=O)n1C)CCNC(=O)CN(CCNC(=O)CCn1c(=O)c(Br)c(Br)c(=O)n1C)CCNC(=O)CCn1c(=O)c(Br)c(Br)c(=O)n1C]

In some embodiments, the compound is selected from one of the following:

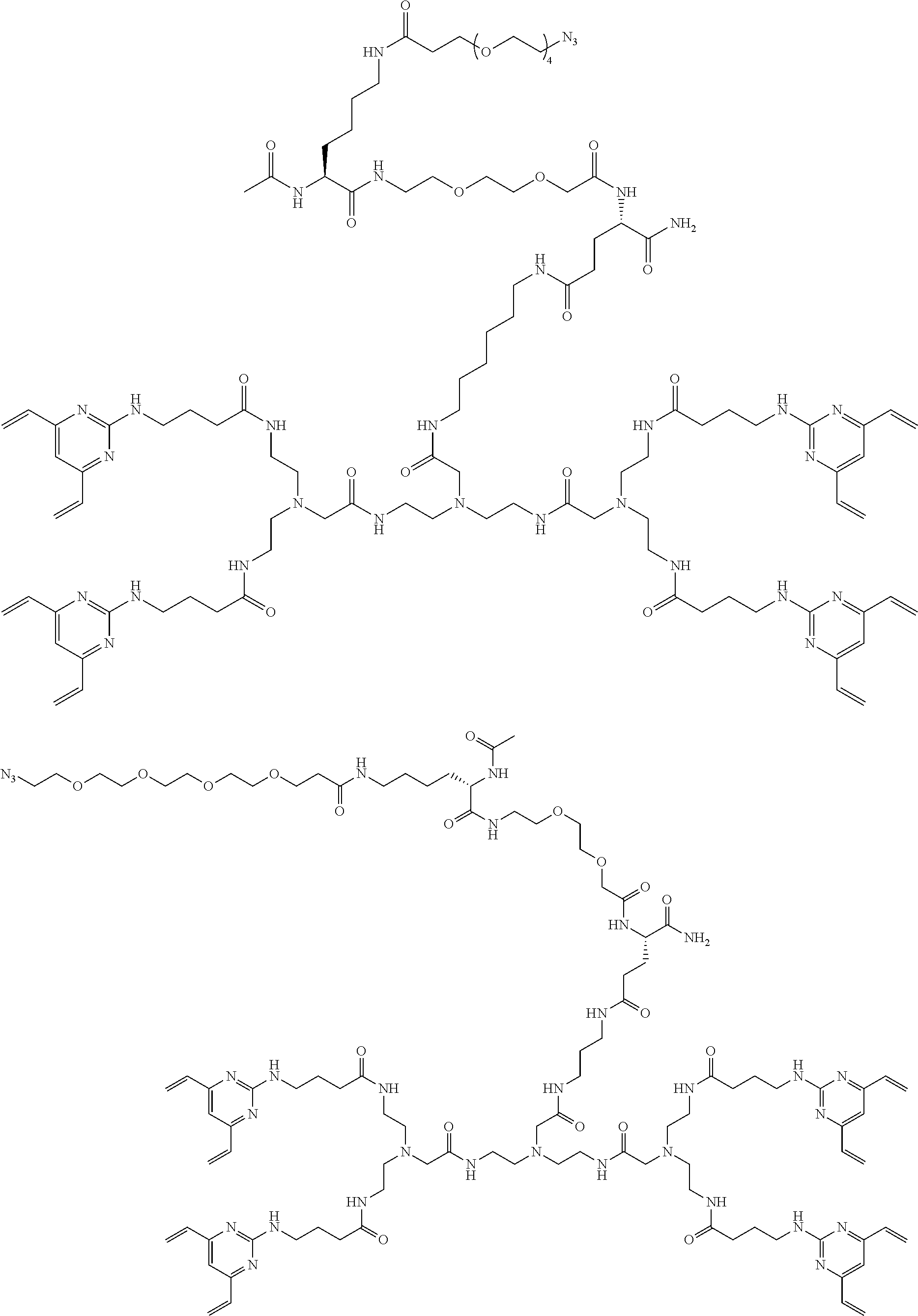

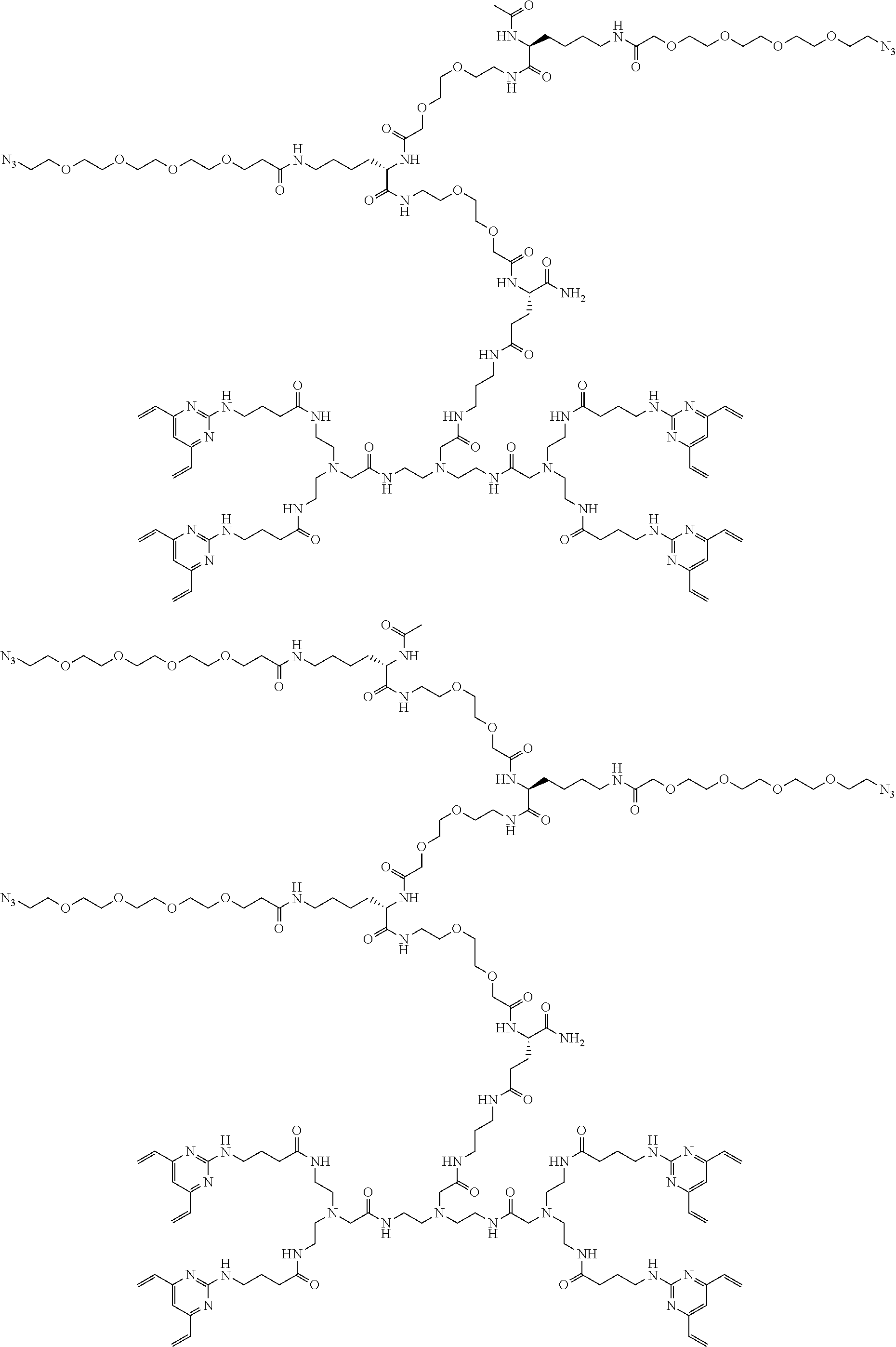

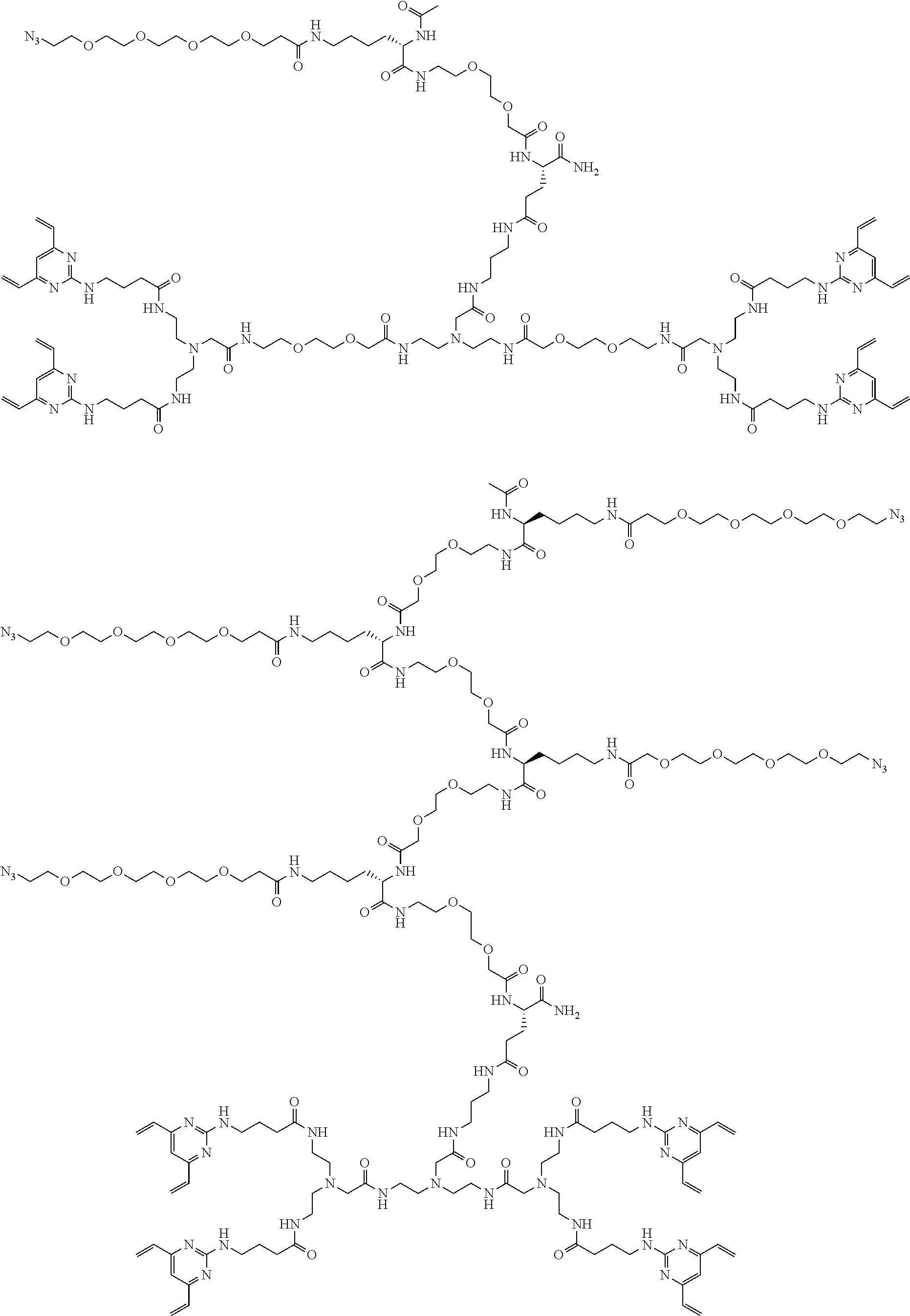

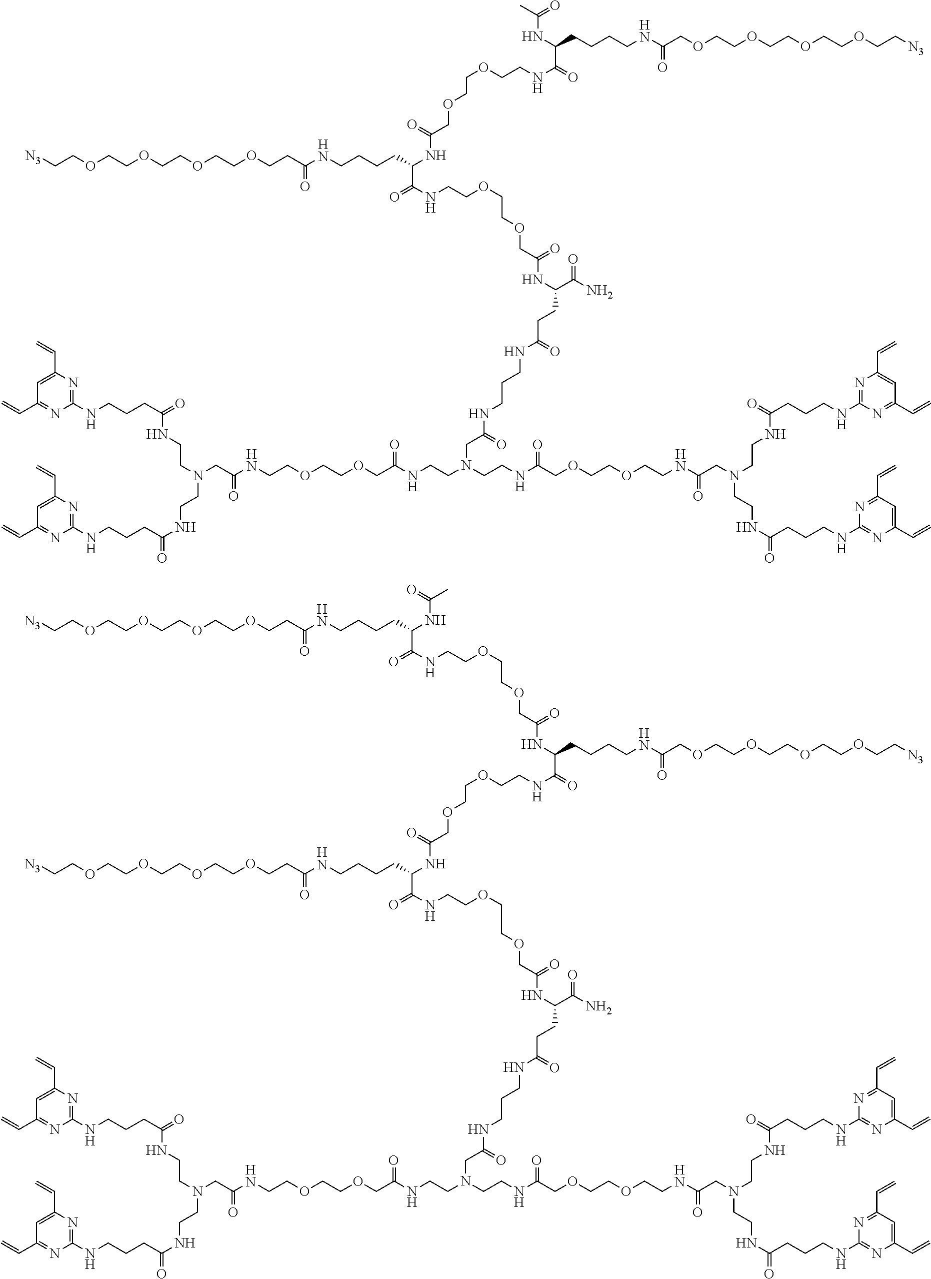

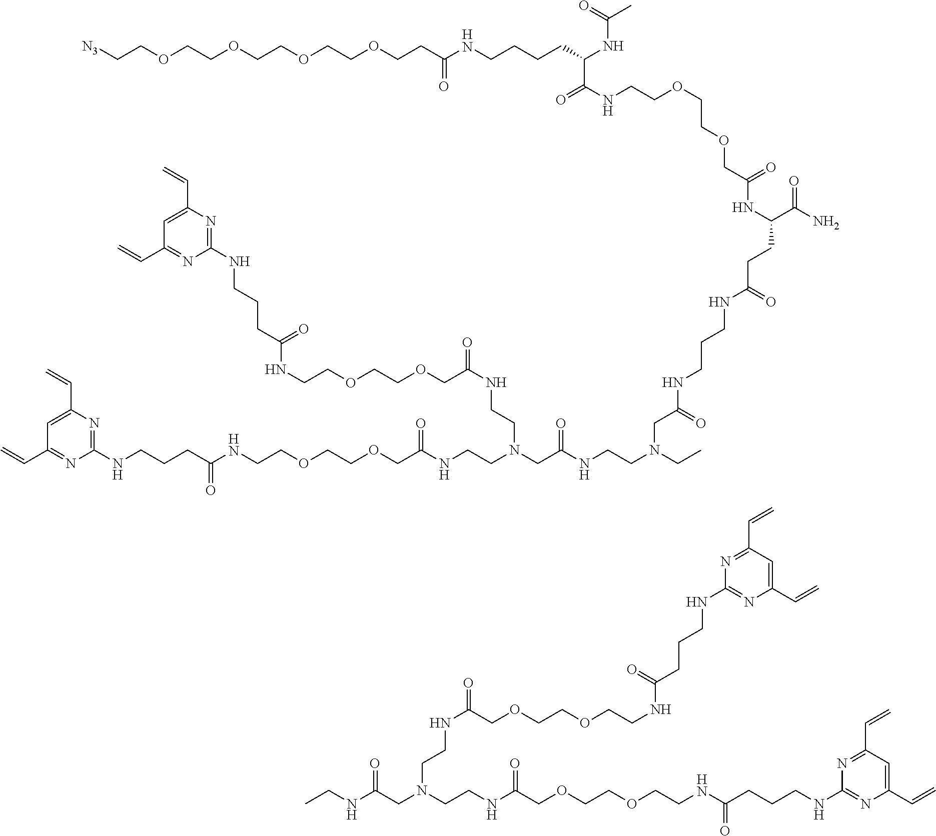 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCN(CC)CC(=O)NCCCNC(=O)CC[C@H](NC(=O)COCCOCCNC(=O)[C@H](CCCCNC(=O)CCOCCOCCOCCOCCN=[N+]=[N-])NC(C)=O)C(N)=O)n1 ; C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCC)n1]

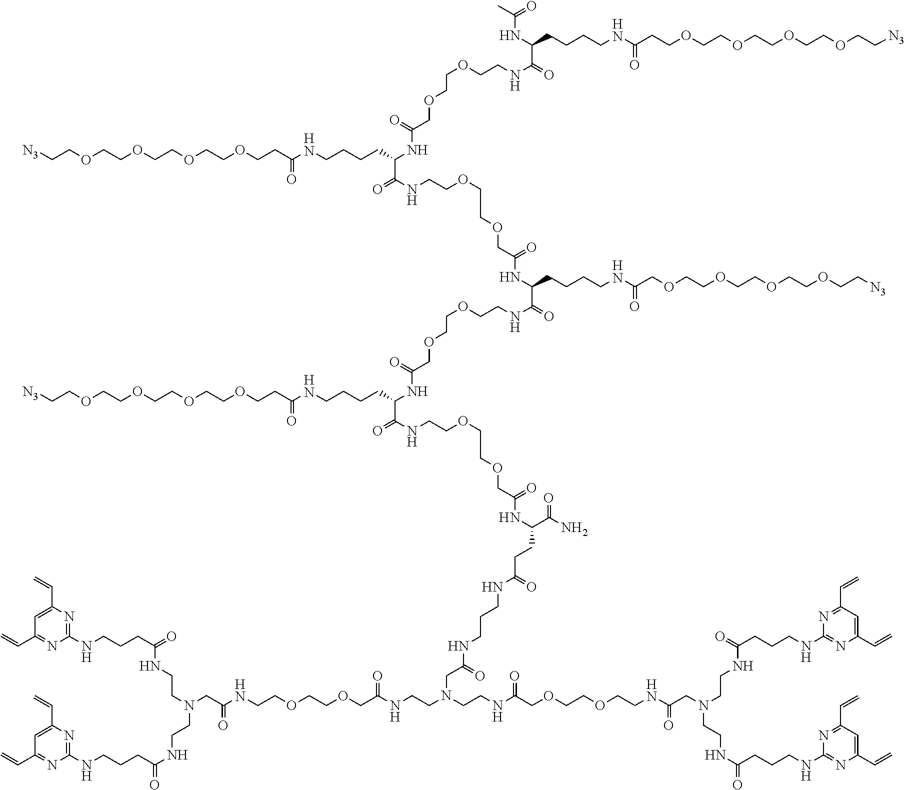 [SMILES: C=Cc1cc(C=C)nc(NCCCC(=O)NCCN(CCNC(=O)CCCNc2nc(C=C)cc(C=C)n2)CC(=O)NCCOCCOCC(=O)NCCN(CCNC(=O)COCCOCCNC(=O)CN(CCNC(=O)CCCNc2nc(C=C)cc(C=C)n2)CCNC(=O)CCCNc2nc(C=C)cc(C=C)n2)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)COCCOCCOCCOCCN=[N+]=[N-])NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(C)=O)C(N)=O)n1]

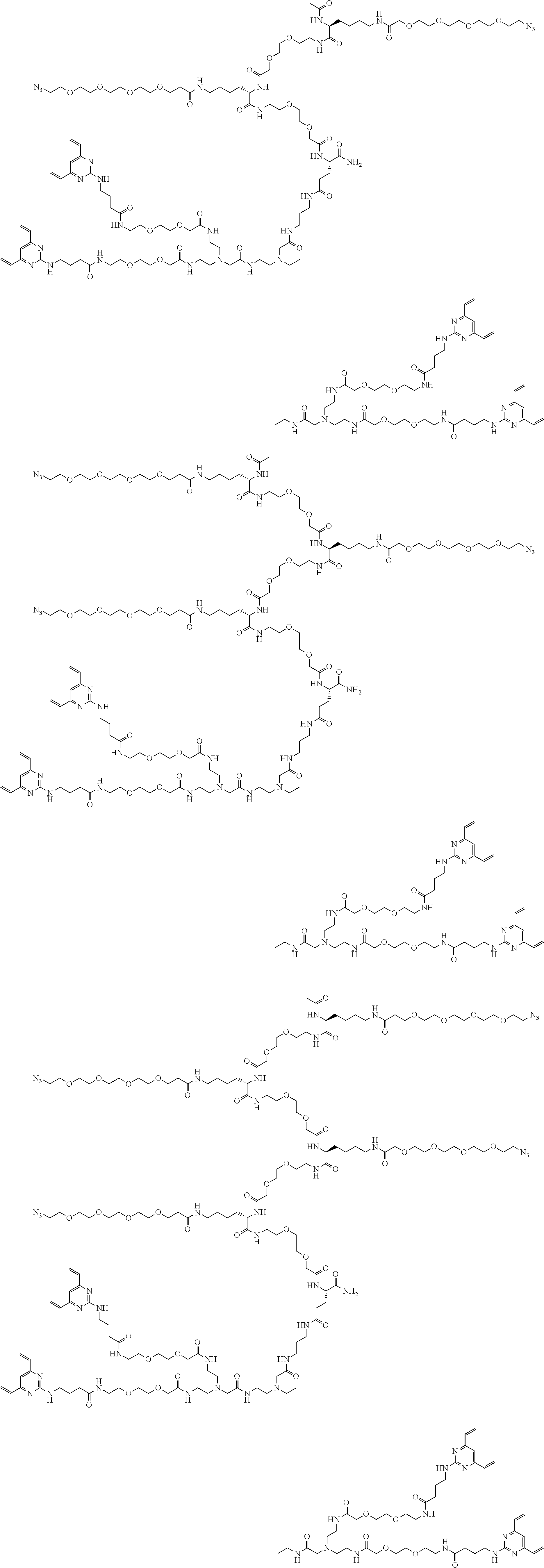

In some embodiments, the compound is selected from one of the following:

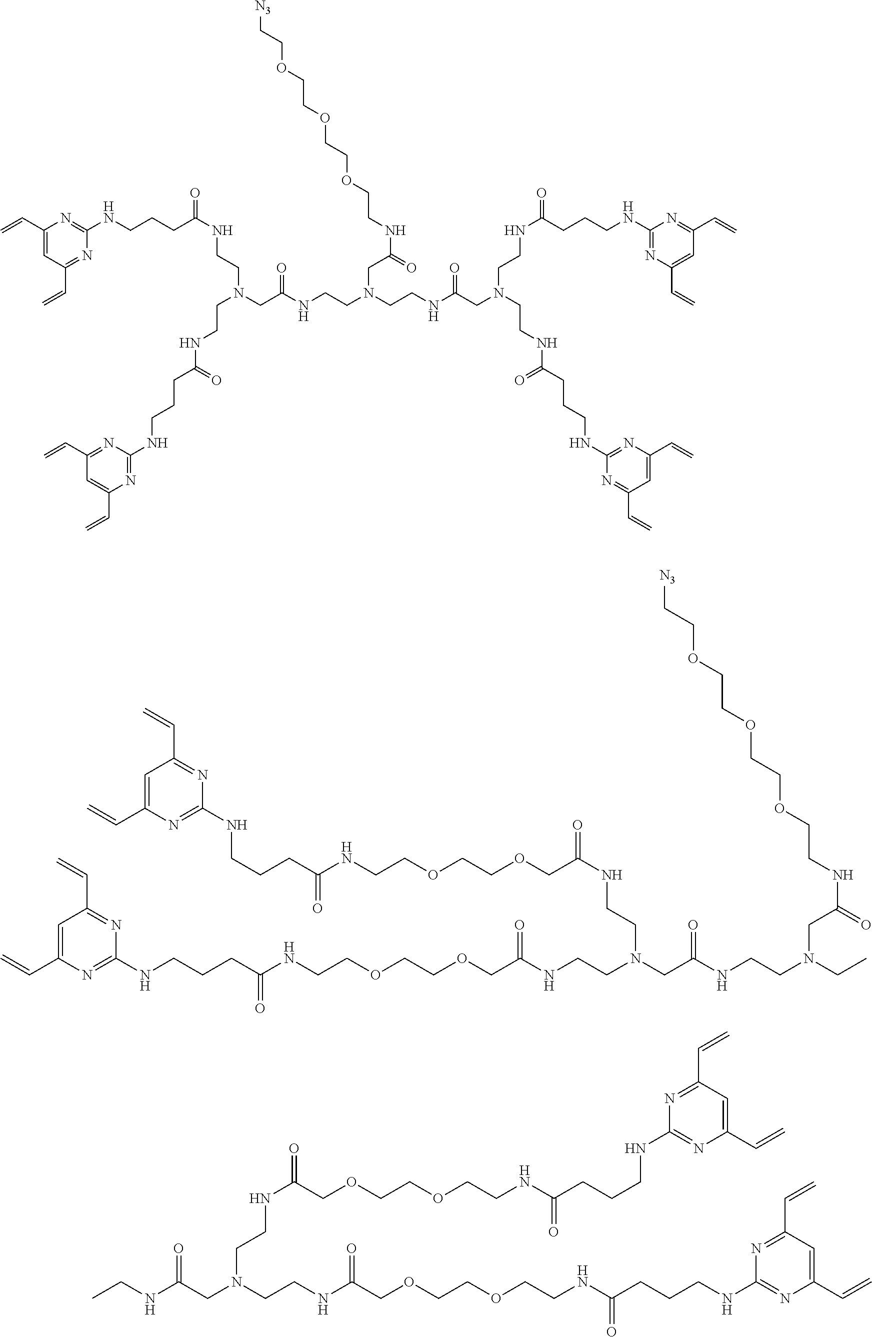

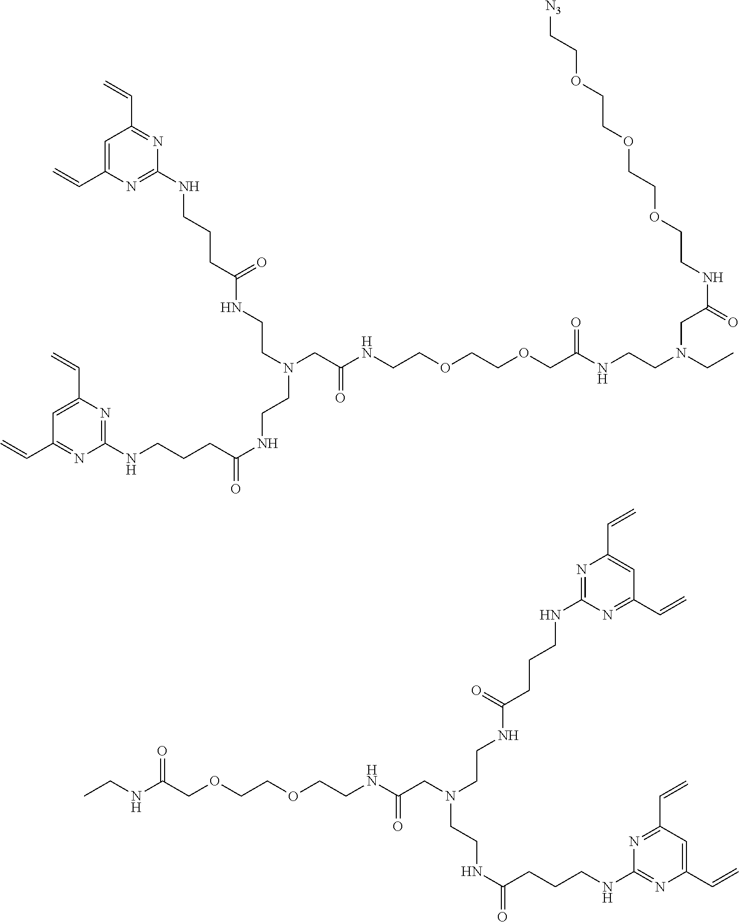 [SMILES: C=Cc1cc(C=C)nc(NCCCC(=O)NCCN(CCNC(=O)CCCNc2nc(C=C)cc(C=C)n2)CC(=O)NCCOCCOCC(=O)NCCN(CC)CC(=O)NCCOCCOCCOCCN=[N+]=[N-])n1 ; C=Cc1cc(C=C)nc(NCCCC(=O)NCCN(CCNC(=O)CCCNc2nc(C=C)cc(C=C)n2)CC(=O)NCCOCCOCC(=O)NCC)n1]

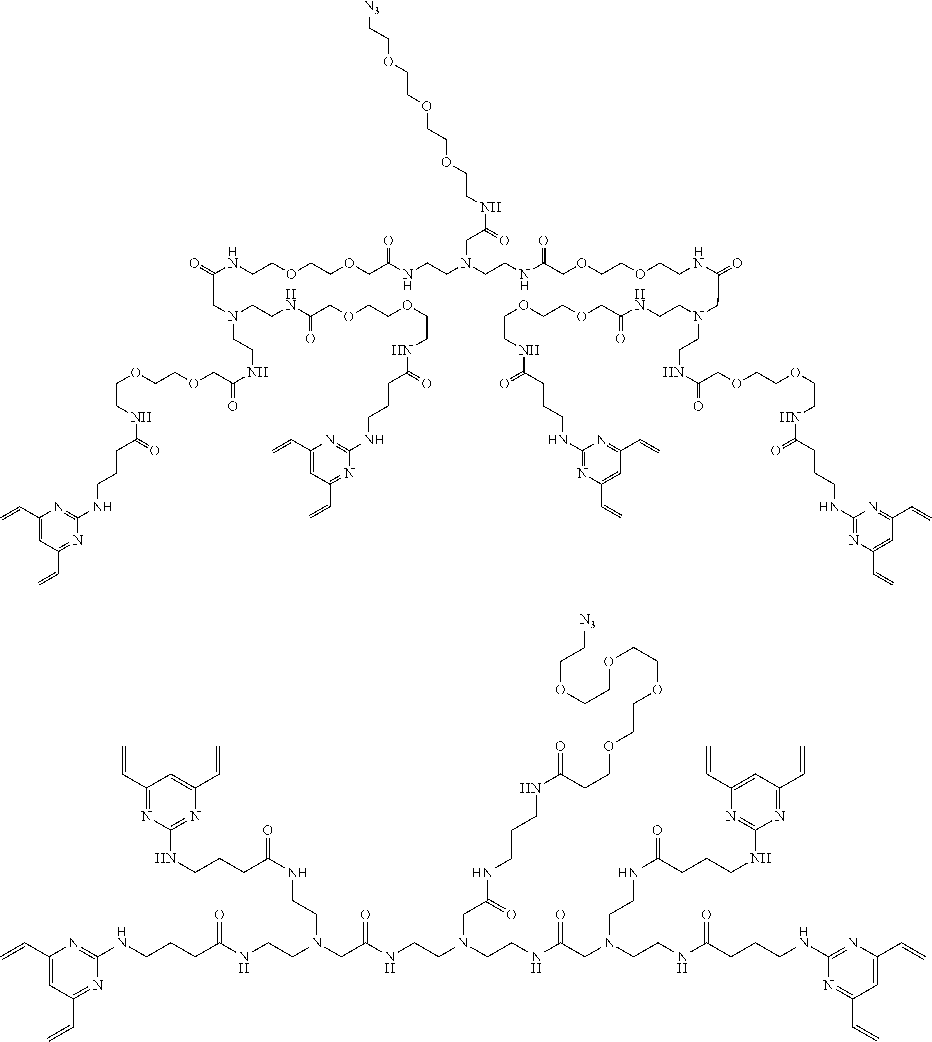

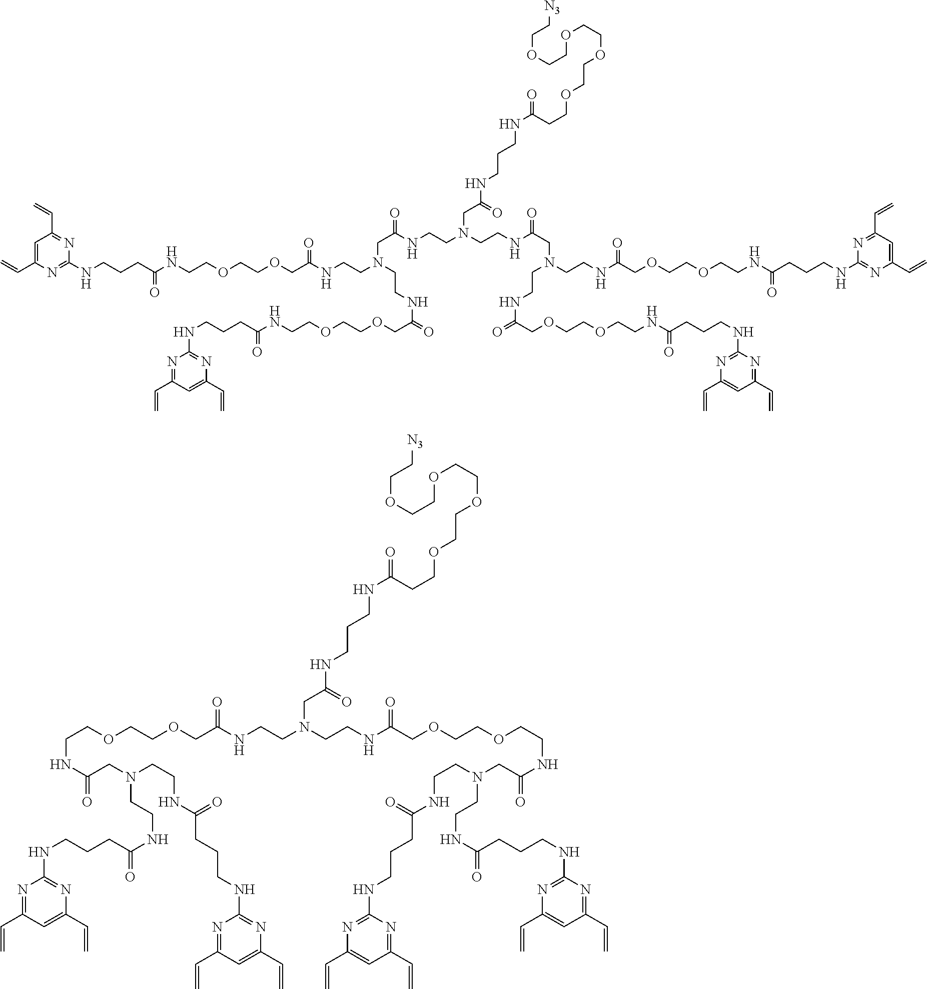 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCN(CCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCCNC(=O)CCOCCOCCOCCOCCN=[N+]=[N-])n1 ; C=Cc1cc(C=C)nc(NCCCC(=O)NCCN(CCNC(=O)CCCNc2nc(C=C)cc(C=C)n2)CC(=O)NCCOCCOCC(=O)NCCN(CCNC(=O)COCCOCCNC(=O)CN(CCNC(=O)CCCNc2nc(C=C)cc(C=C)n2)CCNC(=O)CCCNc2nc(C=C)cc(C=C)n2)CC(=O)NCCCNC(=O)CCOCCOCCOCCOCCN=[N+]=[N-])n1]

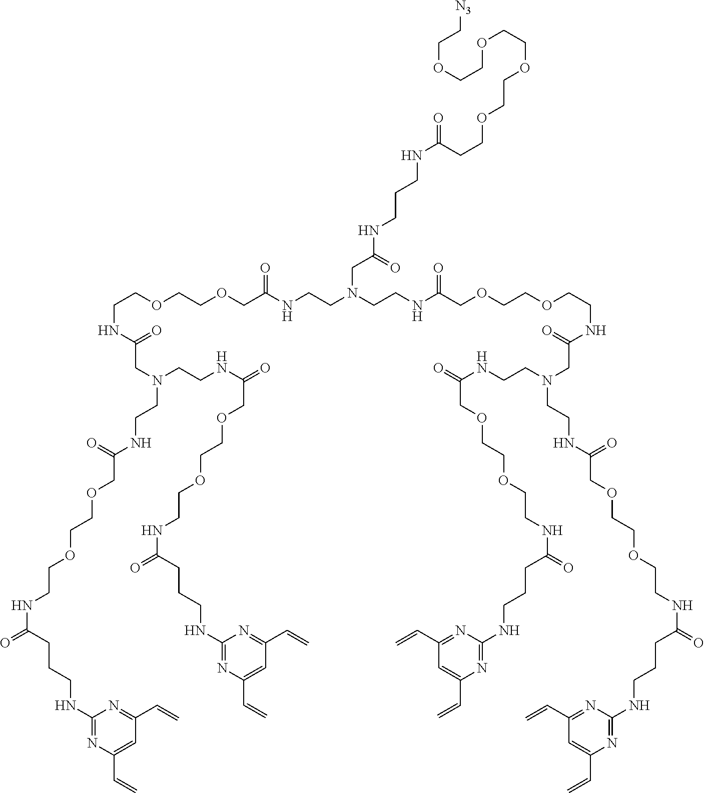 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCOCCOCC(=O)NCCN(CCNC(=O)COCCOCCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCCNC(=O)CCOCCOCCOCCOCCN=[N+]=[N-])n1]

### Intermediate Conjugates of the Invention

According to another aspect of the invention there is provided an intermediate conjugate comprising an antibody, a linker and at least one functional group capable reacting with another moiety to form a functional linking moiety, wherein:
i) the linker connects the at least one functional group to the antibody;
ii) the linker is attached to the antibody through 5 to 8 independent covalent bonds; and
iii) each covalent bond between the linker and the antibody is formed from the reaction between a sulfur atom on the antibody and a functional group on the linker.

In certain embodiments, the intermediate conjugate comprises between one and four (preferably between two and four, more preferably between three and four, and most preferably four) re-bridging moieties of Formula (III), shown below:

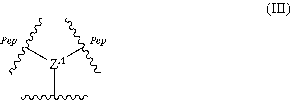

wherein:
   Z^{A} is a functional linking group as defined herein; Pep indicates the position where the moiety is attached to the antibody, either directly or indirectly; and

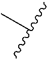

indicates the position where the moiety is attached to linker.

Preferred and suitable ZA groups will be understood to be analogous to the preferred and suitable Z^{A} groups for the conjugates of the invention described hereinabove.

In other embodiments, the intermediate conjugate is a compound of Formula (VI), shown below:

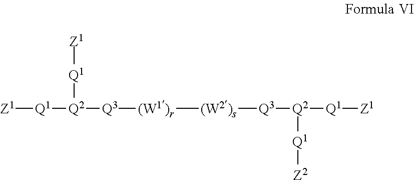

wherein, Z¹, Q¹, Q², Q³, W^{1′}, W^{2′}, r and s are as defined hereinabove.

In other embodiments, the intermediate conjugate is selected from one of the following:

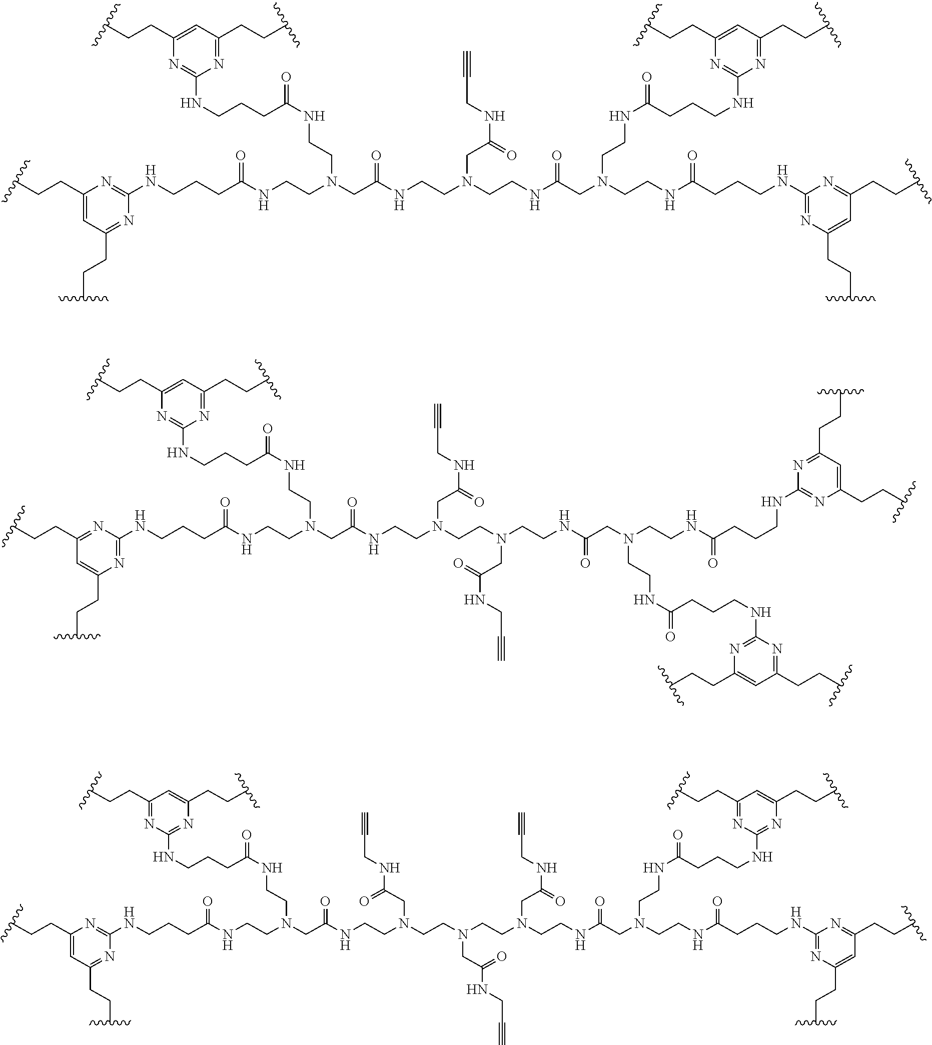

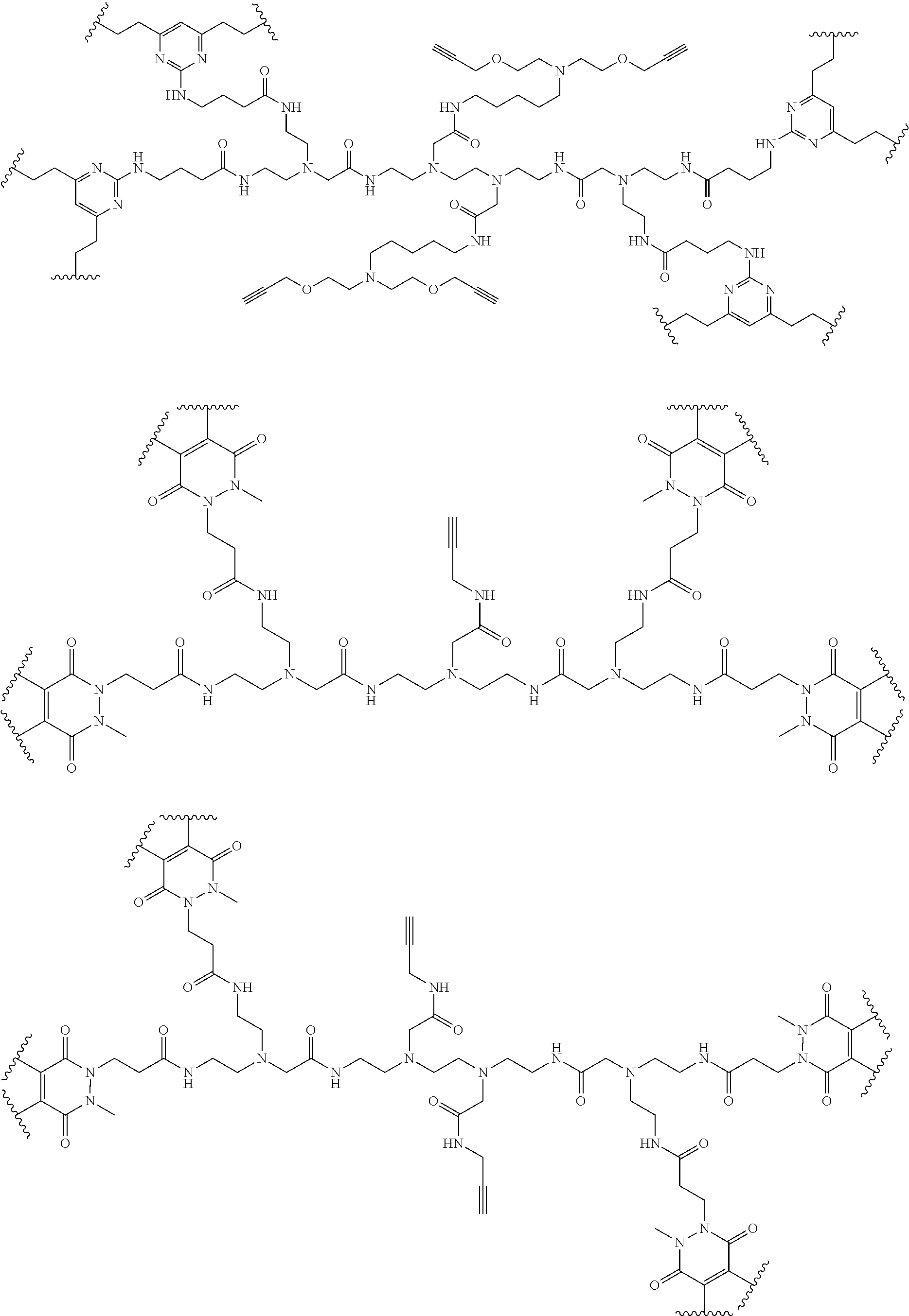

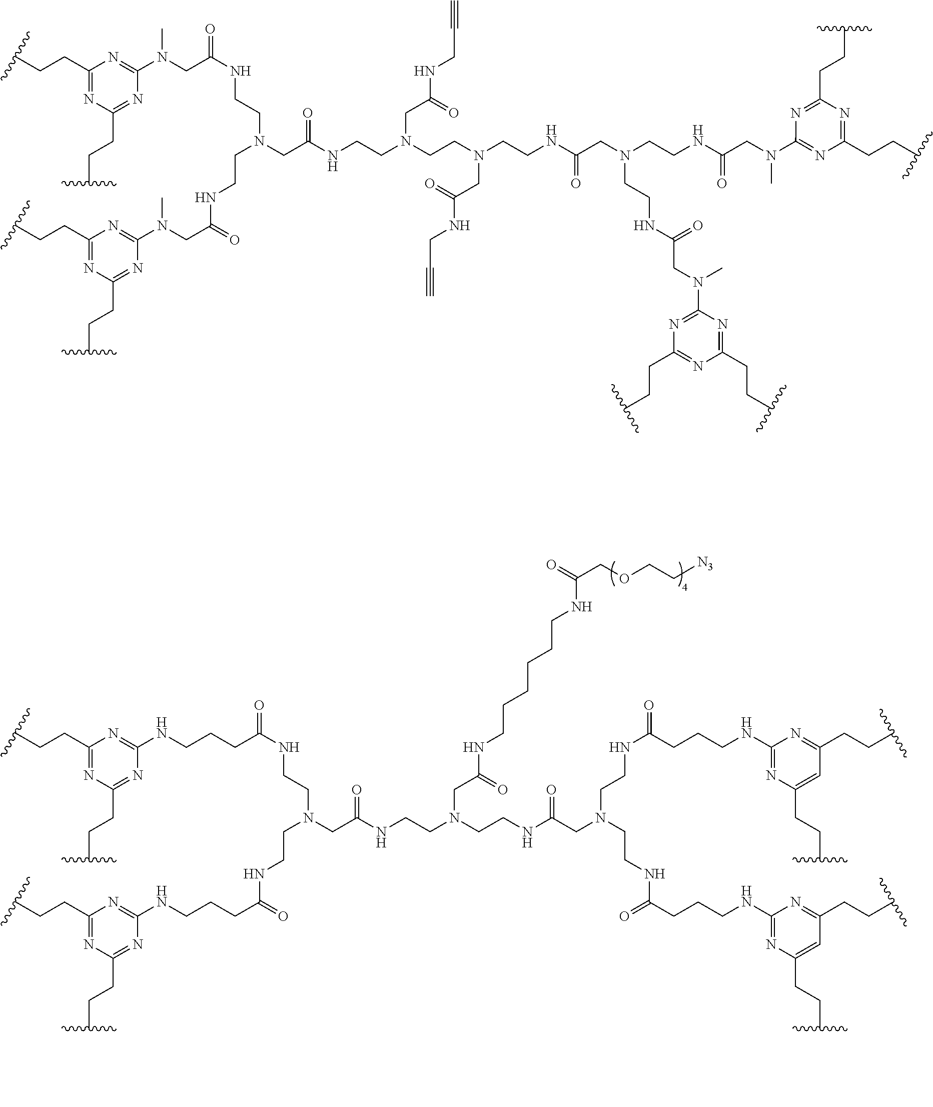

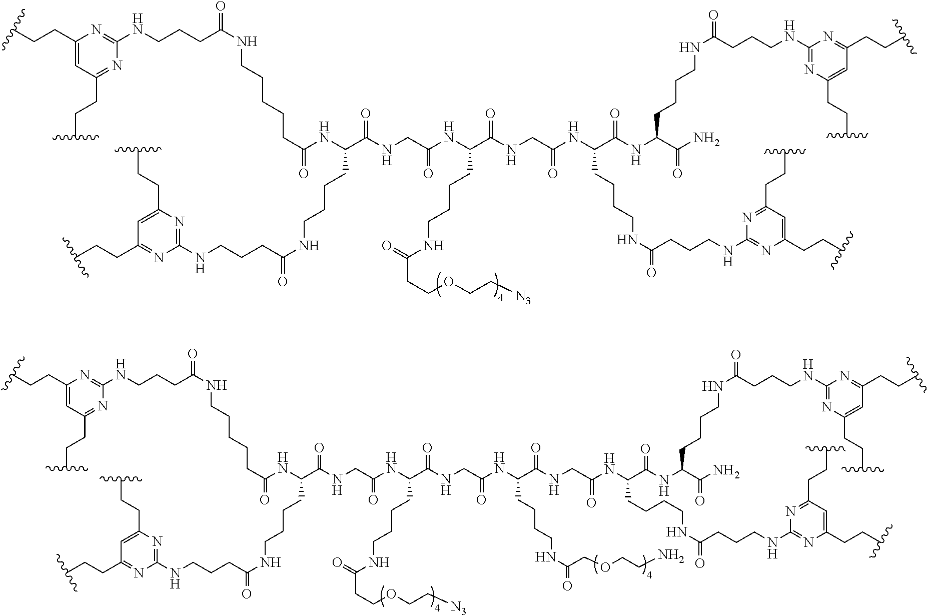

wherein:

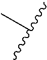

indicates the position where the antibody is attached; and
the antibody is preferably trastuzumab or brentuximab, most preferably trastuzumab.

In other embodiments, the intermediate conjugate is selected from one of the following:

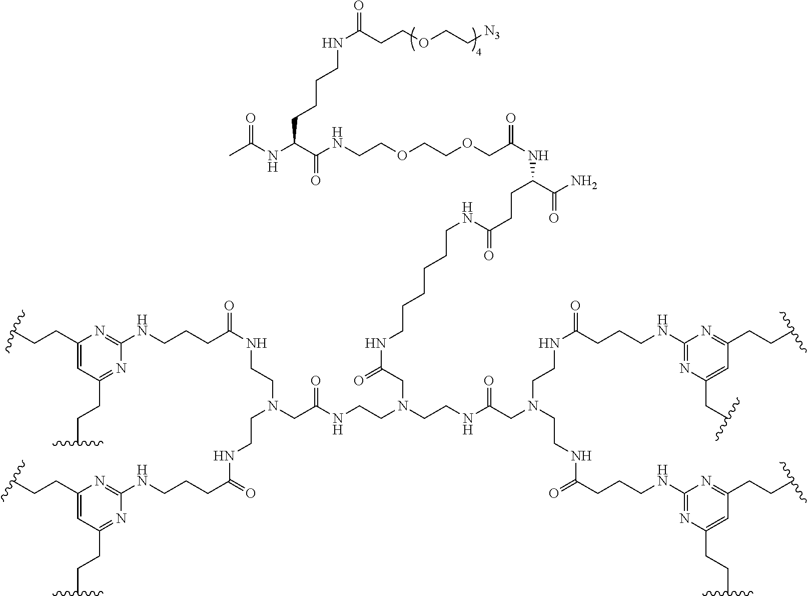

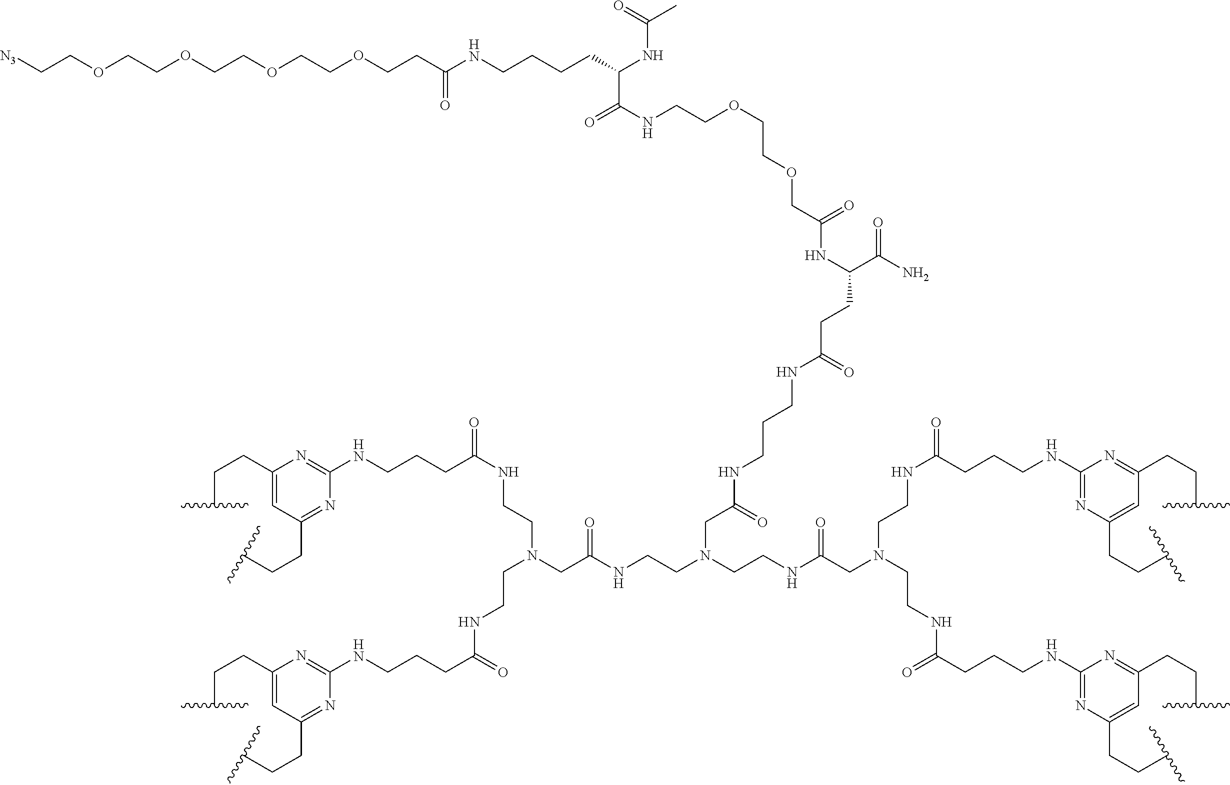 [SMILES: *CCc1cc(CC*)nc(NCCCC(=O)NCCN(CCNC(=O)CCCNc2nc(CC*)cc(CC*)n2)CC(=O)NCCN(CCNC(=O)CN(CCNC(=O)CCCNc2nc(CC*)cc(CC*)n2)CCNC(=O)CCCNc2nc(CC*)cc(CC*)n2)CC(=O)NCCCNC(=O)CC[C@H](NC(=O)COCCOCCNC(=O)[C@H](CCCCNC(=O)CCOCCOCCOCCOCCN=[N+]=[N-])NC(C)=O)C(N)=O)n1]

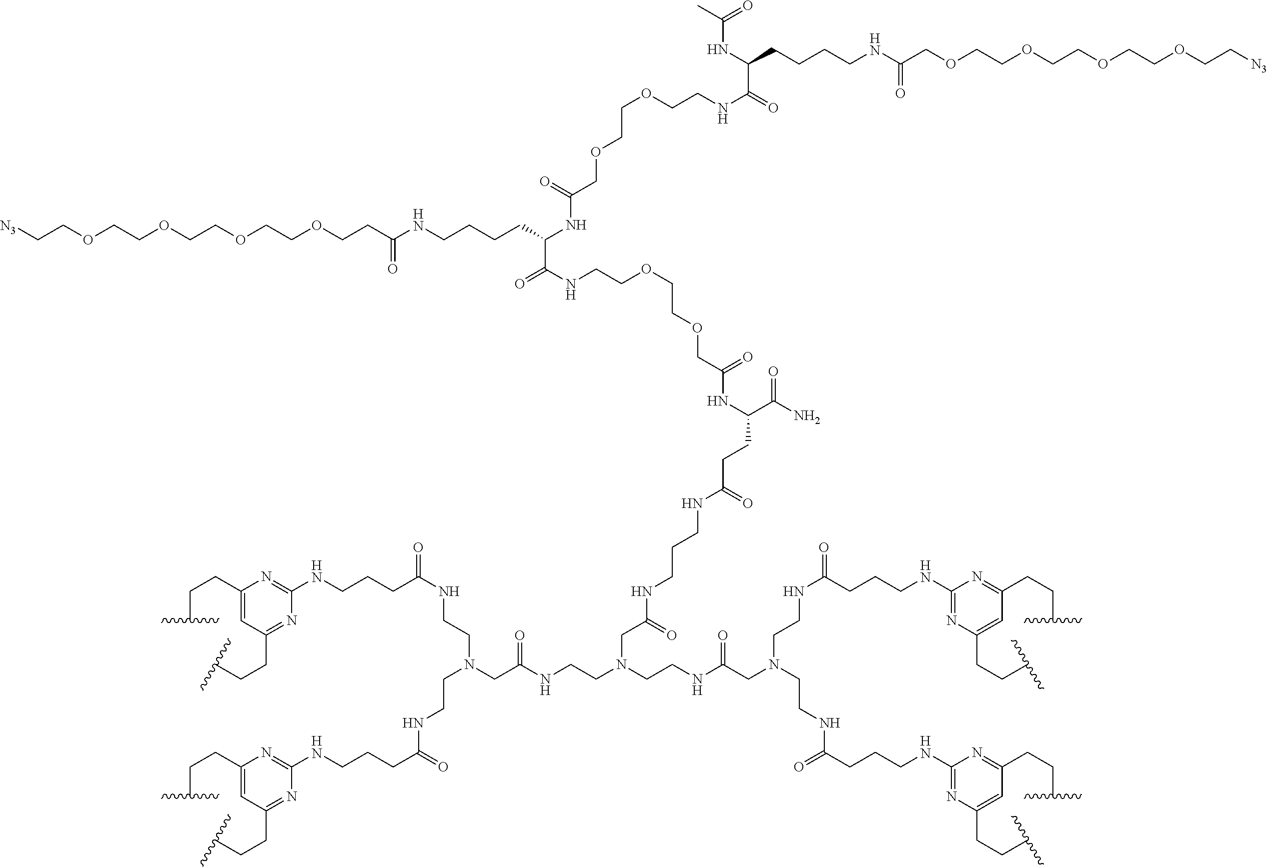 [SMILES: *CCc1cc(CC*)nc(NCCCC(=O)NCCN(CCNC(=O)CCCNc2nc(CC*)cc(CC*)n2)CC(=O)NCCN(CCNC(=O)CN(CCNC(=O)CCCNc2nc(CC*)cc(CC*)n2)CCNC(=O)CCCNc2nc(CC*)cc(CC*)n2)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)COCCOCCOCCOCCN=[N+]=[N-])NC(C)=O)C(N)=O)n1]

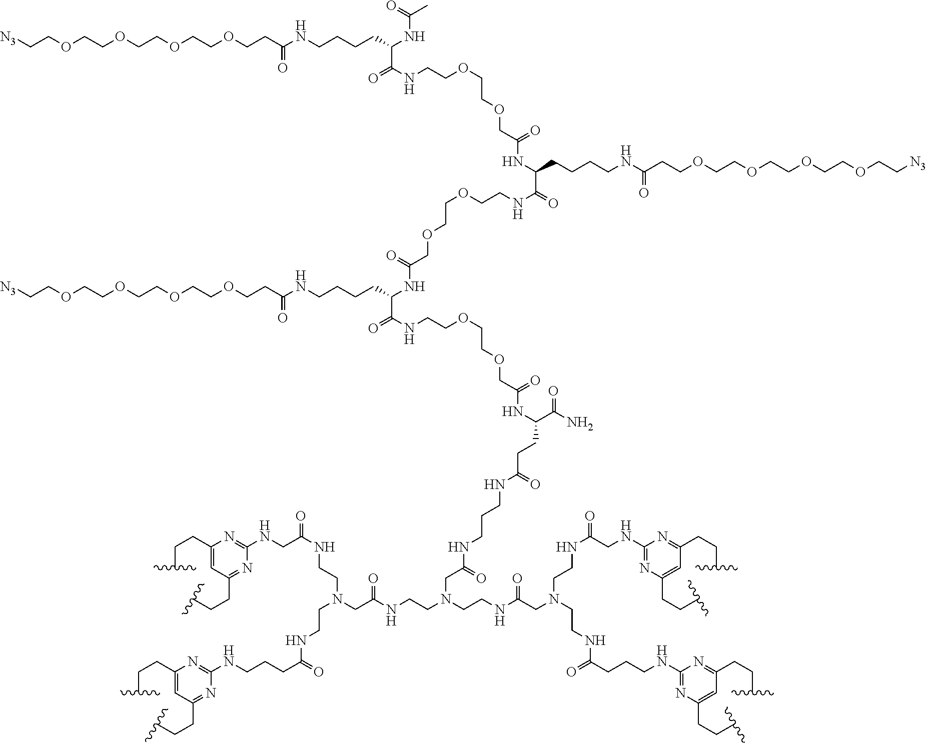 [SMILES: *CCc1cc(CC*)nc(NCCCC(=O)NCCN(CCNC(=O)CNc2nc(CC*)cc(CC*)n2)CC(=O)NCCN(CCNC(=O)CN(CCNC(=O)CCCNc2nc(CC*)cc(CC*)n2)CCNC(=O)CNc2nc(CC*)cc(CC*)n2)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(C)=O)C(N)=O)n1]

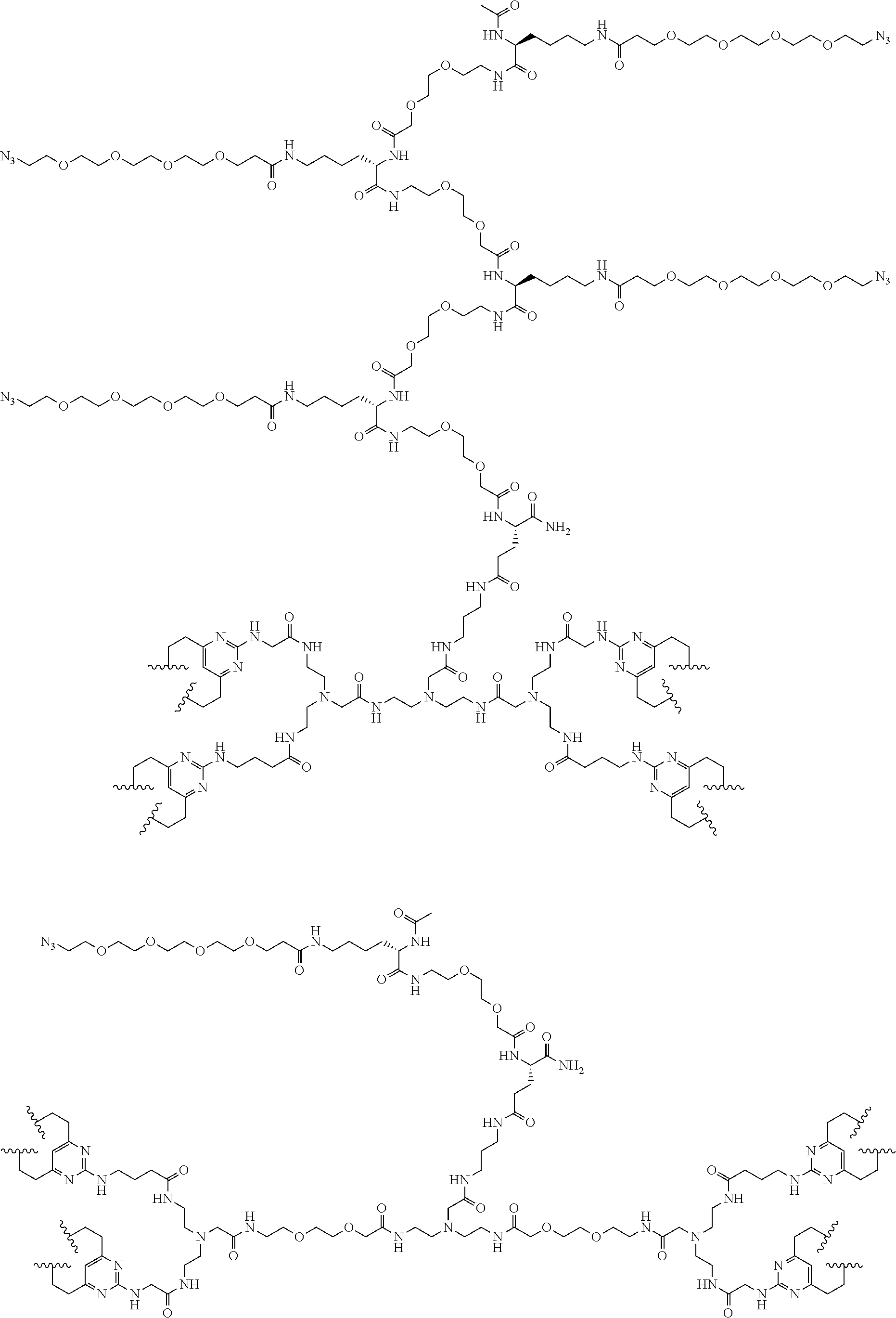

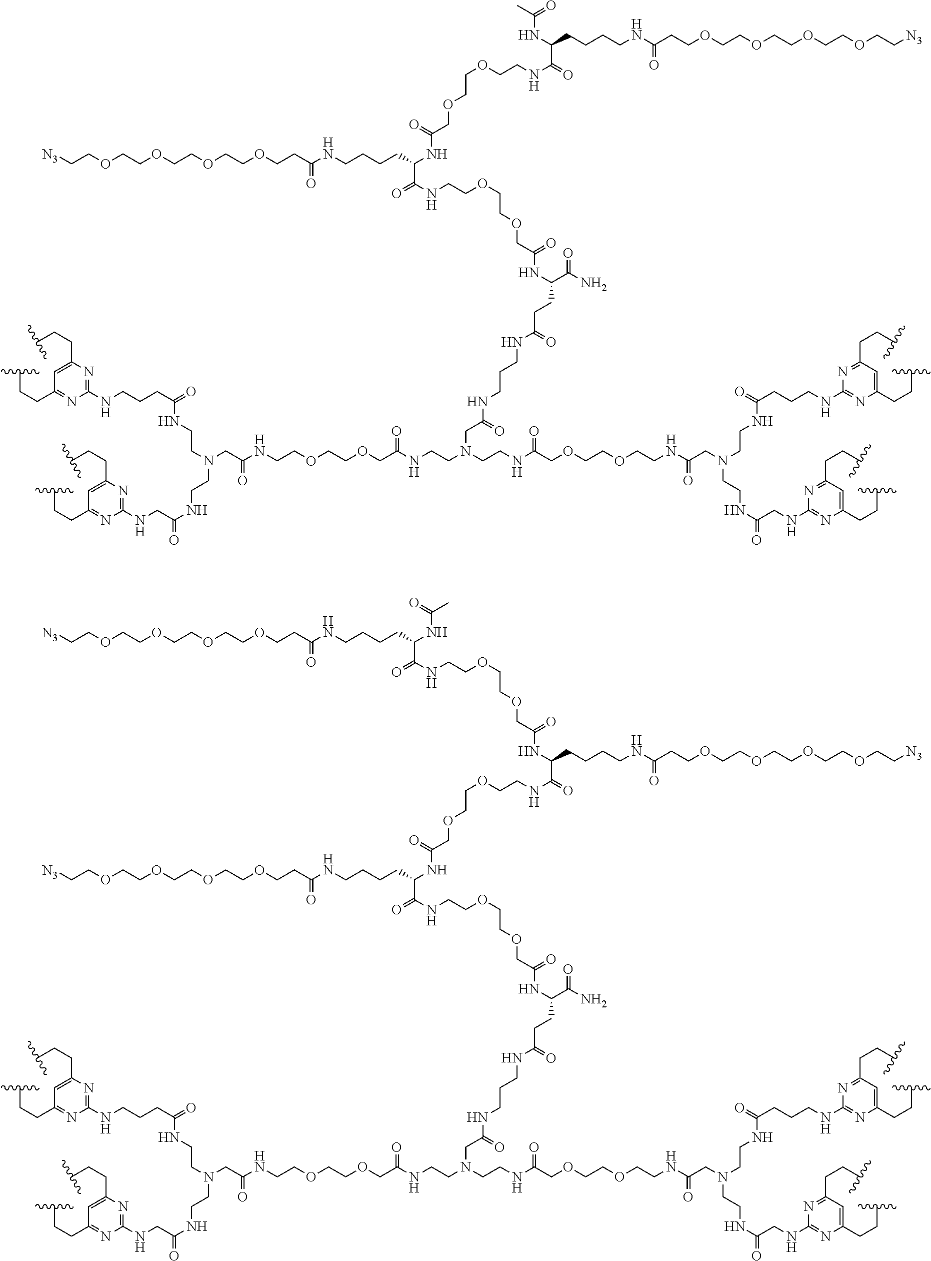

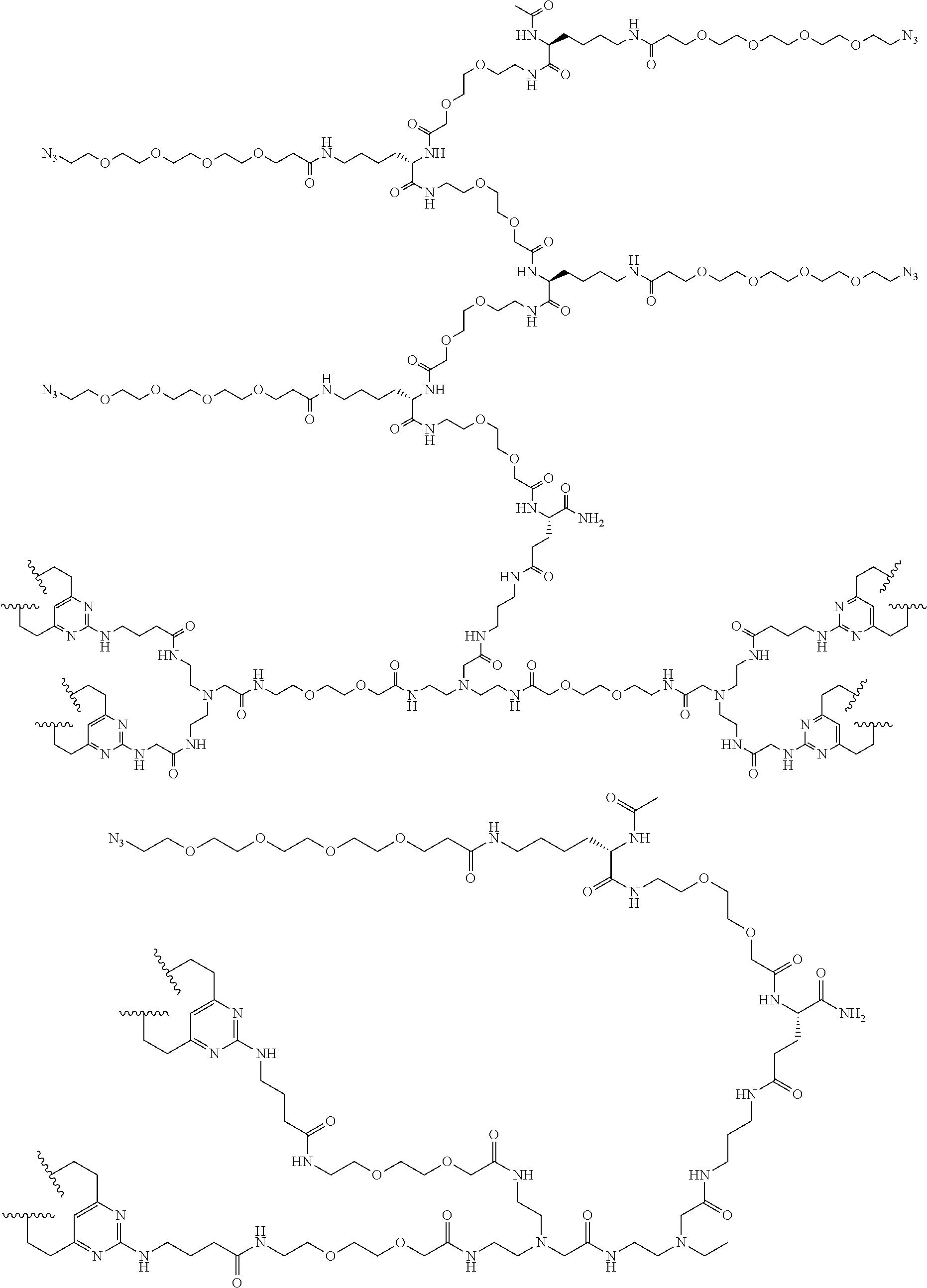

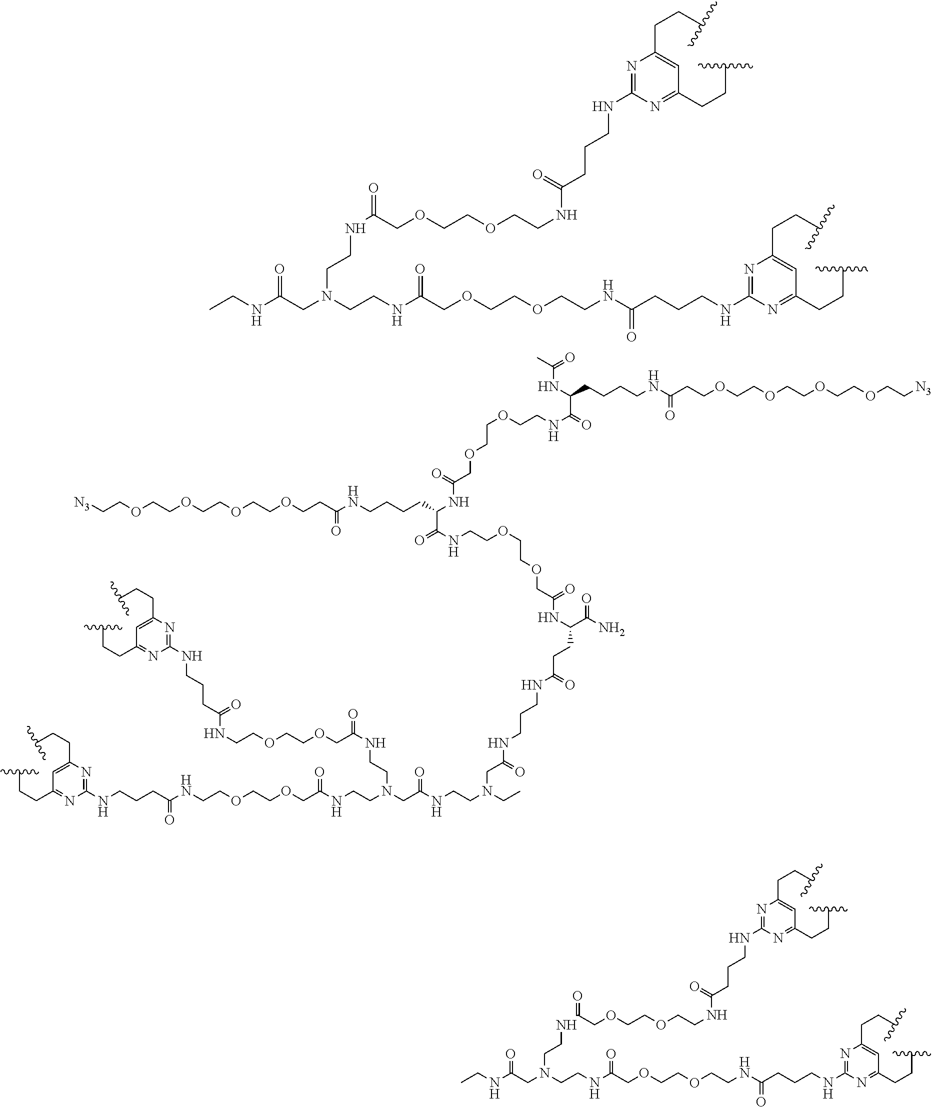 [SMILES: *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCC)n1 ; *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCCN(CC)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(C)=O)C(N)=O)n1 ; *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCC)n1]

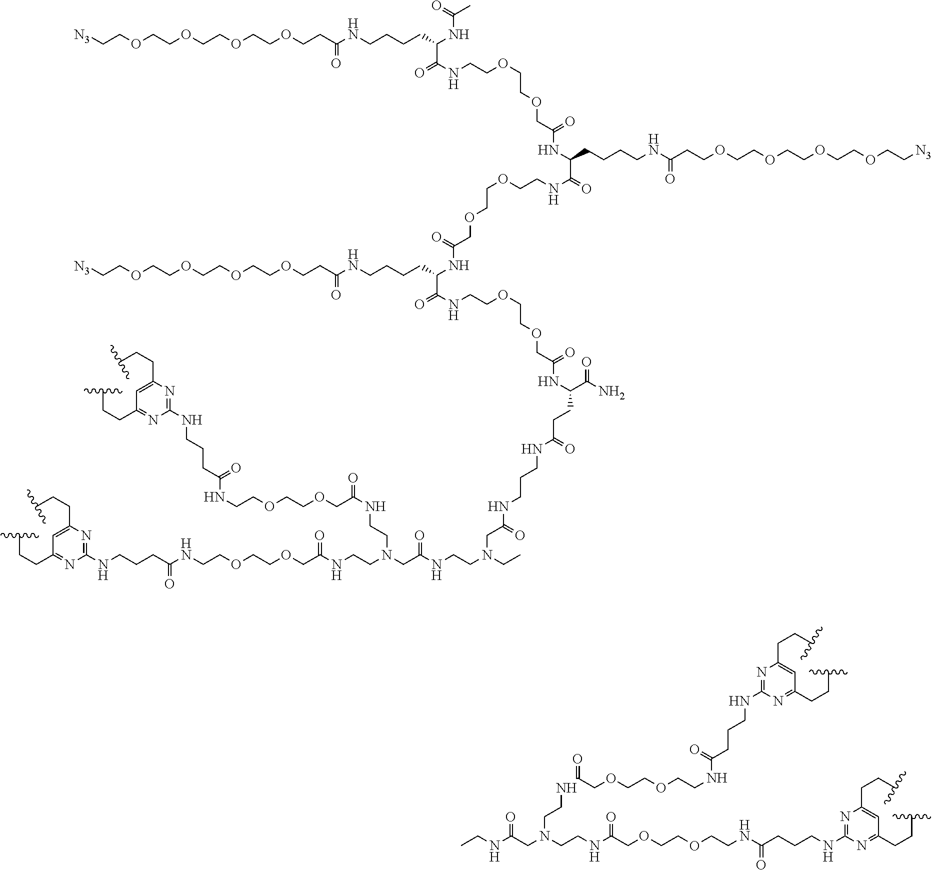 [SMILES: *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCCN(CC)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(C)=O)C(N)=O)n1 ; *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCC)n1]

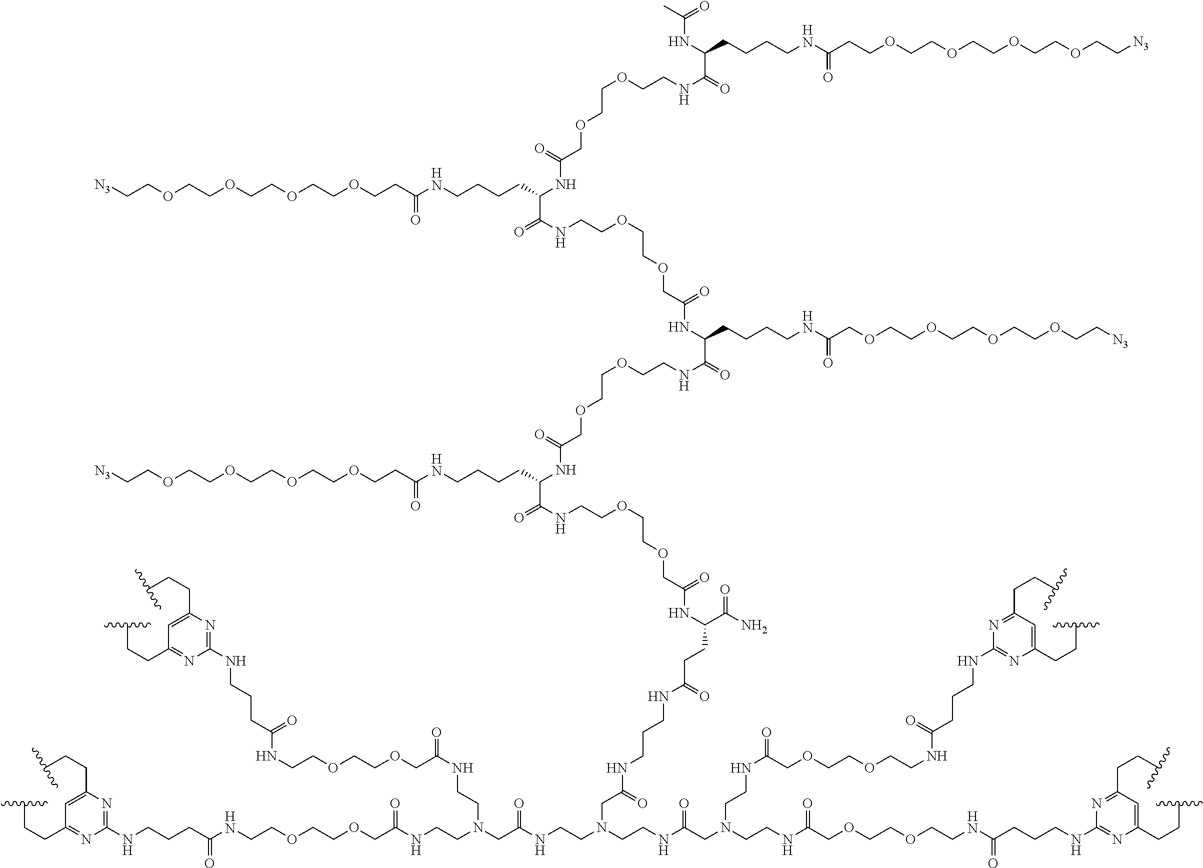 [SMILES: *CCc1cc(CC*)nc(NCCCC(=O)NCCOCCOCC(=O)NCCN(CCNC(=O)COCCOCCNC(=O)CCCNc2nc(CC*)cc(CC*)n2)CC(=O)NCCN(CCNC(=O)CN(CCNC(=O)COCCOCCNC(=O)CCCNc2nc(CC*)cc(CC*)n2)CCNC(=O)COCCOCCNC(=O)CCCNc2nc(CC*)cc(CC*)n2)CC(=O)NCCCNC(=O)CC[C@H](NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)COCCOCCOCCOCCN=[N+]=[N-])NC(=O)COCCOCCNC(=O)[C@H](CCCCNC(=O)CCOCCOCCOCCOCCN=[N+]=[N-])NC(=O)COCCOCCNC(=O)[C@H](CCCCNC(=O)CCOCCOCCOCCOCCN=[N+]=[N-])NC(C)=O)C(N)=O)n1]

wherein:

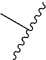

indicates the position where the antibody is attached; and
the antibody is preferably trastuzumab or brentuximab, most preferably trastuzumab.

In other embodiments, the intermediate conjugate is selected from ALC 1 to 12 described below.

### Methods of Treatment

The conjugates of the present invention where the active agent is a drug may be used in a method of therapy. Also provided is a method of treatment, comprising administering to a subject in need of treatment a therapeutically-effective amount of a conjugate according to the first aspect of the invention, where the active agent is a drug. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate according to the first aspect of the invention, where the active agent is a drug, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin. Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmune disorder is a T cell-mediated immunological disorder.

In certain embodiments, the conjugates can be used to treat microbial diseases. Suitably, the conjugates can be used to treat bacterial diseases, such tuberculosis (TB) and methicillin-resistant Staphylococcus aureus (MRSA).

It will also be understood that features, including optional, suitable, and preferred features in relation to any one of the aspects of the present invention detailed above (e.g. conjugates of the present invention) may also be features, including optional, suitable and preferred features in relation to any other aspects of the invention (e.g. the uses of the conjugates of the present invention).

### BRIEF DESCRIPTION OF THE FIGURES

FIG. 1 shows: Analysis of conjugates by SDS-PAGE; (A) Analysis of trastuzumab conjugates 36-39; (B) Analysis of trastuzumab conjugates 40-46; (C) Analysis of brentuximab conjugates 47-50; M=molecular weight marker.

FIG. 2 shows the LC-MS trace from the reaction between trastuzumab and compound 1 to produce conjugate 36, where: A) shows the non-deconvoluted mass spectrometry trace; and B) shows the deconvoluted mass spectrometry trace. Expected MW=146,536 Da, observed MW=146,537 Da.

FIG. 3 shows the LC-MS trace from the reaction between trastuzumab and compound 8 to produce conjugate 37, where: A) shows the non-deconvoluted mass spectrometry trace; and B) shows the deconvoluted mass spectrometry trace. Expected MW=146,674 Da, observed MW=146,615 Da.

FIG. 4 shows the LC-MS trace from the reaction between trastuzumab and compound 11 to produce conjugate 38, where: A) shows the non-deconvoluted mass spectrometry trace; and B) shows the deconvoluted mass spectrometry trace. Expected MW=146,812 Da, observed MW=146,749 Da.

FIG. 5 shows the LCMS trace from the reaction between trastuzumab and compound 14 to produce conjugate 39, where: A) shows the non-deconvoluted mass spectrometry trace; and B) shows the deconvoluted mass spectrometry trace. Expected MW=147,096 Da, observed 147,107 Da.

FIG. 6 shows the LC-MS trace from the reaction between trastuzumab and compound 19 to produce conjugate 40, where: A) shows the non-deconvoluted mass spectrometry trace; and B) shows the deconvoluted mass spectrometry trace. Expected MW=146,369 Da, observed MW=146,369 Da.

FIG. 7 shows the LC-MS trace from the reaction between trastuzumab and compound 21 to produce conjugate 41, where: A) shows the non-deconvoluted mass spectrometry trace; and B) shows the deconvoluted mass spectrometry trace. Expected MW=146,507 Da, observed MW=146,508 Da.

FIG. 8 shows the LC-MS trace from the reaction between trastuzumab and compound 22 to produce conjugate 42, where: A) shows the non-deconvoluted mass spectrometry trace; and B) shows the deconvoluted mass spectrometry trace. Expected MW=146,605 Da, observed MW=146,611 Da.

FIG. 9 shows the LC-MS trace from the reaction between trastuzumab and compound 24 to produce conjugate 43, where: A) shows the non-deconvoluted mass spectrometry trace; and B) shows the deconvoluted mass spectrometry trace. Expected MW=146,838 Da, observed MW=146,845 Da.

FIG. 10 shows the LC-MS trace from the reaction between trastuzumab and compound 28 to produce conjugate 44, where: A) shows the non-deconvoluted mass spectrometry trace; and B) shows the deconvoluted mass spectrometry trace. Expected MW=147,077 Da, observed MW=147,069 Da.

FIG. 11 shows the LC-MS trace from the reaction between trastuzumab and compound 31 to produce conjugate 45, where: A) shows the non-deconvoluted mass spectrometry trace; and B) shows the deconvoluted mass spectrometry trace. Expected MW=147,524 Da, observed MW=147,526 Da.

FIG. 12 shows the LC-MS trace from the reaction between trastuzumab and compound 33 to produce conjugate 46, where: A) shows the non-deconvoluted mass spectrometry trace; and B) shows the deconvoluted mass spectrometry trace. Expected MW=147,295 Da, observed MW=147,301 Da.

FIG. 13 shows the LC-MS trace from the reaction between brentuximab and compound 1 to produce conjugate 47, where: A) shows the non-deconvoluted mass spectrometry trace; and B) shows the deconvoluted mass spectrometry trace. Expected MW=146,549 Da, observed MW=146,550 Da.

FIG. 14 shows the LC-MS trace from the reaction between brentuximab and compound 8 to produce conjugate 48, where: A) shows the non-deconvoluted mass spectrometry trace; and B) shows the deconvoluted mass spectrometry trace. Expected MW=146,687 Da, observed MW=146,688 Da.

FIG. 15 shows the LC-MS trace from the reaction between brentuximab and compound 19 to produce conjugate 49, where: A) shows the non-deconvoluted mass spectrometry trace; and B) shows the deconvoluted mass spectrometry trace. Expected MW=146,393 Da, observed MW=146,395 Da.

FIG. 16 shows the LC-MS trace from the reaction between brentuximab and compound 21 to produce conjugate 50, where: A) shows the non-deconvoluted mass spectrometry trace; and B) shows the deconvoluted mass spectrometry trace. Expected MW=146,531 Da, observed MW=146,533 Da.

FIG. 17 shows (A) Size-exclusion chromatography (SEC) analysis of trastuzumab and trastuzumab conjugates 36-39; and (B) Binding affinity comparison of trastuzumab and conjugates 36-39 via enzyme-linked immunosorbent assay (ELISA). Error bars represent the standard deviation of biological quadruplicates.

FIG. 18 shows UV/vis spectra of conjugates 51-54. Absorbance was normalised at 280 nm. Absorbance at 495 nm was used to calculate the fluorophore-to-antibody ratio (FAR).

FIG. 19 shows stability analysis of conjugates 51-54 in human plasma by SDS-PAGE; M=molecular weight marker, P=human plasma, days of incubation above the representative lane. No transfer of AlexaFluor™ 488 to human serum albumin (66.5 kDa) or any other plasma proteins is observed over the 14-day incubation period.

FIG. 20 shows hydrophobic interaction chromatography (HIC) analysis of ADCs 58-61.

FIG. 21 shows hydrophobic interaction chromatography (HIC) analysis of ADCs 62-65.

FIG. 22 shows the cytotoxicity of ADCs 62-65 in HER2-positive SKBR3 cells, HER2-positive BT474 cells, HER2-negative MDA-MB-468 cells and HER2-negative MCF7 cells. Viability data shows the mean of three independent experiments and error bars represent the standard error of the mean (SEM).

FIG. 23 shows live cell microscopy images of HER2-positive SKBR3 cells and HER2-negative MCF7 cells after treatment with conjugates 51 and 52, trastuzumab or vehicle control.

### EXAMPLES

### General Experimental Details

All solvents and reagents were used as received unless otherwise stated. Ethyl acetate, methanol, dichloromethane, acetonitrile and toluene were distilled from calcium hydride. Diethyl ether was distilled from a mixture of lithium aluminium hydride and calcium hydride. Petroleum ether (PE) refers to the fraction between 40-60° C. upon distillation. Tetrahydrofuran was dried using Na wire and distilled from a mixture of lithium aluminium hydride and calcium hydride with triphenylmethane as indicator.

Non-aqueous reactions were conducted under a stream of dry nitrogen using oven-dried glassware. Temperatures of 0° C. were maintained using an ice-water bath. Room temperature (rt) refers to ambient temperature.

Yields refer to spectroscopically and chromatographically pure compounds unless otherwise stated. Reactions were monitored by thin layer chromatography (TLC) or liquid chromatography mass spectroscopy (LC-MS). TLC was performed using glass plates pre-coated with Merck silica gel 60 F₂₅₄ and visualized by quenching of UV fluorescence (λₘₐₓ=254 nm) or by staining with potassium permanganate. Retention factors (R_{f}) are quoted to 0.01. LC-MS was carried out using a Waters ACQUITY H-Class UPLC with an ESCi Multi-Mode Ionisation Waters SQ Detector 2 spectrometer using MassLynx 4.1 software; ESI refers to the electrospray ionisation technique; LC system: solvent A: 2 mM NH₄OAc in H₂O/MeCN (95:5); solvent B: MeCN; solvent C: 2% formic acid; column: ACQUITY UPLC® CSH C18 (2.1 mm×50 mm, 1.7 μm, 130 Å) at 40° C.; gradient: 5-95% B with constant 5% C over 1 min at flow rate of 0.6 mL/min; detector: PDA eλ Detector 220-800 nm, interval 1.2 nm.

Flash column chromatography was carried out using slurry-packed Merck 9385 Kieselgel 60 SiO₂ (230-400 mesh) or Combiflash Rf200 automated chromatography system with Redisep® reverse-phase C18-silica flash columns (20-40 μm).

HPLC purification was carried out via semi-preparative reverse phase HPLC on an Agilent 1260 Infinity using a SupeIcosil ABZ+PLUS column (250 mm×21.2 mm, 5 μm) eluting with a linear gradient system (solvent A: 0.1% (v/v) TFA in water, solvent B: 0.05% (v/v) TFA in acetonitrile) over 20 minutes at a flow rate of 20 mL/min.

Analytical high performance liquid chromatography (HPLC) was performed on Agilent 1260 Infinity machine, using a SupeIcosil™ ABZ+PLUS column (150 mm×4.6 mm, 3 μm) with a linear gradient system (solvent A: 0.05% (v/v) TFA in H₂O; solvent B: 0.05% (v/v) TFA in MeCN) over 20 min at a flow rate of 1 mL/min, and UV detection (λₘₐₓ=220-254 nm).

Infrared (IR) spectra were recorded neat on a Perkin-Elmer Spectrum One spectrometer with internal referencing. Selected absorption maxima (vₘₐₓ) are reported in wavenumbers (cm⁻¹).

¹H and ¹³C nuclear magnetic resonance (NMR) were recorded using an internal deuterium lock on Bruker DPX-400 (400 MHz, 101 MHz), Bruker Avance 400 QNP (400 MHz, 101 MHz) and Bruker Avance 500 Cryo Ultrashield (500 MHz, 126 MHz). Tetramethylsilane was used as an internal standard. In ¹H NMR, chemical shifts (δ_{H}) are reported in parts per million (ppm), to the nearest 0.01 ppm and are referenced to the residual non-deuterated solvent peak (CDCl₃: 7.26, DMSO-d₆: 2.50, CD₃OD: 3.31, D₂O: 4.79). Coupling constants (J) are reported in Hertz (Hz) to the nearest 0.1 Hz. Data are reported as follows: chemical shift, multiplicity (s=singlet; d=doublet; t=triplet; q=quartet; quint=quintet; m=multiplet; or as a combination of these, e.g. dd, dt etc.), integration and coupling constant(s). In ¹³C NMR, chemical shifts (δ_{C}) are quoted in ppm, to the nearest 0.1 ppm, and are referenced to the residual non-deuterated solvent peak (CDCl₃: 77.16, DMSO-d₆, 39.52, CD₃OD: 49.00).

Unless, otherwise specified, high resolution mass spectrometry (HRMS) measurements were recorded with a Micromass Q-TOF mass spectrometer or a Waters LCT Premier Time of Flight mass spectrometer. Mass values are reported within the error limits of ±5 ppm mass units. ESI refers to the electrospray ionisation technique.

UV-Vis spectroscopy was used to determine protein concentrations and fluorophore-to-antibody ratios (FAR) using a nanodrop ND-1000 spectrophotometer. Sample buffer was used as blank for baseline correction with extinction coefficients ε₂₈₀=215,380 M⁻¹ cm⁻¹ for trastuzumab and ε₄₉₅=71,000 M⁻¹ cm⁻¹ for AlexaFluor 488™ (AF488). The correction factor for AF488 absorption at 280 nm is 0.11. FAR was calculated using the following formula:

$$FAR = \frac{{Abs}_{495} / {ε}_{495}}{( {Abs}_{280} - 0.11 \times {Abs}_{495} ) / {ε}_{280}}$$

Protein LC-MS was performed on a Xevo G2-S TOF mass spectrometer coupled to an Acquity UPLC system using an Acquity UPLC BEH300 C4 column (1.7 μm, 2.1×50 mm). H₂O with 0.1% formic acid (solvent A) and 95% MeCN and 5% H₂O with 0.1% formic acid (solvent B) were used as the mobile phase at a flow rate of 0.2 mL/min. The gradient was programmed as follows: 95% A for 0.93 min, then a gradient to 100% B over 4.28 min, then 100% B for 1.04 minutes, then a gradient to 95% A over 1.04 min. The electrospray source was operated with a capillary voltage of 2.0 kV and a cone voltage of 190 V. Nitrogen was used as the desolvation gas at a total flow of 850 L/h. Total mass spectra were reconstructed from the ion series using the MaxEnt algorithm preinstalled on MassLynx software (v4.2 from Waters) according to the manufacturer's instructions. Trastuzumab samples were deglycosylated with PNGase F (New England Biolabs) prior to LC-MS analysis.

### Example 1—Preparation of Conjugating Reagent 1

### Step 1: Synthesis of 4-((4,6-divinylpyrimidin-2-yl)amino)butanoic acid (2)

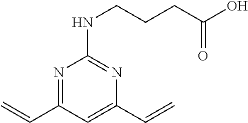 [SMILES: C=Cc1cc(C=C)nc(NCCCC(=O)O)n1]

4-((4,6-divinylpyrimidin-2-yl)amino)butanoic acid (2) was synthesised as previously described (Bargh et al, Chem. Sci., 2020, 11, 2375-2380).

### Step 2: Synthesis of di-tert-butyl (azanediylbis(ethane-2,1-diyl))dicarbamate (3)

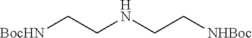 [SMILES: CC(C)(C)OC(=O)NCCNCCNC(=O)OC(C)(C)C]

To a solution of diethylenetriamine (2.16 mL, 20.0 mmol) in THF (25 mL) at 0° C. was slowly added a solution of Boc-ON (9.92 g, 40.0 mmol) in THF (25 mL). The reaction was stirred at 0° C. under nitrogen for 1 h and then concentrated in vacuo. The crude yellow oil was dissolved in CH₂Cl₂ and sequentially washed with 10% aq. NaOH, water and brine. The organic phase was dried over Na₂SO₄ and concentrated in vacuo. Purification by column chromatography (10% MeOH/CH₂Cl₂) yielded the product as a colourless oil (5.61 g, 18.5 mmol, 92% yield). R_{f} 0.08 (SiO₂, 10% MeOH/CH₂Cl₂); vₘₐₓ (neat/cm¹) 3336 (m, N—H), 2975 (m, C—H), 1687 (s, C═0); δ_{H} (400 MHz, CDCl₃) 4.93 (br s, 2H), 3.24-3.19 (m, 4H), 2.73 (t, 4H, J=5.7 Hz), 1.44 (s, 18H); δ_{C} (101 MHz, CDCl₃) 156.3, 79.4, 49.0, 40.4, 28.6; HRMS (ESI) m/z found [M+H]⁺ 304.2221, C₁₄H₃₀O₄N₃⁺ required 304.2231.

### Step 3: Synthesis of methyl bis(2-((tert-butoxycarbonyl)amino)ethyl)glycinate (4)

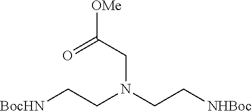 [SMILES: COC(=O)CN(CCNC(=O)OC(C)(C)C)CCNC(=O)OC(C)(C)C]

To a solution of di-tert-butyl (azanediylbis(ethane-2,1-diyl))dicarbamate (3) (5.61 g, 18.5 mmol) in DMF (80 mL) were added DIPEA (3.86 mL, 22.2 mmol) and methyl bromoacetate (2.61 mL, 27.8 mmol). The reaction was stirred at room temperature for 6 h and then concentrated under a stream of nitrogen. The crude residue was purified by column chromatography (40-50% EtOAc/PE) yielding the product as a colourless oil (6.56 g, 17.5 mmol, 94% yield). R_{f} 0.28 (SiO₂, 50% EtOAc/PE); vₘₐₓ (neat/cm⁻¹) 3345 (m, N—H), 2979 (m, C—H), 1740 (s, C═O), 1688 (s, C═O); δ_{H} (400 MHz, CDCl₃) 5.13 (br s, 2H, NH), 3.70 (s, 3H), 3.37 (s, 2H), 3.15 (q, 4H, J=5.6 Hz), 2.72 (t, 4H, J=5.9 Hz), 1.44 (s, 18H); δ_{C} (101 MHz, CDCl₃) 172.3, 156.3, 79.3, 55.1, 54.3, 51.8, 38.7, 28.6; LRMS (ESI) m/z found [M+H]⁺ 376.5.

### Step 4: Synthesis of 2-bromo-N-(prop-2-yn-1-yl)acetamide (5)

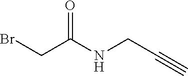

A solution of propargylamine (1.16 mL, 18.2 mmol) in CH₂Cl₂ (33 mL) and sat. NaHCO₃ (33 mL) at maximum stirring was cooled to −10° C. and 2-bromoacetyl bromide (2.42 mL, 27.2 mmol) was added dropwise over 15 min. The reaction mixture was allowed to slowly reach rt, and upon completion the reaction mixture was concentrated. Following addition of water (30 mL), the aqueous solution was extracted with EtOAc (2×80 mL) and the combined organic phases were washed with sat. NaHCO₃ (30 mL), 5% HCl (30 mL) and brine (30 mL). Combined organic phases were dried over Na₂SO₄ and concentrated in vacuo to give the title compound as a light yellow solid (2.97 g, 16.9 mmol, 93%). R_{f} 0.69 (SiO₂, 20% MeOH/CH₂Cl₂); vₘₐₓ (neat/cm⁻¹) 3289 (m, N—H), 3071 (m, C—H), 2120 (w, C≡C), 1645 (s, C═O); δ_{H} (400 MHz, CDCl₃) 6.68 (s, 1H), 4.09 (dd, J=5.3, 2.6 Hz, 2H), 3.90 (s, 2H), 2.28 (t, J=2.6 Hz, 1H); δ_{C} (100 MHz, CDCl₃) 165.1, 78.5, 72.3, 30.0, 28.7; LRMS (ESI) m/z found [M+H]⁺ 178.0.

### Step 5: Synthesis of Compound 6

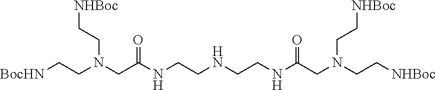 [SMILES: CC(C)(C)OC(=O)NCCN(CCNC(=O)OC(C)(C)C)CC(=O)NCCNCCNC(=O)CN(CCNC(=O)OC(C)(C)C)CCNC(=O)OC(C)(C)C]

In a pre-dried microwave vial containing methyl bis(2-((tert-butoxycarbonyl)amino)ethyl)glycinate (4) (200 mg, 0.53 mmol) dissolved in dry MeOH (0.3 mL) was added diethylenetriamine (10.7 μL, 0.098 mmol), the vial was capped, flushed with nitrogen, and stirred over night at 110° C. The reaction mixture was concentrated and purified by column chromatography (0-10% MeOH/CH₂Cl₂) to give the desired compound as a colourless oil (60.6 mg, 0.077 mmol, 78%). R_{f} 0.14 (SiO₂, 10% MeOH/CH₂Cl₂); vₘₐₓ (neat/cm⁻¹) 3316 (m, N—H), 2974 (m, C—H), 1687 (s, C═O); δ_{H} (400 MHz, CD₃OD) 3.48 (t, 4H, J=5.8 Hz), 3.21 (s, 4H), 3.14 (t, 8H, J=6.3 Hz), 3.06 (t, 4H, J=5.7 Hz), 2.61 (t, 8H, J=6.3 Hz), 1.45 (s, 36H); δ_{C} (101 MHz, CD₃OD) 175.6, 158.6, 80.2, 59.9, 56.4, 49.5, 39.7, 38.4, 28.9; HRMS (ESI) m/z found [M+H]⁺ 790.5425, C₃H₇₂O₁₀N₉⁺ required 790.5402.

### Step 6: Synthesis of Compound 7

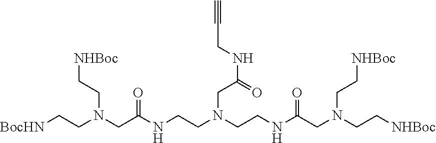 [SMILES: C#CCNC(=O)CN(CCNC(=O)CN(CCNC(=O)OC(C)(C)C)CCNC(=O)OC(C)(C)C)CCNC(=O)CN(CCNC(=O)OC(C)(C)C)CCNC(=O)OC(C)(C)C]

In a pre-dried microwave vial, a suspension of compound 6 (59.3 mg, 0.075 mmol) and K₂CO₃ (20.7 mg, 0.15 mmol) in anhydrous MeCN (0.15 mL) was cooled to 0° C. by means of an ice-bath. A solution of compound 5 (16.5 mg, 0.094 mmol) in MeCN (0.5 mL) was slowly added to the stirring suspension, after which the reaction was brought to rt and stirred overnight under nitrogen. The reaction mixture was concentrated and purified by flash chromatography (0-10% MeOH/CH₂Cl₂) to give the title compound as a white solid (35.9 mg, 0.041 mmol, 54%). R_{f}0.30 (SiO₂, 10% MeOH/CH₂Cl₂); vₘₐₓ (neat/cm⁻¹) 3359 (m, N—H), 2985 (m, C—H), 2101 (m, C≡C), 1724 (s, C═O); δ_{H} (600 MHz, CDCl₃) 7.79 (s, 2H), 5.70 (s, 4H), 4.04 (dd, 2H, J=2.4, 5.4 Hz), 3.37-3.32 (m, 4H), 3.24 (s, 2H), 3.20-3.14 (m, 8H), 3.14 (s, 4H), 2.75-2.70 (m, 4H), 2.60-2.55 (m, 8H), 2.25-2.22 (m, 1H), 2.10 (s, 1H), 1.42 (s, 36H); δ_{C} (101 MHz, CDCl₃) 172.2, 171.5, 156.7, 80.0, 79.4, 71.5, 59.5 (two peaks), 55.8 (two peaks), 38.8, 37.9, 29.0, 28.6; HRMS (ESI) m/z found [M+H]⁺ 885.5760, C₄₁H₇₇O₁₁N₁₀ ⁺ required 885.5768.

### Step 7: Synthesis of Conjugating Reagent 1

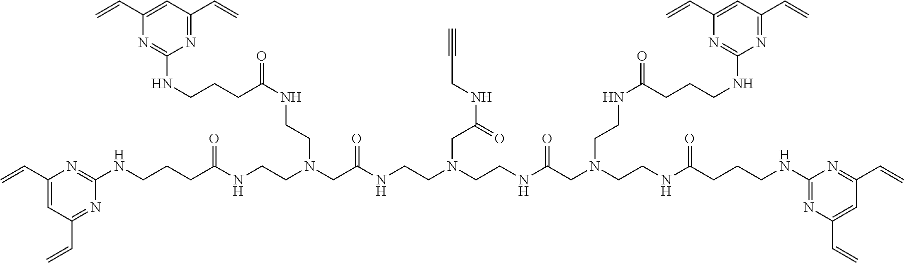 [SMILES: C#CCNC(=O)CN(CCNC(=O)CN(CCNC(=O)CCCNc1nc(C=C)cc(C=C)n1)CCNC(=O)CCCNc1nc(C=C)cc(C=C)n1)CCNC(=O)CN(CCNC(=O)CCCNc1nc(C=C)cc(C=C)n1)CCNC(=O)CCCNc1nc(C=C)cc(C=C)n1]

To a solution of compound 7 (69.0 mg, 0.078 mmol) in CH₂Cl₂ (0.5 mL) at 0° C. was added HCl (4M in dioxane, 1.0 mL). The reaction was stirred under nitrogen for 6 h and then concentrated in vacuo to yield the desired amine hydrochloride salt as a white solid. The amine hydrochloride salt was re-dissolved in CH₂Cl₂ (1 mL) and cooled to 0° C. To this solution, a solution of 4-((4,6-divinylpyrimidin-2-yl)amino)butanoic acid (2) (72.8 mg, 0.312 mmol), triethylamine (217 μL, 1.56 mmol) and HBTU (118 mg, 0.312 mmol) in CH₂Cl₂ (2 mL) at 0° C. was added. The reaction was stirred under nitrogen for 18 h and then concentrated in vacuo. Purification by column chromatography (2.5-10% MeOH/CH₂Cl₂) yielded the product as a colourless oil (29.6 mg, 0.022 mmol, 28% over 2 steps). R_{f} 0.15 (SiO₂, 10% MeOH/CH₂Cl₂); vₘₐₓ (neat/cm⁻¹) 3290 (m, N—H), 2944 (m, C—H), 2187 (m, C≡C), 1633 (s, C═O); δ_{H} (600 MHz, CD₃OD) 6.67 (s, 4H), 6.59 (dd, 8H, J=10.7, 17.4 Hz), 6.35 (d, 8H, J=17.4 Hz), 5.55 (dd, 8H, J=1.5, 10.7 Hz), 3.97 (d, 2H, J=2.5), 3.45 (t, 8H, J=6.8 Hz), 3.27 (t, 4H, J=6.5 Hz), 3.24 (t, 8H, J=6.3 Hz), 3.21 (s, 2H), 3.18 (s, 4H), 2.65 (t, 4H, J=6.4 Hz), 2.62-2.59 (m, 1H), 2.60 (t, 8H, J=6.2 Hz), 2.28 (t, 8H, J=7.5 Hz), 1.90 (quint, 8H, J=7.2 Hz); δ_{C} (101 MHz, CD₃OD) 175.9, 174.1, 173.7, 165.3, 164.0, 137.1, 122.1, 105.8, 80.8, 72.5, 59.7, 59.3, 55.9, 55.8, 44.0, 38.7, 38.5, 34.7, 29.4, 27.0; HRMS (ESI) m/z found [M+H]⁺ 1345.7930, C₆₉H₉₇O₇N₂₂⁺ required 1345.7905.

### Example 2—Preparation of Conjugating Reagent 8

### Step 1: Synthesis of Compound 9

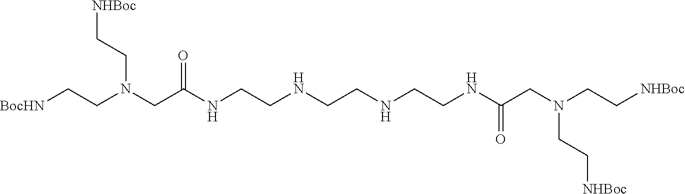 [SMILES: CC(C)(C)OC(=O)NCCN(CCNC(=O)OC(C)(C)C)CC(=O)NCCNCCNCCNC(=O)CN(CCNC(=O)OC(C)(C)C)CCNC(=O)OC(C)(C)C]

In a pre-dried microwave vial containing methyl bis(2-((tert-butoxycarbonyl)amino)ethyl)glycinate (4) (203 mg, 0.54 mmol) dissolved in dry MeOH (0.3 mL) was added triethylenetetramine (14.9 μL, 0.100 mmol), the vial was capped, flushed with nitrogen, and stirred over night at 110° C. The reaction mixture was concentrated and purified by column chromatography (0-30% MeOH/CH₂Cl₂) to give the desired compound as a colourless oil (40.8 mg, 0.049 mmol, 49%). R_{f}0.38 (SiO₂, 20% MeOH/CH₂Cl₂); vₘₐₓ (neat/cm⁻¹) 3335 (m, N—H), 2980 (m, C—H), 1690 (s, C═O); δ_{H} (600 MHz, CD₃OD) 3.38 (t, 4H, J=6.3 Hz), 3.17 (s, 4H), 3.13 (t, 8H, J=6.1 Hz), 2.80-2.77 (m, 4H), 2.76 (s, 4H), 2.60 (t, 8H, J=5.7 Hz), 1.45 (s, 36H); δ_{C} (101 MHz, CD₃OD) 174.5, 158.6, 80.2, 60.1, 56.4, 49.7, 49.4, 39.7, 39.7, 28.9; HRMS (ESI) m/z found [M+H]⁺ 833.5843, C₃₈H₇₇O₁₀N₁₀⁺ required 833.5824.

### Step 2: Synthesis of Compound 10

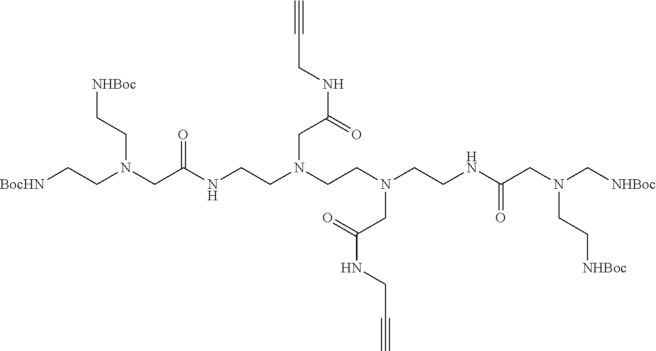 [SMILES: C#CCNC(=O)CN(CCNC(=O)CN(CCNC(=O)OC(C)(C)C)CCNC(=O)OC(C)(C)C)CCN(CCNC(=O)CN(CCNC(=O)OC(C)(C)C)CNC(=O)OC(C)(C)C)CC(=O)NCC#C]

In a pre-dried microwave vial, a suspension of compound 9 (39.2 mg, 0.047 mmol) and K₂CO₃ (26.0 mg, 0.188 mmol) in anhydrous MeCN (0.1 mL) was cooled to 0° C. by means of an ice-bath. A solution of compound 5 (20.8 mg, 0.118 mmol) in MeCN (0.4 mL) was slowly added to the stirring suspension, after which the reaction was brought to rt and stirred overnight under nitrogen. The reaction mixture was concentrated and purified by flash chromatography (0-10% MeOH/CH₂Cl₂) to give the title compound as a white solid (30.0 mg, 0.029 mmol, 62%). R_{f} 0.18 (SiO₂, 10% MeOH/CH₂Cl₂); vₘₐₓ (neat/cm⁻¹) 3296 (m, N—H), 2978 (m, C—H), 2157 (m, C≡C), 1654 (s, C═O); δ_{H} (600 MHz, CD₃OD) 4.20 (s, 4H), 4.08 (d, 4H, J=2.5 Hz), 3.80 (s, 4H), 3.57 (t, 4H, J=6.0 Hz), 3.51-3.44 (m, 16H), 3.27 (s, 4H), 3.21-3.17 (m, 4H), 2.72 (t, 2H, J=2.5 Hz), 1.47 (s, 36H); δ_{C} (101 MHz, CD₃OD) 169.8, 166.3, 159.1, 81.3, 80.3, 73.2, 57.0, 56.1, 56.0, 55.9, 53.3, 37.0, 36.7, 30.0, 28.8; HRMS (ESI) m/z found [M+H]⁺ 1023.6547, C₄₈H₈₇O₁₂N₁₂⁺ required 1023.6566.

### Step 3: Synthesis of Conjugating Reagent 8

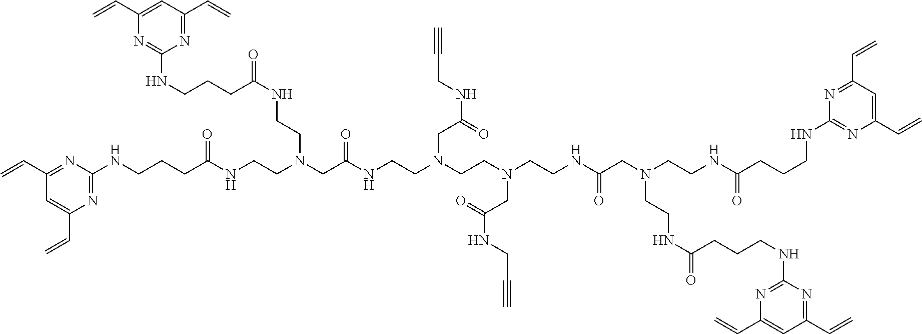 [SMILES: C#CCNC(=O)CN(CCNC(=O)CN(CCNC(=O)CCCNc1nc(C=C)cc(C=C)n1)CCNC(=O)CCCNc1nc(C=C)cc(C=C)n1)CCN(CCNC(=O)CN(CCNC(=O)CCCNc1nc(C=C)cc(C=C)n1)CCNC(=O)CCCNc1nc(C=C)cc(C=C)n1)CC(=O)NCC#C]

To a solution of compound 10 (79.8 mg, 0.078 mmol) in CH₂Cl₂ (0.5 mL) at 0° C. was added HCl (4M in dioxane, 1.0 mL). The reaction was stirred under nitrogen for 6 h and then concentrated in vacuo to yield the desired amine hydrochloride salt as a white solid. The amine hydrochloride salt was re-dissolved in CH₂Cl₂ (1 mL) and cooled to 0° C. To this solution was added a solution of 4-((4,6-divinylpyrimidin-2-yl)amino)butanoic acid (2) (72.8 mg, 0.312 mmol), triethylamine (217 μL, 1.56 mmol) and HBTU (118 mg, 0.312 mmol) in CH₂Cl₂ (2 mL) at 0° C. The reaction was stirred under nitrogen for 18 h and then concentrated in vacuo. Purification by reverse phase flash column chromatography (35-70% solvent B in solvent A. Solvent A: 100 mM NH₄OH (aq). Solvent B: MeCN) and lyophilisation yielded the product as a colourless oil (46.1 mg, 0.031 mmol, 40% over 2 steps). vₘₐₓ (neat/cm⁻¹) 3289 (m, N—H), 2938 (m, C—H), 2186 (m, C≡C), 1637 (s, C═O); δ_{H} (600 MHz, CD₃OD) 6.67 (s, 4H), 6.59 (dd, 8H, J=10.6, 17.4 Hz), 6.35 (d, 8H, J=17.3 Hz), 5.56 (dd, 8H, J=1.2, 10.7 Hz), 3.98 (d, 4H, J=2.3), 3.45 (t, 8H, J=6.7 Hz), 3.27 (t, 4H, J=6.8 Hz), 3.24 (t, 8H, J=6.4 Hz), 3.18 (s, 4H), 3.17 (s, 4H), 2.64-2.57 (m, 4H), 2.64-2.57 (m, 2H), 2.64-2.57 (m, 8H), 2.60 (s, 4H), 2.29 (t, 8H, J=7.5 Hz), 1.91 (quint, 8H, J=7.1 Hz); δ_{C} (101 MHz, CD₃OD) 175.9, 174.1, 173.7, 165.3, 164.0, 137.1, 122.1, 105.8, 80.9, 72.4, 59.7, 59.3, 55.8 (two peaks), 54.4, 41.7, 38.7, 38.4, 34.7, 29.4, 27.0; HRMS (ESI) m/z found [M+H]⁺ 1483.8687, C₇₆H₁₀₇O₈N₂₄⁺ required 1483.8698.

### Example 3—Preparation of Conjugating Reagent 11

### Step 1: Synthesis of Compound 12

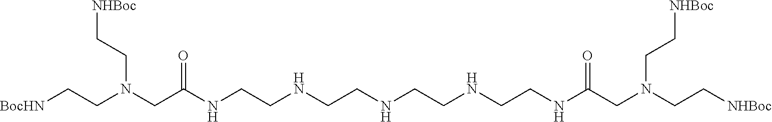 [SMILES: CC(C)(C)OC(=O)NCCN(CCNC(=O)OC(C)(C)C)CC(=O)NCCNCCNCCNCCNC(=O)CN(CCNC(=O)OC(C)(C)C)CCNC(=O)OC(C)(C)C]

In a pre-dried microwave vial containing methyl bis(2-((tert-butoxycarbonyl)amino)ethyl)glycinate (4) (964 mg, 2.57 mmol) dissolved in dry MeOH (1 mL) was added tetraethylenepentamine (90 μL, 0.475 mmol), the vial was capped, flushed with nitrogen, and stirred over night at 110° C. The reaction mixture was concentrated and purified by column chromatography (0-20% MeOH/CH₂Cl₂) to give the desired compound as a white foam (184 mg, 0.21 mmol, 44%). R_{f} 0.06 (BuOH:H₂O:MeCN, 0.8/0.1/0.1); vₘₐₓ (neat/cm⁻¹) 3330 (m, N—H), 2980 (m, C—H), 1689 (s, C═O); δ_{H} (400 MHz, CDCl₃) 3.40-3.30 (m, 4H), 3.16-3.09 (m, 8H), 3.07 (s, 4H), 2.83-2.66 (m, 12H), 2.60-2.52 (m, 8H), 1.40 (s, 36H); δ_{C} (100 MHz, CDCl₃) 171.5, 156.6, 79.3, 59.2, 55.4, 48.6 (three peaks), 38.7, 38.6, 28.6; HRMS (ESI) m/z found [M+H]⁺ 876.6284, C₄₀H₈₂N₁₁NaO₁₀⁺ required 876.6246.

### Step 2: Synthesis of Compound 13

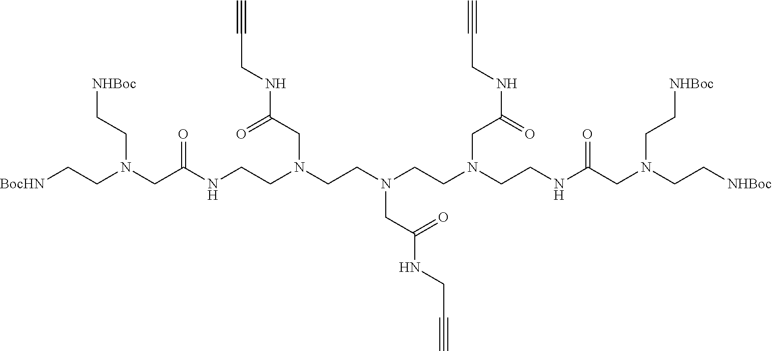 [SMILES: C#CCNC(=O)CN(CCNC(=O)CN(CCNC(=O)OC(C)(C)C)CCNC(=O)OC(C)(C)C)CCN(CCN(CCNC(=O)CN(CCNC(=O)OC(C)(C)C)CCNC(=O)OC(C)(C)C)CC(=O)NCC#C)CC(=O)NCC#C]

In a pre-dried microwave vial, a suspension of compound 12 (184 mg, 0.21 mmol) and K₂CO₃ (174 mg, 1.26 mmol) in anhydrous MeCN (0.26 mL) was cooled to 0° C. by means of an ice-bath. A solution of compound 5 (anh. MeCN, 0.5 M, 1.58 mL) was slowly added to the stirring suspension, after which the reaction was brought to rt and stirred overnight under nitrogen. The reaction mixture was concentrated, re-dissolved in CH₂Cl₂ (40 mL), the organic phase washed with water (2×10 mL), brine (1×20 mL), dried over Na₂SO₄, and purified by flash chromatography (5-10% MeOH/CH₂Cl₂) to give the title compound as a white foam (119 mg, 0.102 mmol, 49%). R_{f} 0.5 (SiO₂, 20% MeOH/CH₂Cl₂); vₘₐₓ (neat/cm⁻¹) 3309 (m, N—H), 2928 (m, C—H), 2158 (m, C≡C), 1658 (s, C═O); δ_{H} (400 MHz, CD₃OD) 4.05 (d, J=2.6 Hz, 4H), 4.03 (d, J=2.5 Hz, 2H), 3.39-3.32 (m, 7H), 3.27 (s, 4H), 3.25 (s, 2H), 3.20 (s, 4H), 3.17-3.13 (m, 8H), 2.77-2.67 (m, 8H), 2.66-2.58 (m, 8H), 1.47 (s, 36H); δ_{C} (100 MHz, CD₃OD) 174.3, 173.7, 173.6, 158.4, 80.9, 80.8, 80.1, 72.6, 72.3, 60.2, 59.3, 59.2, 56.5, 55.7, 54.4, 54.3, 39.7, 38.5, 29.5, 29.3, 28.9; HRMS (ESI) m/z found [M+H]⁺ 1161.7365, C₅₅H₉₇O₁₃N₁₄⁺ required 1161.7360.

### Step 3: Synthesis of Conjugating Reagent 11

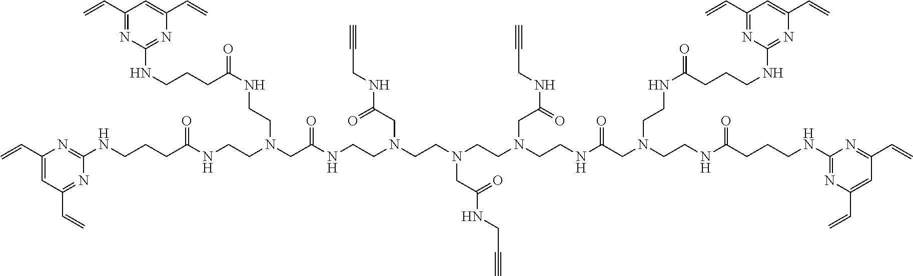 [SMILES: C#CCNC(=O)CN(CCNC(=O)CN(CCNC(=O)CCCNc1nc(C=C)cc(C=C)n1)CCNC(=O)CCCNc1nc(C=C)cc(C=C)n1)CCN(CCN(CCNC(=O)CN(CCNC(=O)CCCNc1nc(C=C)cc(C=C)n1)CCNC(=O)CCCNc1nc(C=C)cc(C=C)n1)CC(=O)NCC#C)CC(=O)NCC#C]

To a solution of compound 13 (25 mg, 22 μmol) in CH₂Cl₂ (150 μL) at 0° C. was added HCl (4 M in dioxane, 270 uL). The reaction was brought to rt and stirred for 1 h. The reaction mixture was concentrated to give the desired amine hydrochloride salt as a white solid. The amine hydrochloride salt (23.0 mg, 22.0 μmol) was suspended in CH₂Cl₂ (150 μL), cooled to 0° C. by means of an ice-bath, and added first DIPEA (74.0 μL, 0.42 mmol) and then 2 (25.0 mg, 0.11 mmol). The solution was stirred at rt for 10 min, after which a solution of BTFFH in CH₂Cl₂ (0.6 M, 205 μL) was added dropwise over 3 h during maximum stirring. After complete addition of BTFFH the reaction was stirred for an additional 12 h at rt, concentrated, and purified by reverse phase flash column chromatography (35-70% solvent B in solvent A. Solvent A: 100 mM NH₄OH (aq). Solvent B: MeCN) to yield the desired product after lyophilization as a pale yellow solid (6.70 mg, 41.4 μmol, 20% over 2 steps). vₘₐₓ (neat/cm¹) 3305 (m, N—H), 2970 (m, C—H), 2156 (m, C≡C), 1637 (s, C═O); δ_{H} (400 MHz, CD₃OD) 6.68 (s, 4H), 6.60 (dd, J=17.4, 10.7 Hz, 8H), 6.36 (d, J=17.3 Hz, 8H), 5.56 (dd, J=10.6, 1.6 Hz, 8H), 4.02-3.96 (m, 6H), 3.45 (t, J=6.8 Hz, 8H), 3.35 (s, 4H), 3.29-3.15 (m, 21H), 2.67-2.56 (m, 20H), 2.29 (t, J=7.5 Hz, 8H), 1.91 (quint, J=7.0 Hz, 8H); δ_{C} (126 MHz, CD₃OD) 175.9, 174.1, 173.8, 173.7, 165.3, 164.0, 137.1, 122.1, 105.8, 81.0 (two peaks), 72.5, 72.4, 59.8, 59.4, 59.3, 55.9, 55.8, 54.5 (two peaks), 54.3 (two peaks), 41.7, 38.7, 38.4, 34.7, 29.4, 29.3, 27.0; HRMS (ESI) m/z found [M+H]⁺ 1621.9491, C₈₃H₁₁₇N₂₆O₉⁺required 1621.9497.

### Example 4—Preparation of Conjugating Reagent 14

### Step 1: Synthesis of tert-butyl bis(2-(prop-2-yn-1-yloxy)ethyl)carbamate (15)

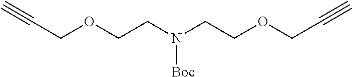 [SMILES: C#CCOCCN(CCOCC#C)C(=O)OC(C)(C)C]

A solution of diethanolamine (2.44 g, 23.0 mmol) in CH₂Cl₂ (25 mL) was cooled to 0° C. by means of an ice-bath and a solution of di-tert-butyl dicarbonate (6.33 g, 29.0 mmol) in anh. CH₂Cl₂ (5 mL) as slowly added. The reaction was stirred for 12 h at rt, diluted in CH₂Cl₂ (40 mL), added water (40 mL), and the aqueous phase was extracted twice with CH₂Cl₂ (2×20 mL). The combined organic phases were washed with brine, dried over Na₂SO₄, and concentrated to give tert-butyl bis(2-hydroxyethyl)carbamate as a clear oil, which was used directly in the next step without further purification. To a 50% NaOH_{(aq)} solution (11 mL) was sequentially added tert-butyl bis(2-hydroxyethyl)carbamate in toluene (11 mL), tetrabutylammonium bisulfate (16.0 mg, 47.0 μmol), and propargyl bromide (80 w % in toluene, 8.7 mL, 78.0 mmol), and the reaction was stirred at rt for 48 h under nitrogen atmosphere. The organic layer was isolated, concentrated and the desired compound was purified by flash chromatography (50% EtOAc/hexane) to give the title compound as a yellow viscous oil (2.30 g, 7.57 mmol, 35% over 2 steps). R_{f} 0.60 (SiO₂, 50% EtOAc/hexane); vₘₐₓ (neat/cm⁻¹) 2975 (m, C—H), 2117 (m, C≡C), 1686 (s, C═O); δ_{H} (400 MHz, CDCl₃) 4.13 (d, J=2.4 Hz, 4H), 3.66-3.57 (m, 4H), 3.52-3.38 (m, 4H), 2.41 (t, J=2.4 Hz, 2H), 1.45 (s, 9H); δ_{C} (100 MHz, CDCl₃) 155.6, 79.9, 79.8, 74.5, 68.7, 58.3, 47.9, 28.6; LRMS (ESI) m/z found [M+Na]⁺ 304.2.

### Step 2: Synthesis of tert-butyl (5-(bis(2-(prop-2-yn-1-yloxy)ethyl)amino)pentyl)carbamate (16)

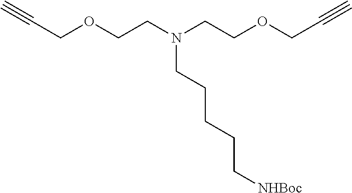 [SMILES: C#CCOCCN(CCCCCNC(=O)OC(C)(C)C)CCOCC#C]

A solution of compound 15 (281 mg, 1.00 mmol) in CH₂Cl₂ (1.5 mL) was cooled to 0° C., followed by addition of HCl (4 M in dioxane, 3 mL). The reaction was brought to rt and stirred for 2 h. The reaction mixture was concentrated to give the desired amine hydrochloride salt as a white solid. The solid was re-suspended in MeCN (10 mL). Sodium carbonate (1.06 g, 10.0 mmol) was added. After stirring for 5 min, a solution of tert-Butyl (5-bromopentyl)carbamate (399 mg, 1.50 mmol) in MeCN (2 mL) was added. The reaction mixture was then refluxed at 70° C. for 48 h. Subsequently, the reaction mixture was diluted with brine and extracted with EtOAc (3×). The combined organic layers were dried over Na₂SO₄, concentrated, and purified by flash chromatography (30-50% EtOAc/hexane) to give the title compound as a pale yellow oil (242 mg, 66% over 2 steps). R_{f} 0.21 (SiO₂, 80% EtOAc/PE); vₘₐₓ (neat/cm⁻¹) 3342 (m, N—H), 2938 (m, C—H), 2111 (m, C≡C), 1676 (s, C═O); δ_{H}(400 MHz, CDCl₃) 4.54 (br s, 1H), 4.16 (d, J=2.3 Hz, 4H), 3.60 (t, J=6.0 Hz, 4H), 3.14-3.06 (m, 2H), 2.73 (t, J=6.0 Hz, 4H), 2.55-2.49 (m, 2H), 2.42 (t, J=2.2 Hz, 2H), 1.51-1.42 (m, 4H), 1.44 (s, 9H), 1.30 (quint, J=7.6 Hz, 2H); Sc (100 MHz, CDCl₃) 156.0, 79.9, 79.0, 74.3, 68.3, 58.2, 55.0, 53.8, 40.5, 29.9, 28.4, 26.7, 24.5; HRMS (ESI) m/z found [M+H]⁺ 367.2587, C₂₀H₃₄O₄N₂⁺ required 367.2597.

### Step 3: Synthesis of N-(5-(bis(2-(prop-2-yn-1-yloxy)ethyl)amino)pentyl)-2-bromoacetamide (17)

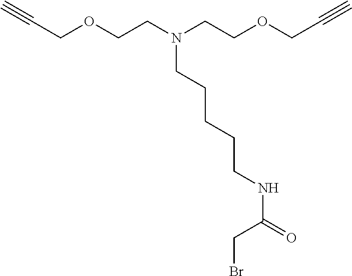 [SMILES: C#CCOCCN(CCCCCNC(=O)CBr)CCOCC#C]

To a solution of compound 16 (100 mg, 0.27 mmol) in CH₂Cl₂ (0.5 mL) at 0° C. was added HCl (4M in dioxane, 1 mL). The reaction was stirred under nitrogen for 2 h and then concentrated in vacuo to yield the desired amine hydrochloride salt as a white solid. The hydrochloride salt was re-dissolved in a mixture of CH₂Cl₂ (1 mL) and sat. NaHCO₃ (1 mL) and cooled to −10° C., followed by dropwise addition of 2-bromoacetyl bromide (35.3 μL, 0.405 mmol). The reaction mixture was stirred for 1 h, and then diluted with brine and extracted with CH₂Cl₂ (×3). Combined organic phases were dried over Na₂SO₄ and concentrated in vacuo. Purification by flash chromatography (0-5% MeOH/CH₂Cl₂) yielded the title compound as a pale yellow oil (57.3 mg, 0.148 mmol, 55% over 2 steps). R_{f} 0.41 (SiO₂, 10% MeOH/CH₂Cl₂); vₘₐₓ (neat/cm⁻¹) 3219 (m, N—H), 2928 (m, C—H), 2111 (m, C≡C), 1672 (s, C═O); δ_{H} (400 MHz, CDCl₃) 6.55 (br s, 1H), 4.15 (d, J=2.4 Hz, 4H), 3.87 (s, 2H), 3.62 (t, J=5.5 Hz, 4H), 3.27 (q, 2H, J=6.7 Hz), 2.78-2.75 (m, 4H), 2.58-2.54 (m, 2H), 2.43 (t, J=2.4 Hz, 2H), 1.55 (quint, J=7.4 Hz, 2H), 1.50 (quint, J=7.4 Hz, 2H), 1.33 (quint, J=7.6 Hz, 2H); δ_{C} (100 MHz, CDCl₃) 165.5, 79.7, 74.8, 67.8, 58.4, 55.0, 53.9, 40.2, 29.8, 29.5, 29.1, 24.5; HRMS (ESI) m/z found [M+H]⁺ 387.1273, C₁₇H₂₈O₃N₂Br⁺ required 387.1283.

### Step 4: Synthesis of Compound 18

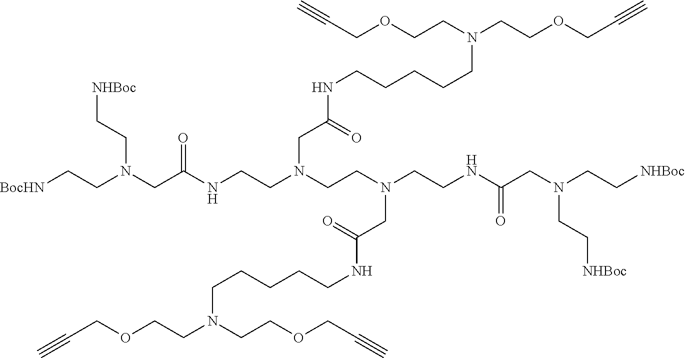 [SMILES: C#CCOCCN(CCCCCNC(=O)CN(CCNC(=O)CN(CCNC(=O)OC(C)(C)C)CCNC(=O)OC(C)(C)C)CCN(CCNC(=O)CN(CCNC(=O)OC(C)(C)C)CCNC(=O)OC(C)(C)C)CC(=O)NCCCCCN(CCOCC#C)CCOCC#C)CCOCC#C]

To a solution of compound 9 (49.3 mg, 59.2 μmol) in MeCN (1 mL) was added anh. K₂CO₃ (32.7 mg, 237 μmol), followed by a solution of N-(5-(bis(2-(prop-2-yn-1-yloxy)ethyl)amino)pentyl)-2-bromoacetamide (17) (57.3 mg, 148 μmol) in MeCN (0.5 mL). The reaction was stirred at room temperature for 48 h. The reaction mixture was concentrated, and the desired compound purified by flash chromatography (0%-15% MeOH/CH₂Cl₂) to give the title compound as a clear oil (38.8 mg, 26.6 μmol, 45%). R_{f} 0.35 (15% MeOH/CH₂Cl₂); vₘₐₓ (neat/cm⁻¹) 3283 (m, N—H), 2936 (m, C—H), 2155 (m, C≡C), 1654 (s, C═O); δ_{H} (400 MHz, CD₃OD) 4.17 (s, 4H), 3.66 (t, J=5.5 Hz, 8H), 3.32-3.29 (m, 8H), 3.26-3.21 (m, 4H), 3.20 (s, 4H), 3.16 (s, 4H), 3.13 (t, J=5.8 Hz, 8H), 2.89-2.84 (m, 8H), 2.70-2.65 (m, 12H), 2.59 (t, J=5.7 Hz, 8H), 1.61-1.50 (m, 8H), 1.44 (s, 36H), 1.38-1.30 (m, 4H); δ_{C} (126 MHz, CD₃OD) 174.3, 173.8, 158.5, 80.7, 80.2, 76.2, 68.3, 60.2, 59.6, 59.0, 56.5 (two peaks), 56.0 (two peaks), 54.6, 40.3, 39.7, 38.6, 30.5, 29.0, 26.8, 25.8; HRMS (ESI) m/z found [M+H]⁺ 1445.9692, C₇₂H₁₂₉N₁₄O₁₆⁺ required 1445.9711.

### Step 5: Synthesis of Conjugating Reagent 14

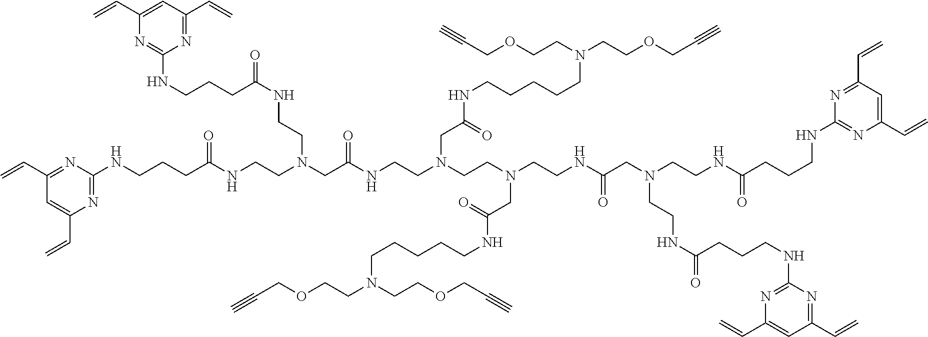 [SMILES: C#CCOCCN(CCCCCNC(=O)CN(CCNC(=O)CN(CCNC(=O)CCCNc1nc(C=C)cc(C=C)n1)CCNC(=O)CCCNc1nc(C=C)cc(C=C)n1)CCN(CCNC(=O)CN(CCNC(=O)CCCNc1nc(C=C)cc(C=C)n1)CCNC(=O)CCCNc1nc(C=C)cc(C=C)n1)CC(=O)NCCCCCN(CCOCC#C)CCOCC#C)CCOCC#C]

To a solution of compound 18 (13.0 mg, 8.99 μmol) in CH₂Cl₂ (0.25 mL) at 0° C. was added HCl (4M in dioxane, 0.5 mL). The reaction was stirred under nitrogen for 2 h and then concentrated in vacuo to yield the desired amine hydrochloride salt as a white solid. The amine hydrochloride salt was re-dissolved in CH₂Cl₂ (0.25 mL) and cooled to 0° C. To this solution was added a solution of 4-((4,6-divinylpyrimidin-2-yl)amino)butanoic acid (2) (12.6 mg, 53.9 μmol), triethylamine (25 μL, 180 μmol) and HBTU (20.5 mg, 53.9 μmol) in CH₂Cl₂ (0.25 mL) at 0° C. The reaction was stirred under nitrogen for 18 h and then concentrated in vacuo. Purification by reverse phase flash column chromatography (35-70% solvent B in solvent A. Solvent A: 100 mM NH₄OH (aq). Solvent B: MeCN) and lyophilisation yielded the product as a white solid (1.80 mg, 0.94 μmol, 10% over 2 steps). vₘₐₓ (neat/cm⁻¹) 3299 (m, N—H), 2928 (m, C—H), 2153 (m, C≡C), 1645 (s, C═O); δ_{H} (400 MHz, CD₃OD) 6.69 (s, 4H), 6.60 (dd, J=17.4, 10.7 Hz, 8H), 6.36 (d, J=17.3 Hz, 8H), 5.57 (dd, J=10.7, 1.5 Hz, 8H), 4.23 (d, J=2.4 Hz, 8H), 3.83 (t, J=4.9 Hz, 8H), 3.45 (t, J=6.8 Hz, 8H), 3.30-3.15 (m, 32H), 3.00 (t, J=2.3 Hz, 4H), 2.65-2.60 (m, 24H), 2.29 (t, J=7.5 Hz, 8H), 1.91 (quint, J=7.1 Hz, 8H), 1.56 (quint, J=7.5 Hz, 4H), 1.39-1.30 (m, 8H); δ_{C} (126 MHz, CD₃OD) 175.9, 174.2 (two peaks), 165.3, 164.0, 137.2, 122.2, 105.9, 89.9, 77.2, 68.8, 59.9 (two peaks), 59.2, 56.1, 56.0, 55.3, 54.0, 47.1, 41.8, 40.4, 38.8 (two peaks), 34.7, 30.2, 26.8, 27.1, 25.0; HRMS (ESI) m/z found [M+H]⁺ 1906.1851, C₁₀₀H₁₄₉N₂₆O₁₂⁺ required 1906.1848.

### Example 5—Preparation of Conjugating Reagent 19

### Step 1: Synthesis of 2,5-dioxopyrrolidin-1-yl 3-(4,5-dibromo-2-methyl-3,6-dioxo-3,6-dihydropyridazin-1(2H)-yl)propanoate (20)

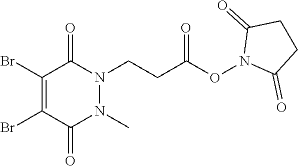 [SMILES: Cn1c(=O)c(Br)c(Br)c(=O)n1CCC(=O)ON1C(=O)CCC1=O]

Dibromopyridazinedione (20) was synthesised as previously described (Bahou et al, Org. Biomol. Chem., 2018, 16, 1359-1366).

### Step 2: Synthesis of Conjugating Reagent 19

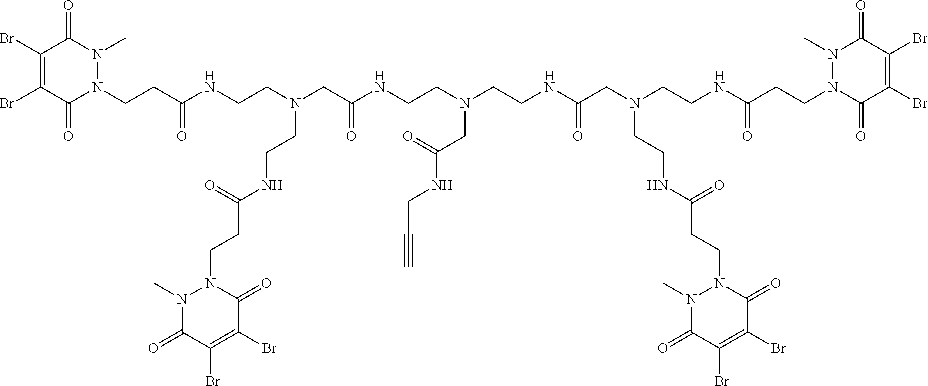 [SMILES: C#CCNC(=O)CN(CCNC(=O)CN(CCNC(=O)CCn1c(=O)c(Br)c(Br)c(=O)n1C)CCNC(=O)CCn1c(=O)c(Br)c(Br)c(=O)n1C)CCNC(=O)CN(CCNC(=O)CCn1c(=O)c(Br)c(Br)c(=O)n1C)CCNC(=O)CCn1c(=O)c(Br)c(Br)c(=O)n1C]

Compound 7 (3.90 mg, 4.40 μmol) was dissolved in CH₂Cl₂ (0.25 mL) and 4M HCl in 1,4-dioxane (0.25 mL) and the solution stirred at rt for 1 h. The reaction mixture then was concentrated under a stream of N₂. To the crude residue was added dibromopyridazinedione 20 (10.0 mg, 22.0 μmol) in CH₂Cl₂DMF (1:1, 1 mL) followed by K₂CO₃ (3.00 mg, 22.0 μmol) and the reaction mixture stirred at rt for 3 h. Upon completion, the reaction was concentrated under a stream of N₂ and purified by preparative RP-HPLC (5-95% B) to yield the title compound (2.50 mg, 1.36 mol, 31%) as a white solid. HPLC (5-95% MeCN/H₂O over 20 min) retention time 7.433 min; LRMS (ESI) m/z found [M+H]⁺ 1837.0, C₅₃H₆₉Br₈N₁₈O₁₅⁺ required 1837.5, m/z found [M+HCOO]⁻ 1881.1, C₅₄H₆₉Br₈N₁₈O₁₇⁻ required 1881.5.

### Example 6—Preparation of Conjugating Reagent 21

### Step 1: Synthesis of Conjugating Reagent 21

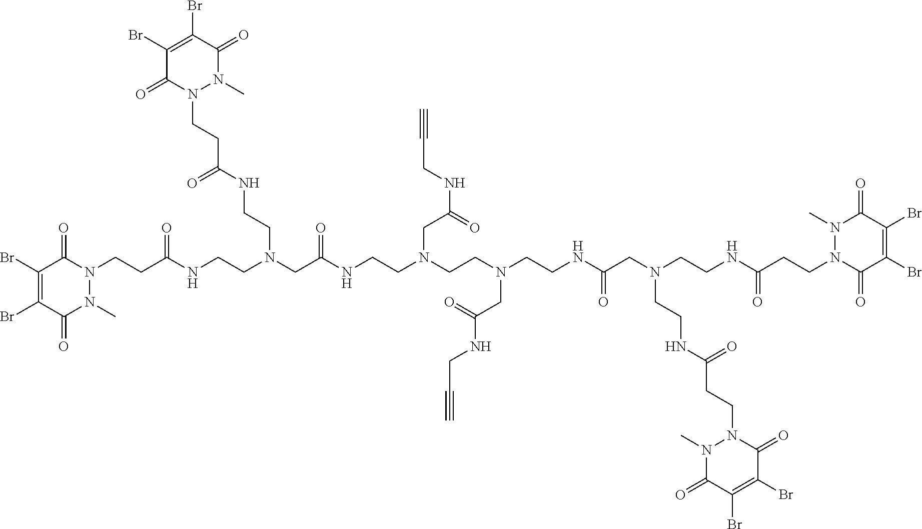 [SMILES: C#CCNC(=O)CN(CCNC(=O)CN(CCNC(=O)CCn1c(=O)c(Br)c(Br)c(=O)n1C)CCNC(=O)CCn1c(=O)c(Br)c(Br)c(=O)n1C)CCN(CCNC(=O)CN(CCNC(=O)CCn1c(=O)c(Br)c(Br)c(=O)n1C)CCNC(=O)CCn1c(=O)c(Br)c(Br)c(=O)n1C)CC(=O)NCC#C]

Compound 10 (10.0 mg, 9.80 μmol) was dissolved in CH₂Cl₂ (0.25 mL) and 4M HCl in 1,4-dioxane (0.25 mL) and the solution stirred at rt for 1 h. The reaction mixture then was concentrated under a stream of N₂. To the crude residue was added dibromopyridazinedione 20 (22.0 mg, 48.9 μmol) in CH₂Cl₂DMF (1:1, 1 mL) followed by K₂CO₃ (6.80 mg, 48.9 μmol) and the reaction mixture stirred at rt for 3 h. Upon completion, the reaction was concentrated under a stream of N₂ and purified by preparative RP-HPLC (5-95% B) to yield the title compound (4.20 mg, 2.13 μmol, 22%) as a white solid. HPLC (5-95% MeCN/H₂O over 20 min) retention time 7.507 min; LRMS (ESI) m/z found [M+H]⁺ 1976.7, C₆₀H₇₉Br₈N₂₀O₁₆⁺ required 1975.7, m/z found [M+HCOO]⁻ 2018.8, C₆₁H₇₉Br₈N₂₀O₁₈⁻ required 2019.7.

### Example 7—Preparation of Conjugating Reagent 22

### Step 1: Synthesis of Divinyltriazine 23

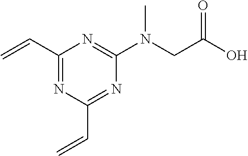 [SMILES: C=Cc1nc(C=C)nc(N(C)CC(=O)O)n1]

Divinyltriazine (23) was synthesised as previously described (Counsell et al, Org. Biomol. Chem., 2020, 18, 4739-4743).

### Step 2: Synthesis of Conjugating Reagent 22

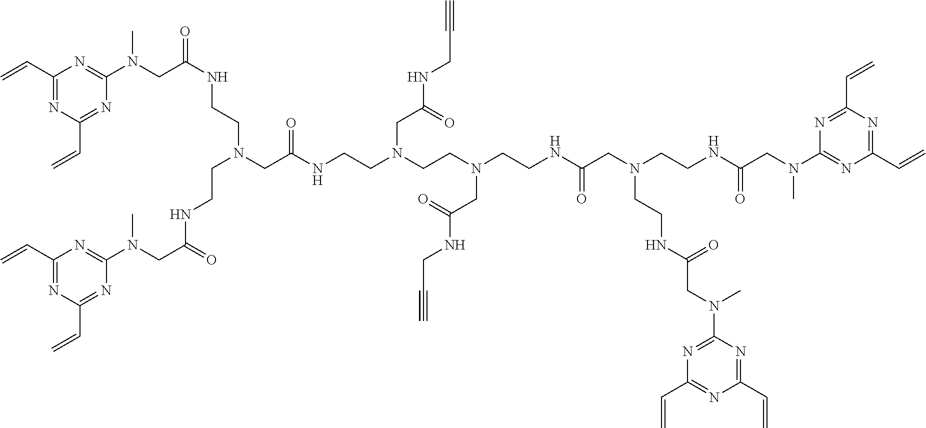 [SMILES: C#CCNC(=O)CN(CCNC(=O)CN(CCNC(=O)CN(C)c1nc(C=C)nc(C=C)n1)CCNC(=O)CN(C)c1nc(C=C)nc(C=C)n1)CCN(CCNC(=O)CN(CCNC(=O)CN(C)c1nc(C=C)nc(C=C)n1)CCNC(=O)CN(C)c1nc(C=C)nc(C=C)n1)CC(=O)NCC#C]

A solution of compound 10 (20.0 mg, 19.5 μmol) in HCl (4 M in 1,4-dioxane, 0.5 mL) and CH₂Cl₂ (0.5 mL) was stirred at rt for 1 h. Upon completion, the reaction mixture was concentrated in vacuo. To this deprotected residue was added HBTU (41.7 mg, 110 μmol), dinvinyltriazine 23 (24.2 mg, 110 μmol) and DMF (1 mL) followed by DIPEA (76.6 μL, 440 μmol) and the mixture stirred at rt for 45 min. Upon completion, the reaction mixture was purified by automated reverse phase FCC (10-60% solvent B in solvent A; Solvent A: 0.1 M NH₄OH/H₂O, Solvent B: MeCN) to yield the title compound (12.2 mg, 8.52 μmol, 44%) as a white solid. HPLC (5-95% solvent B in solvent A) retention time 8.257 min; LRMS (ESI) m/z found [M+H]⁺ 1432.9, C₆₈H₉₅N₂₈O₈⁺ required 1431.8, m/z found [M+HCOO]⁻ 1477.1, C₆₉H₉₅N₂₈O₁₀⁻ required 1475.8.

### Example 8—Preparation of Conjugating Reagent 24

### Step 1: Synthesis of (9H-fluoren-9-yl)methyl (6-(2-bromoacetamido)hexyl)carbamate (25)

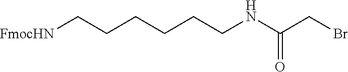

To a solution of Fmoc-hexamethylenediamine hydrochloride (200 mg, 0.533 mmol) and bromoacetyl bromide (116 μL, 1.33 mmol) in CH₂Cl₂ (2 mL) was added saturated aqueous NaHCO₃ (2 mL) and the mixture stirred at rt for 24 h. The reaction mixture was then diluted with H₂O and extracted with CH₂Cl₂ (3×50 mL). The combined organic fractions were washed with 1M HCl (2×50 mL), dried (MgSO₄) and concentrated in vacuo. The crude residue was purified via FCC (50-100% EtOAc/PE) to give the title compound (107 mg, 0.233 mmol, 44%) as a white solid. R_{f} 0.16 (SiO₂; 50% EtOAc/PE); δ_{H} (400 MHz, DMSO-d₆) 8.24 (t, 1H, J=5.2 Hz), 7.89 (d, 2H, J=7.2 Hz), 7.68 (d, 2H, J=7.4 Hz), 7.41 (t, 2H, J=7.4 Hz), 7.31 (td, 2H, J=11.1, 0.7 Hz), 7.25 (t, 1H, J=5.5 Hz), 4.29 (d, 2H, J=7.5 Hz), 4.22-4.18 (m, 1H), 3.82 (s, 2H), 3.05 (q, 2H, J=6.6 Hz), 2.96 (q, 2H, J=6.6 Hz), 1.41-1.36 (m, 4H), 1.25-1.23 (m, 4H); δ_{C} (101 MHz, DMSO-d₆) 165.8, 156.1, 143.9, 140.7, 127.6, 127.0, 125.1, 120.1, 65.1, 46.8, 29.6, 29.3, 28.8, 26.0, 25.9. LRMS (ESI) m/z found [M+H]⁺ 459.3, C₂₃H₂₈Br₁N₂O₃⁺ required 459.1.

### Step 2: Synthesis of Compound 26

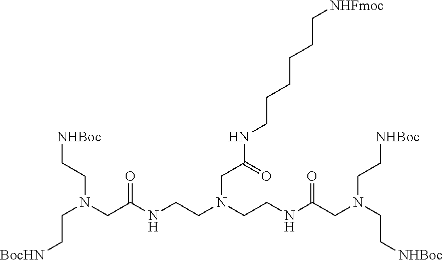

A solution of compound 6 (30.0 mg, 38.0 μmol), compound 25 (29.0 mg, 63.0 μmol) and DIPEA (8.60 μL, 49.4 μmol) in DMF (0.5 mL) was stirred at rt for 21 h. Upon completion, the reaction mixture was concentrated under a stream of N₂. The crude residue was purified by FCC (0-8% MeOH/CH₂Cl₂) to yield the title compound (40.0 mg, 34.0 μmol, 90%) as a white solid. HPLC (5-95% solvent B in solvent A) retention time 10.824 min; LRMS (ESI) m/z found [M+H]⁺ 1169.1, C₅₉H₉₈N₁₁O₁₃⁺ required 1168.7, m/z found [M+HCOO]⁻ 1213.1, C₆₀H₉₈N₁₁O₁₅⁻ required 1212.7.

### Step 3: Synthesis of Compound 27

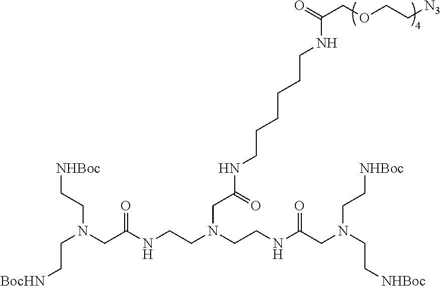

A solution of compound 26 (44.0 mg, 38.0 μmol) and piperidine (7.50 μL, 76.0 μmol) in DMF (1 mL) was stirred at rt for 2 h. Upon completion, the reaction mixture was concentrated under a stream of N₂. To this crude residue was added HBTU (28.8 mg, 76.0 μmol) and DMF (1 mL) followed by N₃-PEG₄-COOH (114 μL, 57.0 μmol, 0.5 M in TBME) and DIPEA (13.2 μL, 76.0 μmol) and the mixture stirred at rt for 20 h. Upon completion, the reaction mixture was concentrated under a stream of N₂ and the crude purified by FCC (0-10% MeOH/CH₂Cl₂) to yield the title compound (38.0 mg, 31.5 μmol, 83%) as a clear oil. HPLC (5-95% solvent B in solvent A) retention time 10.471 min; LRMS (ESI) m/z found [M+H]⁺ 1205.8, C₅₄H₁₀₅N₁₄O₁₆⁺ required 1205.8, m/z found [M+HCOO]⁻ 1250.7, C₅₅H₁₀₅N₁₄O₁₈⁻ required 1249.8.

### Step 4: Synthesis of Conjugating Reagent 24

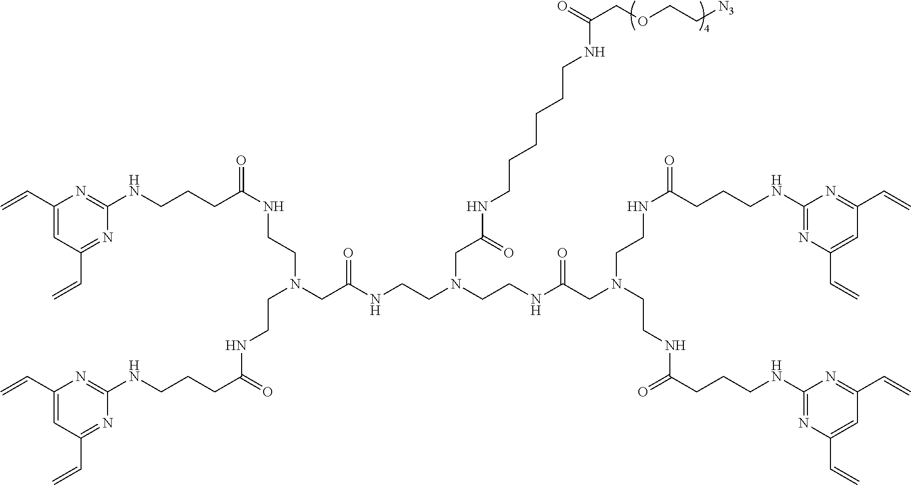

A solution of compound 27 (29.0 mg, 24.0 μmol) in HCl (4 M in 1,4-dioxane, 0.5 mL) and CH₂Cl₂ (0.5 mL) was stirred at rt for 1 h. Upon completion, the reaction mixture was concentrated in vacuo. To this deprotected residue was added HBTU (45.5 mg, 120 μmol), compound 2 (28.0 mg, 120 μmol) and DMF (1 mL) followed by DIPEA (83.8 μL, 480 μmol) and the mixture stirred at rt for 1 h. Upon completion, the reaction mixture was purified by automated reverse phase FCC (10-100% solvent B in solvent A; Solvent A: 0.1 M NH₄OH/H₂O, Solvent B: MeCN) to yield the title compound (12.0 mg, 7.20 μmol, 30%) as a pale yellow solid. HPLC (5-95% solvent B in solvent A) retention time 8.401 min; LRMS (ESI) m/z found [M+Na]⁺ 1689.2, C₈₂H₁₂₄N₂₆²³Na₁O₁₂⁺ required 1689.0, m/z found [M+HCOO]⁻ 1711.1, C₃H₁₂₅N₂₆O₁₄⁻ required 1711.0.

### Example 9—Preparation of Conjugating Reagent 28

### Step 1: Synthesis of Fmoc-Lys(PEG₄-N₃)—OH (29)

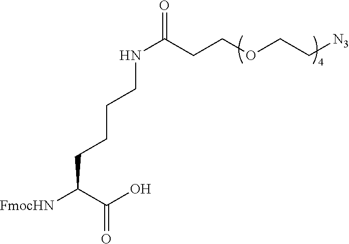

Fmoc-Lys-OH·HCl (500 mg, 1.23 mmol) was suspended in DMF/CH₂Cl₂ (1:1, 3 mL) and a solution of N₃-dPEG₄-NHS ester (480 mg, 1.23 mmol) in DMF/CH₂Cl₂ (1:1, 3 mL) was added dropwise with stirring. K₂CO₃ (324 mg, 2.35 mmol) was added and the mixture was stirred for 23 hours. The reaction mixture was diluted with CH₂Cl₂ and washed with aqueous LiCl (3M, 2×15 mL). The combined aqueous layer was extracted with CH₂Cl₂ (15 mL) and the combined organic extracts concentrated in vacuo. The crude organic fraction was purified by automated reverse phase FCC (10-60% solvent B in solvent A; Solvent A: 0.5% formic acid/H₂O, Solvent B: MeCN) to yield the product, Fmoc-Lys(COdPEG₄-N₃)—OH (560 mg, 0.810 mmol, 66%) as a colourless oil. δ_{H} (400 MHz, CDCl₃) 7.72 (d, 2H, J=7.5 Hz), 7.58 (t, 2H, J=7.3 Hz), 7.36 (t, 2H, J=7.4 Hz), 7.27 (t, 2H, J=7.4 Hz), 6.90 (br s, 1H), 6.23 (br s, 1H), 5.90 (d, 1H, J=7.1 Hz), 4.44-4.30 (m, 3H), 4.18 (t, 1H, J=6.9 Hz), 3.72-3.63 (m, 2H), 3.60 (s, 10H), 3.56 (s, 4H), 3.34 (t, 2H, J=4.9 Hz), 3.23 (qt, 2H, J=6.1 Hz), 2.45 (t, 2H J=5.5 Hz), 1.94-1.82 (m, 1H), 1.81-1.72 (m, 1H), 1.58-1.30 (m, 4H); Sc (101 MHz, CDCl₃) 174.5, 172.6, 171.0, 156.3, 144.0, 143.8, 141.3, 127.7, 127.1, 125.2, 125.2, 120.0, 80.6, 70.6, 70.5, 70.5, 70.3, 70.2, 70.1, 70.0, 67.2, 66.9, 53.8, 50.6, 47.2, 38.9, 36.7, 36.3, 31.7, 28.8, 28.1, 22.1; LRMS (ESI) m/z found [M+H]⁺ 642.5, C₃₂H₄₃N₅O₉⁺ required 642.3.

### Step 2: Synthesis of Ahx-Lyz-Gly-Lys(PEG₄-N₃)-Gly-Lys-Lys-CONH₂ (30)

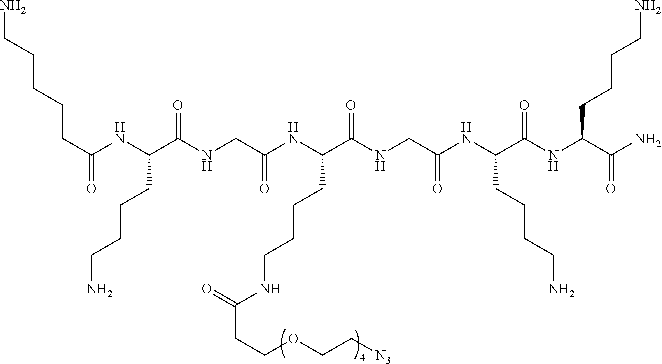

The peptide was synthesised on a 0.1 mmol scale using a general Fmoc solid phase peptide synthesis procedure, producing the desired peptide 30 (40.5 mg, 30.0 μmol, 27%) as a colourless, viscous oil. HPLC (5-95% solvent B in solvent A) retention time 4.313 min; LRMS (ESI) m/z found [M+H]⁺ 1031.3, C₄₅H₈₇N₁₅O₁₂⁺ required 1030.7.

### Step 3: Synthesis of Conjugating Reagent 28

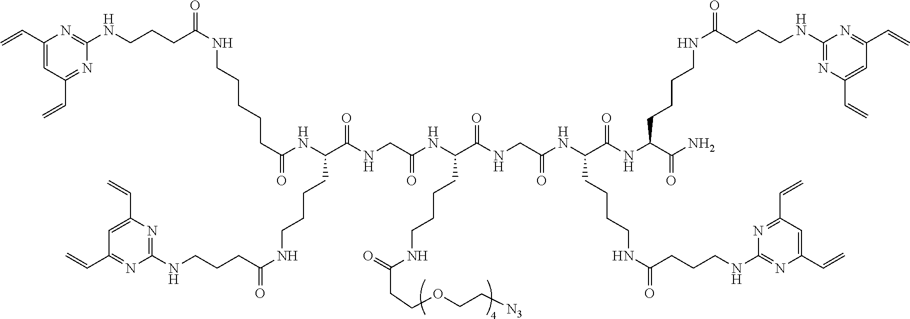

Compound 30 (35.0 mg, 24.0 μmol) was suspended in DMF/CH₂Cl₂ (1:1, 0.4 mL)) and cooled to 0° C. A suspension of compound 2 (27.5 mg, 120 μmol), HBTU (44.7 mg, 120 μmol) and NEt₃ (66.0 uL, 470 μmol) in DMF/CH₂Cl₂ (1:1, 0.4 mL) was added and the mixture stirred for 3 hours. The mixture was purified by automated reverse phase FCC (10-60% solvent B in solvent A; Solvent A: H₂O, Solvent B: MeCN) to yield the title compound (18.3 mg, 9.70 μmol, 40%) as a colourless viscous oil. HPLC (5-95% solvent B in solvent A) retention time 8.084 min; LRMS (ESI) m/z found [M+Na]⁺ 1914.2, C₉₃H₁₃₉²³Na₁N₂₇O₁₆⁺ required 1914.1, m/z found [M+HCOO⁻]⁻ 1936.3, C₉₄H₁₄₀N₂₇O₁₈⁻ required 1936.1.

### Example 10—Preparation of Conjugating Reagent 31

### Step 1: Synthesis of Ahx-Lyz-Gly-Lys(PEG₄-N₃)-Gly-Lys(PEG₄-N₃)-Gly-Lys-Lys-CONH₂ (32)

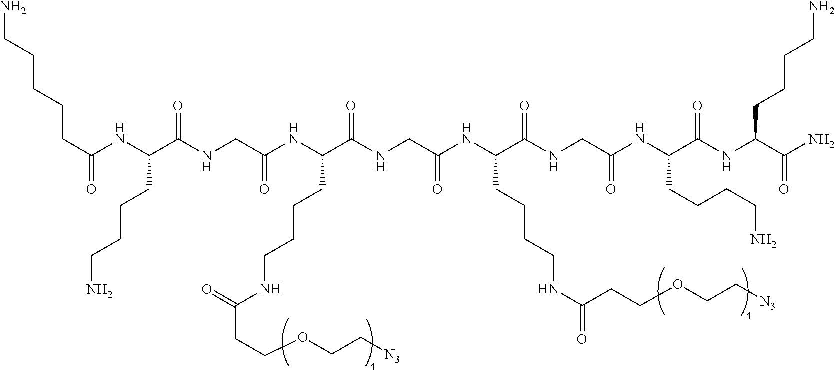

The peptide was synthesised on a 0.1 mmol scale using the general peptide synthesis procedure, producing the product (30.6 mg, 16.0 μmol, 5%) as a colourless, viscous oil. HPLC (5-95% solvent B in solvent A) retention time 5.942 min; LRMS (ESI) m/z found [M+H]⁺ 1489.8, C₆₄H₁₂₂N₂₁O₁₉⁺ required 1488.9, m/z found [M+HCOO⁻]⁻ 1532.9, C₆₅H₁₂₂N₂₁O₂₁⁻ required 1533.7.

### Step 2: Synthesis of Conjugating Reagent 31

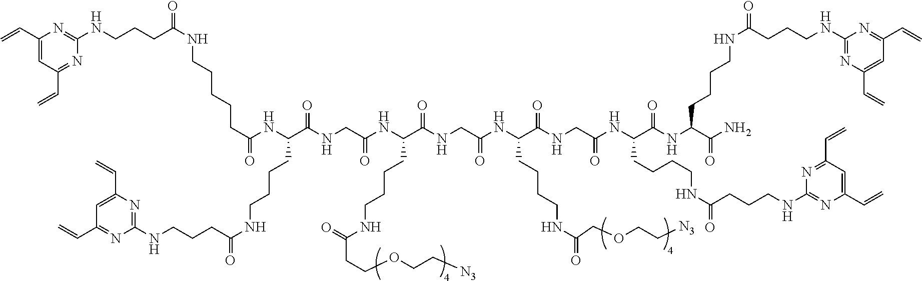

Compound 32 (23.3 mg, 12.0 μmol) was suspended in DMF (0.5 mL) and cooled to 0° C. A suspension of compound 2 (14.0 mg, 60.0 μmol), HBTU (22.7 mg, 60.0 μmol) and NEt₃ (33.4 uL, 240 μmol) in DMF (0.5 mL) was added and the mixture stirred for 5 hours. The mixture was purified by automated reverse phase FCC (40-55% MeCN in H₂O) to yield the title compound (1.81 mg, 0.770 μmol, 5%) as a colourless viscous oil. HPLC (5-95% solvent B in solvent A) retention time 8.376 min; LRMS (ESI) m/z found [M+2H]²⁺ 1176.3, C₁₁₂H₁₇₅N₃₃O₄₃²⁺ required 1175.7.

### Example 11—Preparation of Conjugating Reagent 33

### Step 1: Synthesis of Ac-Lys(PEG₄N₃)-PEG₂Glu-CONH₂ (34)

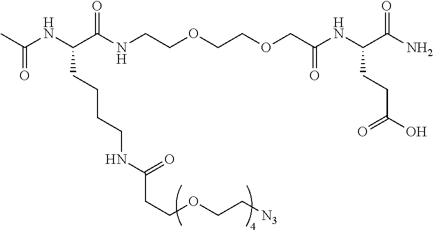

The peptide was synthesised on a 0.3 mmol scale using the general peptide synthesis procedure, producing the desired product (44.1 mg, 60.0 μmol, 60%) as a colourless, viscous oil. HPLC (5-95% solvent B in solvent A) retention time 5.985 min; LRMS (ESI) m/z found [M+H]735.5, C₃₀H₅₄N₈O₁₃⁺ required 735.4.

### Step 2: Synthesis of Compound 35

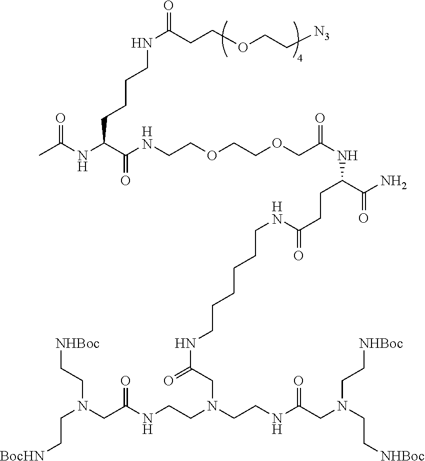

Compound 26 (20.0 mg, 17.0 μmol) was dissolved in DMF (0.5 mL) and piperidine (3.40 uL, 34.0 μmol) was added. The mixture was stirred for 90 minutes, then concentrated under N₂. The crude mixture was redissolved in DMF (0.5 mL) and compound 34 (18.9 mg, 26.0 μmol), HBTU (13.0 mg, 34.0 μmol) and DIPEA (6.00 μL, 34.0 μmol) were added. The reaction mixture was stirred for 3 hours. The mixture was purified by automated reverse phase FCC (10-100% solvent B in solvent A; Solvent A: 0.1 M NH₄OH/H₂O, Solvent B: MeCN) to yield the title compound (11.4 mg, 6.90 μmol, 40%) as a pale yellow powder. HPLC (5-95% solvent B in solvent A) retention time 8.538 min; LRMS (ESI) m/z found [M+2H]²⁺ 832.4, C₇₄H₁₃₉N₁₉O₂₃⁺ required 832.0.

### Step 3: Synthesis of Conjugating Reagent 33

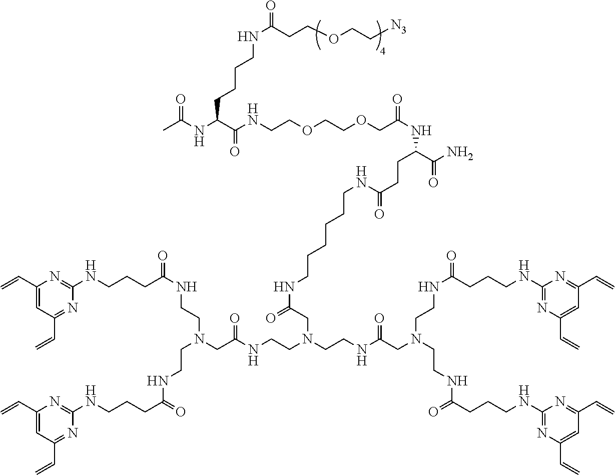

Compound 35 (7.00 mg, 4.20 μmol) was dissolved in CH₂Cl₂ (0.3 mL) and HCl (2M in Et₂O, 0.3 mL) added. The mixture was stirred for 1 hour and then concentrated. The crude mixture was redissolved in DMF (0.5 mL) and compound 2 (7.00 mg, 30.0 μmol), HBTU (11.4 mg, 30.0 μmol) and DIPEA (10.5 μL, 60.0 μmol) were added. The mixture was stirred for 2 hours and then purified twice via automated reverse phase FCC (40-70% MeCN in H₂O) to yield the desired product (0.120 mg, 0.600 μmol, 1.3%) as a yellow residue. HPLC (5-95% solvent B in solvent A) retention time 7.66 min; LRMS (ESI) m/z found [M+Na]⁺ 2124.4, C₁₀₂H₁₆₀N₃₁O₁₉⁺ required 2124.3, m/z found [M+HCOO⁻]⁻ 2169.0, C₁₀₃H₁₆₀N₃₁O₂₁⁻ required 2168.2.

### Example 12—Preparation of Conjugates 36-39

To a solution of trastuzumab (198 μL, 17 μM, 2.5 mg/mL) in TBS (25 mM Tris HCl pH 8, 25 mM NaCl, 0.5 mM EDTA) was added TCEP (10 equiv.). The mixture was vortexed and incubated at 37° C. for 1 h. A solution of conjugating reagent 1, 8, 11 or 14 (10 mM in DMSO) was added (final concentration of 34 μM, 2 equiv.) and the reaction mixture incubated at 37° C. for 4 h. The excess reagents were removed by use of a Zeba™ Spin Desalting Column (40,000 MWCO, Thermo Fisher Scientific), followed by repeated diafiltration into PBS using an Amicon-Ultra centrifugal filter (10,000 MWCO, Merck Millipore). Samples were stored at 4° C. until analysis. LC-MS and SDS-PAGE analysis demonstrated >95% conversion to the corresponding bridged conjugates 36, 37, 38 and 39.

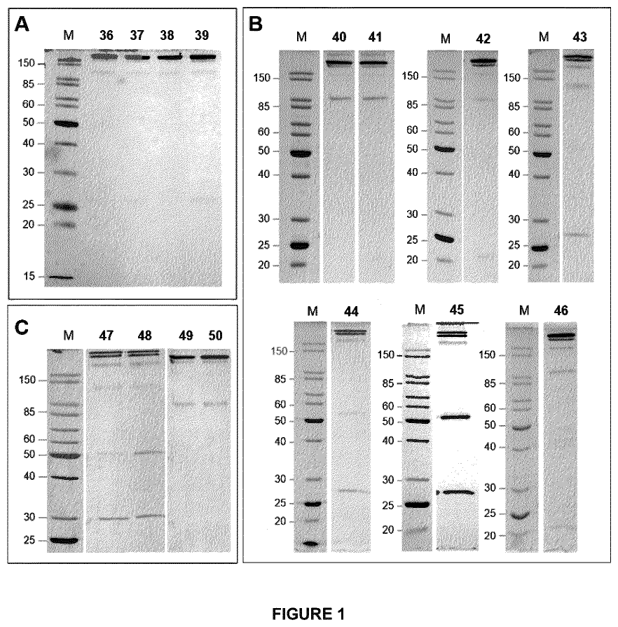

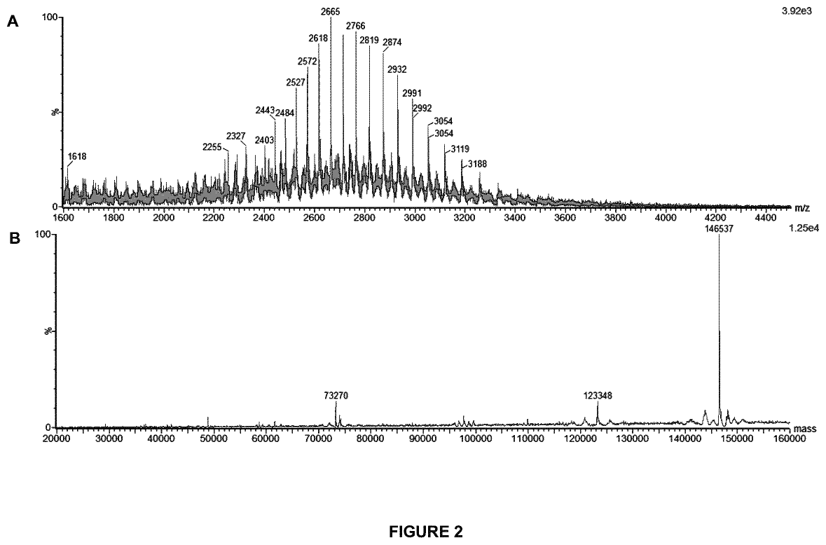

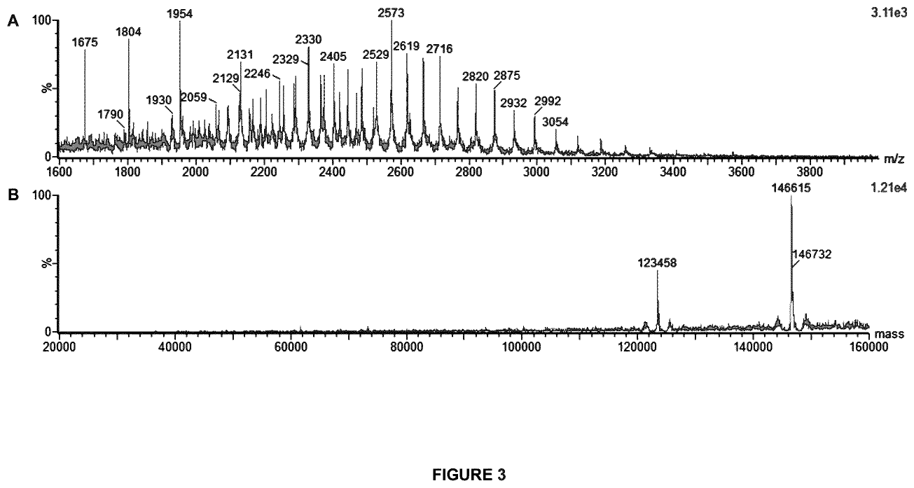

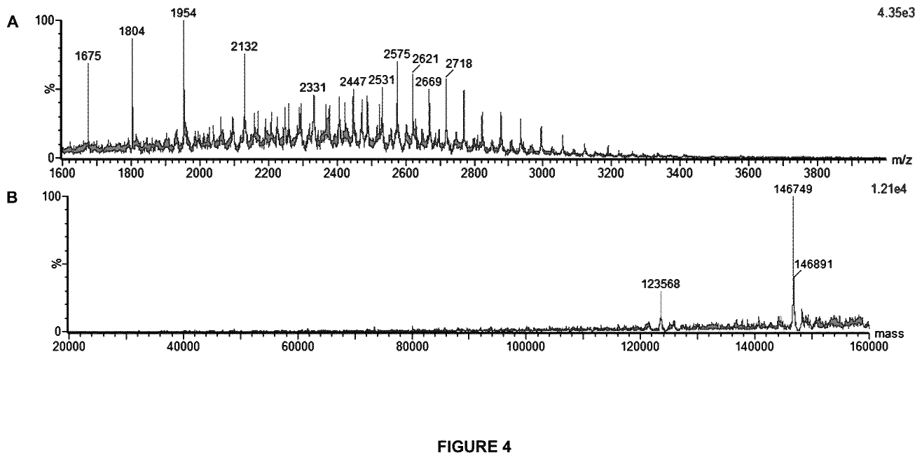

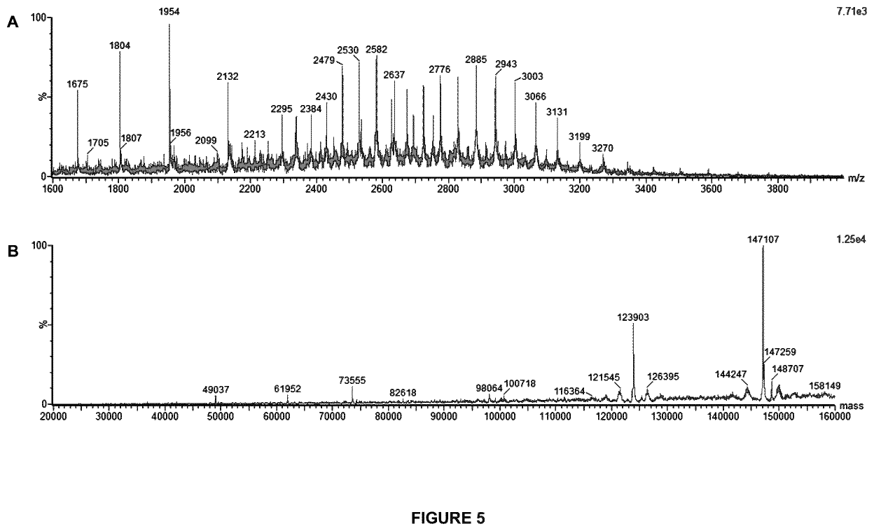

FIG. 1(A) shows the SDS-PAGE traces for conjugates 36, 37, 38 and 39. FIGS. 2 to 5 show the LC-MS traces for conjugates 36, 37, 38 and 39.

### Example 13—Preparation of Conjugates 40-46

To a solution of trastuzumab (50 μL, 17 μM, 2.5 mg/mL) in TBS (25 mM Tris HCl pH 8, 25 mM NaCl, 0.5 mM EDTA) was added TCEP (10 equiv.). The mixture was vortexed and incubated at 37° C. for 1 h. A solution of conjugating reagent 19, 21, 22, 24, 28, 31 or 33 (5 mM in DMSO) was added (final concentration of 34 μM, 2 equiv.) and the reaction mixture incubated at 37° C. for 2 h. The excess reagents were removed by use of a Zeba™ Spin Desalting Column (40,000 MWCO, Thermo Fisher Scientific). Samples were either stored at 4° C. or flash frozen and stored at −20° C. until analysis. LC-MS and SDS-PAGE analysis demonstrated >95% conversion to the corresponding bridged conjugates 40, 41, 42, 43, 44, 45 and 46.

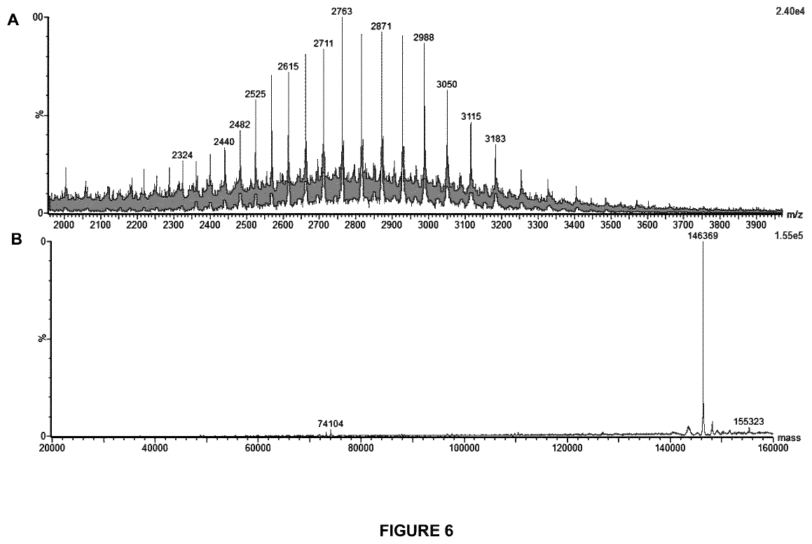

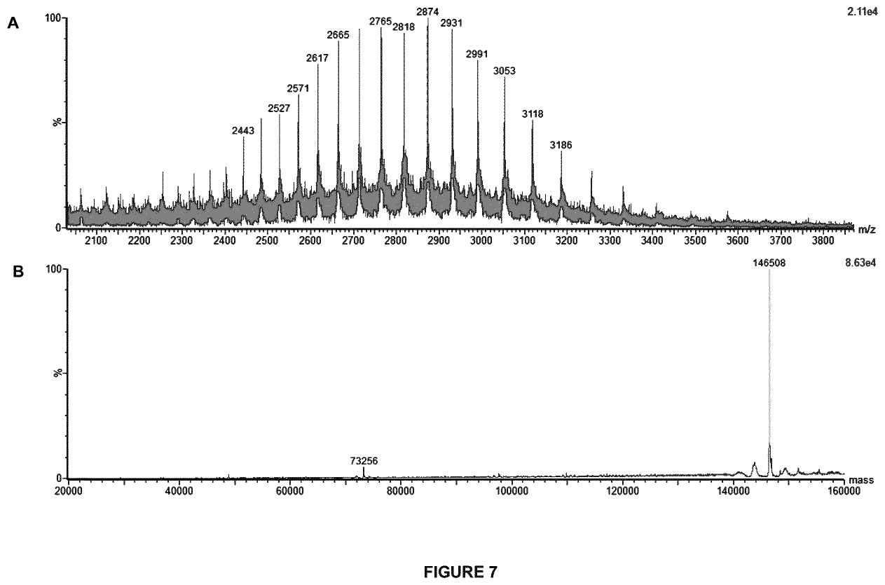

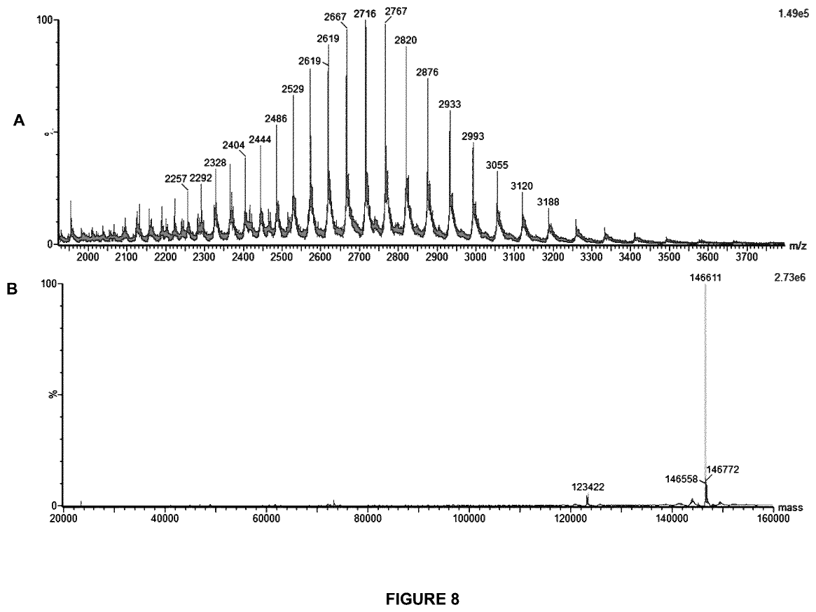

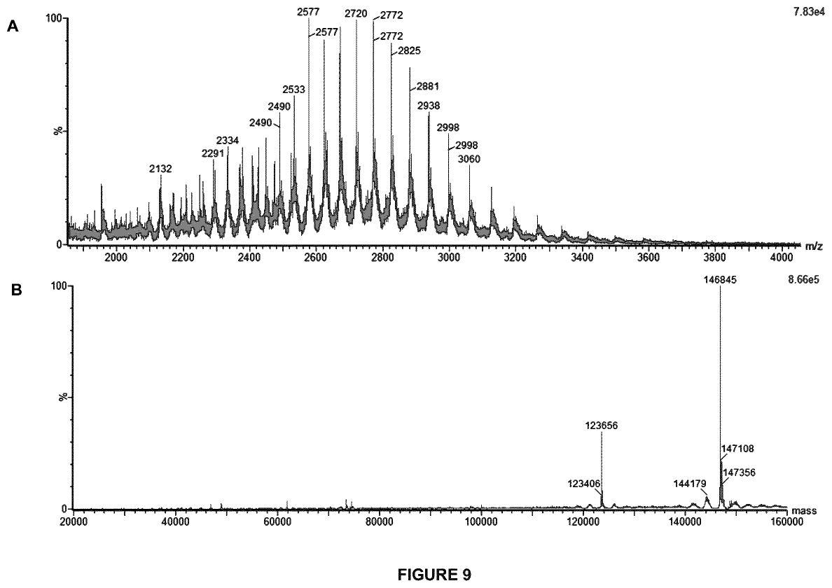

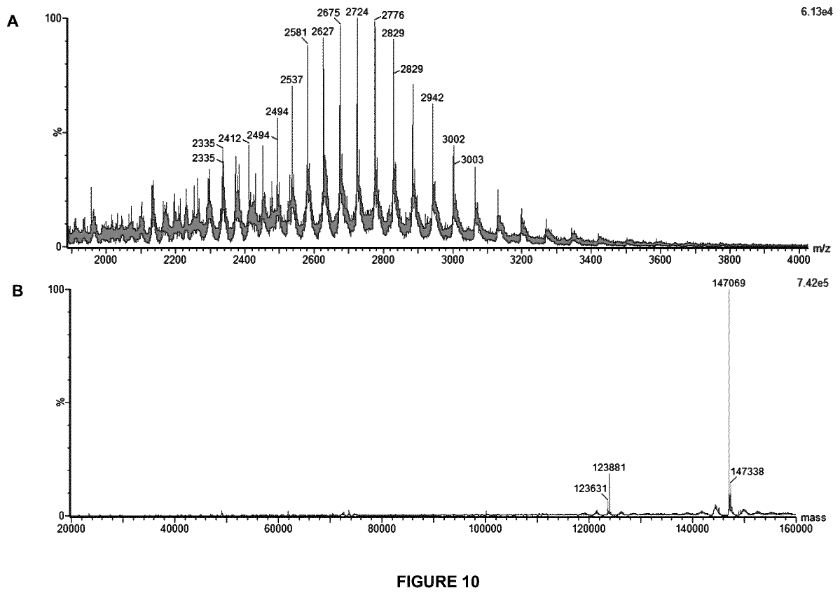

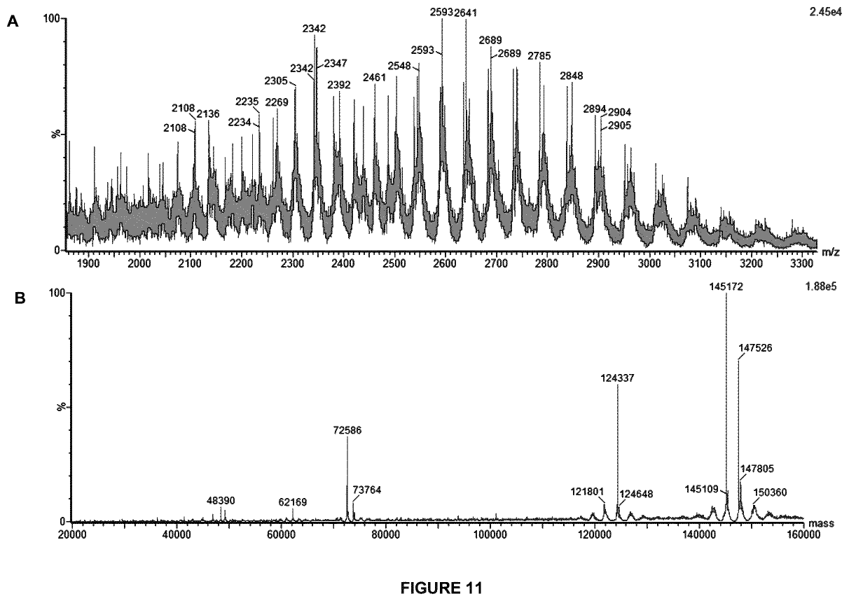

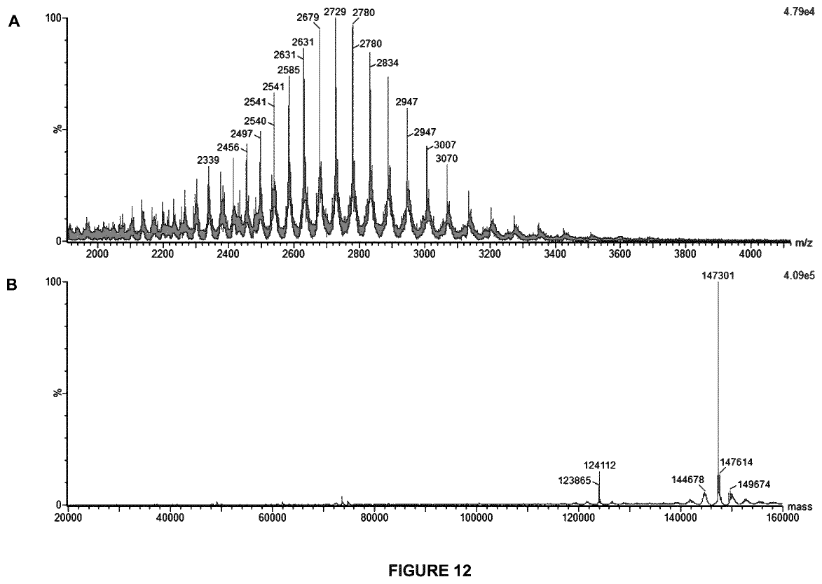

FIG. 1(B) shows the SDS-PAGE traces for conjugates 40, 41, 42, 43, 44, 45 and 46. FIGS. 6 to 12 show the LC-MS traces for conjugates 40, 41, 42, 43, 44, 45 and 46.

### Example 14—Preparation of Conjugates 47-50

To a solution of brentuximab (50 μL, 17 μM, 2.5 mg/mL) in TBS (25 mM Tris HCl pH 8, 25 mM NaCl, 0.5 mM EDTA) was added TCEP (10 equiv.). The mixture was vortexed and incubated at 37° C. for 1 h. A solution of conjugating reagent 1, 8, 19 or 21 (5 mM in DMSO) was added (final concentration of 34 μM, 2 equiv.) and the reaction mixture incubated at 37° C. for 2 h. The excess reagents were removed by use of a Zeba™ Spin Desalting Column (40,000 MWCO, Thermo Fisher Scientific). Samples were either stored at 4° C. or flash frozen and stored at −20° C. until analysis. LC-MS and SDS-PAGE analysis demonstrated >95% conversion to the corresponding bridged conjugates 47, 48, 49 and 50.

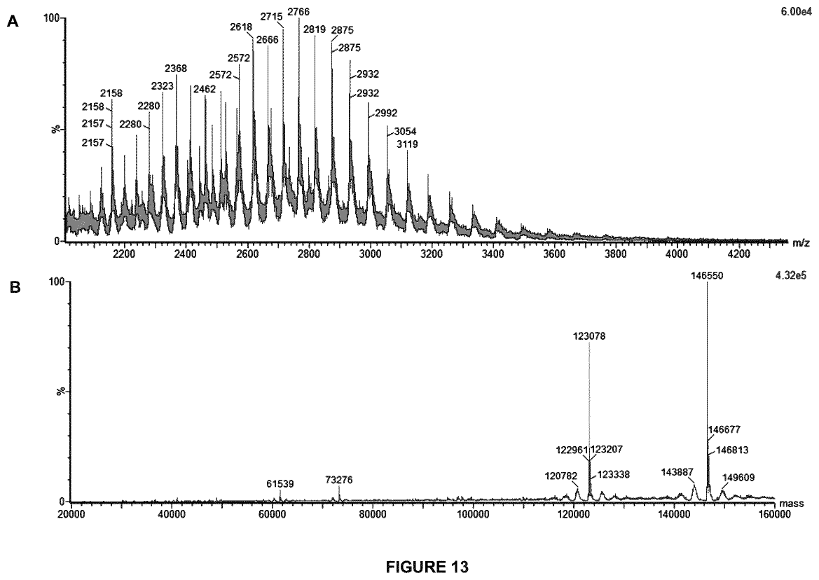

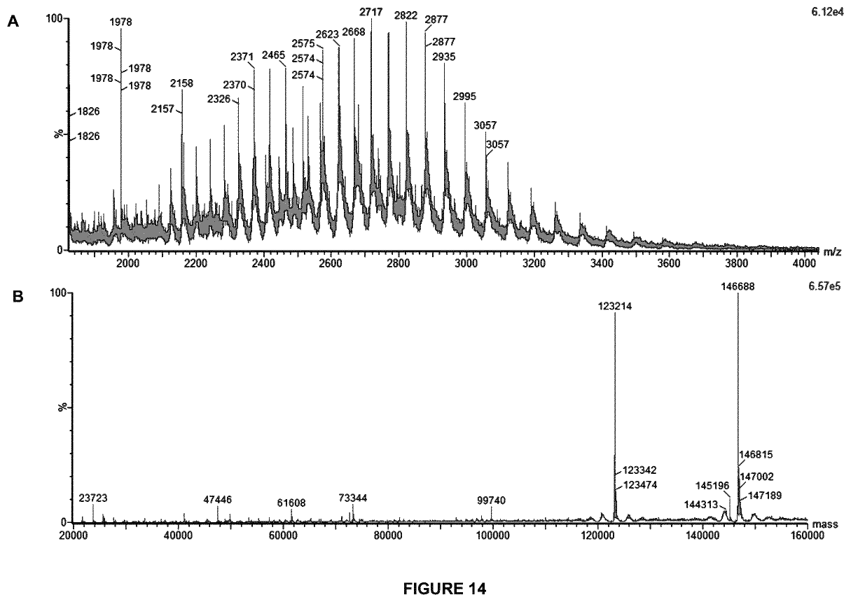

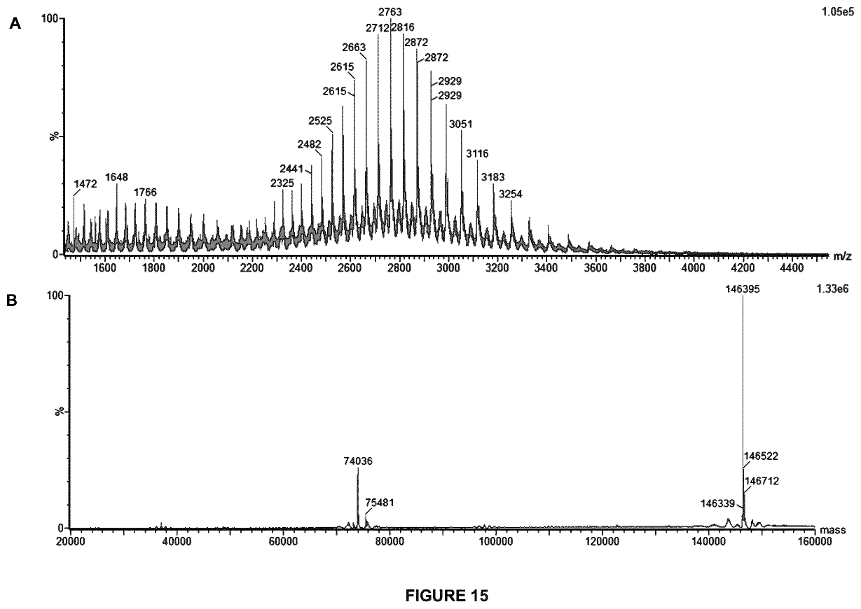

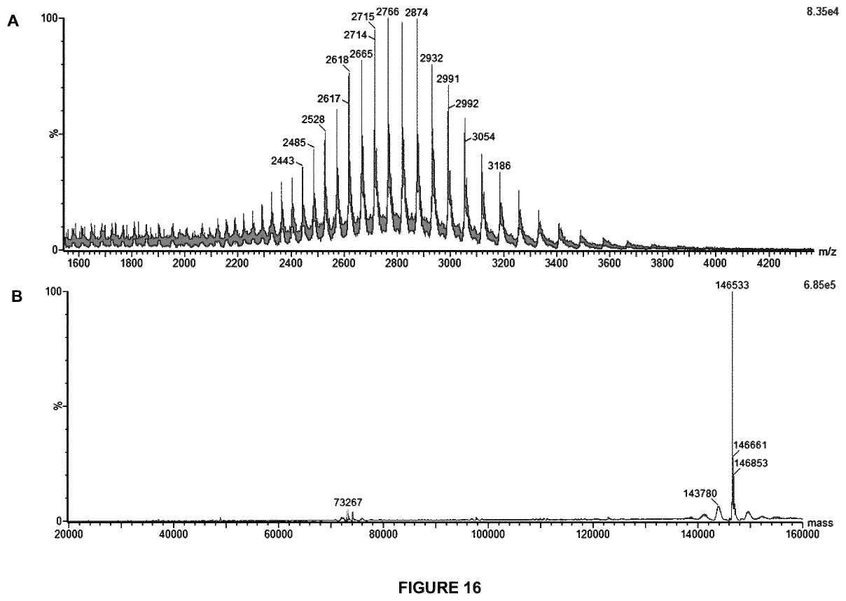

FIG. 1(C) shows the SDS-PAGE traces for conjugates 47, 48, 49 and 50. FIGS. 13 to 16 show the LC-MS traces for conjugates 47, 48, 49 and 50.

### Example 15—Size-Exclusion Chromatography

Size-exclusion chromatography (SEC) was carried out using a Superdex 200 10/300 GL column. Samples were injected at a concentration of 1 mg/mL and eluted with TBS buffer (25 mM Tris HCl pH 8, 200 mM NaCl, 0.5 mM EDTA) at a flow rate of 0.5 mL/min.

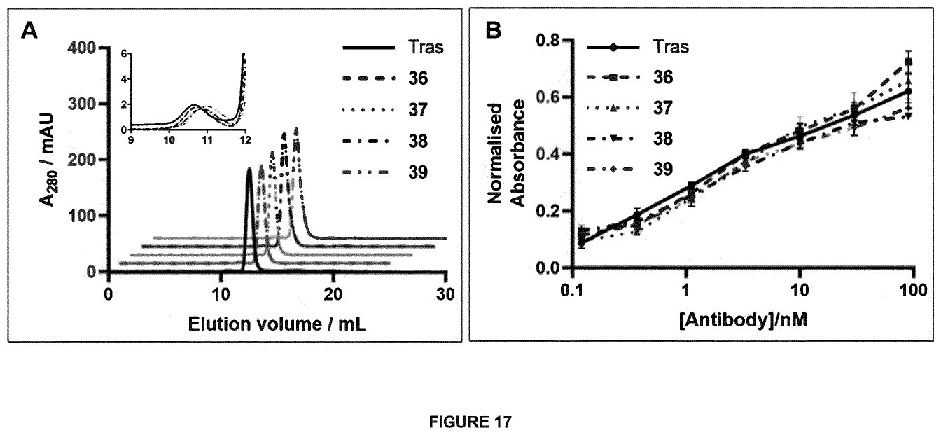

Size exclusion chromatograms for conjugates 36, 37, 38 and 39 are shown in FIG. 17(A).

### Example 16—Enzyme-Linked Immunosorbent Assay (ELISA)

A 96-well plate was coated with 100 μL of a 0.25 μg/mL solution of HER2 (Sino Biological, His-tagged) overnight at 4° C. Coating solutions were removed and each well washed with PBS (2×200 μL). Each well was then blocked with 1% BSA in PBS (200 μL) for 1 h at room temperature. The blocking solution was then removed and each well washed with PBS (3×200 μL). Wells were treated with a serial dilution of trastuzumab and trastuzumab conjugates 36, 37, 38 and 39 in PBS (100 μL of 90 nM, 30 nM, 10 nM, 3.33 nM, 1.11 nM, 0.37 nM, 0.12 nM, 0 nM) and incubated at room temperature for 2 h. The conjugate solutions were removed, and each well was washed with 0.1% Tween 20 in PBS (2×200 μL) followed by PBS (3×200 μL). Next, 100 μL of detection antibody (1:500 dilution of a mouse anti-human IgG-HRP, ThermoFisher) in PBS was added to each well and incubated at room temperature for 1 h. Each well was washed with 0.1% Tween 20 in PBS (2×200 μL) followed by PBS (3×200 μL). Finally, an OPD solution (100 μL of a solution prepared by dissolving 1 capsule in 9 mL H₂O and 1 mL stable peroxide substrate buffer (10×), ThermoFisher) was added to each well. After 15 minutes, 4M HCl (aq.) (50 μL) was added to each well to quench the reaction. Absorbance at 490 nm and 590 nm was measured using a CLARIOstar Microplate Reader. Measurements were performed in quadruplicate and three independent repeats were performed.

ELISA results for conjugates 36, 37, 38 and 39 are shown in FIG. 17(B).

### Example 17—Preparation of Antibody-Fluorophore Conjugates 51-54

To a solution of trastuzumab conjugates 36, 37, 38 or 39 in PBS was added CuSO₄·5H₂O (20 equiv. per alkyne), THPTA (100 equiv. per alkyne), sodium ascorbate (150 equiv. per alkyne) and AlexaFluor™ 488 Azide (Thermo Fisher Scientific) (5 mM in DMSO, 12 equiv. per alkyne). The mixture was vortexed and incubated at 37° C. for 4 h (for 36 and 37) or 6 h (for 38 and 39). The excess reagents were removed by use of a Zeba™ Spin Desalting Column (7,000 MWCO, Thermo Fisher Scientific), followed by repeated diafiltration into PBS using an Amicon-Ultra centrifugal filter (10,000 MWCO, Merck Millipore). UV-vis analysis revealed conversion of 36, 37, 38 and 39 to antibody-fluorophore conjugates 51, 52, 53 and 54 with average fluorophore-to-antibody ratios (FAR) of 1.0, 2.0, 2.9 and 4.0, respectively. Each reaction was repeated at least three times and gave consistent results with FAR values within ±0.1 units of those reported above.

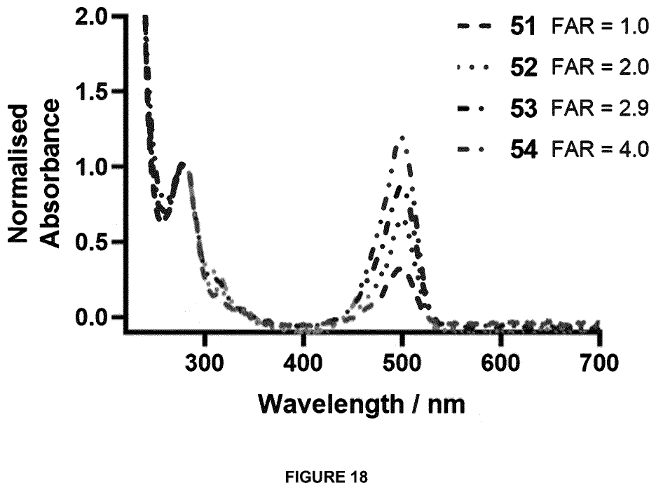

FIG. 18 shows the measured fluorophore-to-antibody ratios (FAR) for conjugates 51, 52, 53 and 54.

### Example 18—Stability Analysis

To a solution of trastuzumab-AlexaFluor488 conjugates 51, 52, 53 and 54 (138 μL, 3.75 μM) in PBS were added 12 μL of reconstituted human plasma (Sigma). The mixture was incubated at 37° C. for 14 days. Aliquots were removed every 2 days, flash frozen, and stored at −80° C. until analysis. SDS-PAGE was followed by in-gel fluorescence and coomassie brilliant blue staining.

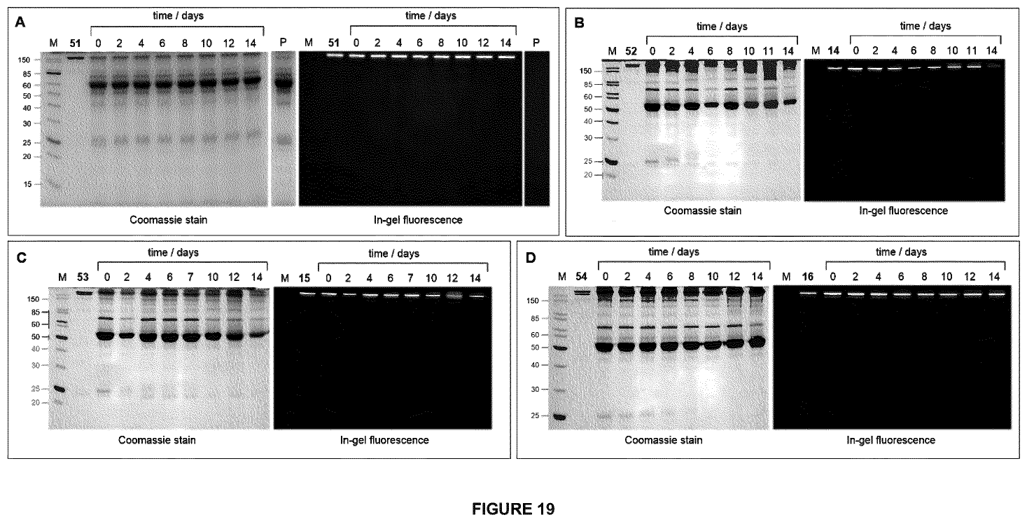

FIG. 19 shows the stability analysis of conjugates 51, 52, 53 and 54 in human plasma by SDS-PAGE. No transfer of the fluorescent payload to plasma proteins was observed over the entire duration of the study, showcasing that the conjugates of the invention are stable under physiological conditions.

### Example 19—Preparation of Payload 55

### Step 1: Synthesis of Payload 55

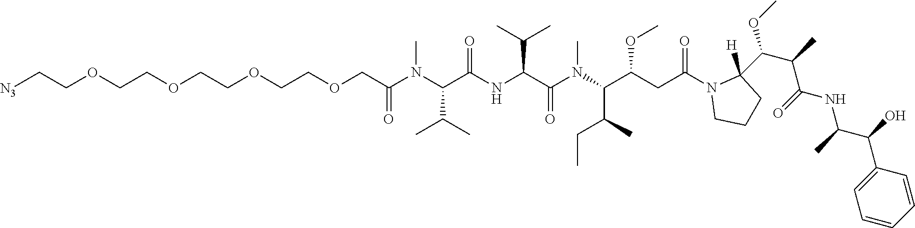 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)COCCOCCOCCOCCN=[N+]=[N-])C(C)C]

Payload 55 was synthesised as previously described (Walsh et al, Chem. Sci., 2019, 10 (3), 694-700).

### Example 20—Preparation of Payload 56

### Step 1: Synthesis of Fmoc-Val-Cit-PABC-MMAE (57)

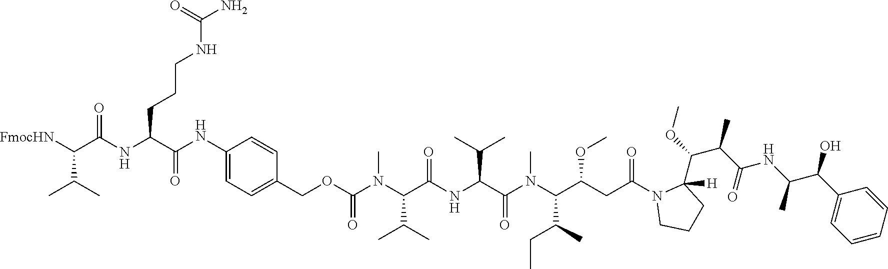

A solution of Fmoc-Val-Cit-PABC-PNP (21.6 mg, 30.1 μmol), MMAE (30.0 mg, 39.1 μmol), HOBt·H₂O (10.1 mg, 60.0 μmol), DIPEA (15.7 μL, 90.0 μmol) and pyridine (24.3 μL, 300 μmol) in DMF (1 mL) was stirred at rt for 24 h. Upon completion, the reaction mixture was purified by automated reverse phase FCC (10-70% solvent B in solvent A. Solvent A: 0.1 M NH₄OH (aq). Solvent B: MeCN) and lyophilised to yield the desired compound (39.0 mg, 29.0 μmol, 96%) as a white solid. HPLC (5-95% solvent B in solvent A) retention time 13.189 min; LRMS (ESI) m/z found [M+Na]⁺ 1368.6, C₇₃H₁₀₄N₁₀²³Na₁O₁₄⁺ required 1367.8.

### Step 2: Synthesis of Payload 56

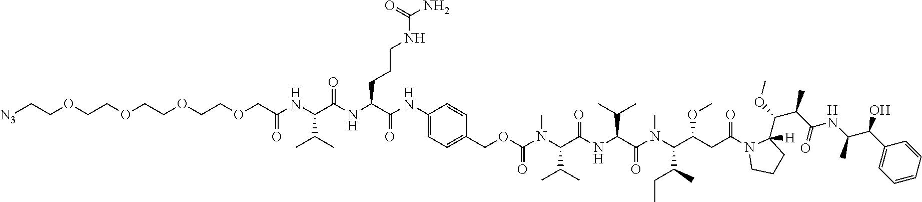 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)COCCOCCOCCOCCN=[N+]=[N-])C(C)C)cc1)C(C)C]

To a solution of Fmoc-Val-Cit-PABC-MMAE (57) (20.0 mg, 14.9 μmol) in DMF (2 mL) was added piperidine (7.30 μL, 74.3 μmol) and the mixture stirred at rt for 2 h. Upon completion, the reaction was concentrated under a stream of N₂. To the free amine was added HBTU (11.3 mg, 29.8 μmol) and DMF (2 mL) followed by N₃-PEG₄-CO₂H (50 μL of a 0.5 M solution in TBME, 22.4 μmol) and DIPEA (7.80 μL, 44.7 μmol) and the reaction mixture stirred at rt for 18 h. Upon completion, the reaction was concentrated under a stream of N₂ and purified by automated reverse phase FCC (10-100% solvent B in solvent A. Solvent A: 0.1 M NH₄OH (aq). Solvent B: MeCN) and lyophilised to yield the title compound (13.6 mg, 9.80 μmol, 66%) as a white solid. HPLC (5-95% solvent B in solvent A) retention time 11.319 min; LRMS (ESI) m/z found [M+H]⁺ 1282.8, C₆₈H₁₁₂N₁₃O₁₇⁺ required 1382.8, m/z found [M+HCOO]⁻ 1428.0, C₆₉H₁₁₂N₁₃O₁₉ ⁻ required 1426.8.

### Example 21—Preparation of Non-Cleavable Antibody-Drug Conjugates 58-61

To a solution of trastuzumab conjugates 36, 37, 38 or 39 in PBS was added CuSO₄·5H₂O (150 equiv.), THPTA (600 equiv.), sodium ascorbate (1000 equiv.) and compound 55 (20 mM in DMSO, 100 equiv.). The mixture was vortexed and incubated at 37° C. for 6 h. The excess reagents were removed by use of a Zeba™ Spin Desalting Column (40,000 MWCO, Thermo Fisher Scientific), followed by repeated diafiltration into PBS using an Amicon-Ultra centrifugal filter (10,000 MWCO, Merck Millipore). Hydrophobic interaction chromatography (HIC) revealed conversion of 36, 37, 38 and 39 to antibody-drug conjugates 58, 59, 60 and 61 with average drug-to-antibody ratios (DAR) of 0.8, 1.4, 2.1 and 2.8, respectively.

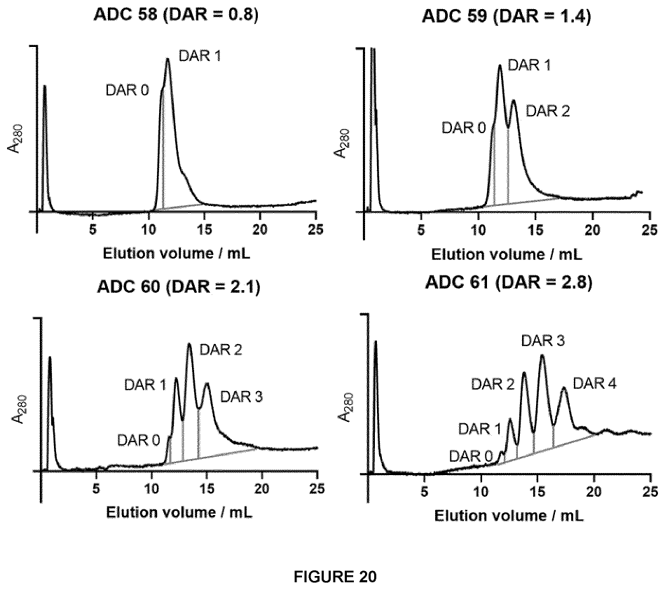

Hydrophobic interaction chromatograms for conjugates 58, 59, 60 and 61 are shown in FIG. 20.

### Example 22—Preparation of Cleavable Antibody-Drug Conjugates 62-65

To a solution of trastuzumab conjugates 36, 37, 38 or 39 in PBS was added CuSO₄·5H₂O (150 equiv.), THPTA (600 equiv.), sodium ascorbate (1000 equiv.) and compound 56 (20 mM in DMSO, 100 equiv.). The mixture was vortexed and incubated at 37° C. for 6 h. The excess reagents were removed by use of a Zeba™ Spin Desalting Column (40,000 MWCO, Thermo Fisher Scientific), followed by repeated diafiltration into PBS using an Amicon-Ultra centrifugal filter (10,000 MWCO, Merck Millipore). Hydrophobic interaction chromatography (HIC) revealed conversion of 36, 37, 38 and 39 to antibody-drug conjugates 62, 63, 64 and 65 with average drug-to-antibody ratios (DAR) of 0.5, 1.0, 1.6 and 2.4, respectively.

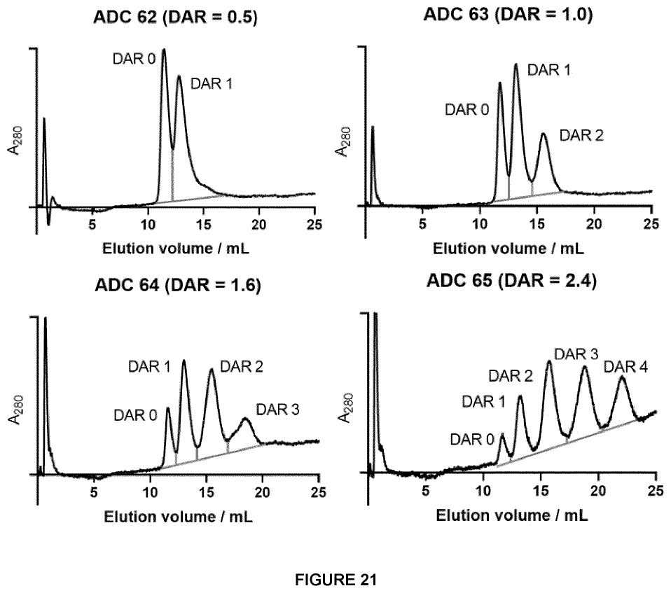

Hydrophobic interaction chromatograms for conjugates 62, 63, 64 and 65 are shown in FIG. 21.

### Example 23—In Vitro Cytotoxicity

### Cell Lines

HER2-positive SKBR3 and BT474 cells were obtained from the American Type Culture Collection (ATCC) and HER2-negative MCF7 and MDA-MB-468 cells were obtained from the European Collection of Authenticated Cell Cultures (ECACC) and ATCC, respectively. SKBR3 cells were maintained in high glucose McCoy's 5A medium, supplemented with 10% heat-inactivated foetal-bovine serum (FBS), GlutaMAX™, 50 U/mL penicillin and 50 μg/mL streptomycin. MCF7 and MDA-MB-468 cells were maintained in Dulbecco's Modified Eagle Medium (DMEM) supplemented with 10% heat-inactivated fetal-bovine serum (FBS), 2 mM L-glutamine, 50 U/mL penicillin and 50 μg/mL streptomycin. BT474 cells were maintained in RPMI1640 medium supplemented with 10% heat-inactivated fetal-bovine serum (FBS), 2 mM L-glutamine, 50 U/mL penicillin and 50 μg/mL streptomycin. All cell lines were incubated at 37° C. with 5% CO₂.

### Cell Viability

Cells were seeded in 96-well plates for 24 h at 37° C. with 5% CO₂. SKBR3 cells were seeded at 15,000 cells/well, BT474 cells were seeded at 20,000 cells/well, MCF7 cells were seeded at 7,500 cells/well and MDA-MB-468 cells were seeded at 10,000 cells/well. Serial dilutions of 62, 63, 64, 65 and trastuzumab were added to the cells in complete growth medium and incubated at 37° C. with 5% CO₂ for 96 h. Cell viability was measured using CellTiter-Glo viability assay (Promega) according to the manufacturer's instructions. Cell viability was plotted as a percentage of untreated cells. Each measurement was taken in triplicate and three independent repeats were performed.

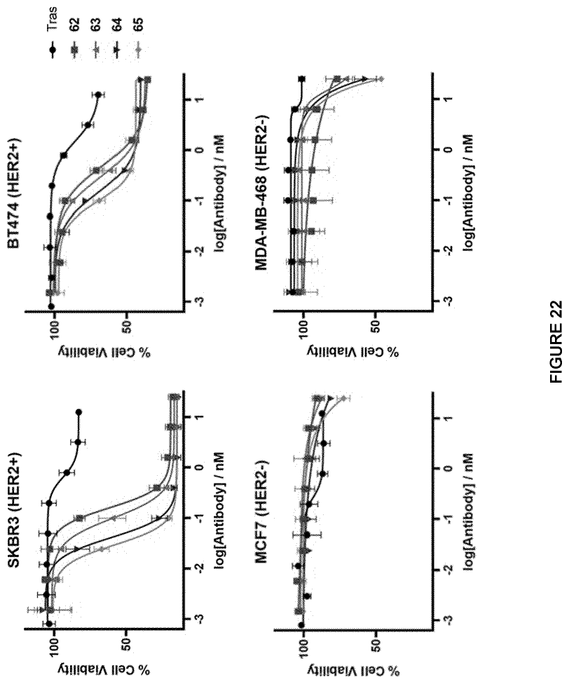

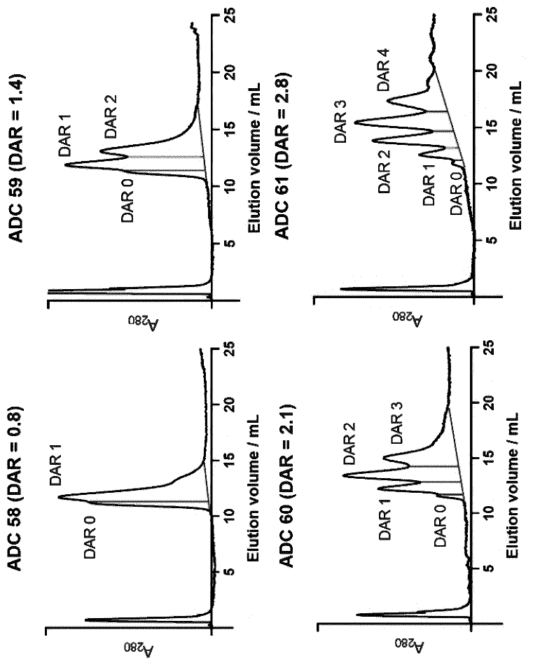

FIG. 22 shows cytotoxicity of conjugates 62, 63, 64, 65 and trastuzumab.

Each of the conjugates displayed a significant increase in cytotoxicity in HER2-positive cells compared to trastuzumab alone. In contrast, the proliferation of HER2-negative cells was not affected compared to vehicle control, thus confirming the selectivity for HER2-positive cells. Also, higher DAR conjugates (conjugates 64 and 65) displayed an increase in cytotoxicity relative to lower DAR conjugates (conjugates 62 and 63). This showcases the value of DAR tunability offered by the present invention.

### Example 24—Live Cell Microscopy

SKBR3 or MCF7 cells were seeded at 40,000 cells/well in 8-well chambered μ-slide (Ibidi, 80826) for 48 h at 37° C. with 5% CO₂. Slides were then placed on ice and washed with Ham's F12 Nutrient Mix media containing 10% FBS, 2 mM L-glutamine, 50 U/mL penicillin and 50 μg/mL streptomycin (3×200 μL). Antibody conjugates 51 and 52 (50 nM), trastuzumab (50 nM) or vehicle (PBS) were added to the cells in complete F12 growth medium and incubated at 4° C. in the dark for 1 h. Cells were placed back on ice and washed with complete F12 growth medium (3×200 μL). Complete F12 growth medium (200 μL) was added and the cells incubated for 3.5 h at 37° C. with 5% CO₂. After 3 h incubation, Hoechst 33342 trihydrochloride trihydrate (1 μg/mL, Invitrogen, H3570) was added. Live cell microscopy was performed on an Operetta CLS confocal microscope (Perkin Elmer) with a 40× water objective. Cells were maintained in a humidified atmosphere at 37° C. and 5% CO₂ throughout analysis. Data analysis was performed using ImageJ (Fiji).

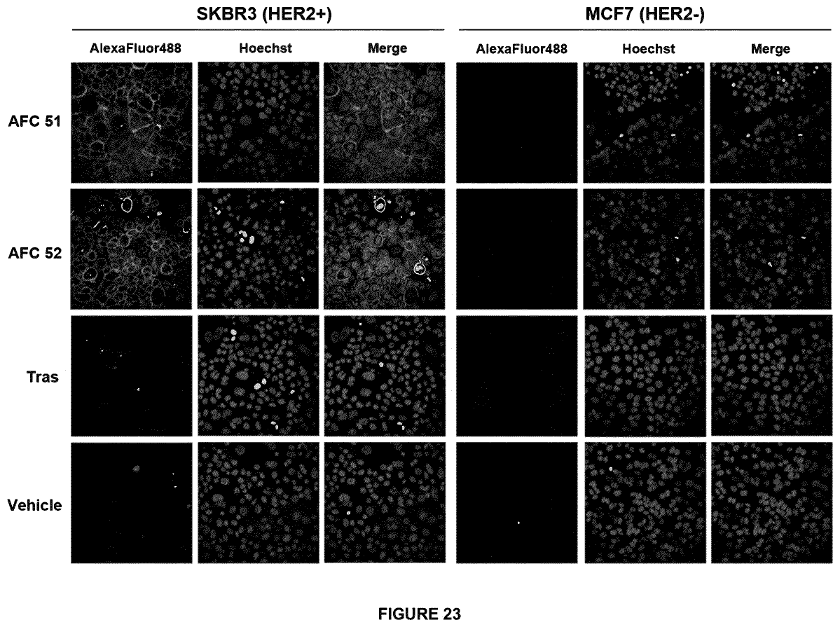

Microscopy images are shown in FIG. 23. Both conjugates can be seen to selectively label and internalise into HER2-positive SKBR3 cells. No labelling is observed for HER2-negative MCF7 cells.

### Example 25—Synthesis of TetraDVP Scaffold A

### Step 1: Synthesis of Compound 202

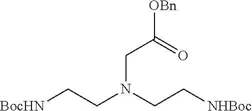 [SMILES: CC(C)(C)OC(=O)NCCN(CCNC(=O)OC(C)(C)C)CC(=O)OCc1ccccc1]

Compound 3 (4.43 g, 14.6 mmol) was dissolved in DMF (49 mL) and benzyl bromoacetate (3.47 mL, 21.9 mmol) and DIPEA (3.05 mL, 17.5 mmol) were added dropwise. The reaction mixture was stirred for 16 hours. The mixture was diluted with water (850 mL) and extracted with EtOAc (3×300 mL). The combined organic extracts were washed with brine (5×350 mL) and aqueous LiCl (3M, 2×350 mL), dried (MgSO₄), filtered and concentrated in vacuo. The crude mixture was submitted to flash column chromatography (0-50% EtOAc/Pet. Ethers 40-60) to yield the product, benzyl bis(2-((tert-butoxycarbonyl)amino)ethyl)glycinate (4.99 g, 11.1 mmol, 76%) as a colourless oil. R_{f}: 0.39 (50% EtOAc/Pet. Ethers 40-60); ¹H NMR (500 MHz, CDCl₃, 25° C.): δ (ppm)=7.39-7.31 (m, 5H), 5.16 (s, 2H), 3.49 (br s, 2H), 3.20 (br s, 4H), 2.81 (br s, 4H), 1.44 (s, 18H); HRMS (ESI) C₂₂H₃₅N₃O₆ m/z: [M+Na]⁺ 474.2576 (calc. 474.2574).

### Step 2: Synthesis of Compound 203

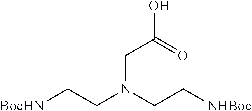 [SMILES: CC(C)(C)OC(=O)NCCN(CCNC(=O)OC(C)(C)C)CC(=O)O]

Benzyl ester 202 (7.78 g, 17.2 mmol) was dissolved in MeOH (170 mL) and degassed with N₂ for 15 minutes. Palladium on carbon (10 wt % Pd, 340 mg) was added and the suspension flushed with H₂ for 10 minutes. The vent needle was removed, and a fresh H₂ balloon applied, and the suspension stirred for 20 hours. The mixture was filtered through Super-Cel®, washed with MeOH (2×125 mL) and concentrated in vacuo to yield the product, bis(2-((tert-butoxycarbonyl)amino)ethyl)glycine (5.81 g, 16.1 mmol, 93%) as a white solid. ¹H NMR (500 MHz, DMSO-d₆, 25° C.): δ (ppm)=6.64 (t, J=5.0 Hz, 2H), 3.27 (s, 2H), 2.95 (q, J=6.0 Hz, 4H), 2.60 (t, J=6.7 Hz, 4H), 1.37 (s, 18H); ¹³C NMR (126 MHz, DMSO-d₆): δ (ppm)=172.6, 155.6, 77.5, 54.9, 53.4, 38.4, 28.2; HRMS (ESI) C₁₆H₃₁N₃O₆ m/z: [M+H]⁺ 362.2271 (calc. 362.2286).

### Step 3: Synthesis of Compound 204

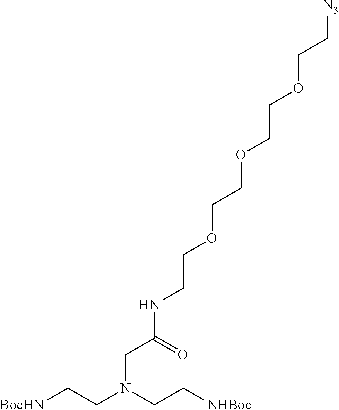 [SMILES: CC(C)(C)OC(=O)NCCN(CCNC(=O)OC(C)(C)C)CC(=O)NCCOCCOCCOCCN=[N+]=[N-]]

Acid 203 (100 mg, 0.28 mmol) was dissolved in DMF (1 mL). 11-Azido-3,6,9-trioxaundecanamine (55 μL, 0.28 mmol), HBTU (105 mg, 0.28 mmol) and DIPEA (96 μL, 0.55 mmol) were added, and the reaction mixture stirred for 71 hours. The mixture was diluted with aqueous HCl (1M, 25 mL) and extracted with EtOAc (3×10 mL). The combined organic layers were washed with brine (25 mL), dried (Na₂SO₄), filtered and concentrated in vacuo. The crude residue was submitted to flash column chromatography (0-6% MeOH/CH₂Cl₂) to yield the product, 204 (94 mg, 0.17 mmol, 60%) as a colourless residue. R_{f}: 0.27 (6% MeOH/CH₂Cl₂); ¹H NMR (400 MHz, CDCl₃, 25° C.): δ (ppm)=7.47 (br s, 1H), 5.68 (br s, 2H), 3.68-3.57 (m, 12H), 3.46 (q, J=3.2 Hz, 2H), 3.37 (t, J=4.9 Hz, 2H), 3.26-2.99 (m, 5H), 2.58 (br s, 3H), 1.44 (s, 18H); ¹³C NMR (101 MHz, CDCl₃): δ (ppm)=171.0, 156.7, 79.5, 70.8, 70.7, 70.6, 70.2, 70.1, 69.6, 55.3, 50.8, 39.2, 38.3, 28.6; HRMS (ESI) C₂₄H₄₇N₇O₈ m/z: [M+Na]⁺ 584.3401 (calc. 584.3378).

### Step 4: Synthesis of Compound 205

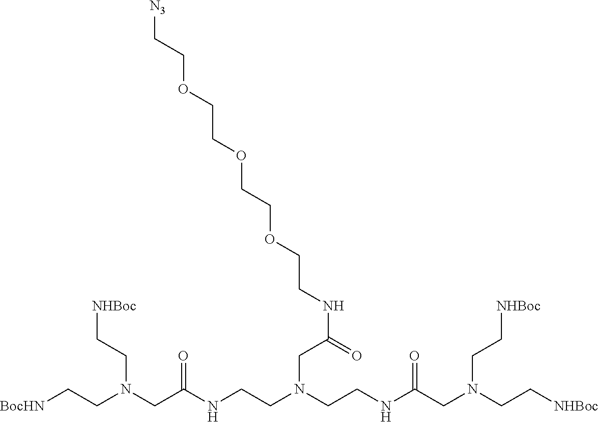 [SMILES: CC(C)(C)OC(=O)NCCN(CCNC(=O)OC(C)(C)C)CC(=O)NCCN(CCNC(=O)CN(CCNC(=O)OC(C)(C)C)CCNC(=O)OC(C)(C)C)CC(=O)NCCOCCOCCOCCN=[N+]=[N-]]

N-Boc amine 204 (64 mg, 0.11 mmol) was suspended in HCl/dioxane (4M, 1 mL) and stirred for 1.5 hours. The solvent was removed under a stream of nitrogen to leave a white solid, which was used without further purification. The intermediate was suspended in DMF (0.5 mL) and DIPEA (119 μL, 0.68 mmol), acid 203 (103 mg, 0.28 mmol) and HBTU (108 mg, 0.28 mmol) were added. The reaction mixture was stirred for 22 hours. The mixture was diluted with water (20 mL) and extracted with EtOAc (2×20 mL). The combined organic extracts were washed with brine (4×50 mL), dried (Na₂SO₄), filtered and concentrated. The crude product was submitted to flash column chromatography (0-8% MeOH/CH₂Cl₂) to yield the product, 205 (53.5 mg, 0.051 mmol, 46%) as a colourless residue. R_{f}: 0.15 (8% MeOH/CH₂Cl₂); ¹H NMR (500 MHz, CDCl₃, 25° C.): δ (ppm)=6.77 (s, 3H), 3.61 (t, J=5.0 Hz, 3H), 3.58-3.51 (m, 8H), 3.47 (t, J=6.0 Hz, 2H), 3.39 (t, J=5.0 Hz, 2H), 3.28 (q, J=5.8 Hz, 2H), 3.24 (s, 1H), 3.12 (s, 1H), 1.39 (s, 36H); ¹³C NMR (125 MHz, CDCl₃): δ (ppm)=155.7, 78.2, 69.8, 69.8, 69.7, 69.6, 69.3, 68.9, 54.1, 50.0, 28.2; LRMS (ESI) C₄₆H₈₉N₁₃O₁₄ m/z: [M+H]⁺ 1048.7 (calc. 1048.7).

### Step 5: Synthesis of Compound 206 (Scaffold A)

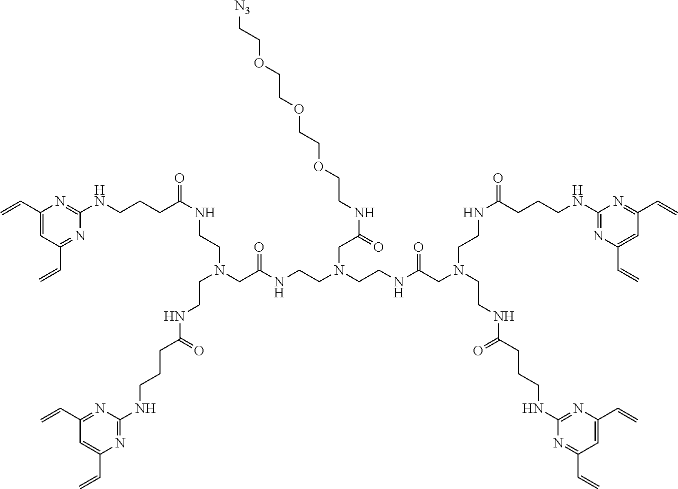 [SMILES: C=Cc1cc(C=C)nc(NCCCC(=O)NCCN(CCNC(=O)CCCNc2nc(C=C)cc(C=C)n2)CC(=O)NCCN(CCNC(=O)CN(CCNC(=O)CCCNc2nc(C=C)cc(C=C)n2)CCNC(=O)CCCNc2nc(C=C)cc(C=C)n2)CC(=O)NCCOCCOCCOCCN=[N+]=[N-])n1]

Tetra-N-Boc amine 205 (20 mg, 0.019 mmol) was dissolved in CH₂Cl₂ (1.5 mL) and HCl in dioxane (4M, 0.5 mL) was added. The reaction mixture was stirred for 1.5 hours and then the solvent was removed in vacuo. The intermediate was redissolved in DMF (1 mL) and compound 2 (22.3 mg, 0.095 mmol), HBTU (36.2 mg, 0.095 mmol) and DIPEA (33 μL, 0.191 mmol) were added. The reaction mixture was stirred for 3.5 hours. LCMS indicated incomplete conversion, so further compound 2 (8.9 mg, 0.038 mmol), HBTU (14.5 mg, 0.038 mmol) and DIPEA (13 μL, 0.076 mmol) were added. The mixture was stirred for a further 1.5 hours and then submitted to reverse-phase Combiflash chromatography (10-70% MeCN in 0.1M aqueous NH₄OH) to yield the product, 206 (9.05 mg, 0.0060 mmol, 32%) as a pale-yellow solid. ¹H NMR (400 MHz, CDCl₃, 25° C.): δ (ppm)=7.73 (br s, 2H; H), 6.55 (dd, J=16.9, 10.1 Hz, 14H), 6.39 (d, J=17.3 Hz, 8H), 5.62 (d, J=10.5 Hz, 8H), 2.38 (t, J=7.1 Hz, 9H), 1.95 (qu, J=6.6 Hz, 9H); ¹³CNMR (125 MHz, CDCl₃): δ (ppm)=174.3, 163.4, 161.6, 136.4, 131.3, 121.3, 107.9, 105.2, 70.7, 70.7, 70.6, 70.6, 70.6, 70.2, 70.1, 70.1, 69.4, 59.0, 55.9, 53.8, 50.8, 48.2, 46.9, 40.9, 39.3, 37.3, 33.9, 26.3, 25.7, 24.8, 24.4; LRMS (ESI) C₇₄H₁₀₉N₂₅O₁₀ m/z: [M+formate]⁻ 1553.0 (calc. 1552.7).

### Example 26—Synthesis of TetraDVP Scaffold B

### Step 1: Synthesis of Compound 207

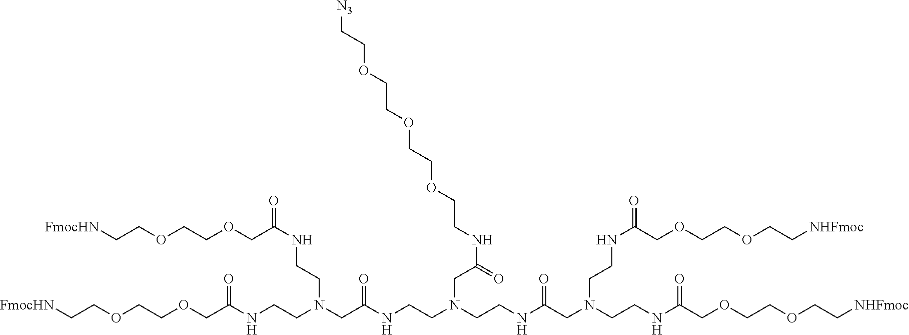

Tetra N-Boc amine 205 (35 mg, 0.033 mmol) was suspended in HCl/dioxane (4M, 1 mL) and stirred for 1.5 hours. The solvent was removed under a stream of nitrogen to leave a white solid, which was used without further purification. The intermediate was suspended in DMF (0.3 mL) and Fmoc-8-amino-3,6-dioxaoctanoic acid (51 mg, 0.13 mmol), HBTU (50 mg, 0.13 mmol) and DIPEA (70 μL, 0.40 mmol) were added. The reaction mixture was stirred for 18 hours. The mixture was diluted with water (20 mL) and extracted with EtOAc (2×20 mL). The combined organic extracts were washed with aqueous LiCl (5 wt %, 30 mL), dried (Na₂SO₄), filtered and concentrated. The crude product was submitted to flash column chromatography (4-10% MeOH/CH₂Cl₂) to yield the product, 207 (13 mg, 0.0059 mmol, 18%) as a white residue. R_{f}: 0.10 (10% MeOH/CH₂Cl₂); ¹H NMR (400 MHz, MeOD, 25° C.): δ (ppm)=7.76 (d, J=7.5 Hz, 8H), 7.61 (d, J=7.4 Hz, 8H), 7.36 (t, J=7.4 Hz, 8H), 7.27 (t, J=7.4 Hz, 8H), 4.58 (s, 2H), 4.46 (s, 1H), 4.32 (d, J=6.8 Hz, 7H), 4.16 (t, J=6.7 Hz, 4H), 3.94 (s, 8H), 3.64-3.57 (m, 17H), 3.56-3.53 (m, 8H), 3.53-3.43 (m, 10H), 3.36 (t, J=5.4 Hz, 2H), 3.27 (t, J=5.9 Hz, 18H), 3.20 (s, 2H), 3.18 (s, 4H), 3.05 (br s, 1H), 2.66 (t, J=6.5 Hz, 4H), 2.61 (t, J=6.2 Hz, 8H); ¹³C NMR (101 MHz, MeOD): δ (ppm)=174.2, 172.7, 158.8, 145.3, 142.6, 128.8, 128.2, 126.2, 121.0, 72.0, 71.6, 71.5, 71.4, 71.2, 71.1, 71.0, 67.7, 59.7, 55.9, 55.6, 48.5, 41.7, 40.0, 38.3; LRMS (ESI) C₁₁₀H₁₄₁N₁₇O₂₆ m/z: [M+formate]⁻ 2161.4 (calc. 2161.0).

### Step 2: Synthesis of Compound 208 (Scaffold B)

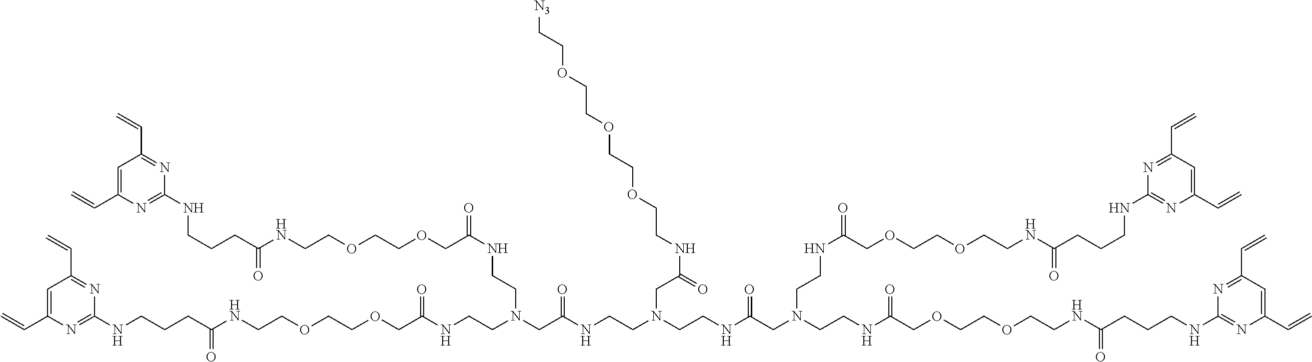 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCN(CCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCOCCOCCOCCN=[N+]=[N-])n1]

Tetra-N-Fmoc amine 207 (10 mg, 0.0047 mmol) was dissolved in DMF (0.3 mL) and piperidine (4.6 μL, 0.047 mmol) was added. The mixture was stirred for 2 hours. The solvent was removed under a stream of nitrogen to leave a white solid, which was used without further purification. The intermediate was suspended in DMF (0.3 mL) and compound 2 (5 mg, 0.021 mmol), HBTU (8 mg, 0.021 mmol) and DIPEA (7.4 μL, 0.043 mmol) were added. The reaction mixture was stirred for 2 hours and then submitted directly to reverse-phase Combiflash chromatography (10-80% MeCN in water). Fractions containing product were combined and lyophilised to yield the product, 208 (1.9 mg, 0.00089 mmol, 19%) as an off-white solid residue. ¹H NMR (400 MHz, DMSO-d₆, 25° C.): δ (ppm)=7.85 (t, J=5.5 Hz, 4H), 7.81-7.74 (m, 3H), 7.69 (t, J=5.6 Hz, 4H), 7.04 (t, J=5.5 Hz, 4H), 6.76 (s, 4H), 6.57 (dd, J=10.7, 17.3 Hz, 8H), 6.34 (d, J=16.1 Hz, 8H), 5.57 (dd, J=1.6, 10.5 Hz, 8H), 3.85 (s, 8H), 3.59-3.46 (m, 29H), 3.40 (t, J=5.7 Hz, 8H), 3.23-3.11 (m, 23H), 3.07 (br s, 6H), 2.67 (t, J=1.8 Hz, 3H), 2.59-2.53 (m, 7H), 2.33 (m, 5H), 2.13 (t, J=7.6 Hz, 11H), 1.74 (t, J=7.4 Hz, 11H); HRMS (ESI) C₉₈H₁₅₃O₂₂N₂₉ m/z: [M+H]⁺ 2089.1782 (calc. 2089.1818).

### Example 27—Synthesis of TetraDVP Scaffold C

### Step 1: Synthesis of Compound 209

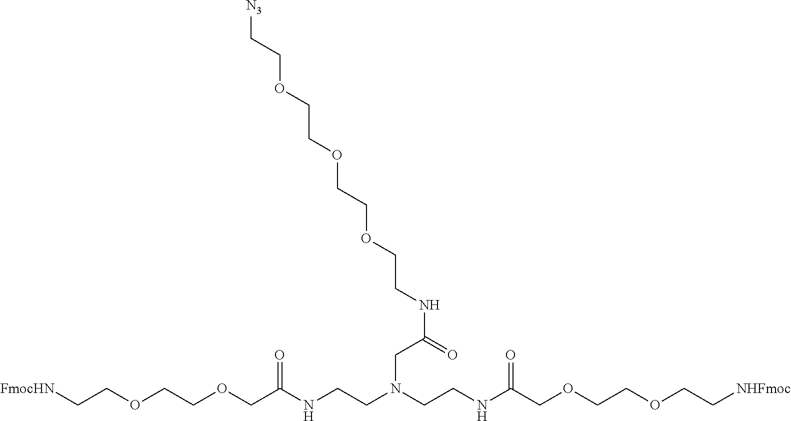

Compound 204 (385 mg, 0.69 mmol) was dissolved in HCl solution (4 M in dioxanes, 4 mL) and CH₂Cl₂ (2 mL) and stirred for 3 hours. The solvent was removed under a stream of nitrogen, and the resulting residue was redissolved in DMF (2.5 mL). Fmoc-8-Amino3,6-dioxaoctonoic acid (532 mg, 1.38 mmol), HBTU (523 mg, 1.38 mmol) and DIPEA (721 μL, 4.14 mmol) were added, and the reaction mixture stirred for 17 hours. Some acid starting material remained, so further HBTU (261 mg, 0.69 mmol) and DIPEA (120 μL, 0.69 mmol) were added and the mixture stirred for a further 1 hour. The mixture was submitted directly to reverse-phase column chromatography (10-100% MeCN in H₂O) to yield the product, 209 (378 mg, 0.34 mmol, 50%) as a viscous orange residue. R_{f}: 0.18 (7% MeOH in CH₂Cl₂); IR(neat): vₘₐₓ(cm⁻¹)=3313 (N—H, medium), 2928 (C—H, medium), 2870 (C—H, medium), 2107 (N₃, medium), 1714 (C═O, strong), 1658 (C═O, strong), 1529 (N—H, strong), 1448 (C—H, strong), 1247 (C—N, strong), 1102 (CO, strong); ¹H NMR (500 MHz, CDCl₃, 25° C.): δ (ppm)=7.75 (d, J=7.5 Hz, 4H), 7.60 (d, J=7.2 Hz, 4H), 7.39 (t, J=7.5 Hz, 4H), 7.30 (t, J=7.4 Hz, 4H), 7.18 (br s, 2H), 5.71 (br s, 2H), 4.40 (d, J=6.4 Hz, 4H), 4.20 (t, J=6.7 Hz, 2H), 3.99 (s, 4H), 3.68-3.51 (m, 24H), 3.43 (q, J=5.6 Hz, 2H), 3.40-3.30 (m, 10H), 3.15 (s, 2H), 2.64 (t, J=6.0 Hz, 4H); ¹³C NMR (125 MHz, CDCl₃): δ (ppm)=171.4, 170.5, 156.8, 144.1, 141.5, 127.8, 127.2, 125.2, 120.1, 77.4, 71.1, 70.8, 70.7, 70.6, 70.6, 70.4, 70.2, 70.1, 70.1, 69.7, 66.7, 59.2, 54.8, 50.8, 47.4, 41.0, 39.0, 37.2; HRMS (ESI) C₅₆H₇₃O₁₄N₉ m/z: [M+H]⁺ 1096.5346 (calc. 1096.5350).

### Step 2: Synthesis of Compound 210

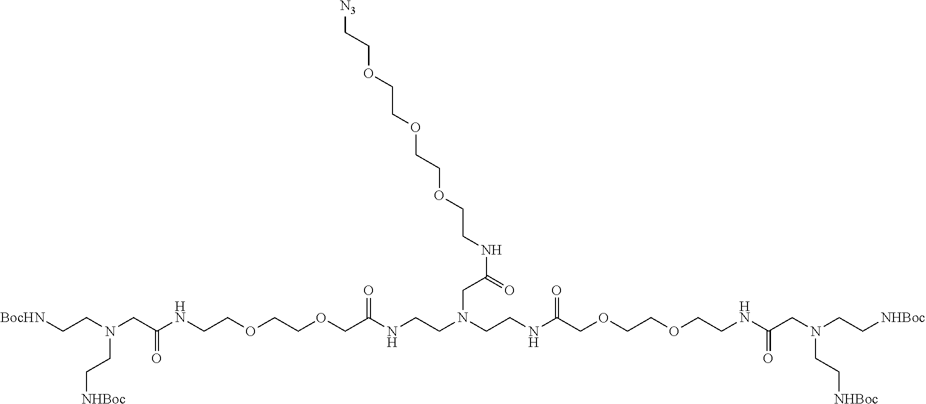 [SMILES: CC(C)(C)OC(=O)NCCN(CCNC(=O)OC(C)(C)C)CC(=O)NCCOCCOCC(=O)NCCN(CCNC(=O)COCCOCCNC(=O)CN(CCNC(=O)OC(C)(C)C)CCNC(=O)OC(C)(C)C)CC(=O)NCCOCCOCCOCCN=[N+]=[N-]]

N-Fmoc amine 209 (161 mg, 0.15 mmol) was dissolved in DMF (2 mL) and piperidine (116 μL, 1.18 mmol) was added. The mixture was stirred for 1 hour. The solvent was removed under a stream of nitrogen to leave a white solid. The residue was suspended in DMF (1.5 mL) and acid 4 (133 mg, 0.37 mmol), HBTU (139 mg, 0.37 mmol) and DIPEA (128 μL, 0.73 mmol) were added. The reaction mixture was stirred for 1.5 hours. The crude mixture was submitted to reverse-phase column chromatography (10-100% MeCN/H₂O) to yield the product, 210 (118 mg, 0.088 mmol, 59%) as a pale-yellow residue. ¹H NMR (500 MHz, CDCl₃, 25° C.): δ (ppm)=7.49 (br s, 2H), 7.22 (br s, 1H), 5.60 (s, 2H), 4.09-3.93 (m, 4H), 3.70-3.59 (m, 25H), 3.57 (t, J=4.8 Hz, 4H), 3.50-3.41 (m, 7H), 3.38 (t, J=5.0 Hz, 6H), 3.27-3.03 (m, 10H), 2.82-2.48 (m, 8H), 2.16 (br s, 2H), 1.44 (s, 36H); ¹³C NMR (125 MHz, CDCl₃): δ (ppm)=171.0, 163.2, 156.6, 152.0, 82.9, 79.7, 71.0, 70.8, 70.7, 70.6, 70.3, 70.1, 70.1, 69.6, 69.5, 63.1, 55.9, 55.6, 55.1, 54.8, 53.7, 50.8, 47.2, 41.0, 39.2, 28.6; HRMS (ESI) C₅₈H₁₁₁N₁₅O₂₀ m/z: [M+2H]²⁺ 669.9153 (calc. 669.9138).

### Step 3: Synthesis of Compound 211 (Scaffold C)

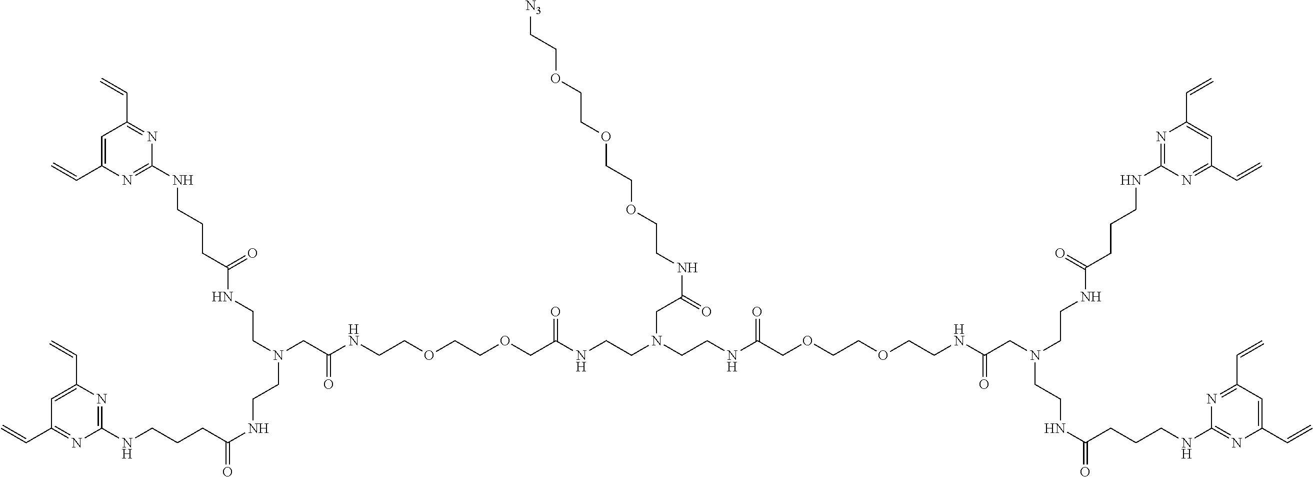 [SMILES: C=Cc1cc(C=C)nc(NCCCC(=O)NCCN(CCNC(=O)CCCNc2nc(C=C)cc(C=C)n2)CC(=O)NCCOCCOCC(=O)NCCN(CCNC(=O)COCCOCCNC(=O)CN(CCNC(=O)CCCNc2nc(C=C)cc(C=C)n2)CCNC(=O)CCCNc2nc(C=C)cc(C=C)n2)CC(=O)NCCOCCOCCOCCN=[N+]=[N-])n1]

Tetra-N-Boc amine 210 (25 mg, 0.019 mmol) was dissolved in HCl/dioxane (4 M, 1 mL) and stirred for 1 hour. The solvent was removed under a stream of nitrogen. Compound 2 (26.1 mg, 0.112 mmol), HBTU (42.5 mg, 0.112 mmol) were added, and the combined solids suspended in DMF (0.75 mL). DIPEA (59 μL, 0.336 mmol) was added and the reaction mixture stirred for 1 hour. The mixture was submitted to reverse-phase column chromatography (10-100% MeCN in 0.1M NH₄OH_{(aq)}) to yield the product, 211 (9.71 mg, 0.0054 mmol, 28%) as a brown solid. ¹H NMR (500 MHz, DMSO-d₆, 25° C.): δ (ppm)=7.80-7.72 (m, 7H), 7.68 (t, J=5.7 Hz, 2H), 7.04 (t, J=5.6 Hz, 4H), 6.76 (s, 4H), 6.58 (dd, J=10.6, 17.3 Hz, 8H), 6.35 (d, J=16.6 Hz, 8H), 5.58 (dd, J=1.4, 10.3 Hz, 8H), 3.85 (s, 4H), 3.61-3.57 (m, 2H), 3.56-3.45 (m, 16H), 3.44-3.36 (m, 9H), 3.29 (q, J=6.6 Hz, 9H), 3.24 (q, J=5.9 Hz, 6H), 3.17 (q, J=6.3 Hz, 4H), 3.11 (q, J=6.2 Hz, 8H), 3.08-3.03 (m, 6H), 2.56 (t, J=6.8 Hz, 4H), 2.14 (t, J=7.7 Hz, 8H), 1.76 (m, J=7.2 Hz, 9H), 0.95 (d, J=6.5 Hz, 4H); ¹³C NMR (125 MHz, DMSO-d₆): δ (ppm)=172.1, 170.6, 170.5, 169.2, 163.1, 162.4, 136.1, 121.4, 104.5, 70.1, 70.0, 69.8, 69.7, 69.7, 69.5, 69.3, 69.2, 69.0, 70.0, 58.3, 58.0, 54.2, 54.1, 50.0, 40.3, 40.1, 39.9, 39.8, 39.6, 38.1, 36.8, 36.4, 33.1; HRMS (ESI) C₈₆H₁₃₂O₁₆N₂₇ m/z: [M+H]⁺ 1799.0355 (calc. 1799.0340).

### Example 28—Synthesis of TetraDVP Scaffold D

### Step 1: Synthesis of Compound 212

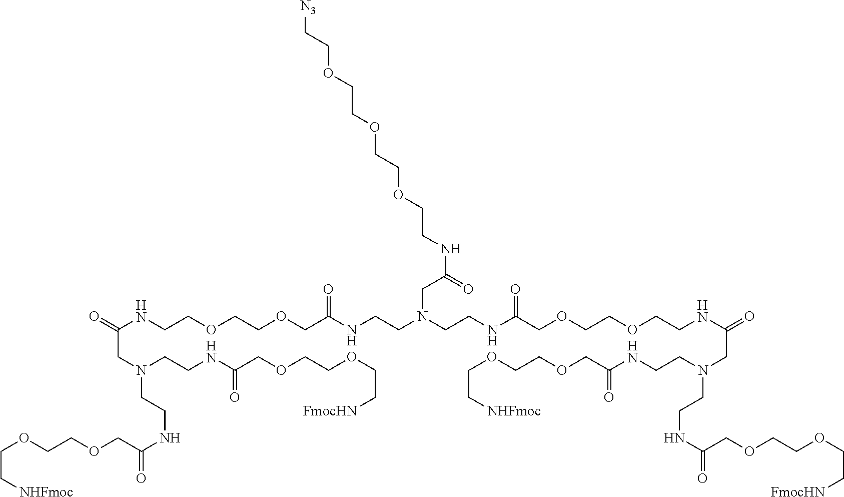

Tetra-N-Boc amine 210 (102 mg, 0.076 mmol) was dissolved in HCl solution (4 M in dioxanes, 2 mL) and stirred for 1 hour. The solvent was removed under a stream of nitrogen, and the resulting white solid redissolved in DMF (2 mL). Fmoc-8-amino-3,6-dioxaoctonoic acid (176 mg, 0.46 mmol), HBTU (173 mg, 0.46 mmol) and DIPEA (239 μL, 1.37 mmol) were added and the reaction mixture stirred for 19.5 hours. The mixture was submitted to reverse-phase column chromatography (10-100% MeCN in H₂O) to yield the product, 212 (50.6 mg, 0.021 mmol, 28%) as a white solid. ¹H NMR (500 MHz, DMSO-d₆, 25° C.): δ (ppm)=7.87 (d, J=7.5 Hz, 8H), 7.77-7.71 (m, 3H), 7.68 (m incl. d, J=7.5 Hz, 13H), 7.40 (t, J=7.4 Hz, 8H), 7.31 (m incl. t, J=7.4 Hz, 11H), 4.28 (d, J=6.9 Hz, 8H), 4.20 (t, J=6.8 Hz, 4H), 3.87-3.84 (m, 11H), 3.58-3.47 (m, 32H), 3.44-3.39 (m incl. t, J=5.2 Hz, 13H), 3.36 (t, J=4.9 Hz, 2H), 3.24 (q, J=5.6 Hz, 6H), 3.15 (m, J=6.4 Hz, 19H), 3.07 (s, 6H), 2.56 (t, J=6.7 Hz, 11H); ¹³C NMR (100 MHz, DMSO-d₆): δ (ppm)=170.6, 169.8, 169.3, 169.2, 142.6, 139.4, 137.4, 128.9, 127.3, 121.4, 120.0, 109.8, 70.2, 70.2, 70.0, 69.8, 69.7, 69.7, 69.5, 69.3, 69.0, 58.0, 54.1, 50.0, 40.3, 40.1, 39.8, 38.1, 36.4; HRMS (ESI) C₁₂₂H₁₆₄O₃₂N₁₉ m/z: [M+H]⁺ 2407.1743 (calc. 2407.1784).

### Step 2: Synthesis of Compound 213 (Scaffold D)

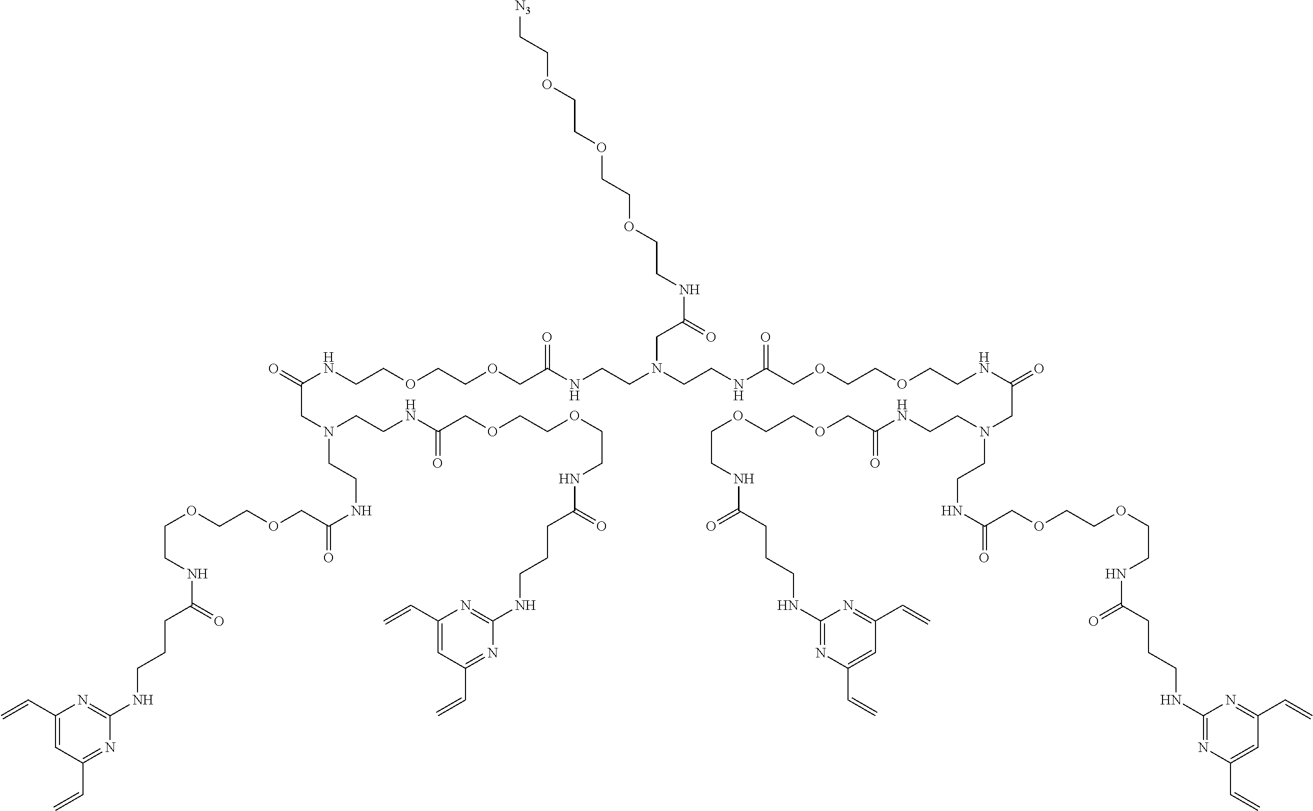 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCOCCOCC(=O)NCCN(CCNC(=O)COCCOCCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCOCCOCCOCCN=[N+]=[N-])n1]

Compound 212 (24 mg, 0.01 mmol) was dissolved in DMF (1 mL) and piperidine (9.9 μL, 0.10 mmol) was added. The mixture was stirred for 30 minutes, and the solvent was removed under a stream of nitrogen. The crude residue was redissolved in DMF (0.75 mL). Acid 2 (14 mg, 0.06 mmol), HBTU (23 mg, 0.06 mmol) and DIPEA (31 μL, 0.18 mmol) were added and the reaction mixture was stirred for 2 hours. The crude mixture was submitted to reverse-phase flash column chromatography (0-100% MeCN in 0.1M NH₄OH_{(aq)}) to yield the product, 213 (8.6 mg, 0.0036 mmol, 36%) as a pale brown solid. ¹H NMR (500 MHz, DMSO-d₆, 25° C.): δ (ppm)=7.86 (t, J=5.5 Hz, 4H), 7.78-7.72 (m, 3H), 7.70-7.63 (m, 6H), 7.04 (t, J=5.7 Hz, 4H), 6.76 (s, 4H), 6.57 (dd, J=10.7, 17.2 Hz, 8H), 6.35 (d, J=16.8 Hz, 8H), 5.57 (dd, J=1.4, 10.7 Hz, 8H), 3.86 (s, 12H), 3.60-3.47 (m, 36H), 3.45-3.36 (m, 17H), 3.30-3.22 (m, 18H), 3.22-3.13 (m, 21H), 3.07 (s, 6H), 2.56 (t, J=6.4 Hz, 10H), 2.13 (t, J=7.5 Hz, 8H), 1.74 (app. qu, J=7.2 Hz, 9H); ¹³C NMR (125 MHz, DMSO-d₆): δ (ppm)=172.1, 170.6, 170.5, 169.2, 169.2, 163.2, 162.4, 136.1, 121.4, 104.5, 70.2, 70.0, 69.8, 69.7, 69.7, 69.5, 69.3, 69.3, 69.2, 69.1, 69.0, 58.0, 54.1, 50.0, 40.3, 40.1, 39.9, 39.8, 39.6, 38.4, 38.1, 38.1, 36.4, 33.0, 28.3, 25.3; HRMS (ESI) C₁₁₀H₁₇₅O₂₈N₃₁ m/z: [M+H]⁺ 2379.3252 (calc. 2379.3296).

### Example 29—Synthesis of TetraDVP Scaffold E

### Step 1: Synthesis of Compound 216

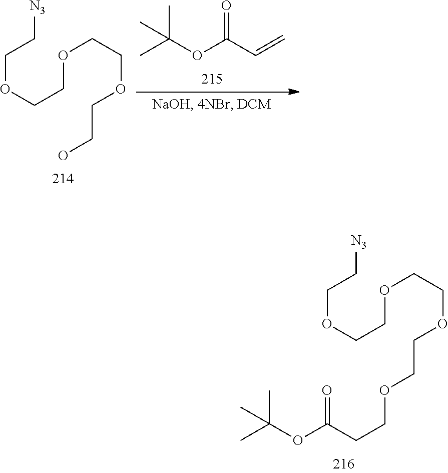

To a mixture of compound 214 (10 g, 45.61 mmol) and compound 215 (4.9 g, 38.23 mmol) in DCM (100 mL) was added Bu₄NBr (2.4 g, 7.44 mmol) and NaOH (9.85 g, 246.31 mmol). The reaction mixture was stirred at 15° C. for 3 hours. The reaction mixture was poured into water (200 mL), extracted with DCM (200 mL*2). The combined organic layers were washed with brine (200 mL), dried over sodium sulfate, concentrated in vacuum to give the residue, which was purified by column (SiO₂, 30% to 40% of EtOAc in Petroleum) to afford compound 216 (5.2 g, 32.8% yield) as yellow oil. ¹H NMR (400 MHz, CHLOROFORM-d) δ 3.73-3.69 (m, 2H), 3.68-3.64 (m, 10H), 3.64-3.59 (m, 4H), 2.04 (s, 2H), 1.44 (s, 9H).

### Step 2: Synthesis of Compound 217

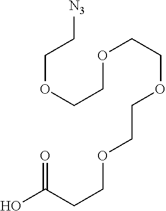 [SMILES: [N-]=[N+]=NCCOCCOCCOCCOCCC(=O)O]

A mixture of compound 216 (5.2 g, 14.97 mmol) in HCl/dioxane (4 M, 50 mL) was stirred at 20° C. for 2 hours. LCMS showed starting material was consumed and desired mass was detected as major product. The reaction mixture was concentrated in vacuum to afford compound 217 (4.3 g, 98.6% yield) as yellow oil. LCMS: rt=0.502 min, (292.2 [M+H]⁺), 74.6% purity.

### Step 3: Synthesis of Compound 219

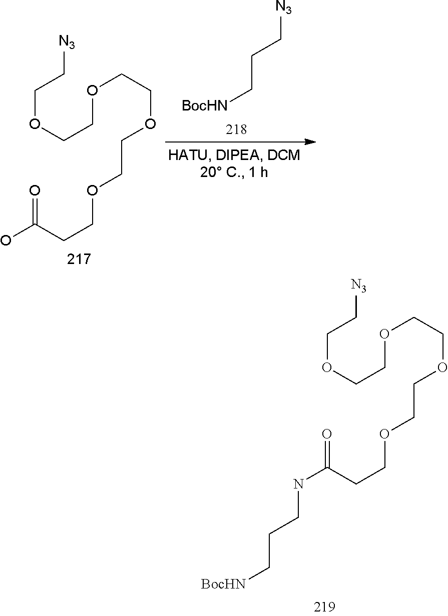 [SMILES: [N-]=[N+]=NCCOCCOCCOCCOCCC(=O)O ; CC(C)(C)OC(=O)NCCCN=[N+]=[N-] ; CC(C)(C)OC(=O)NCCCNC(=O)CCOCCOCCOCCOCCN=[N+]=[N-]]

To a mixture of compound 217 (4.3 g, 14.76 mmol) and compound 218 (2.8 g, 16.24 mmol) in DCM (60 mL) was added HATU (5.6 g, 14.76 mmol) and DPIEA (9.5 g, 73.81 mmol). The reaction mixture was stirred at 20° C. for 1 hour. LCMS showed starting material was consumed and desired mass was detected. The reaction mixture was concentrated in vacuum to give the residue, which was purified by reversed phase Combiflash (water (0.1% FA)-ACN; B %: 30%-50%). The mixture was adjusted to pH=8 by aq. NaHCO₃ solution and concentrated in vacuum to remove CH₃CN. Then the mixture was extracted with EtOAc (200 mL*2). The combined organic layers were washed with brine (200 mL), dried over sodium sulfate, concentrated in vacuum to afford compound 219 (5 g, 75.7% yield) as light-yellow oil. LCMS: rt=0.740 min, (448.3 [M+H]⁺), 100.00% purity. ¹H NMR (400 MHz, METHANOL-d₄) δ 3.72 (t, J=6.0 Hz, 2H), 3.69-3.61 (m, 14H), 3.39-3.36 (m, 2H), 3.21 (t, J=6.8 Hz, 2H), 3.07 (t, J=6.8 Hz, 2H), 2.43 (t, J=6.0 Hz, 2H), 1.66-1.58 (m, 2H), 1.44 (s, 9H).

### Step 4: Synthesis of Compound 220

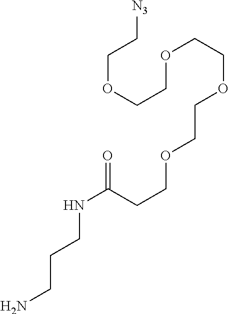 [SMILES: [N-]=[N+]=NCCOCCOCCOCCOCCC(=O)NCCCN]

To a mixture of compound 219 (5 g, 11.17 mmol) in dioxane (30 mL) was added HCl/dioxane (4 M, 30 mL). The reaction mixture was stirred at 20° C. for 1 hour. LCMS showed starting material was consumed and desired mass was detected. The reaction mixture was concentrated in vacuum to afford compound 220 (4.6 g, 97.9% yield) as light-yellow oil. LCMS: rt=0.610 min, (348.2 [M+H]⁺), 100.00% purity.

### Step 5: Synthesis of Compound 221

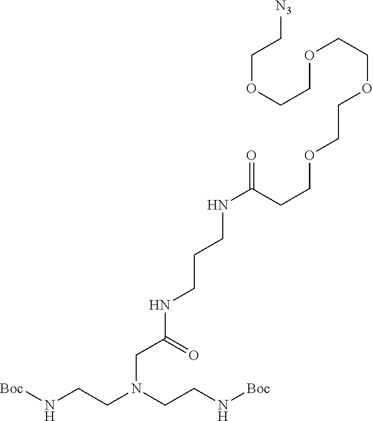 [SMILES: CC(C)(C)OC(=O)NCCN(CCNC(=O)OC(C)(C)C)CC(=O)NCCCNC(=O)CCOCCOCCOCCOCCN=[N+]=[N-]]

To a mixture of compound 220 (2.3 g, 5.99 mmol) and compound 203 (2.17 g, 5.99 mmol) in DCM (50 mL) was added DIPEA (2.3 g, 17.97 mmol) and HATU (2.28 g, 5.99 mmol). The reaction mixture was stirred at 20° C. for 8 hours. LCMS showed the reaction was completed. The reaction mixture was concentrated in vacuum to give the residue, which was purified by reversed phase Combiflash (water (0.1% HCl)-ACN; B % 10%-54%). The mixture was adjusted to pH=8 by aq. NaHCO₃ solution and concentrated in vacuum to remove CH₃CN. Then the mixture was extracted with EtOAc (100 mL*2). The combined organic layers were washed with brine, dried over Na₂SO₄, concentrated in vacuum to afford compound 221 (3.8 g, 88.5% yield) as yellow oil. m/z 691.4 (M+H)+(ES⁺). LCMS: rt=0.530 min, (691.4 [M+H]⁺), 96.41% purity. ¹H NMR (400 MHz, METHANOL-d₄) δ3.76 (t, J=6.0 Hz, 2H), 3.69-3.61 (m, 14H), 3.37 (t, J=5.2 Hz, 2H), 3.29-3.24 (m, 4H), 3.19-3.11 (m, 6H), 2.60 (t, J=6.0 Hz, 2H), 2.48 (t, J=6.0 Hz, 2H), 1.68 (q, J=6.4 Hz, 2H), 1.44 (s, 18H).

### Step 6: Synthesis of Compound 222

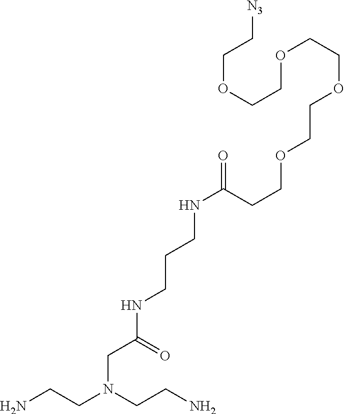 [SMILES: [N-]=[N+]=NCCOCCOCCOCCOCCC(=O)NCCCNC(=O)CN(CCN)CCN]

To a mixture of compound 221 (3.8 g, 5.50 mmol) in dioxane (20 mL) was added HCl/dioxane (4 M, 20 mL). The reaction mixture was stirred at 20° C. for 16 hours. LCMS showed starting material was consumed and desired mass was detected. The reaction mixture was concentrated in vacuum to afford compound 222 (3.2 g, 96.9% yield) as light-yellow oil. LCMS: rt=0.404 min, (491.2 [M+H]⁺), 82.75% purity.

### Step 7: Synthesis of Compound 223

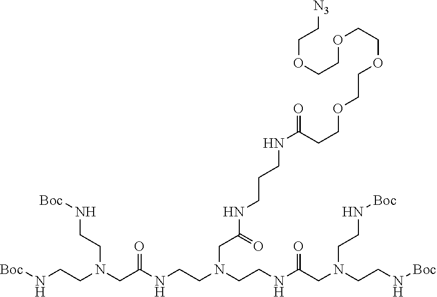 [SMILES: CC(C)(C)OC(=O)NCCN(CCNC(=O)OC(C)(C)C)CC(=O)NCCN(CCNC(=O)CN(CCNC(=O)OC(C)(C)C)CCNC(=O)OC(C)(C)C)CC(=O)NCCCNC(=O)CCOCCOCCOCCOCCN=[N+]=[N-]]

To a mixture of compound 222 (1.65 g, 2.75 mmol) and compound 203 (2.09 g, 5.78 mmol) in DCM (20 mL) was added DIPEA (2.13 g, 16.50 mmol) and HATU (2.20 g, 5.78 mmol). The reaction mixture was stirred at 20° C. for 1 hour. LCMS showed the reaction was completed. The reaction mixture was concentrated in vacuum to give the residue, which was purified by reversed phase Combiflash (water (0.1% FA)-ACN; B % 50%-70%) and then concentrated in vacuum to afford compound 223 (2.7 g, 79.8% yield) as a light-yellow solid. LCMS: rt=0.739 min, (1178.0 [M+H]⁺), 95.74% purity. ¹H NMR (400 MHz, METHANOL-d₄) δ3.73 (t, J=6.0 Hz, 2H), 3.70-3.67 (m, 2H), 3.66-3.65 (m, 4H), 3.64-3.63 (m, 4H), 3.62-3.58 (m, 4H), 3.39-3.33 (m, 6H), 3.29-3.22 (m, 6H), 3.17 (s, 4H), 3.13 (t, J=6.0 Hz, 8H), 2.74 (t, J=6.4 Hz, 4H), 2.60 (t, J=6.0 Hz, 8H), 2.46 (t, J=6.0 Hz, 2H), 1.75-1.67 (m, 2H), 1.44 (s, 36H).

### Step 8: Synthesis of Compound 224

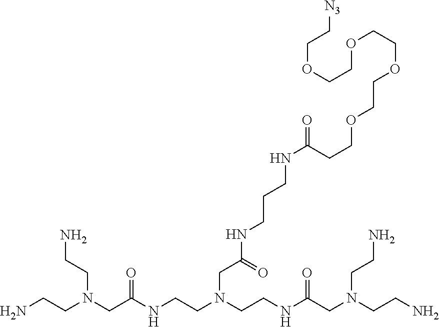 [SMILES: [N-]=[N+]=NCCOCCOCCOCCOCCC(=O)NCCCNC(=O)CN(CCNC(=O)CN(CCN)CCN)CCNC(=O)CN(CCN)CCN]

To a mixture of compound 223 (600 mg, 0.51 mmol) in dioxane (4 mL) was added HCl/dioxane (4 M, 2 mL). The reaction mixture was stirred at 20° C. for 8 hours. LCMS showed starting material was consumed and desired mass was detected. The reaction mixture was concentrated in vacuum to afford compound 224 (525 mg, 99.8% yield) as a light-yellow solid. LCMS: rt=0.390 min, (777.3 [M+H]⁺), 93.39% purity.

### Step 9: Synthesis of Compound 225 (Scaffold E)

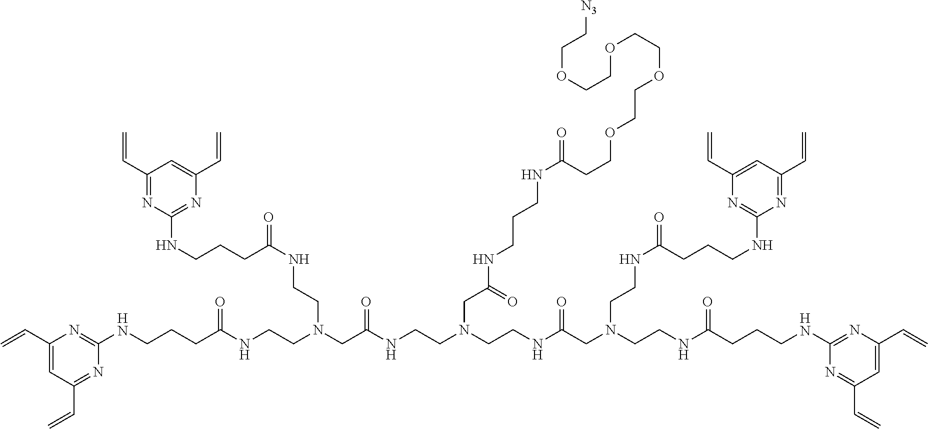 [SMILES: C=Cc1cc(C=C)nc(NCCCC(=O)NCCN(CCNC(=O)CCCNc2nc(C=C)cc(C=C)n2)CC(=O)NCCN(CCNC(=O)CN(CCNC(=O)CCCNc2nc(C=C)cc(C=C)n2)CCNC(=O)CCCNc2nc(C=C)cc(C=C)n2)CC(=O)NCCCNC(=O)CCOCCOCCOCCOCCN=[N+]=[N-])n1]

To a mixture of compound 2 (593.2 mg, 2.54 mmol) and compound 224 (525 mg, 0.51 mmol) in DCM (8 mL) was added DIPEA (1.31 g, 10.17 mmol). Then, HATU (773.58 mg, 2.03 mmol) was slowly added into the above solution. The reaction mixture was stirred at 20° C. for 1 hour. LCMS showed the reaction was completed. The reaction mixture was concentrated in vacuum to give the residue, which was purified by prep-HPLC (column: Waters Xbridge 150 mm*25 mm*5 um; mobile phase: [water (ammonia hydroxide v/v)-ACN]; B %: 37%-67%, 9 min) and lyophilized to afford 225, Scaffold E (236 mg, 25.7% yield) as yellow oil. Then the product was dissolved in DMSO (13 mL) immediately. LCMS: rt=1.022 min, (1638.9 [M+H]⁺), 93.22% purity.

### Example 30—Synthesis of TetraDVP Scaffold F

### Step 1: Synthesis of Compound 226

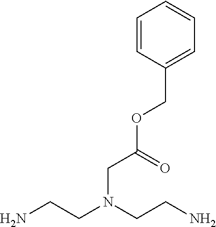 [SMILES: NCCN(CCN)CC(=O)OCc1ccccc1]

To a mixture of compound 202 (10 g, 22.15 mmol) in dioxane (50 mL) was added HCl/dioxane (4 M, 50 mL). The reaction mixture was stirred at 20° C. for 8 hours. LCMS showed starting material was consumed and desired mass was detected. The reaction mixture was concentrated in vacuum to afford compound 226 (7.9 g, 98.9% yield) as a white solid. LCMS: rt=0.435 min, (252.3 [M+H]⁺), 100.00% purity.

### Step 2: Synthesis of Compound 228

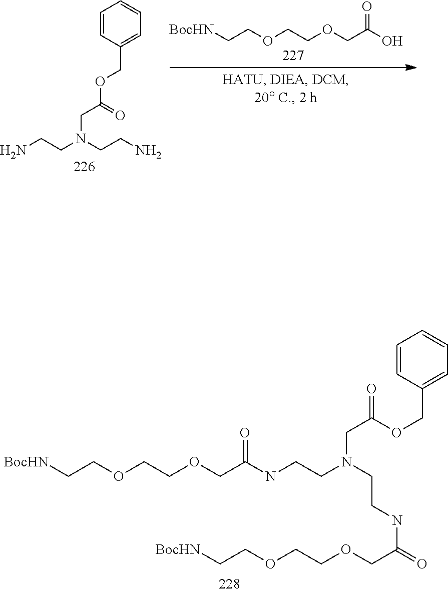 [SMILES: NCCN(CCN)CC(=O)OCc1ccccc1 ; CC(C)(C)OC(=O)NCCOCCOCC(=O)O ; CC(C)(C)OC(=O)NCCOCCOCC(=O)NCCN(CCNC(=O)COCCOCCNC(=O)OC(C)(C)C)CC(=O)OCc1ccccc1]

To a mixture of compound 226 (5 g, 12.59 mmol) and compound 227 (6.96 g, 26.44 mmol) in DCM (100 mL) was added DIPEA (11.39 g, 88.12 mmol) and HATU (10.05 g, 26.44 mmol). The reaction mixture was stirred at 20° C. for 2 hours. LCMS showed starting material was consumed and desired mass was detected. The reaction mixture was concentrated in vacuum to give a residue, which was purified by reversed phase combiflash (water (0.1% FA)-ACN; B % 70%-90%). The mixture was adjusted to pH=8 by aq. NaHCO₃ solution and concentrated in vacuum to remove CH₃CN. Then the mixture was extracted with EtOAc (100 mL*2). The combined organic layer was washed with brine (100 mL), dried over sodium sulfate, concentrated in vacuum to afford compound 228 (6.1 g, 57.4% yield) as yellow oil. LCMS: rt=0.555 min, (742.6 [M+H]⁺), 87.94% purity. ¹H NMR (400 MHz, METHANOL-d₄) δ 7.39-7.31 (m, 5H), 5.16 (s, 2H), 3.99 (s, 4H), 3.63-3.69 (m, 8H), 3.55 (s, 2H) 3.51 (t, J=5.6 Hz, 4H), 3.34-3.31 (m, 4H), 3.22 (t, J=5.6 Hz, 4H), 2.79 (t, J=6.0 Hz, 4H), 1.43 (s, 18H).

### Step 3: Synthesis of Compound 229

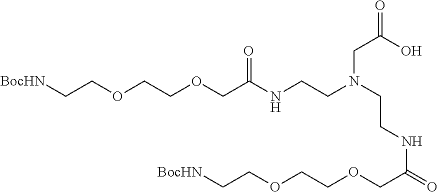 [SMILES: CC(C)(C)OC(=O)NCCOCCOCC(=O)NCCN(CCNC(=O)COCCOCCNC(=O)OC(C)(C)C)CC(=O)O]

To a mixture of compound 228 (6.1 g, 8.22 mmol) in MeOH (200 mL) was added Pd/C (800 mg, 10% purity) under N₂. The reaction mixture was stirred at 20° C. for 3 hours under H₂ (15 psi). LCMS showed the reaction was completed. The catalyst was filtered. The filtrate was concentrated in vacuum to afford compound 229 (5.3 g, 98.9% yield) as colourless oil. LCMS: rt=0.823 min, (652.5 [M+H]⁺), 100.00% purity. ¹H NMR (400 MHz, METHANOL-d₄) δ4.04 (s, 4H), 3.74-3.68 (m, 4H), 3.68-3.62 (m, 6H), 3.55 (t, J=6.0 Hz, 8H), 3.26-3.17 (m, 8H), 1.44 (s, 18H).

### Step 4: Synthesis of Compound 230

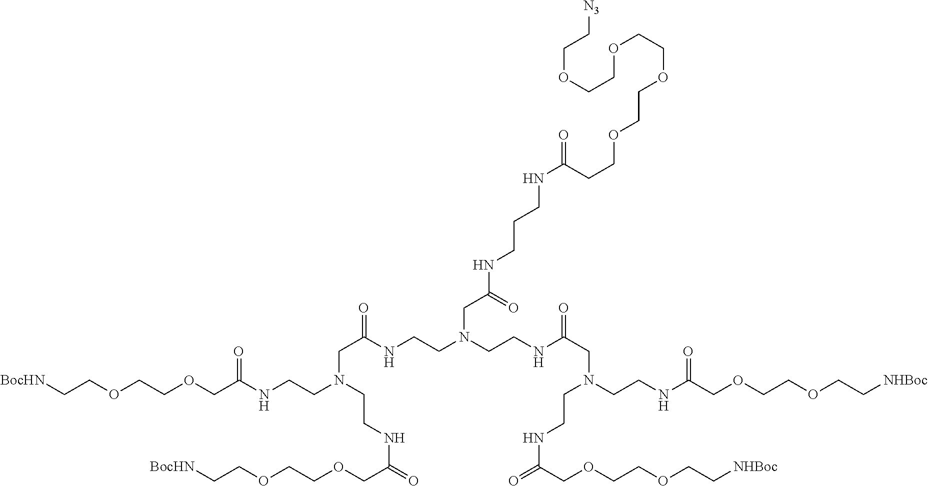 [SMILES: CC(C)(C)OC(=O)NCCOCCOCC(=O)NCCN(CCNC(=O)COCCOCCNC(=O)OC(C)(C)C)CC(=O)NCCN(CCNC(=O)CN(CCNC(=O)COCCOCCNC(=O)OC(C)(C)C)CCNC(=O)COCCOCCNC(=O)OC(C)(C)C)CC(=O)NCCCNC(=O)CCOCCOCCOCCOCCN=[N+]=[N-]]

To a mixture of compound 229 (700 mg, 1.17 mmol) and compound 221 (1.52 g, 2.33 mmol) in DCM (20 mL) was added DIPEA (904.73 mg, 7.00 mmol) and HATU (887.23 mg, 2.33 mmol). The reaction mixture was stirred at 20° C. for 2 hours. LCMS showed starting material was consumed and desired mass was detected. The reaction mixture was concentrated in vacuum to give a residue, which was purified by reversed phase Combiflash (water (0.1% FA)-ACN; B % 50%-70%) and lyophilized to afford compound 230 (800 mg, 39.0% yield) as yellow oil. ¹H NMR (400 MHz, METHANOL-d₄) δ 4.02 (s, 8H), 3.73 (t, J=6.0 Hz, 2H), 3.71-3.68 (m, 10H), 3.67-3.66 (m, 10H), 3.65-3.61 (m, 10H), 3.53 (t, J=6.0 Hz, 8H), 3.43-3.40 (m, 2H), 3.35 (t, J=6.0 Hz, 12H), 3.27-3.22 (m, 18H), 2.75-2.67 (m, 12H), 2.47 (t, J=6.0 Hz, 2H), 1.74-1.67 (m, 2H), 1.44 (s, 36H).

### Step 5: Synthesis of Compound 231

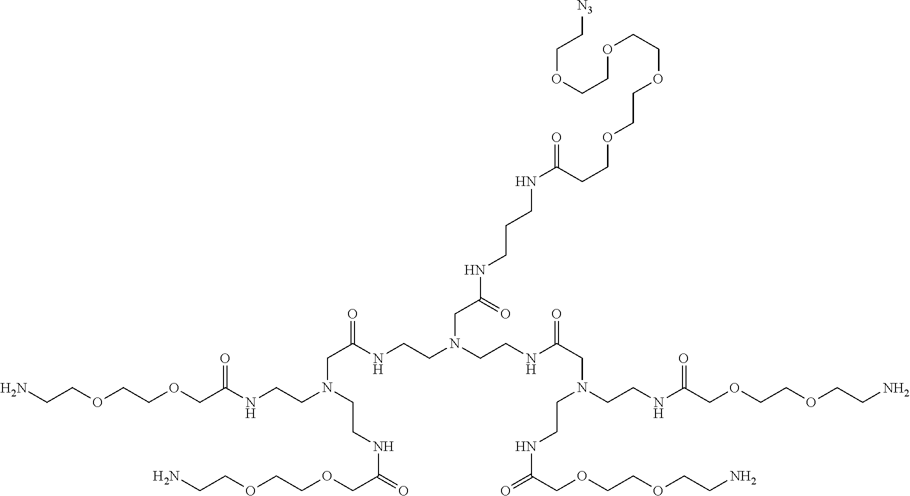 [SMILES: [N-]=[N+]=NCCOCCOCCOCCOCCC(=O)NCCCNC(=O)CN(CCNC(=O)CN(CCNC(=O)COCCOCCN)CCNC(=O)COCCOCCN)CCNC(=O)CN(CCNC(=O)COCCOCCN)CCNC(=O)COCCOCCN]

To a mixture of compound 230 (800 mg, 0.46 mmol) in dioxane (5 mL) was added HCl/dioxane (4 M, 5 mL). The reaction mixture was stirred at 20° C. for 4 hours. LCMS starting material was consumed and desired mass was detected. The reaction mixture was concentrated in vacuum to afford compound 231 (700 mg, 95.4% yield) as yellow oil. LCMS: rt=0.471 min, (1357.7 [M+H]⁺), 59.49% purity.

### Step 6: Synthesis of Compound 232 (Scaffold F)

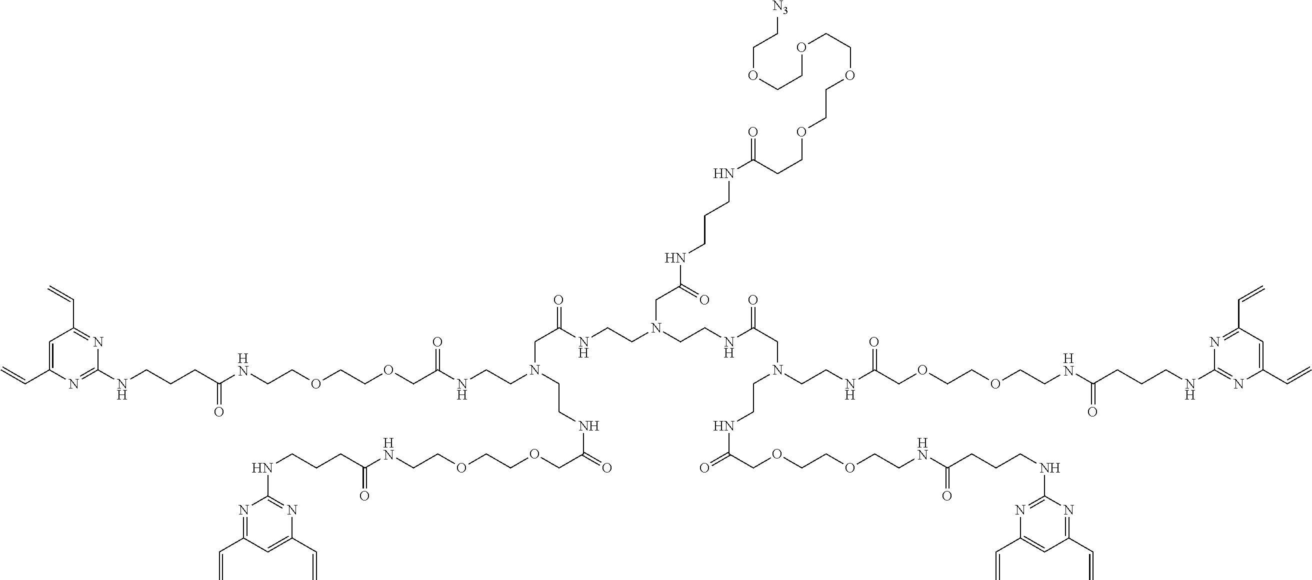 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCN(CCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCCNC(=O)CCOCCOCCOCCOCCN=[N+]=[N-])n1]

To a mixture of compound 2 (343.6 mg, 1.47 mmol) and compound 231 (400 mg, 294.64 umol) in DCM (20 mL) was added DIPEA (571.2 mg, 4.42 mmol). Then HATU (448.1 mg, 1.18 mmol) was slowly added into the above solution. The reaction mixture was stirred at 20° C. for 1 hour. LCMS showed starting material was consumed and desired mass was detected. The reaction mixture was concentrated in vacuum to give a residue, which was purified by prep-HPLC (column: Waters Xbridge 150 mm*25 mm*5 um; mobile phase: [water (ammonia hydroxide v/v)-ACN]; B %: 30%-60%, 9 min) and lyophilized to afford 232, Scaffold F (155 mg, 22.7% yield) as yellow oil. Then the product was dissolved in DMSO (7 mL) immediately. LCMS: rt=0.961 min, (1110.3 [[M+H]/2]⁺), 97.65% purity.

### Example 31—Synthesis of TetraDVP Scaffold G

### Step 1: Synthesis of Compound 233

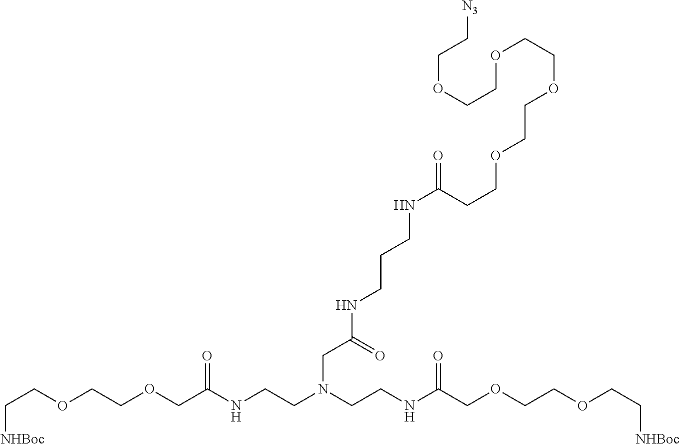 [SMILES: CC(C)(C)OC(=O)NCCOCCOCC(=O)NCCN(CCNC(=O)COCCOCCNC(=O)OC(C)(C)C)CC(=O)NCCCNC(=O)CCOCCOCCOCCOCCN=[N+]=[N-]]

To a mixture of compound 229 (3 g, 4.60 mmol) and compound 219 (1.93 g, 4.60 mmol) in DCM (30 mL) was added DIPEA (2.97 g, 23.02 mmol) and HATU (1.75 g, 4.60 mmol). The reaction mixture was stirred at 20° C. for 1 hour. LCMS showed starting material was consumed and desired mass was detected. The reaction mixture was concentrated in vacuum to give a residue, which was purified by reversed phase Combiflash (water (0.1% FA)-ACN; B % 40%-60%). The mixture was adjusted to pH=8 by aq. NaHCO₃ solution and concentrated in vacuum to remove CH₃CN. Then the mixture was extracted with EtOAc (200 mL*2). The combined organic layers were washed with brine (200 mL), dried over sodium sulfate, concentrated in vacuum to afford compound 233 (2.0 g, 44.3% yield) as yellow oil. LCMS: rt=0.533 min, (981.4 [M+H]⁺), 94.19% purity. ¹H NMR (400 MHz, METHANOL-d₄) δ 4.00 (s, 4H), 3.74 (t, J=6.0 Hz, 2H), 3.69-3.61 (m, 22H), 3.53 (t, J=6.0 Hz, 4H), 3.40-3.34 (m, 6H), 3.28-3.22 (m, 10H), 2.71 (t, J=6.8 Hz, 4H), 2.46 (t, J=6.0 Hz, 2H), 1.70 (q, J=6.4 Hz, 2H) 1.44 (s, 18H).

### Step 2: Synthesis of Compound 234

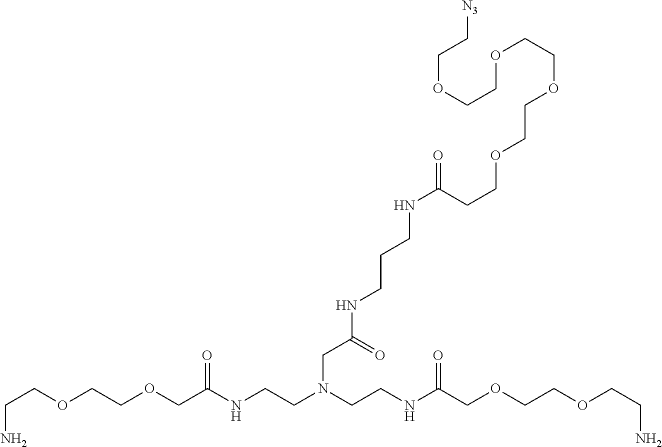 [SMILES: [N-]=[N+]=NCCOCCOCCOCCOCCC(=O)NCCCNC(=O)CN(CCNC(=O)COCCOCCN)CCNC(=O)COCCOCCN]

To a mixture of compound 233 (2 g, 2.04 mmol) in dioxane (10 mL) was added HCl/dioxane (4 M, 9.1 mL). The reaction mixture was stirred at 20° C. for 4 hours. LCMS showed starting material was consumed and desired mass was detected. The reaction mixture was concentrated in vacuum to afford compound 234 (1.81 g, 100% yield) as yellow oil. LCMS: rt=0.354 min, (781.5 [M+H]⁺), 55.4% purity.

### Step 3: Synthesis of Compound 235

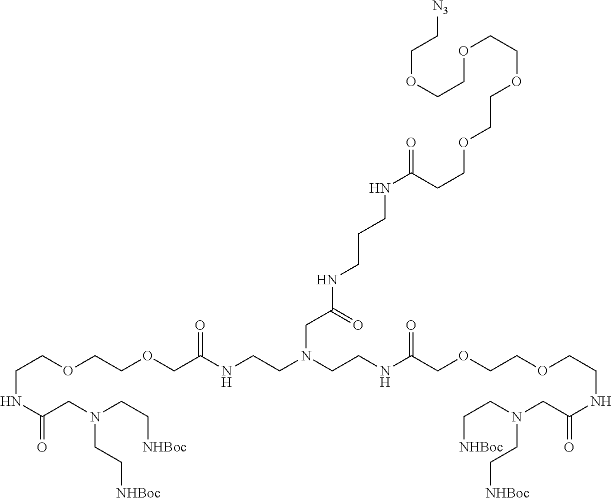 [SMILES: CC(C)(C)OC(=O)NCCN(CCNC(=O)OC(C)(C)C)CC(=O)NCCOCCOCC(=O)NCCN(CCNC(=O)COCCOCCNC(=O)CN(CCNC(=O)OC(C)(C)C)CCNC(=O)OC(C)(C)C)CC(=O)NCCCNC(=O)CCOCCOCCOCCOCCN=[N+]=[N-]]

To a mixture of compound 203 (487.1 mg, 1.35 mmol) in DCM (5 mL) was added HATU (512.5 mg, 1.35 mmol) and DIPEA (348.4 mg, 2.70 mmol). The reaction mixture was stirred at 20° C. for 0.5 hour. Then, a solution of compound 234 (600 mg, 0.67 mmol) and DIPEA (522.6 mg, 4.04 mmol) in DCM (5 mL) was added dropwise into the above solution. The reaction mixture was stirred at 20° C. for another 0.5 hour. LCMS showed the reaction was completed. The reaction mixture was concentrated in vacuum to give a residue, which was purified by reversed phase Combiflash (water (0.1% FA)-ACN; B % 40%-60%). The mixture was adjusted to pH=8 by aq. NaHCO₃ solution and concentrated in vacuum to remove CH₃CN. Then the mixture was extracted with EtOAc (100 mL*2). The combined organic layer was washed with brine (100 mL), dried over sodium sulfate, concentrated in vacuum to afford compound 235 (360 mg, 34.5% yield) as yellow oil. LCMS: rt=0.739 min, (1468.1 [M+H]⁺), 100.00% purity. ¹H NMR (400 MHz, METHANOL-d₄) δ 4.01 (s, 4H), 3.76-3.70 (m, 4H), 3.70-3.59 (m, 28H), 3.45 (t, J=5.6 Hz, 4H), 3.40-3.34 (m, 6H), 3.29-3.22 (m, 6H), 3.18-3.10 (m, 14H), 2.71 (t, J=6.6 Hz, 4H), 2.60 (br t, J=6.0 Hz, 8H), 2.46 (t, J=6.0 Hz, 2H), 1.74-1.66 (m, 2H), 1.45 (s, 36H).

### Step 4: Synthesis of Compound 236

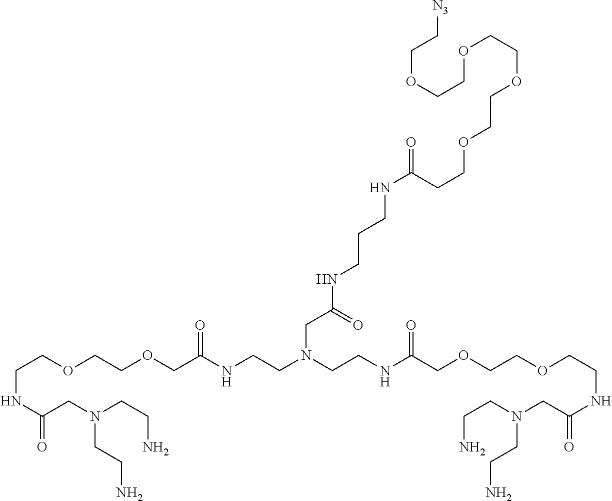 [SMILES: [N-]=[N+]=NCCOCCOCCOCCOCCC(=O)NCCCNC(=O)CN(CCNC(=O)COCCOCCNC(=O)CN(CCN)CCN)CCNC(=O)COCCOCCNC(=O)CN(CCN)CCN]

To a mixture of compound 235 (250 mg, 0.17 mmol) in dioxane (4 mL) was added HCl/dioxane (4 M, 1 mL). The reaction mixture was stirred at 20° C. for 2 hours. LCMS showed starting material was consumed and desired mass was detected. The reaction mixture was concentrated in vacuum to afford compound 236 (225 mg, 99% yield) as yellow oil. LCMS: rt=0.354 min, (1167.8 [M+H]⁺), 75.55% purity.

### Step 5: Synthesis of Compound 237 (Scaffold G)

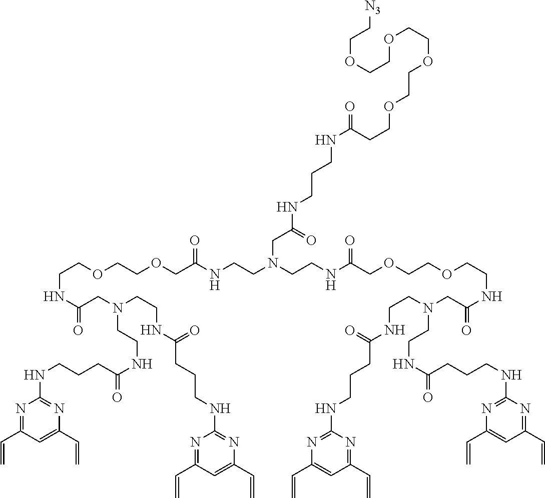 [SMILES: C=Cc1cc(C=C)nc(NCCCC(=O)NCCN(CCNC(=O)CCCNc2nc(C=C)cc(C=C)n2)CC(=O)NCCOCCOCC(=O)NCCN(CCNC(=O)COCCOCCNC(=O)CN(CCNC(=O)CCCNc2nc(C=C)cc(C=C)n2)CCNC(=O)CCCNc2nc(C=C)cc(C=C)n2)CC(=O)NCCCNC(=O)CCOCCOCCOCCOCCN=[N+]=[N-])n1]

To a mixture of compound 236 (225 mg, 0.17 mmol) in DCM (10 mL) was added DIPEA (439.7 mg, 3.40 mmol) and compound 2 (198.4 mg, 0.85 mmol). Then HATU (323.5 mg, 0.85 mmol) was slowly added into the above solution in potions. The reaction mixture was stirred at 20° C. for 1 hour. LCMS showed starting material was consumed and desired mass was detected. The reaction mixture was concentrated in vacuum to give a residue, which was purified by prep-HPLC (column: Waters Xbridge 150 mm*25 mm*5 μm; mobile phase: [water (ammonia hydroxide v/v)-ACN]; B %: 36%-66%, 9 min) and lyophilized to afford 237, Scaffold G (278 mg, 78.2% yield) as yellow oil. Then the product was dissolved in DMSO (13 mL) immediately. LCMS: rt=0.958 min, (1929.4 [M+H]⁺), 95.17% purity.

### Example 32—Synthesis of TetraDVP Scaffold H

### Step 1: Synthesis of Compound 238

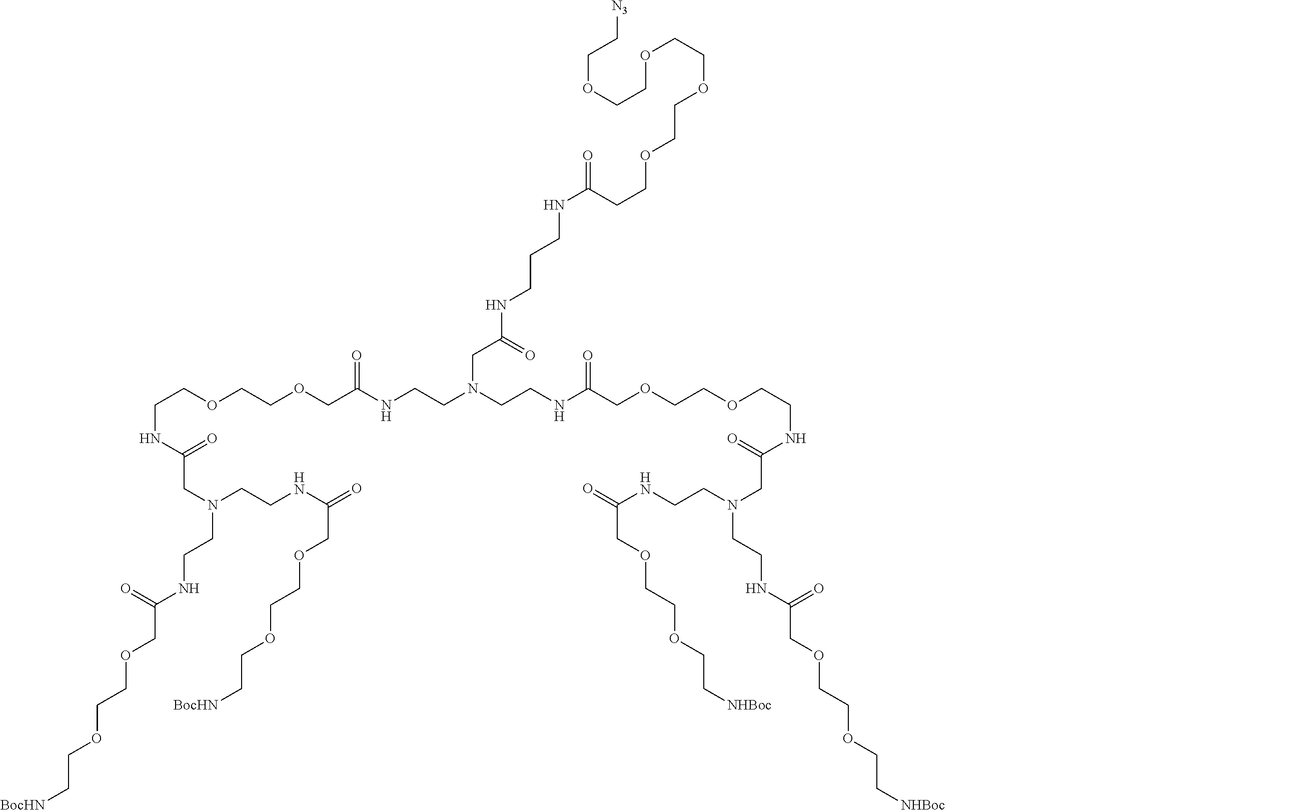 [SMILES: CC(C)(C)OC(=O)NCCOCCOCC(=O)NCCN(CCNC(=O)COCCOCCNC(=O)OC(C)(C)C)CC(=O)NCCOCCOCC(=O)NCCN(CCNC(=O)COCCOCCNC(=O)CN(CCNC(=O)COCCOCCNC(=O)OC(C)(C)C)CCNC(=O)COCCOCCNC(=O)OC(C)(C)C)CC(=O)NCCCNC(=O)CCOCCOCCOCCOCCN=[N+]=[N-]]

To a mixture of compound 229 (878.5 mg, 1.35 mmol) in DCM (10 mL) was added DIEA (174.2 mg, 1.35 mmol) and HATU (512.5 mg, 1.35 mmol). The reaction mixture was stirred at 20° C. for 0.5 hour. Then a solution of compound 234 (600 mg, 0.67 mmol) and DIEA (522.61 mg, 4.04 mmol) in DCM (5 mL) was added into the above solution. The reaction mixture was stirred at 20° C. for another 0.5 hour. LCMS showed the reaction was completed. The reaction mixture was concentrated in vacuum to give a residue, which was purified by reversed phase Combiflash (water (0.1% FA)-ACN; B % 60%-80%). The mixture was adjusted to pH=8 by aq. NaHCO₃ solution and concentrated in vacuum to remove CH₃CN. Then the mixture was extracted with EtOAc (100 mL*2). The combined organic layers were washed with brine (100 mL), dried over sodium sulfate, concentrated in vacuum to afford compound 238 (360 mg, 26.1% yield) as yellow oil. LCMS: rt=0.740 min, (1025.2 [[M+H]/2]⁺), 100.00% purity. ¹H NM R (400 MHz, METHANOL-d₄) δ 4.02 (s, 12H), 3.76-3.71 (m, 4H), 3.70-3.64 (m, 32H), 3.63-3.58 (m, 8H), 3.53 (t. J=5.6 Hz, 8H), 3.47-3.42 (m, 4H), 3.41-3.34 (m, 14H), 3.28-3.22 (m, 18H), 2.71 (t, J=6.4 Hz, 12H), 2.47 (t, J=6.0 Hz, 2H), 1.74-1.67 (m, J=6.4 Hz, 2H), 1.44 (s, 36H).

### Step 2: Synthesis of Compound 239

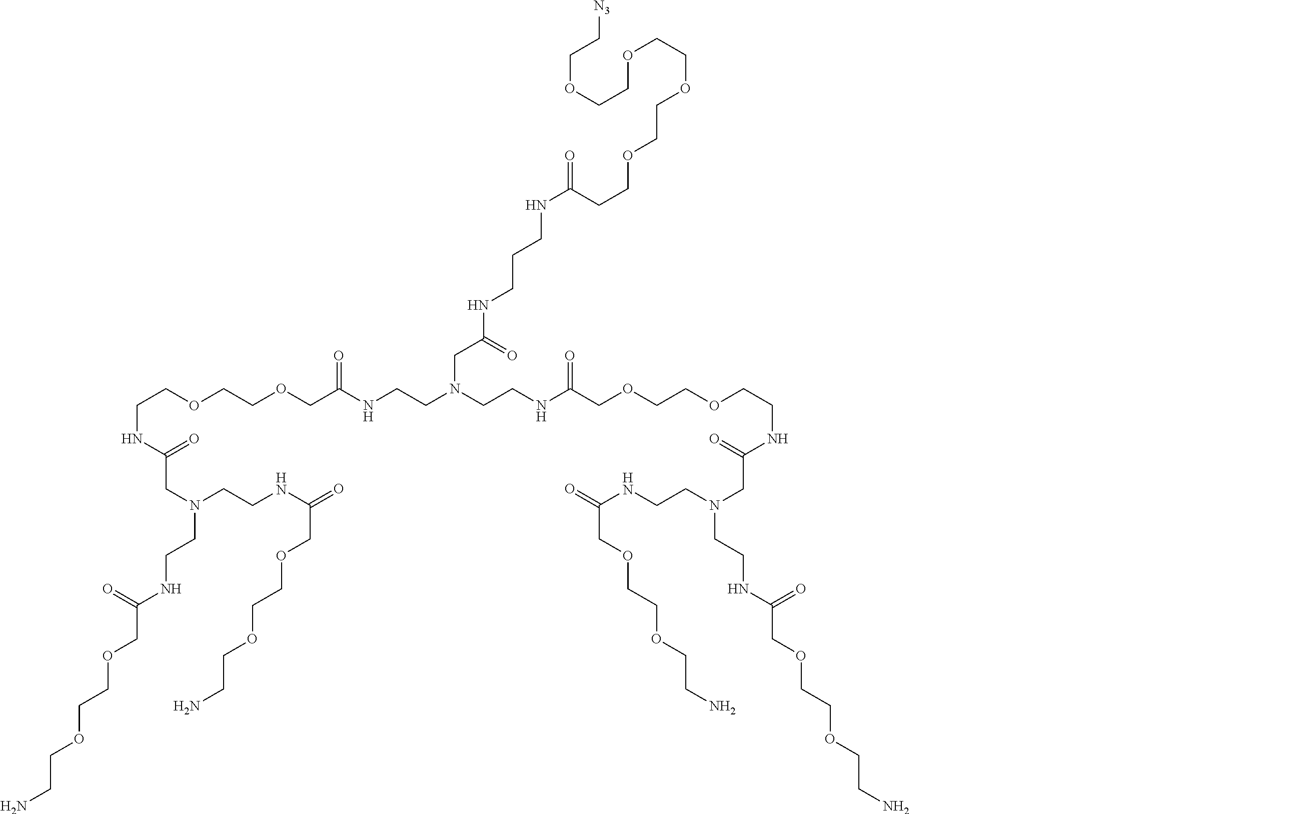 [SMILES: [N-]=[N+]=NCCOCCOCCOCCOCCC(=O)NCCCNC(=O)CN(CCNC(=O)COCCOCCNC(=O)CN(CCNC(=O)COCCOCCN)CCNC(=O)COCCOCCN)CCNC(=O)COCCOCCNC(=O)CN(CCNC(=O)COCCOCCN)CCNC(=O)COCCOCCN]

To a mixture of compound 238 (300 mg, 0.15 mmol) in dioxane (5 mL) was added HCl/dioxane (4 M, 5 mL). The reaction mixture was stirred at 20° C. for 4 hours. The reaction mixture was concentrated in vacuum to afford compound 239 (270 mg, 96.9% yield) as yellow oil. LCMS: rt=0.414 min, (1647.9 [M+H]⁺), 44.49% purity.

### Step 2: Synthesis of Compound 240 (Scaffold H)

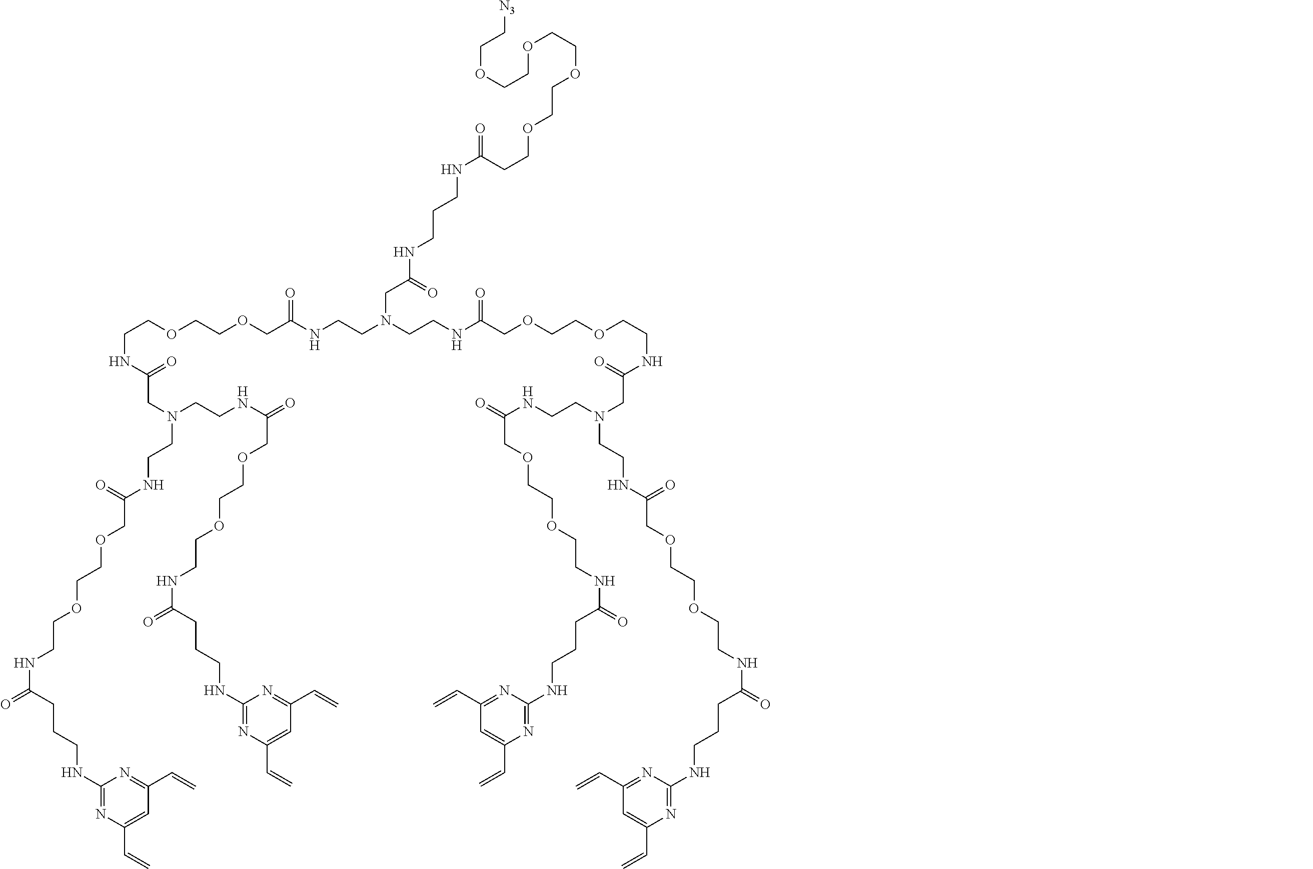 [SMILES: C=Cc1cc(C=C)nc(NCCCC(=O)NCCOCCOCC(=O)NCCN(CCNC(=O)COCCOCCNC(=O)CCCNc2nc(C=C)cc(C=C)n2)CC(=O)NCCOCCOCC(=O)NCCN(CCNC(=O)COCCOCCNC(=O)CN(CCNC(=O)COCCOCCNC(=O)CCCNc2nc(C=C)cc(C=C)n2)CCNC(=O)COCCOCCNC(=O)CCCNc2nc(C=C)cc(C=C)n2)CC(=O)NCCCNC(=O)CCOCCOCCOCCOCCN=[N+]=[N-])n1]

To a mixture of compound 239 (270 mg, 0.14 mmol) and compound 2 (165.5 mg, 0.71 mmol) in DCM (5 mL) was added DIEA (366.7 mg, 2.84 mmol). Then HATU (215.8 mg, 0.57 mmol) was added into the above solution. The reaction mixture was stirred at 20° C. for 1 hour. LCMS showed the reaction was completed. The reaction mixture was concentrated in vacuum to give a residue, which was purified by prep-HPLC (column: Waters Xbridge 150 mm*25 mm*5 um; mobile phase: [water (ammonia hydroxide v/v)-ACN]; B %: 34%-64%, 9 min) and lyophilized to afford to afford 240, Scaffold H (131 mg, 34.4% yield) as yellow oil. Then the product was dissolved in DMSO (4.7 mL) immediately. LCMS: rt=0.946 min, (1255.3 [[M+H]/2]⁺), 100.00% purity.

### Example 32—Synthesis of DBCO Reagent 247

### Step 1: Synthesis of Fmoc-Val-Cit-OH (241)

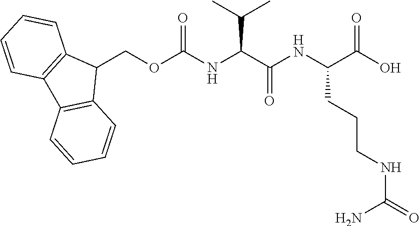 [SMILES: CC(C)[C@H](NC(=O)OCC1c2ccccc2-c2ccccc21)C(=O)N[C@@H](CCCNC(N)=O)C(=O)O]

L-citrulline (85 mg, 0.486 mmol) in DME (1.5 mL) was added to a solution of Fmoc-Val-OSu (202 mg, 0.463 mmol) and NaHCO₃ (42.8 mg, 0.509 mmol) in H₂O (3 mL) and THF (4 mL) at 0° C. The reaction warmed to rt and stirred for 48 h. Upon completion, the reaction was adjusted to pH 10 with sat. aq. K₂CO₃ and washed with EtOAc (2×20 mL). The aqueous layer was acidified to pH 4 with 10% aq. citric acid and the formed gelatinous mixture was filtered and dried in vacuo to yield Fmoc-Val-Cit-OH 241 (141 mg, 0.284 mmol, 61%) as an off-white solid. ¹H NMR (700 MHz, DMSO-d₆) δ ppm: 7.89 (d, 2H, J=7.6 Hz), 7.75 (dd, 2H, J=11.7, 7.8 Hz), 7.41 (t, 2H, J=7.4 Hz), 7.34-7.30 (m, 2H), 6.00 (s, 1H), 4.31-4.26 (m, 1H), 4.27-4.19 (m, 2H), 4.17-4.13 (m, 1H), 3.94-3.87 (m, 1H), 2.99-2.92 (m, 2H), 1.98 (app sx, 1H, J=6.8 Hz), 1.74-1.66 (m, 1H), 1.61-1.53 (m, 1H), 1.46-1.35 (m, 2H), 0.89 (d, 3H, J=6.8 Hz), 0.86 (d, 3H, J=6.8 Hz). ¹³C NMR (176 MHz, DMSO-d₆) δ ppm: 173.4, 171.3, 158.8, 156.1, 143.9, 143.9, 140.7, 127.7, 127.1, 125.4, 120.1, 65.7, 59.8, 51.9, 46.7, 38.8, 30.6, 28.4, 26.6, 19.2, 18.2. HRMS (ESI) m/z found [M+H]⁺ 497.2394, C₂₆H₃₃N₄O₆ required 497.2395.

### Step 2: Synthesis of Fmoc-Val-Cit-PABA (242)

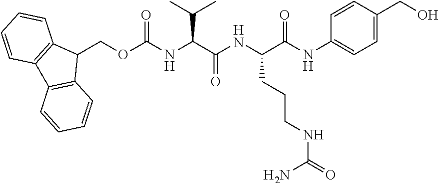 [SMILES: CC(C)[C@H](NC(=O)OCC1c2ccccc2-c2ccccc21)C(=O)N[C@@H](CCCNC(N)=O)C(=O)Nc1ccc(CO)cc1]

A solution of Fmoc-Val-Cit-OH 241 (104 mg, 0.210 mmol), 4-aminobenzyl alcohol (PABA, 52.0 mg, 0.419 mmol) and 2-ethoxy-1-ethoxycarbonyl-1,2-dihydroquinoline (EEDQ, 104 mg, 0.419 mmol) in DCM (2.5 mL) and MeOH (1.5 mL) was stirred at rt for 22 h. Upon completion, the mixture was diluted with Et₂O (10 mL), filtered and washed with Et₂O (2×4 mL) to yield Fmoc-Val-Cit-PABA 242 (66.4 mg, 0.110 mmol, 53%) as a white solid. ¹H NMR (700 MHz, DMSO-d₆) δ ppm: 9.98 (s, 1H), 8.12 (d, 1H, J=7.5 Hz), 7.89 (d, 2H, J=7.6 Hz), 7.74 (t, 2H, J=8.1 Hz), 7.54 (d, 2H, J=8.4 Hz), 7.46-7.39 (m, 3H), 7.34-7.30 (m, 2H), 7.23 (d, 2H, J=8.4 Hz), 6.00 (t, 1H, J=5.8 Hz), 5.41 (s, 2H), 5.09 (t, 1H, J=5.5 Hz), 4.43-4.39 (m, 3H), 4.33-4.20 (m, 3H), 3.95-3.91 (m, 1H), 3.06-2.89 (m, 2H), 2.04-1.95 (m, 1H), 1.74-1.55 (m, 2H), 1.50-1.32 (m, 2H), 0.89-0.84 (m, 6H); ¹³C NMR (176 MHz, DMSO-d₆) δ ppm: 171.3, 170.4, 158.9, 156.1, 143.9, 140.7, 137.5, 137.4, 127.7, 127.1, 126.9, 125.4, 120.1, 118.9, 65.7, 62.6, 60.1, 53.1, 46.7, 38.6, 30.5, 29.5, 26.8, 19.2, 18.3; HRMS (ESI) m/z found [M+H]⁺ 602.2968, C₃₃H₄₀N₅O₆⁺ required 602.2973.

### Step 3: Synthesis of Boc-PEG4-Val-Cit-PABA (243)

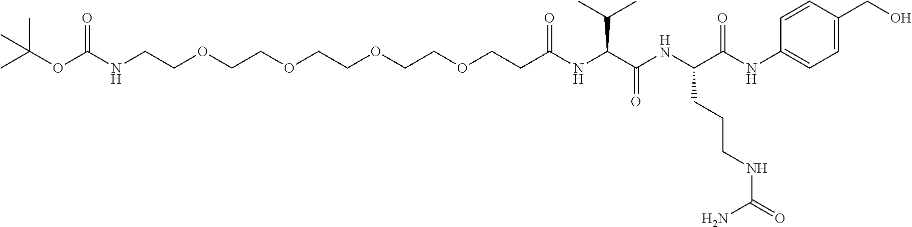 [SMILES: CC(C)[C@H](NC(=O)CCOCCOCCOCCOCCNC(=O)OC(C)(C)C)C(=O)N[C@@H](CCCNC(N)=O)C(=O)Nc1ccc(CO)cc1]

A solution of Fmoc-Val-Cit-PABA 242 (100 mg, 0.166 mmol) and diethylamine (343 μL, 3.33 mmol) in DMF (1.5 mL) was stirred at rt for 19 h. Upon completion, the solvent was evaporated under reduced pressure, the product was crashed out with DCM, filtered, and washed with EtOAc and Et₂O to give H-Val-Cit-PABA (52.3 mg, 0.138 mmol, 83%) as a white solid, which was carried through without further purification. A solution of H-Val-Cit-PABA (52.3 mg, 0.138 mmol), Boc-PEG₄-COOH (50.0 mg, 0.138 mmol), HBTU (52.5 mg, 0.138 μmol) and DIPEA (48.0 μL, 0.276 μmol) in DMF (3 mL) was stirred at rt for 2 h. Upon completion, the reaction was concentrated under a stream of N₂ and the crude residue purified by reverse-phase flash column chromatography (5-100% solvent B in solvent A; Solvent A: 0.1 M NH₄OH (aq), Solvent B: MeCN) and lyophilised to yield Boc-PEG₄-Val-Cit-PABA 243 (50.5 mg, 69.4 μmol, 51%) as a white solid. ¹H NMR (700 MHz, DMSO-d₆) δ ppm: 9.87 (s, 1H), 8.07 (d, 1H, J=7.6 Hz), 7.86 (d, 1H, J=8.7 Hz), 7.53 (d, 2H, J=8.5 Hz), 7.21 (d, 2H, J=8.6 Hz), 6.73 (t, 1H, J=5.0 Hz), 6.00 (t, 1H, J=5.8 Hz), 5.39 (s, 2H), 5.09 (t, 1H, J=5.7 Hz), 4.41 (d, 2H, J=5.5 Hz), 4.39-4.31 (m, 1H), 4.23-4.17 (m, 1H), 3.63-3.53 (m, 2H), 3.50-3.43 (m, 12H), 3.07-2.96 (m, 3H), 2.96-2.87 (m, 1H), 2.47-2.42 (m, 1H), 2.40-2.31 (m, 1H), 1.95 (h, 1H, J=6.8 Hz), 1.76-1.63 (m, 1H), 1.62-1.52 (m, 1H), 1.45-1.38 (m, 2H), 1.37-1.35 (s, 9H), 0.82 (dd, 6H, J=6.8 Hz); ¹³C NMR (176 MHz, DMSO-d₆) δ ppm: 171.6, 170.8, 159.3, 156.0, 137.9, 127.4, 119.3, 78.1, 70.2, 70.2, 70.1, 69.9, 69.9 69.6, 67.4, 63.0, 58.0, 53.5, 39.0, 36.4, 31.0, 29.8, 28.7, 27.3, 19.6, 18.5; LRMS (ESI) m/z found [M+H]⁺ 726.8, C₃₄H₅₉N₅₉O₁₁⁺ required 727.4273.

### Step 4: Synthesis of Boc-PEG4-Val-Cit-PABC-MMAE (244)

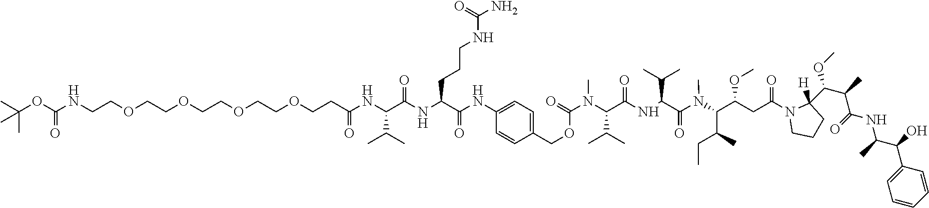 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCNC(=O)OC(C)(C)C)C(C)C)cc1)C(C)C]

A solution of Boc-PEG₄-Val-Cit-PABA 243 (60.0 mg, 82.5 μmol), bis(4-nitrophenyl) carbonate (50.2 mg, 0.165 mmol) and DIPEA (43.0 μL, 0.248 mmol) in DMF (2 mL) was stirred at rt for 24 h. Upon completion, the mixture was diluted with Na₂CO₃ (10 mL) and extracted with DCM (10 mL). The aqueous layer was washed with DCM (4×20 mL), the organic layers combined and washed with Na₂CO₃ (2×10 mL). The combined organic fractions were dried (MgSO₄), concentrated in vacuo to yield Boc-PEG₄-Val-Cit-PABC-PNP as a brown oil, which was carried through without further purification. A solution of MMAE (25.0 mg, 34.8 μmol), Boc-PEG₄-Val-Cit-PABC-PNP (25.9 mg, 29.0 μmol), HOBt·H₂O (4.70 mg, 34.8 μmol) and DIPEA (12.1 μL, 69.6 μmol) in DMF (2 mL) was stirred at rt for 21 h. Upon completion, the reaction mixture was concentrated under a stream of N₂. The crude product was purified by reverse-phase flash column chromatography (5-100% solvent B in solvent A; Solvent A: 0.1 M NH₄OH (aq), Solvent B: MeCN) and lyophilised to yield Boc-PEG₄-Val-Cit-PABC-MMAE 244 (45.6 mg, 31.0 μmol, 89%) as a light-yellow solid. HPLC (5-95% MeCN/H₂O over 20 min) retention time 10.370 min; HRMS (ESI) m/z found [M+H]⁺ 1470.9042, C₇₄H₂₄N₁₁O₁₉⁺ required 1470.9075.

### Step 5: Synthesis of Fmoc-PEG2-Glu(-PEG4-Val-Cit-PABC-MMAE)-PEG2-Glu(-PEG4-Val-Cit-PABC-MMAE)-NH2 (245)

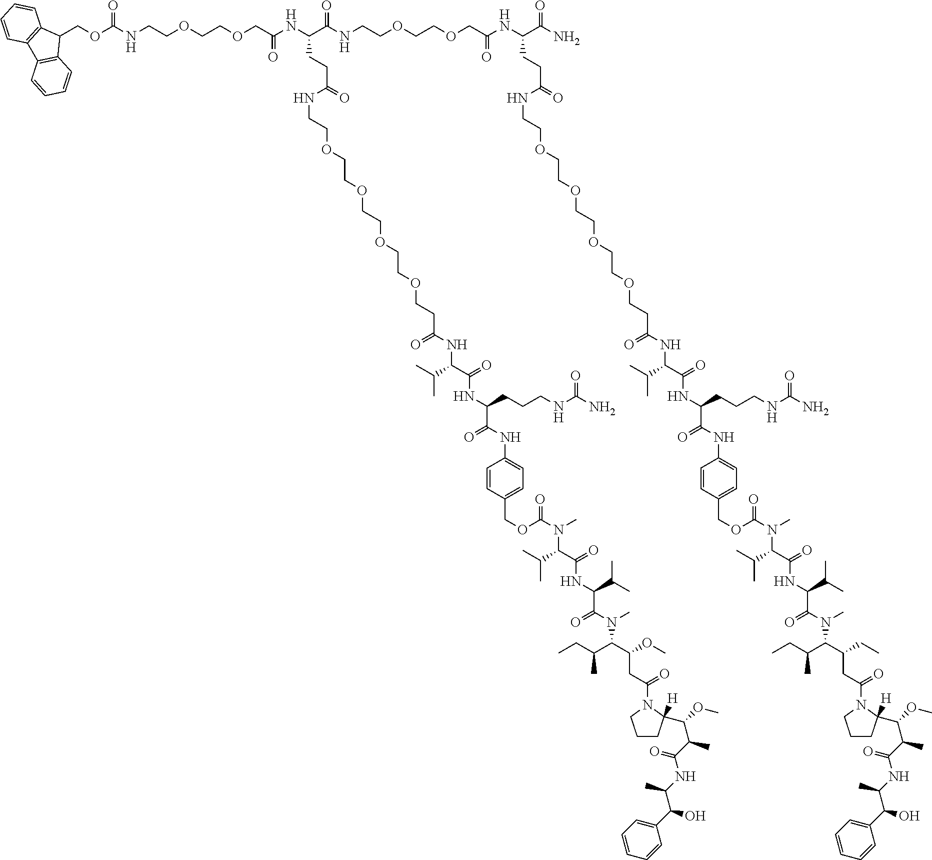

A solution of Boc-PEG4-Val-Cit-PABC-M MAE 244 (40.0 mg, 27.2 μmol), TIPS (100 μL), TFA (500 μL) and DCM (950 μL) was stirred at rt for 20 min. Upon completion, the reaction mixture was concentrated under a stream of N₂ and carried forward without further purification. A solution of H-PEG₄-Val-Cit-PABC-M MAE (37.3 mg, 27.2 μmol), Fmoc-PEG₂-Glu-PEG₂-Glu-NH₂ (8.57 mg, 10.9 μmol), HATU (18.7 mg, 49.1 μmol) and DIPEA (12.3 μL, 70.9 μmol) in DMF (2 mL) was stirred at rt for 48 h. Upon completion, the reaction was concentrated under a stream of N₂, and the crude product purified by preparative HPLC and lyophilised to yield Fmoc-PEG₂-Glu(-PEG₄-Val-Cit-PABC-M MAE)-PEG₂-Glu(-PEG₄-Val-Cit-PABC-MMAE) 245 (1.10 mg, 0.315 μmol, 3%) as a white solid. HPLC (5-95% MeCN/H₂O over 20 min) retention time 12.721 min; LRMS (ESI) m/z found [M+2H]⁺ 1165.3, [M+3H]⁺ 1747.7, [M+4H]⁺ 873.9, C₁₇₅H₂₇₆N₂₇O₄₆⁺ required 3492.0073.

### Step 6: Synthesis of Fmoc-PEG2-Glu(-PEG4-Val-Cit-PABC-MMAE)-PEG2-PEG2-Glu(-PEG4-Val-Cit-PABC-MMAE)-NH2 (246)

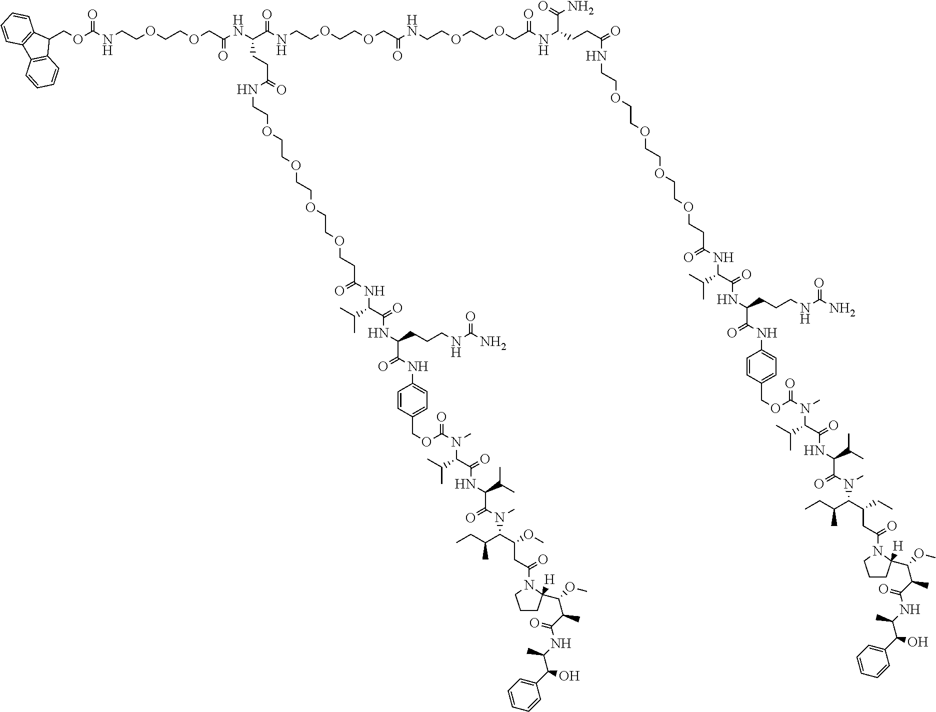

A solution of Boc-PEG₄-Val-Cit-PABC-MMAE 244 (38.0 mg, 25.8 μmol), TIPS (100 μL), TFA (500 μL) and DCM (950 μL) was stirred at rt for 20 min. Upon completion, the reaction mixture was concentrated under a stream of N₂, dissolved in H₂O (5 mL), lyophilised, and carried forward without further purification. A solution of Fmoc-PEG₂-Glu-PEG₂-PEG₂-Glu-NH₂ (10.9 mg, 11.7 μmol), BTTFH (7.77 mg, 24.6 μmol) and DIPEA (8.55 μL, 49.2 μmol) in DMF (0.5 mL) was stirred at 0° C. for 1.5 h. To the solution, TFA.HN-PEG₄-Val-Cit-PABC-MMAE (33.7 mg, 23.0 μmol) and DIPEA (6.09 μL, 35.0 μmol) in DMF (0.5 mL) were added and the reaction mixture stirred at rt for 24 h. Upon completion, the reaction was concentrated under a stream of N₂, and the crude product purified by preparative HPLC and lyophilised to yield Fmoc-PEG₂-Glu(-PEG₄-Val-Cit-PABC-MMAE)-PEG₂-PEG₂-Glu(-PEG₄-Val-Cit-PABC-MMAE)-NH₂ 246 (6.39 mg, 1.76 μmol, 15%) as a white solid. HPLC (5-95% MeCN/H₂O over 20 min) retention time 12.720 min; HRMS (ESI) m/z found [M+H]⁺ 3637.140, C₁₈₁H₃₈₇N₂₈O₄₉⁺ required 3637.0773.

### Step 7: Synthesis of DBCO-PEG5-PEG2-Glu(-PEG4-Val-Cit-PABC-MMAE)-PEG2-PEG2-Glu(-PEG4-Val-Cit-PABC-MMAE)-NH2 (247)

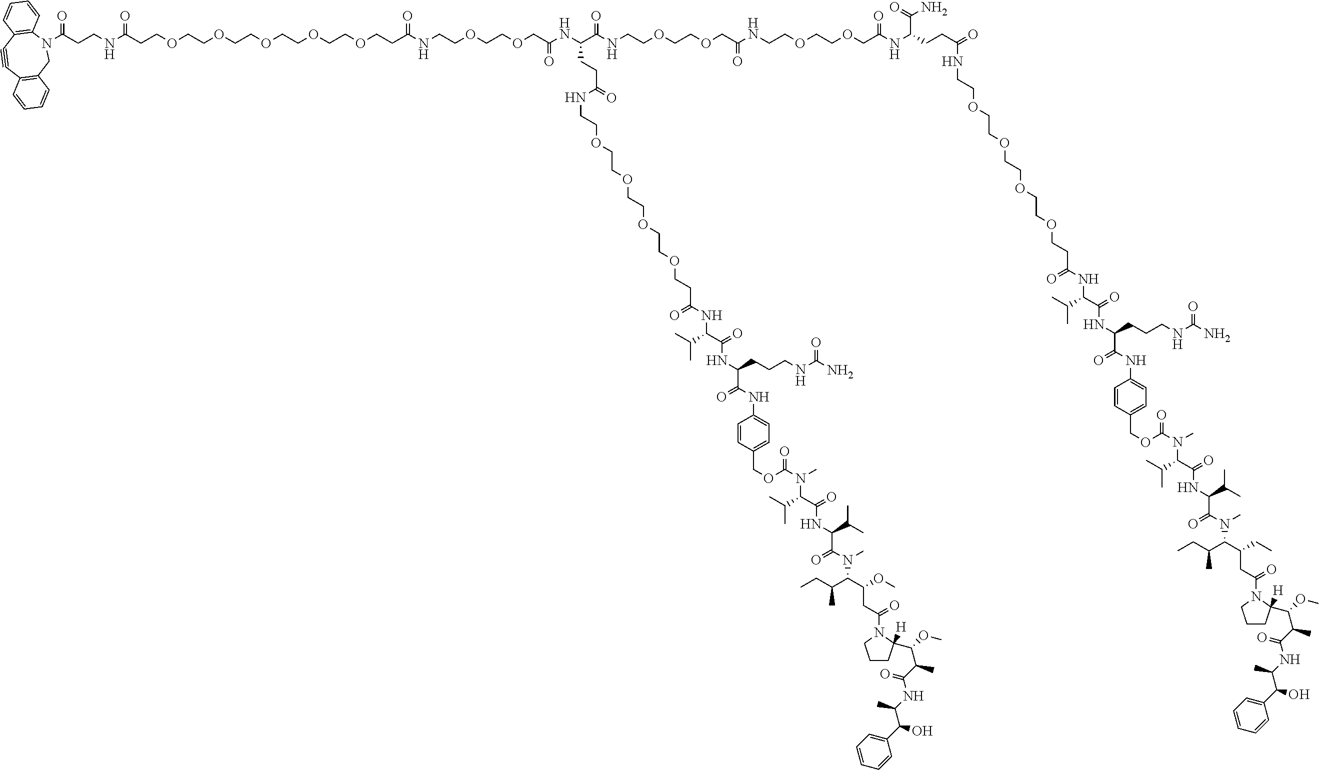

Polymer-bound piperazine (30 mg, mmol, 200-400 mesh, 1.0-2.0 mmol/g loading, 2% cross-linked with divinylbenzene) was added to a solution of Fmoc-PEG₂-Glu(-PEG₄-Val-Cit-PABC-MMAE)-PEG₂-PEG₂-Glu(-PEG₄-Val-Cit-PABC-MMAE)-NH₂ 246 (3.60 mg, 0.989 μmol) in DMF (200 μL), followed by the addition of 0.1% DBU. The resulting mixture was stirred for 2 h at room temperature. Upon completion, the solvent was evaporated under N₂ stream, and the residue taken forward without further purification. A solution of H-PEG₂-Glu(-PEG₄-Val-Cit-PABC-M MAE)-PEG₂-PEG₂-Glu(-PEG₄-Val-Cit-PABC-M MAE) (3.38 mg, 0.989 μmol), DBCO-PEG₅-COOH (1.60 mg, 2.68 μmol), HATU (1.02 mg, 2.68 μmol) and DIPEA (0.932 μL, 5.36 μmol) in DMF (100 μL) was stirred at rt for 24 h. Upon completion, the reaction was concentrated under a stream of N₂, and the crude product purified by preparative HPLC to yield DBCO-PEG₅-PEG₂-Glu(-PEG₄-Val-Cit-PABC-MMAE)-PEG₂-PEG₂-Glu(-PEG₄-Val-Cit-PABC-MMAE)-NH₂ 247 (0.60 mg, 0.150 μmol, 16%) as a white solid. HPLC (5-95% MeCN/H₂O over 20 min) retention time 13.208 min; H RMS (ESI) m/z found [M+H]⁺ 3995.4700, [M+2H]⁺ 1997.1381, [M+3H]⁺ 1331.7620 and [M+4H]⁺ 999.0757, C₁₉₈H₃₁₅N₃₀O₅₅⁺ required 3993.2773.

### Example 33—Peptide Synthesis

Manual peptide synthesis: Manual peptide synthesis was carried out on Merck LL MHBA low-loading Rink amide resin (0.308 mmol/g, 100-200 mesh, 1 eq) to afford C-terminal amides. Amino acid coupling was performed using Fmoc-protected amino acids (3 eq), HATU (3 eq) and DIPEA (6 eq) in DMF for 1-3 h. Fmoc deprotection was carried out using 20% piperidine in DMF for 10 min.

Resin cleavage: Side chain deprotection and cleavage from resin were achieved using a TFA cleavage cocktail containing TFA/TIPS/H₂O (95:2.5:2.5) at rt for 3 h. Upon filtration and evaporation under a stream of N₂, the peptides were precipitated in ice-cold Et₂O. The crude peptides were lyophilised, the mass analysed by LCMS and the purity determined by analytical HPLC. The crude peptides were carried through without further purification.

### LCMS and Purity of Peptides

The peptide sequence, mass observed LCMS, peptide purity and yield, together with the retention time determined by analytical HPLC are shown in Table 1 and 2 below.

TABLE 1
Peptide sequence, mass observed and purity.

| | | | m/z | m/z | | Purity |
|---|---|---|---|---|---|---|
| Peptide | Sequence | Mass | found | calc. | Species | (%) |
| P1 | Fmoc-XEXE- | 787.3 | 787.8 | 788.3 | M + H | 81 |
| | NH₂ | | | | | |
| P2 | Fmoc-XEXXE- | 932.4 | 933.0 | 933.4 | M + H | 78 |
| | NH₂ | | | | | |

X = Fmoc-8-amino-3,6-dioxaoctanoic acid.

TABLE 2
Peptide sequence, analytical HPLC retention time and yield.

| Peptide | Sequence | Rₜ (min)† | Rₜ (min)‡ | Yield (%) |
|---|---|---|---|---|
| P1 | Fmoc-XEXE-NH₂ | 8.637 | 8.960 | 64 |
| P2 | Fmoc-XEXXE-NH₂ | 8.614 | 8.938 | 79 |

†Rₜ on a 5-95% B gradient (A: 0.05% (v/v) TFA in H₂O, B: 0.05% (v/v) TFA in MeCN) over 20 min;
‡Rₜ on a 10-80% B gradient (A: 0.05% (v/v) TFA in H₂O, B: 0.05% (v/v) TFA in MeCN) over 20 min;
X = Fmoc-8-amino-3,6-dioxaoctanoic acid.

### Bioconjugation

### General Method for Trastuzumab Bioconjugation

To a solution of trastuzumab in TBS buffer (1×, pH 8, [Tras]=2.5 mgml⁻¹, 200 μL, 3.4 nmol) was added TCEP (5 mM stock in TBS, 6.8 μL, 34 nmol, 10 equiv.) and the mixture was incubated at 37° C. for 1 hour on a thermal shaker at 400 rpm. DMSO was added to ensure a final organic solvent concentration of 5-10%. Linker/scaffold (1-10 mM stock in DMSO, 2-10 equiv.) was added, and the mixture incubated at 37° C. for 4 hours on a thermal shaker at 400 rpm. The conjugate was purified by Zeba™ Spin Desalting Column (MW cut-off 40,000 Da, Thermo Fisher Scientific) which had been equilibrated into PBS (3×300 μL). Organic solvent was reduced to <0.01% by PBS diafiltration using an Amicon-Ultra centrifugal filter (MW cut-off 10,000 Da, Merck Millipore).

### General Method for Click Chemistry

To a solution of trastuzumab-linker conjugate in PBS ([Tras]=1.0 mgml⁻¹, 60 μL, 0.4 nmol) was added a solution of DBCO click reagent in DMSO (10 mM stock concentration, 0.4 μL, 4 nmol, 10 equiv.) and the mixture was incubated at 37° C. for 4-24 hours on a thermal shaker at 400 rpm. The conjugate was purified by Zeba™ Spin Desalting Column (MW cut-off 40,000 Da, Thermo Fisher Scientific) which had been equilibrated into PBS (3×300 μL). Organic solvent was reduced to <0.01% by PBS diafiltration using an Amicon-Ultra centrifugal filter (MW cut-off 10,000 Da, Merck Millipore).

### Example 34—Synthesis of ALC 1

Bioconjugation was carried out using scaffold A (206). SDS-PAGE (12% polyacrylamide gel, run at 200V over 50 minutes, visualised with Coomassie Brilliant Blue stain) indicated that the fully re-bridged antibody was the major product. HRMS (ESI) [M+H]⁺ 146,687 Da (calc. 146,687 Da).

### Example 35—Synthesis of ALC 2

Bioconjugation was carried out using scaffold B (208). SDS-PAGE (12% polyacrylamide gel, run at 200V over 50 minutes, visualised with Coomassie Brilliant Blue stain) indicated that the fully re-bridged antibody was the major product. HRMS (ESI) [M+H]⁺ 147,269 Da (calc. 147,267 Da).

### Example 36—Synthesis of ALC 3

Bioconjugation was carried out using scaffold C (211). SDS-PAGE (12% polyacrylamide gel, run at 200V over 50 minutes, visualised with Coomassie Brilliant Blue stain) indicated that the fully re-bridged antibody was the major product. HRMS (ESI) [M+H]⁺ 146,979 Da (calc. 146,978 Da).

### Example 37—Synthesis of ALC 4

Bioconjugation was carried out using linker scaffold D (213). SDS-PAGE (12% polyacrylamide gel, run at 200V over 50 minutes, visualised with Coomassie Brilliant Blue stain) indicated that the fully re-bridged antibody was the major product. HRMS (ESI) [M+H]⁺ 147,559 Da (calc. 147,557 Da).

### Examples 38-41—Synthesis of ADC 1-4

Click reaction was carried out using ALC 1, ALC 2, ALC 3 and ALC 4 respectively. DBCO-PEG₅-Val-Cit-PAB-MMAE (248) as the click partner in each reaction, and the mixture was incubated for 24 hours to produce the desired ADC.
ADC 1: HRMS (ESI) [M+H]+ 148,390 Da (calc. 148,388 Da).
ADC 2: HRMS (ESI) [M+H]+ 148,971 Da (calc. 148,968 Da).
ADC 3: HRMS (ESI) [M+H]+ 148,681 Da (calc. 148,679 Da).
ADC 4: HRMS (ESI) [M+H]+ 149,262 Da (calc. 149,258 Da).

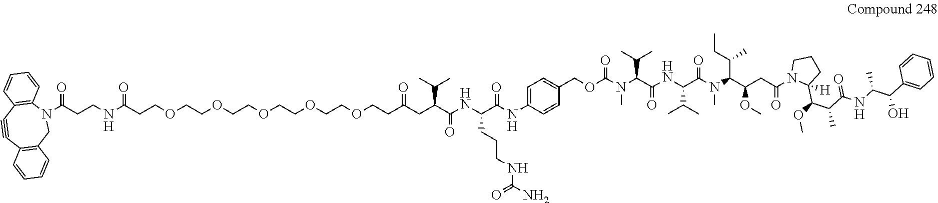 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](CC(=O)CCOCCOCCOCCOCCOCCC(=O)NCCC(=O)N2Cc3ccccc3C#Cc3ccccc32)C(C)C)cc1)C(C)C]

### Examples 42-45—Synthesis of ALC 5-8

A solution of brentuximab (5.7 mg/mL) in PBS was pH adjusted with a 5% v/v addition of 500 mM Tris and 25 mM EDTA (pH8.5). The mixture was reduced by the addition of TCEP (10 eq) for 90 minutes at room temperature. The solution was then further diluted to 2.5 mg/mL with 50 mM Tris (pH 8.0). 5 eq of a 10 mM solution of conjugating reagent (Scaffolds E, F, G and H) in DMSO was added as well as additional solvent to a final concentration of 5% v/v. The reaction was allowed to proceed at room temperature for 24 hours. The mixture was then desalted into PBS (pH 7.4) using NAP 25 desalting columns to remove excess reagents, and re-bridging confirmed by RP-HPLC (PLRP) and HRMS to yield ALC 5, ALC 6, ALC 7 and ALC 8 respectively.
ALC 5: HPLC retention time 13.59 min; HRMS observed Mw 149,727 Da (calc. 149,728 Da).
ALC 6: HPLC retention time 13.60 min; HRMS observed Mw 150,308 Da (calc. 150,308 Da).
ALC 7: HPLC retention time 13.59 min; HRMS observed Mw 150,017 Da (calc. 150,018 Da).
ALC 8: HPLC retention time 13.58 min; HRMS observed Mw 150,599 Da (calc. 150,599 Da).

### Examples 46-49—Synthesis of ADC 5-8

Each re-bridged mAb sample (ALC 5, ALC 6, ALC 7 and ALC 8) was conjugated with commercially available DBCO-VC-MMAE using 10 eq of the DBCO-linker-warhead and incubated at room temperature for 24 hours. Conjugation was confirmed by RP-HPLC (PLRP) and HRMS to yield ADC 5-8 respectively.
ADC 5: HPLC retention time 13.96 min; HRMS observed Mw 151,170 Da (calc. 151,167 Da).
ADC 6: HPLC retention time 13.96 min; HRMS observed Mw 151,751 Da (calc. 151,747 Da).
ADC 7: HPLC retention time 13.97 min; HRMS observed Mw 151,459 Da (calc. 151,650 Da).
ADC 8: HPLC retention time 13.98 min; HRMS observed Mw 152,041 Da (calc. 152,038 Da).

### Examples 50-53—Synthesis of ADC 9-12

Each re-bridged mAb sample (ALC 5, ALC 6, ALC 7 and ALC 8) was conjugated with commercially available DBCO-PBD using 10 eq of the DBCO-linker-warhead and incubated at room temperature for 24 hours. Conjugation was confirmed RP-HPLC (PLRP) and HRMS to yield ADC 9-12 respectively.
ADC 9: HPLC retention time 13.92 min; HRMS observed Mw 151,368 Da (calc. 151,360 Da).
ADC 10: HPLC retention time 13.92 min; HRMS observed Mw 151,948 Da (calc. 151,942 Da).
ADC 11: HPLC retention time 13.94 min; HRMS observed Mw 151,658 Da (calc. 151,650 Da).
ADC 12: HPLC retention time 13.93 min; HRMS observed Mw 152,239 Da (calc. 152,232 Da).

### Examples 54-57—Synthesis of ALC 9-12

A solution of trastuzumab (25.6 mg/mL) in PBS was pH adjusted with a 5% v/v addition of 500 mM Tris and 25 mM EDTA (pH8.5). The mixture was reduced by the addition of TCEP (6 eq) for 90 minutes at room temperature. The solution was then further diluted to 2.5 mg/mL with 50 mM Tris (pH8.0). 5 eq of a 10 mM solution of conjugating reagent (Scaffolds E, F, G and H) in DMSO was added as well as additional solvent to a final concentration of 5% v/v. The reaction was allowed to proceed at room temperature for 24 hours. The mixture was then desalted into PBS (pH7.4) using NAP 25 desalting columns to remove excess reagents, and re-bridging confirmed by RP-HPLC (PLRP) and HRMS to yield ALC 9, ALC 10, ALC 11 and ALC 12 respectively.
ALC 9: HPLC retention time 12.64 min; HRMS observed Mw 149,866 Da (calc. 149,867 Da).
ALC 10: HPLC retention time 12.63 min; HRMS observed Mw 150,447 Da (calc. 150,447 Da).
ALC 11: HPLC retention time 12.60 min; HRMS observed Mw 150,157 Da (calc. 150,157 Da).
ALC 12: HPLC retention time 12.62 min; HRMS observed Mw 150,738 Da (calc. 150,738 Da).

### Examples 58-61—Synthesis of ADC 13-16

Each re-bridged mAb sample (ALC 9, ALC 10, ALC 11 and ALC 12) was conjugated with commercially available DBCO-VC-MMAE using 10 eq of the DBCO-linker-warhead and incubated at room temperature for 24 hours. Conjugation was confirmed by SEC, HIC, PLRP and MS to yield ADC 13-16 respectively.
ADC 13: HPLC retention time 13.15 min; HRMS observed Mw 151,308 Da (calc. 151,306 Da).
ADC 14: HPLC retention time 13.20 min; HRMS observed Mw 151,888 Da (calc. 151,886 Da).
ADC 15: HPLC retention time 13.20 min; HRMS observed Mw 151,598 Da (calc. 151,596 Da).
ADC 16: HPLC retention time 13.19 min; HRMS observed Mw 152,179 Da (calc. 152,177 Da).

### Examples 50-53—Synthesis of ADC 17-20

Each re-bridged mAb sample (ALC 9, ALC 10, ALC 11 and ALC 2) was conjugated with commercially available DBCO-PBD using 10 eq of the DBCO-linker-warhead and incubated at room temperature for 24 hours. Conjugation was confirmed by SEC, HIC, PLRP and MS to yield ADC 17-20 respectively.
ADC 17: HPLC retention time 13.07 min; HRMS observed Mw 151,506 Da (calc. 151,500 Da).
ADC 18: HPLC retention time 13.08 min; HRMS observed Mw 152,086 Da (calc. 152,080 Da).
ADC 19: HPLC retention time 13.10 min; HRMS observed Mw 151,796 Da (calc. 151,790 Da).
ADC 20: HPLC retention time 13.10 min; HRMS observed Mw 152,377 Da (calc. 152,371 Da).

### DBCO-VC-MMAE:

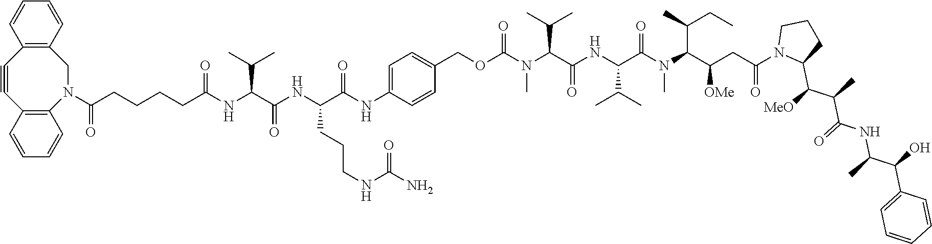 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCC(=O)N2Cc3ccccc3C#Cc3ccccc32)C(C)C)cc1)C(C)C]

DBCO-VC-MMAE was purchased from Syntabio LLC.

### DBCO-PBD:

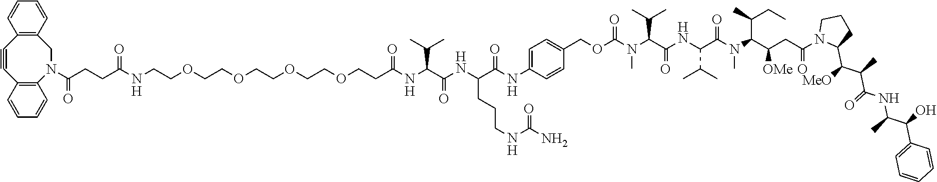 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)[C@@H](NC(=O)CCOCCOCCOCCOCCNC(=O)CCC(=O)N2Cc3ccccc3C#Cc3ccccc32)C(C)C)cc1)C(C)C]

DBCO-PBD purchased from Levena biopharma, catalogue code SET0317.

For each of Examples 42 to 61 the following reverse phase-HPLC (PLRP) conditions were used:
Column: Polymer Labs PLRP-S 2.1 mm×50 mm, 5 μm, 1000 Å
Mobile phase A: 0.1% v/v TFA/water
Mobile phase B: 0.1% v/v TFA/Acetonitrile
Column Temperature: 80° C.
Detection Wavelength: 214 nm
Reference: 440 nm, 80 nm
Peak width: >0.4 min, slit 8 nm
Report: 214 nm
Collect spectrum between 200 and 600 nm. Step 1.2 nm

For each of Examples 42 to 61 the following HRMS conditions were used:
Mass Spectrometer: SCIEX X500B Q-ToF
Software: SCIEX OS
UHPLC: ExionLC AD
LC Column: bioZen™ 3.6 μm Intact XB-C8, 50×2.1 mm

The measured mass of the major glycoform for each antibody was also used to calculate the theoretical (calculated) molecular weights.

### Examples 54—Re-Bridging and Conjugation Trials with Brentuximab

Brentuximab at 5.7 mg/ml was pH adjusted with a 5% v/v addition of 500 mM Tris, 25 mM EDTA pH 8.5 then reduced with 10 equivalents of tris(2-carboxyethyl)phosphine hydrochloride (TCEP) for 90 minutes at room temperature. RP-HPLC analysis showed complete reduction achieved. Reduced Brentuximab diluted to 2.5 mg/ml with 50 mM Tris pH 8.0 was then re-bridged by the addition of 5 equivs of 10 mM of scaffold (Scaffold E, F, G and H respectively) in DMSO plus additional solvent to 5% v/v total. The re-bridging reaction was allowed to proceed at room temperature for 24 hours. Th re-bridged antibody samples were then desalted into PBS pH 7.4 using NAP 25 desalting columns to remove solvent and residual scaffold. UV analysis showed all samples to have an approx. [P]=2 mg/ml post desalting. Analysis was performed using SEC, HIC, PLRP (intact) and MS (intact). Each re-bridged antibody sample was split and conjugated with the following click compounds: DBCO-Sulfo-Gly3, DBCO-VC-MMAE and DBCO-PBD, using 10 equivalents of the DBCO-compounds in 10% v/v DMA. Conjugation reactions were incubated at room temperature overnight and SEC, HIC, PLRP (intact) and MS (intact) analysis was repeated.

The results are summarised in Table 1 below.

In the tables below:
“L” refers to the relative amount of antibody light chain;
“HHL” refers to the relative amount of antibody heavy-heavy-light chain;
“intact” refers to the relative amount of fully re-bridged (intact) antibody; and
“[P]” refers to the protein concentration as measured post-desalting by UV analysis.

| | Scaffold | Scaffold | Scaffold | Scaffold | | | | | | | | |
|---|---|---|---|---|---|---|---|---|---|---|---|---|
| Re-Bridging | E | F | G | H | | | | | | | | |
| L | 11.7 | 7.1 | 10.9 | 4.3 | | | | | | | | |
| HHL | 15.8 | 6.2 | 13.9 | 6.2 | | | | | | | | |
| Intact | 72.6 | 86.7 | 75.2 | 89.5 | | | | | | | | |
| % monomer | 97.3 | 98.1 | 98.2 | 97.9 | | | | | | | | |
| [P] mg/ml | 1.6 | 2.0 | 2.0 | 1.9 | | | | | | | | |
| | DBCO- | DBCO- | DBCO- | DBCO | DBCO- | DBCO- | DBCO- | DBCO- | DBCO- | DBCO- | DBCO- | DBCO- |
| Conjugation | Gly3 | vcE | PBD | Gly3 | vcE | PBD | Gly3 | vcE | PBD | Gly3 | vcE | PBD |
| DAR | N/A | 0.60 | 0.78 | N/A | 0.77 | 0.88 | N/A | 0.65 | 0.83 | N/A | 0.76 | 0.88 |
| by HIC | | | | | | | | | | | | |
| DAR | N/A | 0.71 | 0.76 | N/A | 0.87 | 0.89 | N/A | 0.77 | 0.82 | N/A | 0.85 | 0.87 |
| by PLRP | | | | | | | | | | | | |
| DAR | 1.0 | >0.89 | >0.9 | 1.0 | >0.9 | >0.9 | 1.0 | >0.9 | >0.9 | 1.0 | >0.9 | >0.9 |
| by MS | | | | | | | | | | | | |
| % monomer | N/A | 98.1 | 97.5 | N/A | 98.1 | 98.1 | N/A | 98.0 | 97.6 | N/A | 98.1 | 98.0 |

### Examples 55—Re-Bridging and Conjugation Trials with Trastuzumab

Trastuzumab at 25.6 mg/ml was pH adjusted with a 5% v/v addition of 500 mM Tris, 25 mM EDTA pH 8.5 then reduced with 6 equivalents of TCEP for 90 minutes at room temperature. RP-HPLC analysis showed complete reduction achieved. The reduced trastuzumab was then diluted to 2.5 mg/ml with 50 mM Tris pH 8.0 and then re-bridged by the addition of 5 equivs of 10 mM scaffold (Scaffold E, F, G and H respectively) in DMSO plus additional solvent to 5% v/v total. The re-bridging reaction was allowed to proceed at RT for 23 hours. Re-bridged antibody samples were then desalted into PBS pH 7.4 using NAP 25 desalting columns to remove solvent and residual scaffold. UV analysis showed all samples to have a [P]=1.8 mg/mi post desalting. Analysis was performed using SEC, HIC, PLRP (intact) and MS (intact). Each re-bridged antibody sample was split and conjugated with the following click compounds: DBCO-Sulfo-Gly3, DBCO-vcE and DBCO-PBD, using 10 equivalents of DECO-compounds in 10% v/v DMA. Conjugation reactions were incubated at room temperature overnight and SEC, HIC, PLRP (intact) and MS (intact) analysis was repeated.

The results are summarised in Table 2 below:

| | Scaffold | Scaffold | Scaffold | Scaffold | | | | | | | | |
|---|---|---|---|---|---|---|---|---|---|---|---|---|
| Re-Bridging | E | F | G | H | | | | | | | | |
| L | 6.7 | 2.9 | 5.2 | 4.1 | | | | | | | | |
| HHL | 8.2 | 2.2 | 5.7 | 2.5 | | | | | | | | |
| Intact | 85.1 | 94.9 | 89.2 | 93.4 | | | | | | | | |
| % monomer | 98.7 | 98.7 | 98.7 | 98.6 | | | | | | | | |
| [P] mg/ml | 1.8 | 1.9 | 1.8 | 1.8 | | | | | | | | |
| | DBCO- | DBCO- | DBCO- | DBCO- | DBCO- | DBCO- | DBCO- | DBCO- | DBCO- | DBCO- | DBCO- | DBCO- |
| Conjugation | Gly3 | vcE | PBD | Gly3 | vcE | PBD | Gly3 | vcE | PBD | Gly3 | vcE | PBD |
| DAR | N/A | 0.72 | 0.84 | N/A | 0.84 | 0.94 | N/A | 0.79 | 0.90 | N/A | 0.82 | |
| by HIC | | | | | | | | | | | | |
| DAR | N/A | 0.75 | 0.85 | N/A | 0.87 | 0.94 | N/A | 0.80 | 0.90 | N/A | 0.87 | 0.93 |
| by PLRP | | | | | | | | | | | | |
| DAR | 1.0 | >0.9 | >0.9 | 1.0 | >0.9 | 1.0 | 1.0 | >0.9 | 1.0 | 1.0 | >0.9 | 1.0 |
| by MS | | | | | | | | | | | | |
| % monomer | N/A | 98.3 | 98.4 | N/A | 98.6 | 98.7 | N/A | 98.70 | 98.60 | N/A | 98.4 | 98.5 |

While specific embodiments of the invention have been described for the purpose of reference and illustration, various modifications will be apparent to a person skilled in the art without departing from the scope of the invention as defined by the appended claims

### Numbered Paragraphs

The following numbered paragraphs describe certain aspects and embodiments of the invention:
(1) A conjugate comprising an antibody, a linker and at least one active agent, wherein:
   i) the linker connects the at least one active agent(s) to the antibody;
   ii) the linker is attached to the antibody through 5 to 8 independent covalent bonds; and
   iii) each covalent bond between the linker and the antibody is formed from the reaction between a sulfur atom on the antibody and a functional group on the linker.
(2) The conjugate according to numbered paragraph (1), wherein the sulfur atoms on the antibody are sulfur atoms present on cysteine residues of the antibody.
(3) The conjugate according to numbered paragraph (1) or (2), wherein the functional groups that reacts with the sulfur atoms on the antibody are Michael Acceptors.
(4) The conjugate according to any one of numbered paragraphs (1) to (3), wherein the linker is attached to the antibody through 6 to 8 (preferably 7 to 8) independent covalent bonds such that the linker re-bridges between two and four (preferably three and four) reduced interchain disulfide bonds in the antibody.
(5) The conjugate according to any one of numbered paragraphs (1) to (4), wherein the functional groups that reacts with the sulfur atoms on the antibody are selected from an alkene, an alkyne, a maleimide, a halo-maleimide, a sulfone, an arylene-propiolonitrile, a pyridazinedione or a group comprising a β-unsaturated ketone.
(6) The conjugate according to any one of numbered paragraphs (1) to (5), wherein the conjugate comprises between one and four (preferably between two and four or three and four) re-bridging moieties of Formula (III), shown below:

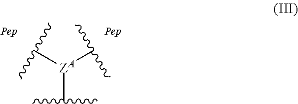

wherein:
   Z^{A} is a functional linking group;
   Pep indicates the position where the moiety is attached to the antibody, either directly or indirectly; and

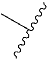

indicates the position where the moiety is attached to linker.
(7) The conjugate according to numbered paragraph (6), wherein the re-bridging moieties of Formula (III) are independently selected from the one of the following groups:

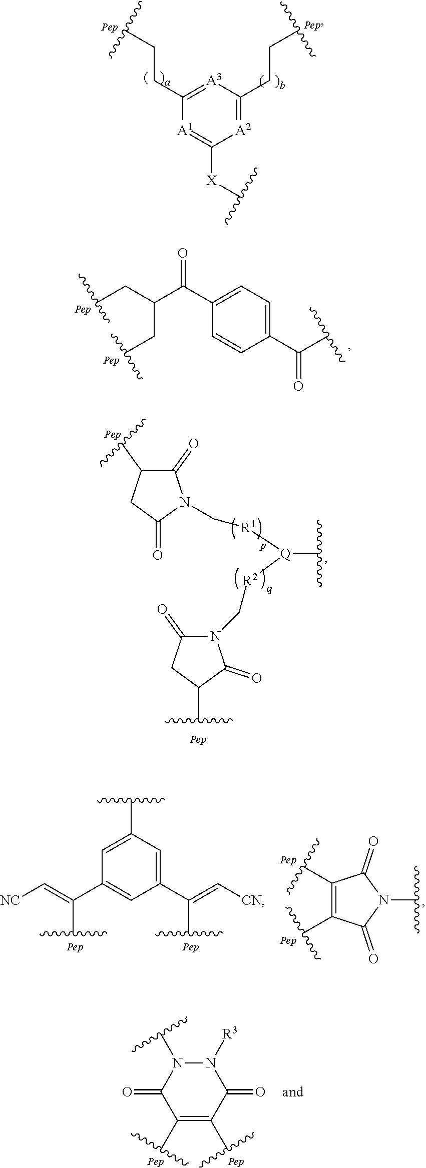

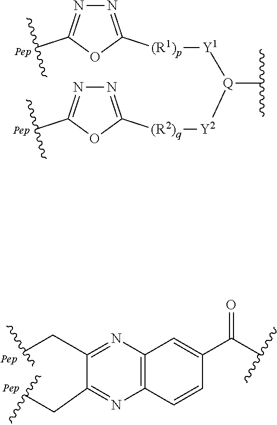

wherein:
   one or two of A¹, A² and A³ are N and the other one or two of A¹, A² and A³ are CH, or all three of A¹, A² and A³ are N or OH;
   a and bare integers selected from 0 or 1
   X is selected from N, NR^{N}O and S, wherein R^{N} is H or C₁₋₂alkyl;
   R¹ and R² are independently selected from C₁-C₆ alkylene and C₁-C₆ alkylene containing O in the backbone;
   R³ is selected from hydrogen or a C₁-C₄ alkyl:
   Y¹ and Y² are independently absent or selected from O, NR⁴, C(═O), C(═O)NR⁴ or NR⁵C(═O);
   p and q are independently integers selected from 0 or 1;
   Q is CR⁶, N or aryl;
   R⁴, R⁵ and R⁶ are independently selected from hydrogen and C₁-C₄alkyl;
   Pep indicates the position where the moiety is linked to the antibody, either directly or indirectly; and

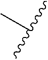

indicates the position where the moiety is attached to linker.
(8) The conjugate according to any one of numbered paragraph (1) to (7), wherein the re-bridging moieties each have the general formula (IIIa), shown below:

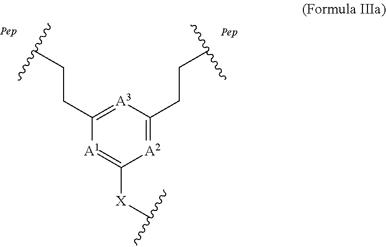

wherein each of A¹, A², A³, X, Pep and

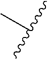

are as defined in numbered paragraph (7).
(9) The conjugate according to any one of numbered paragraphs (1) to (8), wherein the antibody is a monoclonal antibody.
(10) The conjugate according to any one of numbered paragraphs (1) to (9), wherein the antibody is directed to a tumour associated antigen.
(11) The conjugate according to any one of numbered paragraphs (1) to (10), wherein the active agent is a labelling moiety.
(12) The conjugate according to numbered paragraph (11), wherein the labelling moiety is a fluorophore, a biotin tag or a PET tracer.
(13) The conjugate according to any one of numbered paragraphs (1) to (10), wherein the active agent is a drug.
(14) The conjugate according to numbered paragraph (13), wherein the drug is a cytotoxin.
(15) The conjugate according to numbered paragraph (14), wherein the cytotoxin is selected from the group comprising auristatins, maytansinoids, tubulysins, calicheamicins, duocarmycins, pyrrolobenzodiazepines, camptothecin analogues and doxorubicin.
(16) The conjugate according to any one of numbered paragraphs (1) to (15), wherein the conjugate comprises between 1 and 8 active agents.
(17) The conjugate according to any one of numbered paragraph (6) to (16), wherein at least two of the re-bridging moieties are separated by between 10 and 60 atoms.
(18) The conjugate according to any one of numbered paragraphs (6) to (17), wherein at least two of the re-bridging moieties are separated by between 20 and 40 atoms.
(19) The conjugate according to any one of numbered paragraphs (1) to (18), wherein the linker comprises one or more groups selected from one or more groups selected from an alkylenediamine moiety, a polyethylene glycol moiety, an amino acid residue, an arylene-containing moiety, a heteroarylene-containing moiety, a heterocyclyl-containing moiety, a cycloalkyl-containing moiety and combinations thereof.
(20) The conjugate according to any one of numbered paragraphs (1) to (19), wherein the linker comprises one or more groups selected from an alkylenediamine moiety, a polyethylene glycol moiety and an amino acid residue.
(21) The conjugate according to any one of numbered paragraphs (1) to (20), wherein the conjugate is of Formula (IIIA), shown below:

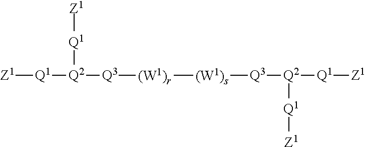

wherein:
   Z¹ is a re-bridging moiety of Formula (III) as defined in numbered paragraph (7) or (8) above that is attached to an antibody at the positions shown in Formula (III);
   each Q¹ is independently a bond or a group of Formula IVa:

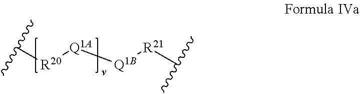

wherein:
   Q^{1A} is absent or selected from a C(═O), OC(═O), NR⁷C(═O), C(═O)NR⁷, C(═NR³) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, a C₁-C₁₀ alkylene and a C₁-C₁₀ alkylene containing O in the backbone; and
   v is an integer selected from 0, 1 or 2;
each Q² is independently selected from N and CR¹¹, wherein R₁₁ is selected from hydrogen and C₁-C₄alkyl;
each Q³ is independently a group of Formula IVb:

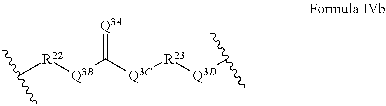

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, a C₁-C₁₀alkylene and a C₁-C₁₀alkylene containing O in the backbone;
   Q^{3D} is absent or selected from C(═O), OC(═O), NR¹²C(═O) and C(═O)NR¹²
W¹ is a group of Formula IVc or Formula IVe:

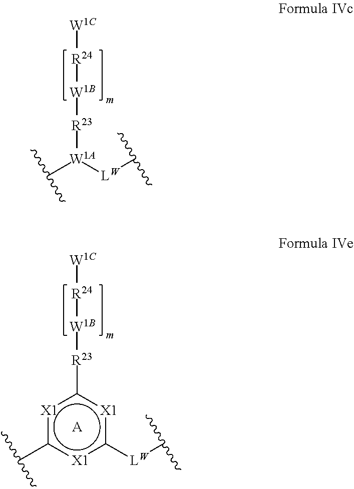

wherein:
   W^{1A} is selected from N and CR¹⁴, wherein R¹⁴ is a selected from hydrogen and C₁-C₄alkyl;
   L^{W} is absent or selected from C₁-C₆alkylene and C₁-C₆alkylene containing O or N in the backbone;
   Ring A is a 6-mebered aryl, a 6-membered heteroaryl, a 6-membered heterocyclyl or a 6-membered cycloalkyl, wherein each X₁ is independently selected from CH or N;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₁₂alkylene and C₁-C₁₂alkylene containing O in the backbone;
   m is an integer selected from 1 or 2;
   W^{1C} is selected from Formula W^{C1} or Formula W^{C2}:

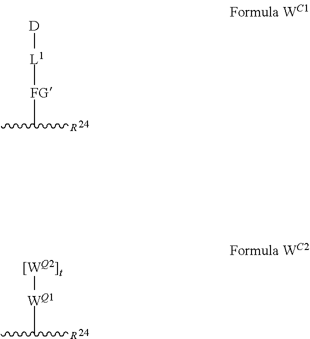

wherein:
   W^{Q1} is a group of Formula W^{C3}:

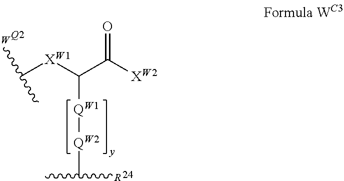

wherein:
Q^{W1} is absent or selected from C₁-C₁₂alkylene and C₁-C₁₂alkylene containing an O or an N in the backbone; and
Q^{W2} is selected from —C(═O)—, —OC(═O)—, —C(═O)O—, —NHC(═O)—, and —C(═O)NH—;
y is an integer selected from 0 or 1;
X^{W1} is selected from O or NH;
X^{W2} is selected from hydrogen, C₁-C₄alkyl, OR^{x1} and NR^{x1}R^{x2}, wherein R^{x1} and R^{x2} are independently selected from hydrogen and C₁-C₄alkyl;

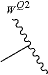

indicates the position where X^{W1} is attached to a group of formula W^{Q2};

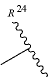

indicates the position where W^{Q1} is attached to a group of formula R²⁴
W^{Q2} is a group of Formula W^{C4}:

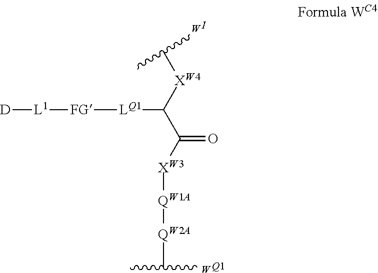

wherein:
Q^{W1A} is absent or selected from a C₁-C₁₂alkylene and a C₁-C₁₂alkylene containing O or N in the backbone; and
Q^{W2A} is selected from —C(═O)—, —OC(═O)—, —C(═O)O—, —NHC(═O)—, and —C(═O)NH—;
X^{W3} is selected from O or NH;

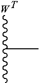

indicates the position where X^{W4} is attached to another group of formula W^{Q2} via the Q^{W2A} substituent group, or

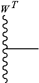

indicates the position where X^{W4} is attached to one of the following groups hydrogen or —C(═O)R^{X3}, wherein R^{x3} is selected from hydrogen and C₁-C₄alkyl;

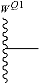

indicates the position where Q^{W2A} is attached to the group of formula W^{Q1} via the X^{W1} group; X^{W4} is selected from O or NH;
L^{Q1} is a linker comprising one or more groups selected from a C₁-C₂₀alkylene, an alkylenediamine moiety, a (poly)ethylene glycol moiety, an amino acid residue and combinations thereof;
t is an integer selected from 1 to 8;
FG′ is a functional linking moiety;
L¹ is a linker; and
D is an active agent as defined in any one of numbered paragraphs (13) to (15);
W² is a group of Formula IVd:

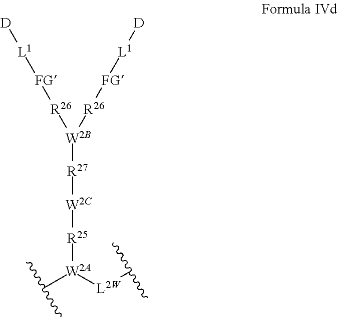

wherein:
   W^{2A} is selected from N and CR¹⁷;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from C₁-C₆alkylene and C₁-C₆alkylene containing O or N in the backbone;
   R¹⁷ and R¹⁸ are independently selected from hydrogen and C₁-C₄alkyl;
   R²⁵, R²⁶ and R²⁷ are independently selected from a bond, C₁-C₁₀alkylene and C₁-C₁₀alkylene containing O in the backbone; and
   FG′, L¹ and D are as defined above;
r is an integer selected from 0 to 12;
s is an integer selected from 0 to 6; and
wherein r and s are selected such that the total number of active agents per conjugate is between 1 and 12.
(22) The conjugate according to any one of numbered paragraphs (1) to (21), wherein the conjugate is of Formula (IIIA), shown below:

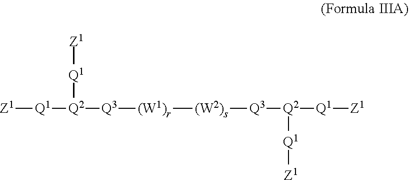

wherein:
   Z¹ is a re-bridging moiety of Formula (III) as defined in numbered paragraph (7) or (8) above that is attached to an antibody at the positions shown in Formula (III);
   each Q¹ is independently a bond or a group of Formula IVa:

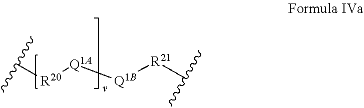

wherein:
   Q^{1A} is absent or selected from a C(═O), OC(═O), NR⁷C(═O), C(═O)NR⁷, C(═NR³) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈ alkylene and C₁-C₈ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently a group of Formula IVb:

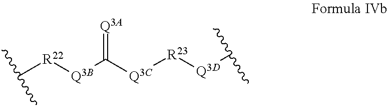

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₆alkylene and C₁-C₃ alkylene containing O in the backbone;
   Q^{3D} is absent or selected from C(═O), OC(═O), NR¹²C(═O) and C(═O)NR¹²
W¹ is a group of Formula IVc₁ or Formula IVe₁:

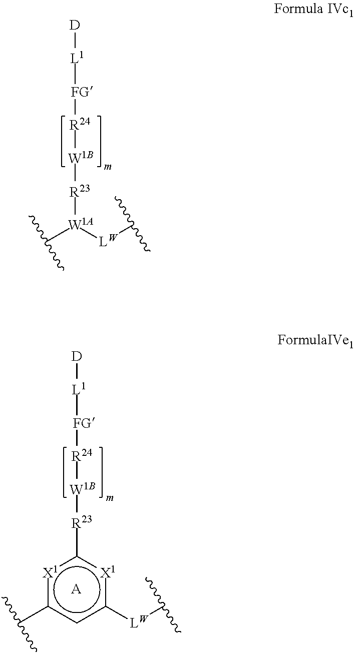

wherein:
   W^{1A} is selected from N and CR¹⁴, wherein R¹⁴ is a selected from hydrogen and C₁-C₄alkyl;
   L^{W} is absent or selected from a C₁-C₆alkylene and a C₁-C₆alkylene containing amido, O or N in the backbone;
   Ring A is a 6-membered aryl, a 6-membered heteroaryl, a 6-membered heterocyclyl or a 6-membered cycloalkyl, wherein X, is selected from CH or N;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₆alkylene containing O in the backbone;
   m is an integer selected from 1 or 2;
   FG′ is a functional linking moiety;
   L¹ is a linker; and
   D is an active agent as defined in any one of numbered paragraphs (13) to (15);
W² is a group of Formula IVd:

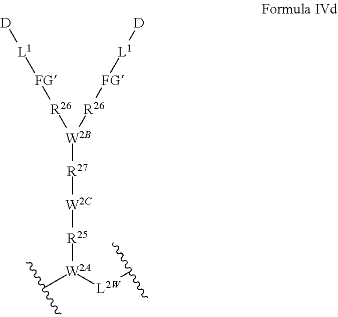

wherein:
   W^{2A} is selected from N and CR¹⁷;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from a C₁-C₆alkylene and a C₁-C₆alkylene containing O or N in the backbone;
   R¹⁷ and R¹⁸ are independently selected from hydrogen and C₁-C₄alkyl;
   R²⁵, R²⁶ and R²⁷ are independently selected from a bond, C₁-C₈alkylene and C₁-C₆alkylene containing O in the backbone; and
   FG′, L¹ and D are as defined above;
r is an integer selected from 0 to 12;
s is an integer selected from 0 to 6; and
wherein r and s are selected such that the total number of active agents per conjugate is between 1 and 12.
(23) The conjugate according to any one of numbered paragraphs (1) to (22), wherein the conjugate is of Formula (IIIA), shown below:

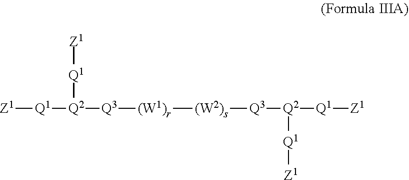

wherein:
   Z¹ is a re-bridging moiety of Formula (III) as defined in numbered paragraph (7) or (8) above that is attached to an antibody at the positions shown in Formula (III);
   each Q¹ is independently a bond or a group of Formula IVa:

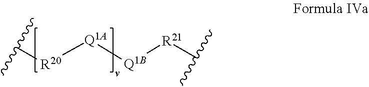

wherein:
   Q^{1A} is absent or selected from a C(═O), OC(═O), NR⁷C(═O), C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈ alkylene and C₁-C₈ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently a group of Formula IVb:

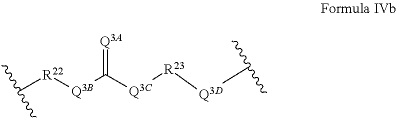

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₈alkylene and C₁-C₈alkylene containing O in the backbone;
   Q^{3D} is absent or selected from C(═O), OC(═O), NR¹²C(═O) and C(═O)NR¹²;
W¹ is a group of Formula IVc₁:

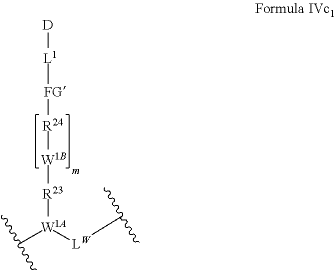

wherein:
   W^{1A} is selected from N and CR¹⁴, wherein R¹⁴ is a selected from hydrogen and C₁-C₄alkyl;
   L^{W} is absent or selected from C₁-C₆alkylene and C₁-C₆alkylene containing O or N in the backbone;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₈alkylene containing O in the backbone;
   m is an integer selected from 1 or 2 (suitably m is 1); and
   FG′ is a functional linking moiety;
   L¹ is a linker; and
   D is an active agent as defined in any one of numbered paragraphs (13) to (15);
W² is a group of Formula IVd:

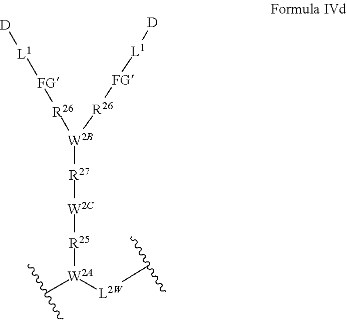

wherein:
   W^{2A} is selected from N and CR¹⁷;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from C₁-C₆alkylene and C₁-C₆alkylene containing O or N in the backbone;
   R¹⁷ and R¹⁸ are independently selected from hydrogen and C₁-C₄alkyl;
   R²⁵, R²⁶ and R²⁷ are independently selected from a bond, C₁-C₈alkylene and C₁-C₆alkylene containing O in the backbone;
   m is an integer selected from 1 or 2 (suitably m is 1); and
   FG′, L¹ and D are as defined above;
r is an integer selected from 0 to 12;
s is an integer selected from 0 to 6; and
wherein r and s are selected such that the total number of active agents per conjugate is between 1 and 12.
(24) The conjugate according to numbered paragraphs (21) or (23), wherein FG′ is selected from a bond,

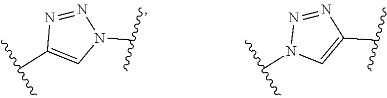 [SMILES: *c1cn(*)nn1 ; *c1cn(*)nn1]

and —C(═O)NH—.
(25) The conjugate according to any one of numbered paragraphs (21) to (24), wherein L¹ is selected from a bond, C₁-C₁₀alkylene and C₁-C₁₀alkylene containing O in the backbone and a group of Formula Va¹:

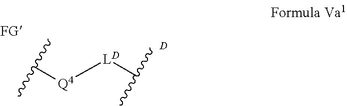

wherein:
   Q⁴ is a single bond, or

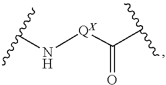

Where Q^{X} is such that Q⁴ is an amino-acid residue, a dipeptide residue or a tripeptide residue; and
L^{D} is a group for attachment to the active agent.
(26) The conjugate according to any one of numbered paragraphs (21) to (25), wherein:
   Q¹ is a group of Formula IVa:
   Q^{1A} is selected from C(═O) and NR⁷C(═O);
   Q^{1B} is selected from N and CR⁹CR¹⁰;
   R⁷, R⁹ and R¹⁰ are independently selected from hydrogen or C₁-C₄alkyl;
   R²⁰ and R²¹ are independently selected from C₁-C₆ alkylene and C₁-C₈ alkylene containing O in the backbone; and
   v is 1;
   Q² is selected from N or CR¹¹, wherein R¹¹ is selected from hydrogen and C₁-C₄alkyl;
   Q³ is a group of Formula IVb:
   Q^{3A} is O;
   Q^{3B} is a bond;
   Q^{3C} is NR¹³;
   R¹³ is selected from hydrogen and C₁-C₄alkyl;
   R²² and R²³ are independently selected from C₁-C₈alkylene and C₁-C₈alkylene containing O in the backbone;
   m is an integer selected from 1 or 2 (suitable m is 1);
   W^{1A} is N:
   L^{W} is a C₁-C₄alkylene;
   W^{1B} is a group selected from —NR¹⁵C(═O)— and —C(═O)NR¹⁵;
   R¹⁵ is selected from hydrogen and C₁-C₄alkyl;
   R²³ and R²⁴ are independently selected from a bond, C₁-C₈alkylene and C₁-C₈alkylene containing O in the backbone; and
   W^{2A} is N;
   W^{2B} is N;
   W^{2C} is —C(═O)NH—
   L^{2W} is a C₁-C₄alkylene;
   R²⁵ and R²⁶ are independently selected from a bond, C₁-C₈alkylene and C₁-C₈alkylene containing O in the backbone;
   r is an integer selected from 0 to 10;
   s is an integer selected from 0 to 5;
   wherein r and s are selected such that the total number of active agents per conjugate is between 1 and 10.
(27) The conjugate according to any one of numbered paragraph (21) to (26), wherein Z¹ is a re-bridging moiety of Formula (IIIa), shown below:

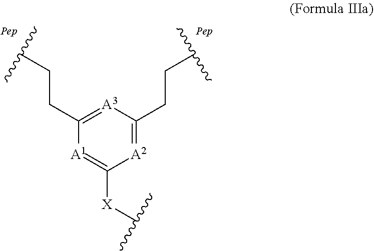

wherein each of A¹, A², A³, X, Pep and

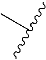

are as defined in numbered paragraph (7).
(28) The conjugate according to any one of numbered paragraphs (1) to (27), wherein the conjugate is selected from any one of the conjugate compounds the listed on pages 49 to 60 (preferably pages 49 to 54) above.
(29) A compound of Formula (I), shown below:

(FG)ₙ-L-(Z)₈ (Formula I)
wherein:
   FG is a functional group capable reacting with another moiety to form a functional linking moiety;
   n is an integer selected from 0 to 20;
   L is a linker; and
   Z is a functional group capable of reaction with a sulfur atom from a cysteine moiety of an antibody;
or a pharmaceutically acceptable salt, solvate or hydrate thereof.
(30) A compound according to numbered paragraph (29), wherein at least two of the Z groups are separated by between 10 and 60 atoms.
(31) A compound according to numbered paragraph (29) or (30), wherein at least two of the Z groups are separated by between 20 and 40 atoms.
(32) A compound according to any one of numbered paragraphs (29) to (31), wherein the linker comprises one or more groups selected from an alkylenediamine moiety, a polyethylene glycol moiety and an amino acid moiety.
(33) A compound according to any one of numbered paragraphs (29) to (32), wherein FG is a functional group selected from an alkene, an alkyne, an azide, a hydroxyl, an amine, a carboxylic acid, an aldehyde, an acyl halide, a tetrazine, an alkoxyamine (e.g. hydroxylamine), a hydrazine, an electron-rich dienophile (e.g. a 1,3-nitrone alkene) and an electron-poor diene (e.g. tetrazine), nitrone, an isocyanate and an isothiocyanate.
(34) A compound according to any one of numbered paragraphs (29) to (33), wherein FG is a functional group selected from an alkyne, an azide, hydroxyl, amine, carboxylic acid, aldehyde and acyl halide.
(35) A compound according to any one of numbered paragraphs (29) to (34), wherein FG is a functional group selected from an amine, carboxylic acid, azide and an alkyne.
(36) A compound according to any one of numbered paragraphs (29) to (35), Z is a Michael Acceptor.
(37) A compound according to any one of numbered paragraphs (29) to (36), wherein Z is selected from an alkene, an alkyne, a maleimide, a halo-maleimide, a sulfone, an arylene-propiolonitrile, a pyridazinedione, a dibromomethylene-pyridine or a group comprising a β-unsaturated ketone
(38) A compound according to any one of numbered paragraphs (29) to (37), wherein the compound is of Formula (Ia), shown below:

(FG)ₙ-L-(Z²)₄ (Formula Ia)
wherein:
   Z² is a re-bridging linking group comprising two functional groups capable of reaction with a sulfur atom from a cysteine moiety of an antibody; and L, FG and n are as defined in numbered paragraph (29) above.
(39) A compound according to any one of numbered paragraphs (29) to (38), wherein the compound is selected from one of the following groups:

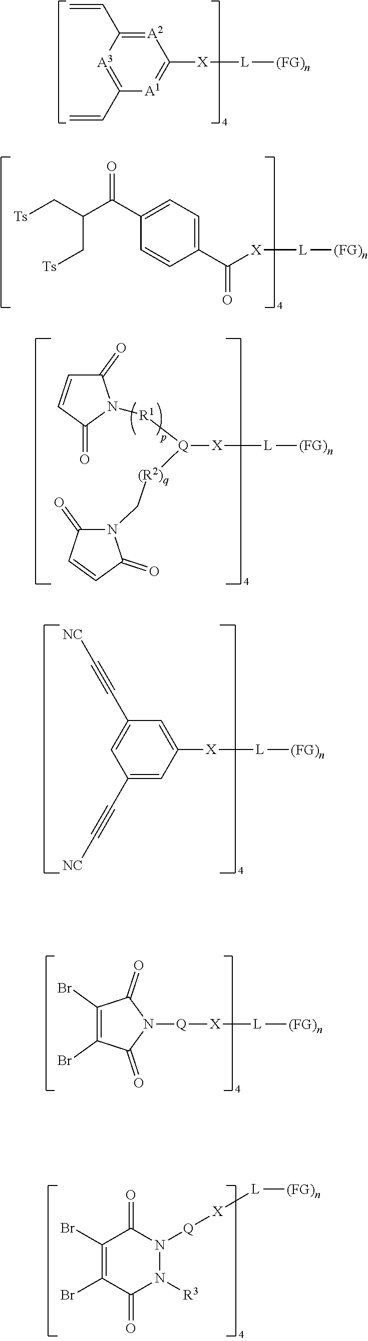

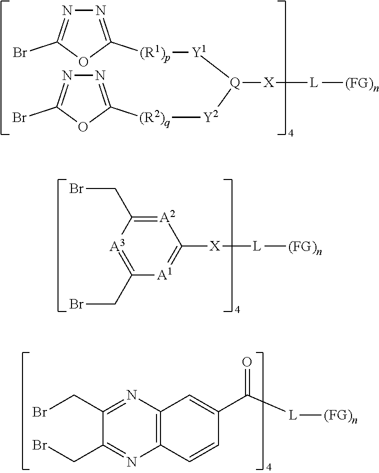

wherein:
   one or two of A¹, A² and A³ are N and the other one or two of A¹, A² and A³ are CH, or all three of A¹, A² and A³ are N;
   X is selected from N, NR^{N}, O and S, wherein R^{N} is H or C₁₋₂alkyl;
   R¹ and R² are independently selected from C₁-C₆ alkylene and C₁-C₆ alkylene containing O in the backbone;
   R³ is selected from hydrogen or a C₁-C₄ alkyl;
   Y¹ and Y² are independently absent or selected from O, NR¹⁰, C(═O), C(═O)NR¹⁰ or NR¹¹C(═O);
   Q is CR¹², N or aryl;
   p and q are independently integers selected from 0 or 1;
   R¹⁰, R¹¹ and R¹² are independently selected from hydrogen and C₁-C₄alkyl;
   Ts is a tosylate;
   L and FG are as defined in numbered paragraph (29) above.
(40) A compound according to any one of numbered paragraphs (29) to (39), wherein the compound is of Formula (Ib), shown below:

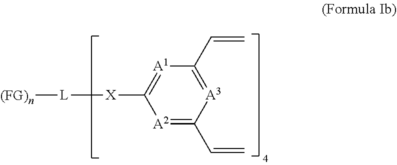

wherein:
   two of A¹, A² and A³ are N and the other of A¹, A² and A³ is CH;
   X is selected from N, O and S; and
   FG, L and n are as defined in numbered paragraph (29).
(41) A compound according to any one of numbered paragraphs (29) to (40), wherein the compound is of Formula (Ic), shown below:

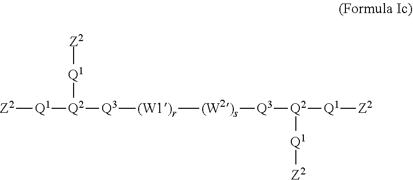

wherein:
   Z² is a re-bridging linking group comprising two functional groups capable of reaction with a sulfur atom from a cysteine moiety of an antibody;
   each Q¹ is independently a bond or a group of Formula IVa:

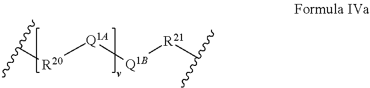

wherein:
   Q^{1A} is absent or selected from a C(═O), OC(═O), NR⁷C(═O), C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈ alkylene and C₁-C₈ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently a group of Formula IVb:

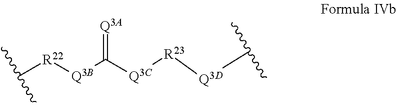

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₈alkylene and C₁-C₈alkylene containing O in the backbone;
   Q^{3D} is absent or selected from C(═O), OC(═O), NR¹²C(═O) and C(═O)NR¹²;
W¹ is a group of Formula IVc₂, Formula IVe₂ or Formula IVc:

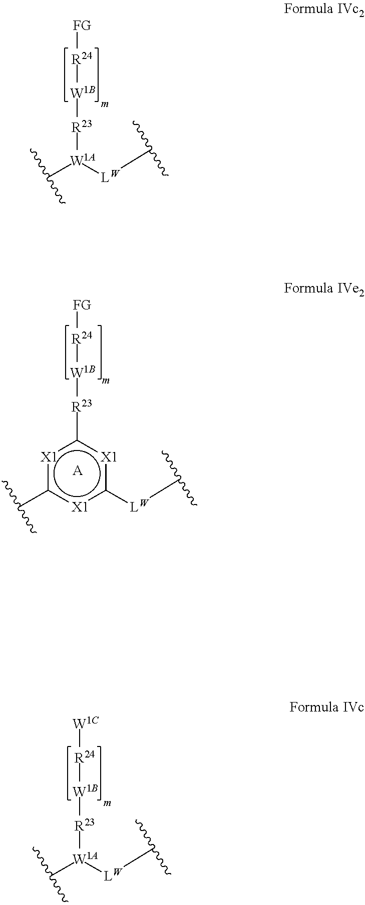

wherein:
   W^{1A} and W^{1C} are independently selected from N and CR¹⁴, wherein R¹⁴ is a selected from hydrogen and C₁-C₄alkyl;
   L^{W} is absent or selected from a C₁-C₆alkylene and a C₁-C₆alkylene containing amino, O or N in the backbone;
   Ring A is a 6-membered aryl, a 6-membered heteroaryl, a 6-membered heterocyclyl or a 6-membered cycloalkyl, wherein X, is selected from CH or N;
   WB is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₆alkylene and C₁-C₆alkylene containing O in the backbone;
   m is an integer selected from 1 or 2;
   W^{1C} is a group of Formula W^{C2}:

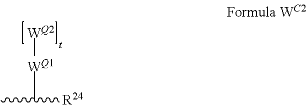

wherein:
   W^{Q1} is a group of Formula W^{C3}:

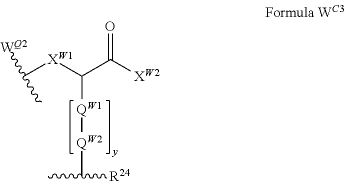

wherein:
Q^{W1} is absent or selected from a C₁-C₁₂alkylene and a
C₁-C₁₂alkylene containing O or N in the backbone; and
Q^{W2} is selected from —C(═O)—, —OC(═O)—, —C(═O)O—, —NHC(═O)—, and —C(═O)NH—;
y is an integer selected from 0 or 1;
X^{W1} is selected from O or NH;
X^{W2} is selected from hydrogen, C₁-C₄alkyl, OR^{x1} and
NR^{x1}R^{x2}, wherein R^{x1} and R^{x2} are independently selected from hydrogen and C₁-C₄alkyl;

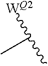

indicates the position where X^{W1} is attached to a group of formula W^{Q2};

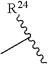

indicates the position where W^{Q1} is attached to a group of formula R²⁴
W^{Q2} is a group of Formula W^{C4B}:

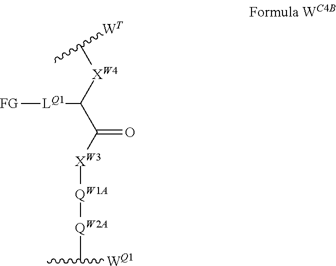

wherein:
Q^{W1A} is absent or selected from C₁-C₁₂alkylene and C₁-C₁₂alkylene containing O or N in the backbone;
Q^{W2A} is selected from —C(═O)—, —OC(═O)—, —C(═O)O—, —NHC(═O)—, and —C(═O)NH—;
X^{W3} is selected from O or NH;

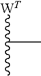

indicates the position where X^{W4} is attached to another group of formula W^{Q2} via the Q^{W2A} substituent group, or

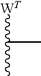

indicates the position where X^{W4} is attached to one of the following groups hydrogen or —C(═O)R^{X3}, wherein R^{x3} is selected from hydrogen and C₁-C₄alkyl;

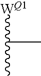

indicates the position where Q^{W2A} is attached to the group of formula W¹ via the X^{W1} group;
X^{W4} is selected from O or NH;
L^{Q1} is a linker comprising one or more groups selected from a C₁-C₂₀alkylene, an alkylenediamine moiety, a (poly)ethylene glycol moiety, an amino acid residue and combinations thereof;
t is an integer selected from 1 to 8;
FG is as in any one of numbered paragraphs (29) or (33) to (35);
r is an integer selected from 0 to 12;
s is an integer selected from 0 to 6; and
wherein r and s are selected such that the total number of active agents per conjugate is between 1 and 12.
(42) A compound according to any one of numbered paragraphs (29) to (41), wherein the compound is of Formula (Ic), shown below:

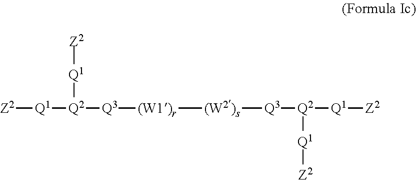

wherein:
   Z² is a re-bridging linking group comprising two functional groups capable of reaction with a sulfur atom from a cysteine moiety of an antibody;
   each Q¹ is independently a bond or a group of Formula IVa:

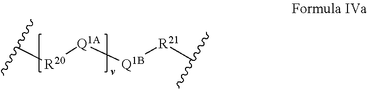

wherein:
   Q^{1A} is absent or selected from a C(═O), OC(═O), NR⁷C(═O), C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈ alkylene and C₁-C₈ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently a group of Formula IVb:

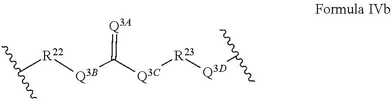

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₈alkylene and C₁-C₈alkylene containing O in the backbone;
   Q^{3D} is absent or selected from C(═O), OC(═O), NR¹²C(═O) and C(═O)NR¹²;
W^{1′} is a group of Formula IVc₁:

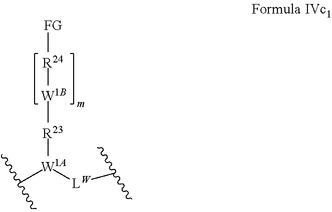

wherein:
   W^{1A} is selected from N and CR¹⁴, wherein R¹⁴ is a selected from hydrogen and C₁-C₄alkyl;
   L^{W} is absent or selected from a C₁-C₆alkylene and a C₁-C₆alkylene containing O or N in the backbone;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₆alkylene and C₁-C₆alkylene containing O in the backbone;
   m is an integer selected from 1 or 2 (suitable m is 1); and
   FG is as defined in any one of numbered paragraphs (29) or (33) to (35);
W^{2′} is a group of Formula IVd:

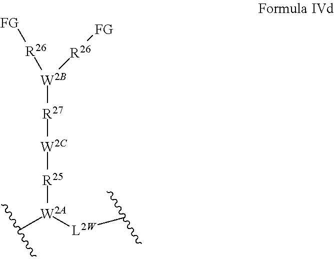

wherein:
   W^{2A} is selected from N and CR¹⁷, wherein R¹⁷ is a selected from hydrogen and C₁-C₄alkyl;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from a C₁-C₆alkylene and a C₁-C₆alkylene containing O or N in the backbone;
   R¹⁷ and R¹⁸ are independently selected from hydrogen and C₁-C₄alkyl;
   R²⁵, R²⁶ and R²⁷ are independently selected from a bond, C₁-C₈alkylene and C₁-C₆alkylene containing O in the backbone; and
   FG is as in numbered paragraph (30);
r is an integer selected from 0 to 12;
s is an integer selected from 0 to 6; and
wherein r and s are selected such that the total number of active agents per conjugate is between 1 and 12.
(43) A compound according to numbered paragraphs (41) or (42), wherein Z² is a group of Formula III, shown below:

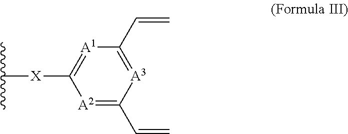

wherein:
   two of A¹, A² and A³ are N and the other of A¹, A² and A³ is CH;
   X is selected from N, NR^{N}, O and S, wherein R^{N} is H or C₁₋₂alkyl; and

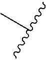

denotes the point of attachment to Q¹.
(44) A compound according to any one of numbered paragraphs (29) to (43), wherein the compound is selected from any one of the compounds listed on pages 95 to 103 (preferably 95 to 97) above.
(45) A conjugating reagent of Formula (II), shown below:

(D-L¹-FG′)ₙ-L-(Z)₈ (Formula II)
wherein:
   FG′ is a functional linking moiety;
   n is an integer selected from 0 to 20;
   L and L′ are independently linkers;
   Z is a functional group capable of reaction with a sulfur atom from a cysteine moiety of an antibody; and
   D is an active agent;
or a pharmaceutically acceptable salt, solvate or hydrate thereof.
(46) A conjugating reagent according to numbered paragraph (45), wherein at least two of the Z groups are separated by between 10 and 60 atoms.
(47) A conjugating reagent according to any one of numbered paragraphs (45) to (46), wherein the linker comprises one or more groups selected from an alkylenediamine moiety, a polyethylene glycol moiety and an amino acid moiety.
(48) A conjugating reagent according to any one of numbered paragraphs (45) to (47), wherein FG′ is selected from

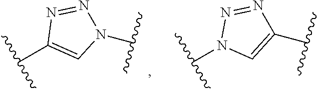 [SMILES: *c1cn(*)nn1 ; *c1cn(*)nn1]

and —C(═O)NH—.
(49) The conjugating reagent according to any one of numbered paragraphs (45) to (48), wherein L¹ is selected from a bond, C₁-C₁₀alkylene and C₁-C₁₀alkylene containing O in the backbone and a group of formula Va:

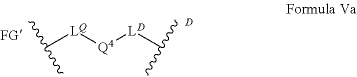

wherein:
   L^{Q} is a selected from a bond, C₁-C₁₀alkylene and a C₁-C₁₀alkylene containing O or NH in the backbone;

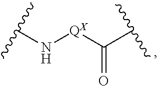

Q⁴ is a single bond, or, where Q^{X} is such that Q⁴ is an amino-acid residue, a dipeptide residue or a tripeptide residue; and
L^{D} is a group for attachment to the active agent.
(50) A conjugating reagent according to any one of numbered paragraphs (45) to (49), wherein Z is a Michael Acceptor.
(51) A conjugating reagent according to any one of numbered paragraphs (45) to (50), wherein the conjugating reagent is of Formula (IIa), shown below:

(D-L¹-FG′)ₙ-L-(Z²)₄ (Formula IIa)
wherein:
   Z² is a re-bridging linking group comprising two functional groups capable of reaction with a sulfur atom from a cysteine moiety of an antibody;
   L, L¹, FG′ and n are as defined in any one of numbered paragraphs (45) to (49).
(52) A conjugating reagent according to any one of numbered paragraphs (45) to (51), wherein the conjugating reagent is selected from one of the following groups:

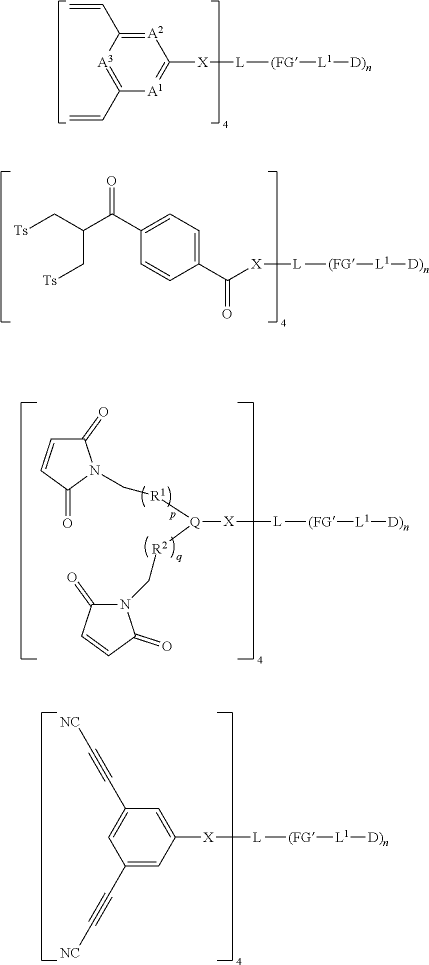

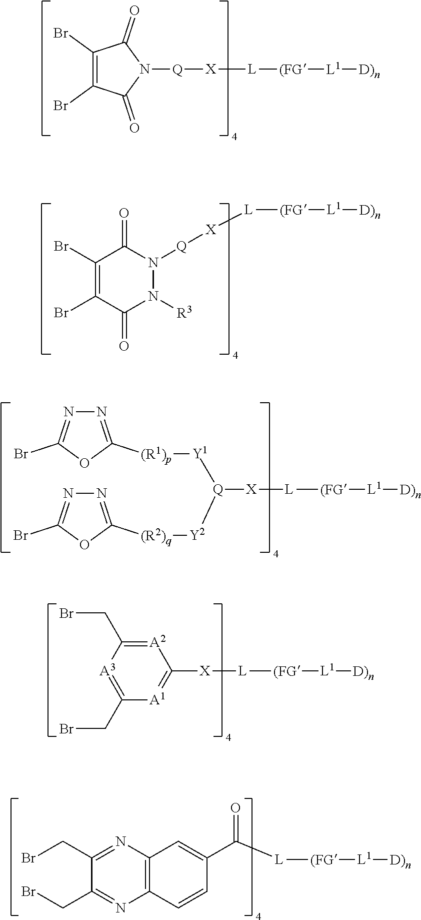

wherein:
   one or two of A¹, A² and A³ are N and the other one or two of A¹, A² and A³ are CH, or all three of A¹, A² and A³ are N;
   X is selected from N, NR^{N}, O and S, wherein R^{N} is H or C₁₋₂alkyl;
   R¹ and R² are independently selected from C₁-C₆ alkylene and C₁-C₆ alkylene containing O in the backbone;
   R³ is selected from hydrogen or a C₁-C₄ alkyl;
   Y¹ and Y² are independently absent or selected from O, NR¹⁰, C(═O), C(═O)NR¹⁰ or NR¹¹C(═O);
   Q is CR¹², N or aryl;
   p and q are independently integers selected from 0 or 1;
   R¹⁰, R¹¹ and R¹² are independently selected from hydrogen and C₁-C₄alkyl; Ts is a tosylate;
   FG′, L, L¹, n and D are as defined in any one of numbered paragraphs (45) to (51).
(53) A conjugating reagent according to any one of numbered paragraphs (45) to (52), wherein the conjugating reagent is of Formula (IIb), shown below:

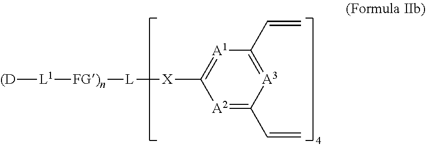

wherein:
   two of A¹, A² and A³ are N and the other of A¹, A² and A³ is CH;
   X is selected from NR^{N}, O and S, wherein R^{N} is H or C₁₋₂ alkyl; and
   FG′, L, L¹, n and D are as defined in any one of numbered paragraphs (45) to (51).
(54) A conjugating reagent according to any one of numbered paragraphs (45) to (53), wherein the conjugating reagent is of Formula (IIc), shown below:

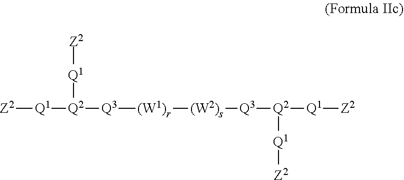

wherein:
   Z² is a re-bridging linking group comprising two functional groups capable of reaction with a sulfur atom from a cysteine moiety of an antibody;
   each Q¹ is independently a bond or a group of Formula IVa:

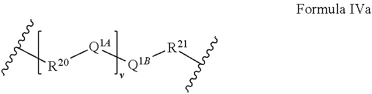

wherein:
   Q^{1A} is absent or selected from a C(═O), OC(═O), NR⁷C(═O), C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈ alkylene and C₁-C₈ alkylene containing O in the backbone; and
   v is an integer selected from 0, 1 or 2;
each Q² is independently selected from N and CR¹¹, wherein R¹¹ is selected from hydrogen and C₁-C₄alkyl;
each Q³ is independently a group of Formula IVb:

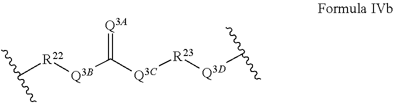

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₈alkylene and C₁-C₈alkylene containing O in the backbone;
   Q^{3D} is absent or selected from C(═O), OC(═O), NR¹²C(═O) and C(═O)NR¹²;
W¹ is a group of Formula IVc or Formula IVe:

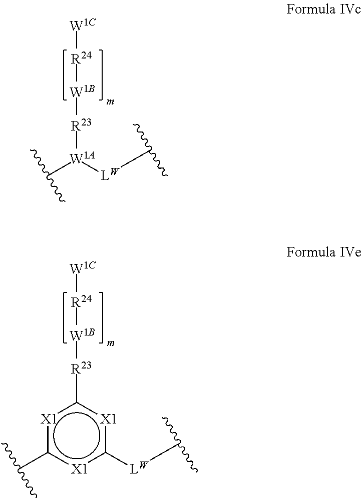

wherein:
   W^{1A} and W^{1C} are independently selected from N and CR¹⁴, wherein R¹⁴ is a selected from hydrogen and C₁-C₄alkyl;
   L^{W} is absent or selected from a C₁-C₆alkylene and a C₁-C₆alkylene containing amino, O or N in the backbone;
   Ring A is a 6-membered aryl, a 6-membered heteroaryl, a 6-membered heterocyclyl or a 6-membered cycloalkyl, wherein X₁ is selected from CH or N;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₈alkylene containing O in the backbone;
   m is an integer selected from 1 or 2;
   W^{1C} is selected from Formula W^{C1} or Formula W^{C2}:

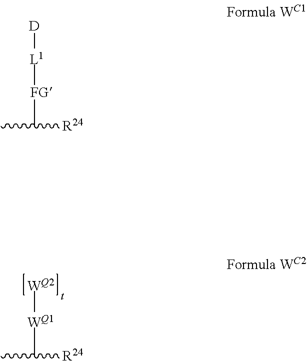

wherein:
   W^{Q1} is a group of Formula W^{C3}:

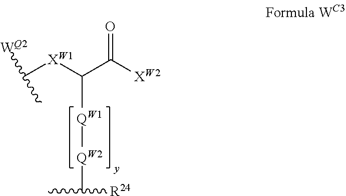

wherein:
Q^{W1} is absent or selected from C₁-C₁₂alkylene and C₁-C₁₂alkylene containing O or N in the backbone; and
Q^{W2} is selected from —C(═O)—, —OC(═O)—, —C(═O)O—, —NHC(═O)—, and —C(═O)NH—;
y is an integer selected from 0 or 1;
X^{W1} is selected from O or NH;
X^{W2} is selected from hydrogen, C₁-C₄alkyl, OR^{x1} and
NR^{x1}R^{x2}, wherein R^{x1} and R^{x2} are independently selected from hydrogen and C₁-C₄alkyl;

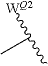

indicates the position where X^{W1} is attached to a group of formula W^{Q2};

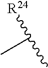

indicates the position where W^{Q1} is attached to a group of formula R²⁴
W^{Q2} is a group of Formula W^{C4}:

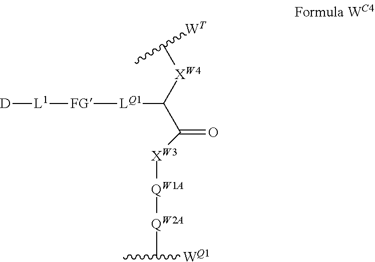

wherein:
Q^{W1A} is absent or selected from C₁-C₁₂alkylene and C₁-C₁₂alkylene containing O or N in the backbone; and
Q^{W2A} is selected from —C(═O)—, —OC(═O)—, —C(═O)O—, —
NHC(═O)—, and —C(═O)NH—;
X^{W3} is selected from O or NH;

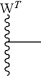

indicates the position where X^{W4} is attached to another group of formula W^{Q2} via the Q^{W2A} substituent group, or

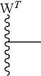

indicates the position where X^{W4} is attached to one of the following groups hydrogen or —C(═O)R^{X3}, wherein R^{x3} is selected from hydrogen and C₁-C₄alkyl;

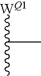

indicates the position where Q^{W2A} is attached to the group of formula W^{Q1} via the X^{W1} group; X^{W4} is selected from O or NH:
L^{Q1} is a linker comprising one or more groups selected from a C₁-C₂₀alkylene, an alkylenediamine moiety, a (poly)ethylene glycol moiety, an amino acid residue and combinations thereof;
t is an integer selected from 1 to 4;
FG′, L¹ and D are as defined in any one of numbered paragraphs (45) to (50);
W^{2′} is a group of Formula IVd:

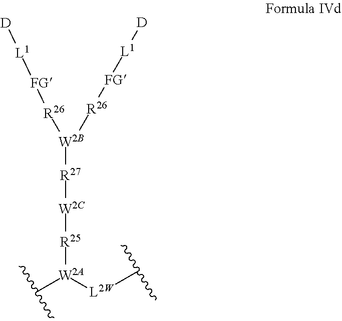

wherein:
   W^{2A} is selected from N and CR¹⁷, wherein R¹⁷ is a selected from hydrogen and C₁-C₄alkyl;
   W^{2B} is a group selected from N, CR¹⁸, —C(═O)N— and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from a C₁-C₆alkylene and a C₁-C₆alkylene containing amino, O or N in the backbone;
   R¹⁷ and R¹⁸ are independently selected from hydrogen and C₁-C₄alkyl;
   R²⁵ and R²⁶ are independently selected from a bond, C₁-C₆alkylene and C₁-C₆alkylene containing O in the backbone; and
   FG′, L¹ and D are as defined in any one of numbered paragraphs (45) to (50);
r is an integer selected from 0 to 12;
s is an integer selected from 0 to 6; and
wherein r and s are selected such that the total number of active agents per conjugating reagent is between 1 and 12.
(55) A conjugating reagent according to any one of numbered paragraphs (45) to (53), wherein the conjugating reagent is of Formula (IIc), shown below:

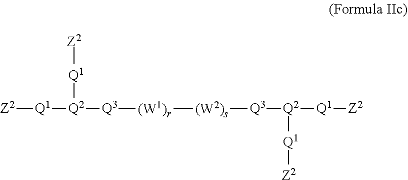

wherein:
   Z² is a re-bridging linking group comprising two functional groups capable of reaction with a sulfur atom from a cysteine moiety of an antibody;
   each Q¹ is a bond or a group of Formula IVa:

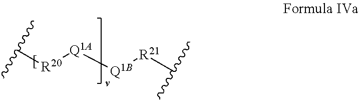

wherein:
   Q^{1A} is absent or selected from a C(═O), OC(═O), NR⁷C(═O), C(═NR⁸) and C(═S);
   Q^{1B} is selected from O, N, S and CR⁹CR¹⁰;
   R⁷, R⁸, R⁹ and R¹⁰ are independently selected from hydrogen and C₁-C₄alkyl;
   R²⁰ and R²¹ are independently selected from a bond, C₁-C₈alkylene and C₁-C₈alkylene containing O in the backbone; and
   v is an integer selected from 0, 1 or 2;
each Q² is selected from N and CR¹¹, wherein R¹¹ is selected from hydrogen and C₁-C₄alkyl;
each Q³ is a group of Formula IVb:

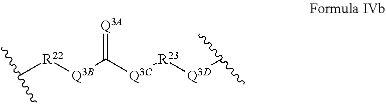

wherein:
   Q^{3A} is selected from NR¹², O and S;
   Q^{3B} and Q^{3C} are independently selected from a bond, O and NR¹³;
   R¹² and R¹³ are independently selected from hydrogen and C₁-C₄alkyl;
   R²² and R²³ are independently selected from a bond, C₁-C₈alkylene and C₁-C₈alkylene containing O in the backbone;
   Q^{3D} is absent or selected from C(═O), OC(═O), NR¹²C(═O) and C(═O)NR¹²;
W^{1′} is a group of Formula IVc₁ or Formula IVe₁:

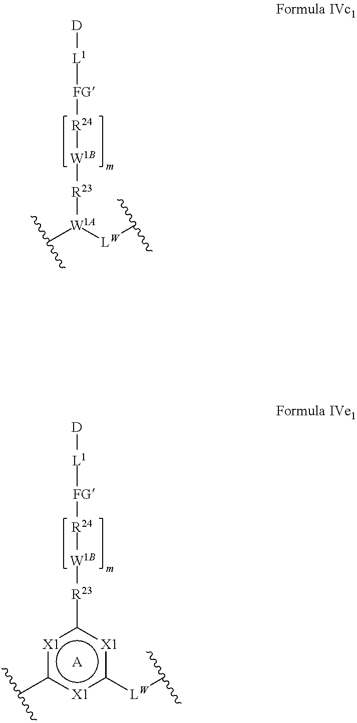

wherein:
   W^{1A} and W^{1C} are independently selected from N and CR¹⁴, wherein R¹⁴ is a selected from hydrogen and C₁-C₄alkyl;
   L^{W} is absent or selected from C₁-C₆alkylene and C₁-C₆alkylene containing 0 or N in the backbone:
   Ring A is a 6-membered aryl, a 6-membered heteroaryl, a 6-membered heterocyclyl or a 6-membered cycloalkyl, wherein X, is selected from CH or N;
   W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
   R¹⁵ and R¹⁶ are independently selected from hydrogen and C₁-C₄alkyl;
   R²³ and R²⁴ are independently selected from a bond, C₁-C₈alkylene and C₁-C₈alkylene containing O in the backbone;
   m is an integer selected from 1 or 2 (suitably m is 1); and
   FG′, L¹ and D are as defined in any one of numbered paragraphs (45) to (50);
W^{2′} is a group of Formula IVd:

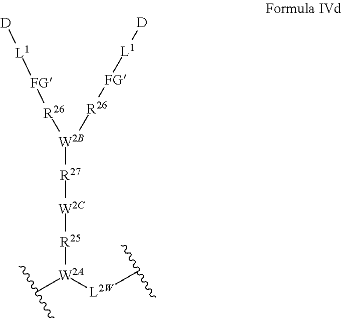

wherein:
   W^{2A} is selected from N and CR¹⁷, wherein R¹⁷ is a selected from hydrogen and C₁-C₄alkyl;
   W^{2B} is a group selected from N, CR¹⁸, —C(═O)N- and —C(═O)CR¹⁸—;
   W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
   L^{2W} is absent or selected from C₁-C₆alkylene and C₁-C₆alkylene containing one or more O or N in the backbone;
   R¹⁷ and R¹⁸ are independently selected from hydrogen and C₁-C₄alkyl;
   R²⁵ and R²⁶ are independently selected from a bond, C₁-C₈alkylene and C₁-C₈alkylene containing O in the backbone; and
   FG′, L¹ and D are as defined in any one of numbered paragraphs (45) to (50);
r is an integer selected from 0 to 12;
s is an integer selected from 0 to 6; and
wherein r and s are selected such that the total number of active agents per conjugating reagent is between 1 and 12.
(56) A conjugating reagent according to numbered paragraphs (54) or (55), wherein Z² is a group of Formula III, shown below:

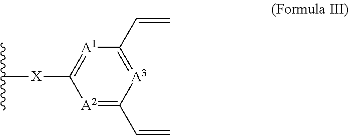

wherein:
   two of A¹, A² and A³ are N and the other of A¹, A² and A³ is CH;
   X is selected from NR^{N}, O and S, wherein R^{N} is H or C₁₋₂ alkyl; and

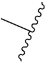

denotes the point of attachment to Q¹.
(57) A conjugating reagent according to any one of numbered paragraphs (45) to (56), wherein the conjugating reagent is selected any one of the conjugating reagents listed on pages 74 to 86 (preferably 74 to 79) above.
(58) An intermediate conjugate comprising an antibody, a linker and at least one functional group capable reacting with another moiety to form a functional linking moiety, wherein:
   i) the linker connects the at least one functional group to the antibody;
   ii) the linker is attached to the antibody through 5 to 8 independent covalent bonds; and
   iii) each covalent bond between the linker and the antibody is formed from the reaction between a sulfur atom on the antibody and a functional group on the linker.
(59) An intermediate conjugate according to numbered paragraph (58), wherein the intermediate conjugate comprises between one and four (preferably between two and four or three or four) re-bridging moieties of Formula (III), shown below:

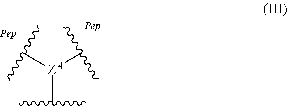

wherein:
   Z^{A} is a functional linking group as defined in any one of numbered paragraphs (6) to (8) above;
   Pep indicates the position where the moiety is attached to the antibody, either directly or indirectly; and

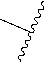

indicates the position where the moiety is attached to linker.
(60) An intermediate conjugate according to numbered paragraph (58) or (59), wherein the intermediate conjugate is a compound of Formula (VI), shown below:

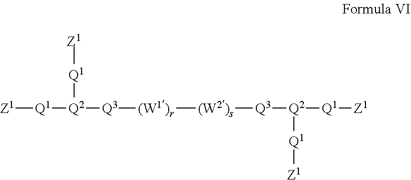

wherein, Z¹, Q¹, Q², Q³, as defined in any one of numbered paragraphs (21) to (23) above; and
W^{1′}, W^{2′}, r and s are as defined any one of numbered paragraphs (41) to (42) above.
(61) An intermediate conjugate according to any one of numbered paragraphs (58) to (60), wherein the intermediate conjugate is selected from any one of the intermediate conjugates listed on pages 105 to 1134 (preferably pages 105 to 107) above.
(62) A pharmaceutical composition comprising, a conjugate according to any one of numbered paragraphs (1) to (28), and a pharmaceutically acceptable carrier, excipient or diluent.
(63) A conjugate according to any one of numbered paragraphs (1) to (28), for use in the treatment of a proliferative disease.
(64) A conjugate according to numbered paragraph (63), wherein the proliferative disease is cancer.

References

| | | DOI |
|---|---|---|
| Beck 2014 | A. Beck and J. M. Reichert, MAbs, | 10.4161/mabs.27436 |
| | 2014, 6, 15-17 | |
| Teicher 2012 | B. A. Teicher and J. H. Doroshow, N. | 10.1056/nejme1211736 |
| | Engl. J. Med., 2012, 367, 1847-1848 | |
| Challita-Eid 2016 | P. M. Challita-Eid, D. Satpayev, P. | 10.1158/0008-5472.CAN-15-1313 |
| | Yang, Z. An, K. Morrison, Y. Shostak, A. | |
| | Raitano, R. Nadell, W. Liu, D. R. Lortie, | |
| | L. Capo, A. Verlinsky, M. Leavitt, F. | |
| | Malik, H. Avina, C. I. Guevara, N. Dinh, | |
| | S. Karki, B. S. Anand, D. S. Pereira, I. | |
| | B. J. Joseph, F. Donate, K. Morrison | |
| | and D. R. Stover, Cancer Res., 2016, | |
| | 76, 3003-3013 | |
| Modi 2020 | S. Modi, C. Saura, T. Yamashita, Y. H. | 10.1056/NEJMoa1914510 |
| | Park, S. B. Kim, K. Tamura, F. Andre, | |
| | H. Iwata, Y. Ito, J. Tsurutani, J. Sohn, N. | |
| | Denduluri, C. Perrin, K. Aogi, E. | |
| | Tokunaga, S. A. Im, K. S. Lee, S. A. | |
| | Hurvitz, J. Cortes, C. Lee, S. Chen, L. | |
| | Zhang, J. Shahidi, A. Yver and I. Krop, | |
| | N. Engl. J. Med., 2020, 382, 610-621 | |
| Kaplon 2020 | H. Kaplon, M. Muralidharan, Z. | 10.1080/19420862.2019.1703531 |
| | Schneider and J. M. Reichert, MAbs, | |
| | 2020, 12, 1703531 | |
| Syed 2020 | Y. Y. Syed, Drugs, 2020, 80, 1019- | 10.1007/s40265-020-01337-5 |
| | 1025. | |
| Kim 2014 | M. T. Kim, Y. Chen, J. Marhoul and F. | 10.1021/bc5000109 |
| | Jacobson, Bioconjug. Chem., 2014, 25, | |
| | 1223-1232. | |
| Jackson 2016 | D. Y. Jackson, Org. Process Res. Dev., | 10.1021/acs.oprd.6b00067 |
| | 2016, 20, 852-866 | |
| Freedy 2016 | A. M. Freedy, P. Akkapeddi, S.-A. Azizi, | 10.1039/C6SC00170J |
| | G. J. L. Bernardes, P. M. S. D. Cal and | |
| | P. M. P. Gois, Chem. Sci., 2016, 7, | |
| | 2954-2963 | |
| Chudasama | V. Chudasama, A. Maruani and S. | 10.1038/nchem.2415 |
| 2016 | Caddick, Nat. Chem., 2016, 8, 114-119 | |
| Hamblett 2004 | K. J. Hamblett, P. D. Senter, D. F. | 10.1158/1078-0432.CCR-04-0789 |
| | Chace, M. M. C. Sun, J. Lenox, C. G. | |
| | Cerveny, K. M. Kissler, S. X. Bernhardt, | |
| | A. K. Kopcha, R. F. Zabinski, D. L. | |
| | Meyer and J. A. Francisco, Clin. Cancer | |
| | Res., 2004, 10, 7063-7070 | |
| Shen 2012 | B.-Q. Shen, K. Xu, L. Liu, H. Raab, S. | 10.1038/nbt.2108 |
| | Bhakta, M. Kenrick, K. L. Parsons- | |
| | Reponte, J. Tien, S.-F. Yu, E. Mai, D. Li, | |
| | J. Tibbitts, J. Baudys, O. M. Saad, S. J. | |
| | Scales, P. J. McDonald, P. E. Hass, C. | |
| | Eigenbrot, T. Nguyen, W. A. Solis, R. N. | |
| | Fuji, K. M. Flagella, D. Patel, S. D. | |
| | Spencer, L. A. Khawli, A. Ebens, W. L. | |
| | Wong, R. Vandlen, S. Kaur, M. X. | |
| | Sliwkowski, R. H. Scheller, P. Polakis | |
| | and J. R. Junutula, Nat. Biotechnol., | |
| | 2012, 30, 184-189 | |
| Strop 2013 | P. Strop, S. H. Liu, M. Dorywalska, K. | 10.1016/j.chembiol.2013.01.010 |
| | Delaria, R. G. Dushin, T. T. Tran, W. H. | |
| | Ho, S. Farias, M. G. Casas, Y. Abdiche, | |
| | D. Zhou, R. Chandrasekaran, C. | |
| | Samain, C. Loo, A. Rossi, M. Rickert, S. | |
| | Krimm, T. Wong, S. M. Chin, J. Yu, J. | |
| | Dilley, J. Chaparro-Riggers, G. F. | |
| | Filzen, C. J. O'Donnell, F. Wang, J. S. | |
| | Myers, J. Pons, D. L. Shelton and A. | |
| | Rajpal, Chem. Biol., 2013, 20, 161-167 | |
| Agarwal 2015 | P. Agarwal and C. R. Bertozzi, | 10.1021/bc5004982 |
| | Bioconjug. Chem., 2015, 26, 176-192 | |
| Godwin 2017 | C. D. Godwin, R. P. Gale and R. B. | 10.1038/leu.2017.187 |
| | Walter, Leukemia, 2017, 31, 1855- | |
| | 1868 | |
| Bross 2001 | P. F. Bross, J. Beitz, G. Chen, X. H. | |
| | Chen, E. Duffy, L. Kieffer, S. Roy, R. | |
| | Sridhara, A. Rahman, G. Williams and | |
| | R. Pazdur, Clin. Cancer Res., 2001, 7, | |
| | 1490-1496 | |
| Hedrich 2018 | W. D. Hedrich, T. E. Fandy, H. M. | |
| | Ashour, H. Wang and H. E. Hassan, | |
| | Clin. Pharmacokinet., 2017, 57, 1-17 | |
| Alley 2008 | S. C. Alley, D. R. Benjamin, S. C. | 10.1021/bc7004329 |
| | Jeffrey, N. M. Okeley, D. L. Meyer, R. J. | |
| | Sanderson and P. D. Senter, Bioconjug. | |
| | Chem., 2008, 19, 759-765 | |
| Lyon 2014 | R. P. Lyon, J. R. Setter, T. D. Bovee, S. | 10.1038/nbt.2968 |
| | O. Doronina, J. H. Hunter, M. E. | |
| | Anderson, C. L. Balasubramanian, S. | |
| | M. Duniho, C. I. Leiske, F. Li and P. D. | |
| | Senter, Nat. Biotechnol., 2014, 32, | |
| | 1059-1062 | |
| Li 2014 | X. Li, T. Fang and G.-J. Boons, Angew. | 10.1002/anie.201402606 |
| | Chemie Int. Ed., 2014, 53, 7179-7182 | |
| Amant 2019 | A. H. St. Amant, F. Huang, J. Lin, K. | 10.1002/anie.201903494 |
| | Rickert, V. Oganesyan, D. Lemen, S. | |
| | Mao, J. Harper, M. Marelli, H. Wu, C. | |
| | Gao, J. Read de Alaniz and R. J. | |
| | Christie, Angew. Chemie Int. Ed., 2019, | |
| | 58, 8489-8493 | |
| Amant 2019* | A. H. St. Amant, F. Huang, J. Lin, D. | 10.1021/acs.bioconjchem.9b00436 |
| | Lemen, C. Chakiath, S. Mao, C. | |
| | Fazenbaker, H. Zhong, J. Harper, W. | |
| | Xu, N. Patel, L. Adams, B. | |
| | Vijayakrishnan, P. W. Howard, M. | |
| | Marelli, H. Wu, C. Gao, J. Read de | |
| | Alaniz and R. J. Christie, Bioconjug. | |
| | Chem., 2019, 30, 2340-2348 | |
| Walker 2019 | J. A. Walker, J. J. Bohn, F. Ledesma, M. | 10.1021/acs.bioconjchem.9b00522 |
| | R. Sorkin, S. R. Kabaria, D. N. Thornlow | |
| | and C. A. Alabi, Bioconjug. Chem., | |
| | 2019, 30, 2452-2457 | |
| Axup 2012 | J. Y. Axup, K. M. Bajjuri, M. Ritland, B. | 10.1073/pnas.1211023109 |
| | M. Hutchins, C. H. Kim, S. A. Kazane, | |
| | R. Halder, J. S. Forsyth, A. F. | |
| | Santidrian, K. Stafin, Y. Lu, H. Tran, A. | |
| | J. Seller, S. L. Biroc, A. Szydlik, J. K. | |
| | Pinkstaff, F. Tian, S. C. Sinha, B. | |
| | Felding-Habermann, V. V Smider and | |
| | P. G. Schultz, Proc. Natl. Acad. Sci. | |
| | U. S. A., 2012, 109, 16101-6 | |
| Zimmerman | E. S. Zimmerman, T. H. Heibeck, A. Gill, | 10.1021/bc400490z |
| 2014 | X. Li, C. J. Murray, M. R. Madlansacay, | |
| | C. Tran, N. T. Uter, G. Yin, P. J. Rivers, | |
| | A. Y. Yam, W. D. Wang, A. R. Steiner, | |
| | S. U. Bajad, K. Penta, W. Yang, T. J. | |
| | Hallam, C. D. Thanos and A. K. Sato, | |
| | Bioconjug. Chem., 2014, 25, 351-361 | |
| Forte 2018 | N. Forte, V. Chudasama and J. R. | 10.1016/j.ddtec.2018.09.004 |
| | Baker, Drug Discov. Today Technol., | |
| | 2018, 30, 11-20 | |
| Behrens 2015 | C. R. Behrens, E. H. Ha, L. L. Chinn, S. | 10.1021/acs.molpharmaceut.5b00432 |
| | Bowers, G. Probst, M. Fitch-Bruhns, J. | |
| | Monteon, A. Valdiosera, A. Bermudez, | |
| | S. Liao-Chan, T. Wong, J. Melnick, J. | |
| | W. Theunissen, M. R. Flory, D. Houser, | |
| | K. Venstrom, Z. Levashova, P. Sauer, | |
| | T. S. Migone, E. H. Van Der Horst, R. L. | |
| | Halcomb and D. Y. Jackson, Mol. | |
| | Pharm., 2015, 12, 3986-3998 | |
| Smith 2010 | M. E. B. Smith, F. F. Schumacher, C. P. | 10.1021/ja908610s |
| | Ryan, L. M. Tedaldi, D. Papaioannou, | |
| | G. Waksman, S. Caddick and J. R. | |
| | Baker, J. Am. Chem. Soc., 2010, 132, | |
| | 1960-1965 | |
| Robinson 2017 | E. Robinson, J. P. M. Nunes, V. | 10.1039/C7RA00788D |
| | Vassileva, A. Maruani, J. C. F. | |
| | Nogueira, M. E. B. Smith, R. B. Pedley, | |
| | S. Caddick, J. R. Baker and V. | |
| | Chudasama, RSC Adv., 2017, 7, 9073- | |
| | 9077 | |
| Badescu 2014 | G. Badescu, P. Bryant, M. Bird, K. | 10.1021/bc500148x |
| | Henseleit, J. Swierkosz, V. Parekh, R. | |
| | Tommasi, E. Pawlisz, K. Jurlewicz, M. | |
| | Farys, N. Camper, X. Sheng, M. Fisher, | |
| | R. Grygorash, A. Kyle, A. Abhilash, M. | |
| | Frigerio, J. Edwards and A. Godwin, | |
| | Bioconjug. Chem., 2014, 25, 1124- | |
| | 1136 | |
| Koniev 2018 | O. Koniev, I. Dovgan, B. Renoux, A. | 10.1039/C8MD00141C |
| | Ehkirch, J. Eberova, S. Cianférani, S. | |
| | Kolodych, S. Papot and A. Wagner, | |
| | Medchemcomm, 2018, 9, 827-830 | |
| Walsh 2019 | S. J. Walsh, S. Omarjee, W. R. J. D. | 10.1039/C8SC04645J |
| | Galloway, T. T. L. Kwan, H. F. Sore, J. | |
| | S. Parker, M. Hyvönen, J. S. Carroll and | |
| | D. R. Spring, Chem. Sci., 2019, 10, | |
| | 694-700 | |
| Walsh 2020 | S. J. Walsh, J. legre, H. Seki, J. D. | 10.1039/D0OB00907E |
| | Bargh, H. F. Sore, J. S. Parker, J. S. | |
| | Carroll and D. R. Spring, Org. Biomol. | |
| | Chem., 2020, 18, 4224-4230 | |
| Bahou 2019 | C. Bahou, E. A. Love, S. Leonard, R. J. | 10.1021/acs.bioconjchem.9b00174 |
| | Spears, A. Maruani, K. Armour, J. R. | |
| | Baker and V. Chudasama, Bioconjug. | |
| | Chem., 2019, 30, 1048-1054 | |
| Bargh 2020 | J. D. Bargh, S. J. Walsh, A. Isidro- | 10.1039/C9SC06410A |
| | Llobet, S. Omarjee, J. S. Carroll and D. | |
| | R. Spring, Chem. Sci., 2020, 11, 2375- | |
| | 2380 | |
| Pittelkow 2002 | M. Pittelkow, R. Lewinsky and J. B. | 10.1055/s-2002-34859 |
| | Christensen, Synthesis (Stuttg)., 2002, | |
| | 2195-2202 | |
| Makino 2014 | A. Makino, E. Hara, I. Hara, E. Ozeki | 10.1021/la404488n |
| | and S. Kimura, Langmuir, 2014, 30, | |
| | 669-674 | |
| Goswami 2015 | L. N. Goswami, Q. Cai, L. Ma, S. S. | 10.1039/C5OB00876J |
| | Jalisatgi and M. F. Hawthorne, Org. | |
| | Biomol. Chem., 2015, 13, 8912-8918 | |
| Collins 2011 | C. G. Collins, J. M. Baumes and B. D. | 10.1039/C1CC15550D |
| | Smith, Chem. Commun., 2011, 47, | |
| | 12352-12354 | |
| Pasupuleti 2020 | B. G. Pasupuleti, K. Khongsti, B. Das | 10.1016/j.ejmech.2019.111908 |
| | and G. Bez, Eur. J. Med. Chem., 2020, | |
| | 186, 111908 | |
| Xu 2021 | Xu L, Kuan SL, Weil T. Chloromethyl | 10.26434/chemrxiv.14769873.v1 |
| | Acryl Reagents for Simple and Site- | |
| | Selective Cysteine and Disulfide | |
| | Modification. ChemRxiv. Cambridge: | |
| | Cambridge Open Engage; 2021, 1-17 | |
| Stieger 2021 | Christian E. Stieger, Luise Franz, | 10.1002/anie.202100683 |
| | Frieder Körlin, Christian P. R. | |
| | Hackenberger, Angew. Chemie Int. Ed., | |
| | 2021, 60, 1-7 | |
| Walsh 2021 | Stephen J. Walsh, Jonathan D. Bargh, | https://doi.org/10.1039/D0CS00310G |
| | Friederike M. Dannheim, Abigail R. | |
| | Hanby, Hikaru Seki, Andrew J. | |
| | Counsell, Xiaoxu Ou, Elaine Fowler, | |
| | Nicola Ashman, Yuri Takada, Albert | |
| | Isidro-Llobet, Jeremy S. Parker, Jason | |
| | S. Carroll and David R. Spring, Chem. | |
| | Soc. Rev. 2021, 50, 1305-1353 | |

1. A conjugate comprising an antibody, a linker and at least one active agent, wherein:
i) the linker connects the at least one active agent(s) to the antibody;
ii) the linker is attached to the antibody through 5 to 8 independent covalent bonds; and
iii) each covalent bond between the linker and the antibody is formed from the reaction between a sulfur atom on the antibody and a functional group on the linker.

2. The conjugate according to claim 1, wherein linker is attached to the antibody through 6 to 8 independent covalent bonds such that the linker re-bridges between two and four reduced interchain disulfide bonds in the antibody.

3. The conjugate according to claim 1 or claim 2, wherein linker is attached to the antibody through 8 independent covalent bonds such that the linker re-bridges four reduced interchain disulfide bonds in the antibody.

4. The conjugate according to any one of claims 1 to 3, wherein the conjugate comprises between one and four re-bridging moieties of Formula (III), shown below: 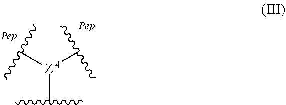
wherein:
Z^{A} is a functional linking group;
Pep indicates the position where the moiety is attached to the antibody, either directly or indirectly; and 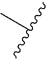
indicates the position where the moiety is attached to linker.

5. The conjugate according to claim 4, wherein the conjugate comprises between three and four re-bridging moieties of Formula (III).

6. The conjugate according to claim 4 or 5, wherein the re-bridging moieties of Formula (III) are independently selected from the one of the following groups: 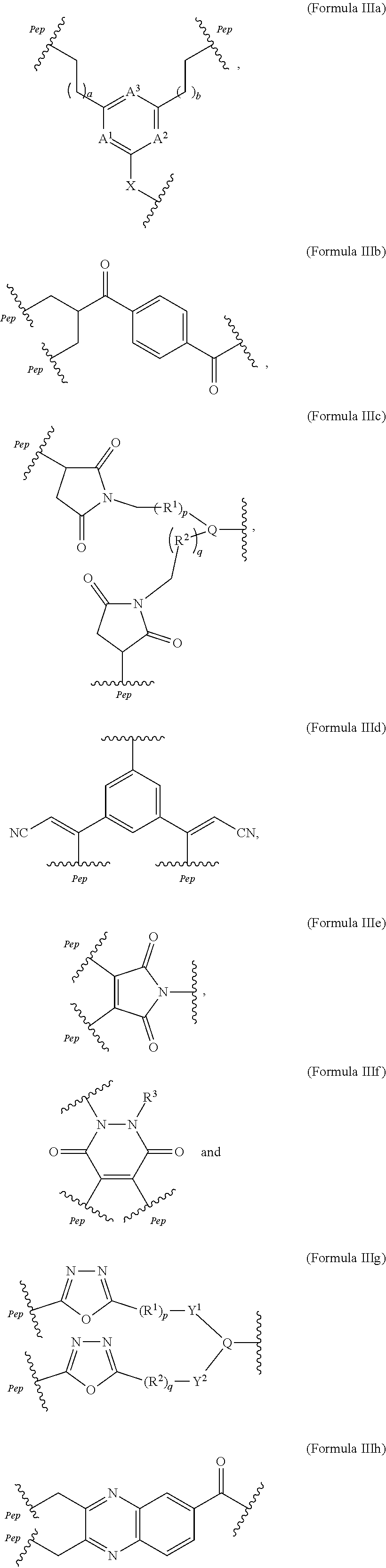
wherein:
one or two of A¹, A² and A³ are N and the other one or two of A¹, A² and A³ are CH, or all three of A¹, A² and A³ are N or CH;
a and b are integers selected from 0 or 1;
X is selected from N, NR^{N}, O and S, wherein R^{N} is H or C₁₋₂alkyl;
R¹ and R² are independently selected from C₁-C₆ alkylene and C₁-C₆ alkylene containing O in the backbone;
R³ is selected from hydrogen or a C₁-C₄ alkyl;
Y¹ and Y² are independently absent or selected from O, NR⁴, C(═O), C(═O)NR⁴ or NR⁵C(═O);
p and q are independently integers selected from 0 or 1;
Q is CR⁶, N or aryl;
R⁴, R⁵ and R⁶ are independently selected from hydrogen and C₁-C₄alkyl;
Pep indicates the position where the moiety is linked to the antibody, either directly or indirectly; and 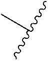
indicates the position where the moiety is attached to linker.

7. The conjugate according to any one of claims 4 to 6, wherein the re-bridging moieties each have the general Formula (IIIa), shown below: 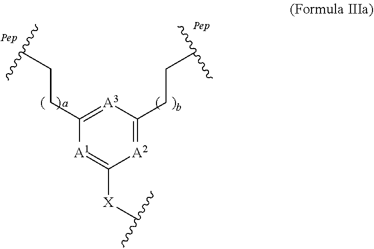
wherein each of A¹, A², A³, X, Pep and 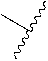
are as defined in claim 6.

8. The conjugate according to any one of claims 1 to 7, wherein the conjugate is of Formula (IIIA), shown below: 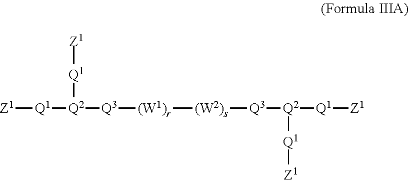
wherein:
Z¹ is a re-bridging moiety of Formula (III) as defined in claim 6 or claim 7 above that is attached to an antibody at the positions shown in Formula (III);
each Q¹ is independently a bond or a group of Formula IVa: 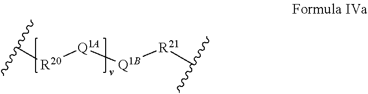
wherein:
Q^{1A} is absent or selected from a C(═O), OC(═O), NR⁷C(═O), C(═NR⁸) and C(═S);
Q^{1B} is selected from O, N, S and CR⁹CR¹⁰;
R⁷, R⁸, R⁹ and R¹⁰ are independently selected from hydrogen and C₁-C₄alkyl;
R²⁰ and R²¹ are independently selected from a bond, a C₁-C₁₀ alkylene and a C₁-C₁₀ alkylene containing O in the backbone; and
v is an integer selected from 0, 1 or 2;
each Q² is independently selected from N and CR¹¹, wherein R¹¹ is selected from hydrogen and C₁-C₄alkyl;
each Q³ is independently a group of Formula IVb: 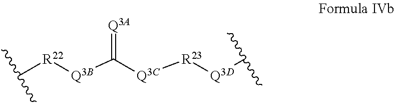
wherein:
Q^{3A} is selected from NR¹², O and S;
Q^{3B} and Q^{3C} are independently selected from a bond, O and NR¹³;
R¹² and R¹³ are independently selected from hydrogen and C₁-C₄alkyl;
R²² and R²³ are independently selected from a bond, a C₁-C₁₀alkylene and a C₁-C₁₀alkylene containing O in the backbone;
Q^{3D} is absent or selected from C(═O), OC(═O), NR¹²C(═O) and C(═O)NR¹²;
W¹ is a group of Formula IVc or Formula IVe: 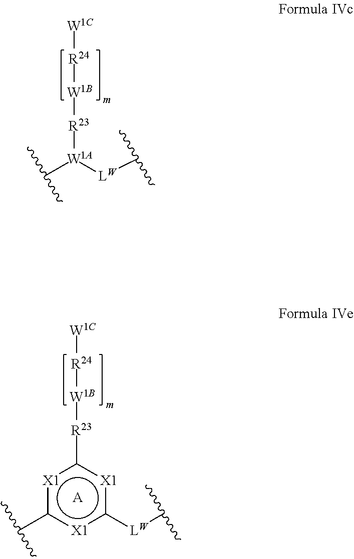
wherein:
W^{1A} is selected from N and CR¹⁴, wherein R¹⁴ is a selected from hydrogen and C₁-C₄alkyl;
L^{W} is absent or selected from a C₁-C₆alkylene and a C₁-C₆alkylene containing amino, O or N in the backbone;
Ring A is a 6-membered aryl, a 6-membered heteroaryl, a 6-membered heterocyclyl or a 6-membered cycloalkyl, wherein X, is selected from CH or N;
W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
R¹⁵ and R¹⁶ are independently selected from hydrogen and C₁-C₄alkyl;
R²³ and R²⁴ are independently selected from a bond, C₁-C₁₂alkylene and C₁-C₁₂alkylene containing O in the backbone;
m is an integer selected from 1 or 2;
W^{1C} is selected from Formula W^{Q1} or Formula W^{C2}: 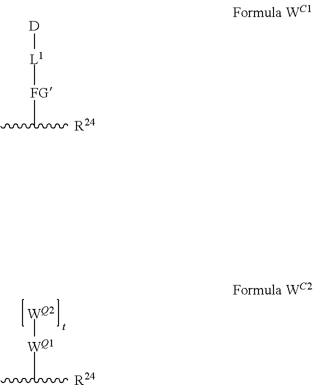
wherein:
W^{Q1} is a group of Formula W^{C3}: 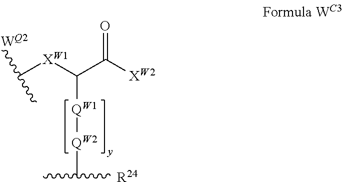
wherein:
Q^{W1} is absent or selected from a C₁-C₁₂alkylene and a C₁-C₁₂alkylene containing O or N in the backbone; and
Q^{W2} is selected from —C(═O)—, —OC(═O)—, —C(═O)O—, —NHC(═O)—, and —C(═O)NH—;
y is an integer selected from 0 or 1;
X^{W1} is selected from O or NH;
X^{W2} is selected from hydrogen, C₁-C₄alkyl, OR^{x1} and NR^{x1}R^{x2}, wherein R^{x1} and R^{x2} are independently selected from hydrogen and C₁-C₄alkyl; 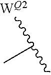
indicates the position where X^{W1} is attached to a group of formula W^{Q2}; 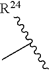
indicates the position where W^{Q1} is attached to a group of formula R²⁴
W^{Q2} is a group of Formula W^{C4}: 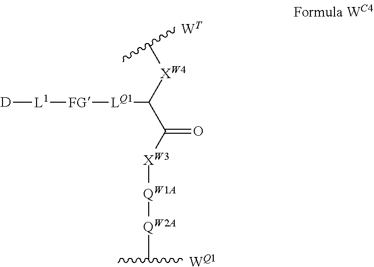
wherein:
Q^{W1A} is absent or selected from a C₁-C₁₂alkylene and a C₁-C₁₂alkylene containing O or N in the backbone; and
Q^{W2A} is selected from —C(═O)—, —OC(═O)—, —C(═O)O—, —NHC(═O)—, and —C(═O)NH—; L^{Q1} is a linker comprising one or more groups selected from a C₁-C₂₀alkylene, an alkylenediamine moiety, a (poly)ethylene glycol moiety, an amino acid residue and combinations thereof;
X^{W3} is selected from O or NH: 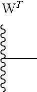
indicates the position where X^{W4} is attached to another group of formula W^{Q2} via the Q^{W2A} substituent group, or 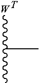
indicates the position where X^{W4} is attached to one of the following groups: a hydrogen or —C(═O)R^{X3}, wherein R^{x3} is selected from hydrogen and C₁-C₄alkyl; 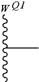
indicates the position where Q^{W2A} is attached to the group of formula W^{Q1} via the X^{W1} group;
X^{W4} is selected from O or NH;
L^{Q1} is a linker comprising one or more groups selected from a C₁-C₂₀alkylene, an alkylenediamine moiety, a (poly)ethylene glycol moiety, an amino acid residue and combinations thereof
t is an integer selected from 1 to 8;
FG′ is a functional linking moiety;
L¹ is a linker; and
D is an active agent;
W² is a group of Formula IVd: 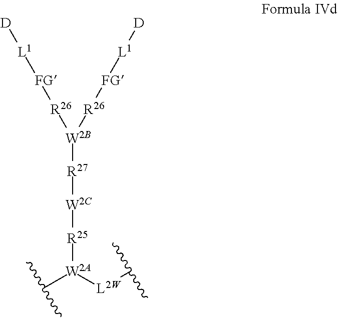
wherein:
W^{2A} is selected from N and CR¹⁷;
W^{2B} is a group selected from N, CR¹⁸, —C(═O)N— and —C(═O)CR¹⁸—;
W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
L^{2W} is absent or selected from a C₁-C₆alkylene and a C₁-C₆alkylene containing amino, O or N in the backbone;
R¹⁷ and R¹⁸ are independently selected from hydrogen and C₁-C₄alkyl;
R²⁵ and R²⁶ are independently selected from a bond, C₁-C₆alkylene and C₁-C₆alkylene containing O in the backbone; and
FG′, L¹ and D are as defined above;
r is an integer selected from 0 to 12;
s is an integer selected from 0 to 6; and
wherein r and s are selected such that the total number of active agents per conjugate is between 1 and 12.

9. The conjugate according to any one of claims 1 to 8, wherein the conjugate is of Formula (IIIA), shown below: 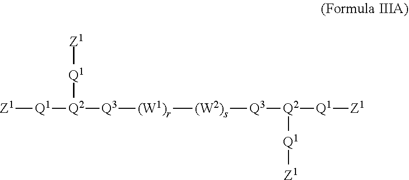
wherein:
Z¹ is a re-bridging moiety of Formula (III) as defined in claim 6 or claim 7 above that is attached to a antibody at the positions shown in Formula (III);
each Q¹ is independently a bond or a group of Formula IVa: 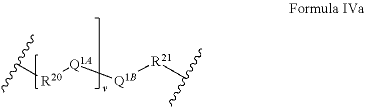
wherein:
Q^{1A} is absent or selected from a C(═O), OC(═O), NR⁷C(═O), C(═NR⁸) and C(═S);
Q^{1B} is selected from O, N, S and CR⁹CR¹⁰;
R⁷, R⁸, R⁹ and R¹⁰ are independently selected from hydrogen and C₁-C₄alkyl;
R²⁰ and R²¹ are independently selected from a bond, a C₁-C₈ alkylene and a C₁-C₈alkylene containing O in the backbone; and
v is an integer selected from 0, 1 or 2;
each Q² is independently selected from N and CR¹¹, wherein R¹¹ is selected from hydrogen and C₁-C₄alkyl;
each Q³ is independently a group of Formula IVb: 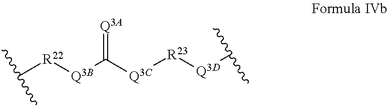
wherein:
Q^{3A} is selected from NR¹², O and S;
Q^{3B} and Q^{3C} are independently selected from a bond, O and NR¹³;
R¹² and R¹³ are independently selected from hydrogen and C₁-C₄alkyl;
R²² and R²³ are independently selected from a bond, a C₁-C₈alkylene and a C₁-C₈alkylene containing O in the backbone;
Q^{3D} is absent or selected from C(═O), OC(═O), NR¹²C(═O) and C(═O)NR¹²,
W¹ is a group of Formula IVc₁ or Formula IVe₁: 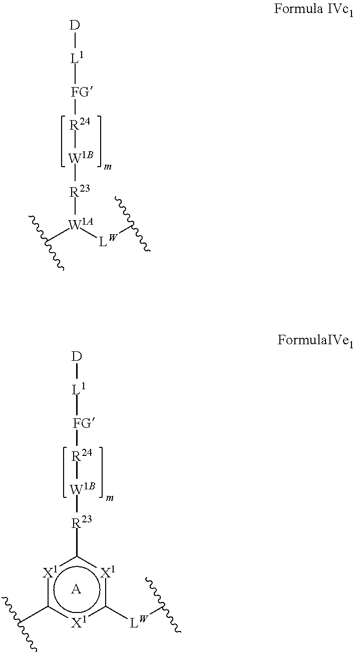
wherein:
W^{1A} is selected from N and CR¹⁴, wherein R¹⁴ is a selected from hydrogen and C₁-C₄alkyl;
L^{W} is absent or selected from a C₁-C₆alkylene and a C₁-C₆alkylene containing O or N in the backbone:
Ring A is a 6-membered aryl, a 6-membered heteroaryl, a 6-membered heterocyclyl or a 6-membered cycloalkyl, wherein X, is selected from CH or N;
W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
R¹⁵ and R¹⁶ are independently selected from hydrogen and C₁-C₄alkyl;
R²³ and R²⁴ are independently selected from a bond, a C₁-C₈alkylene and a C₁-C₆alkylene containing O in the backbone;
m is an integer selected from 1 or 2; and
FG′ is a functional linking moiety;
L¹ is a linker; and
D is an active agent;
W² is a group of Formula IVd: 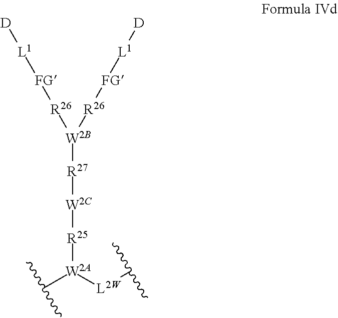
wherein:
W^{2A} is selected from N and CR¹⁷;
W^{2B} is a group selected from N, CR¹⁸, —C(═O)N— and —C(═O)CR¹⁸—;
W^{C2} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
L^{2W} is absent or selected from a C₁-C₆alkylene and a C₁-C₆alkylene containing O or N in the backbone;
R¹⁷ and R¹⁸ are independently selected from hydrogen and C₁-C₄alkyl;
R²⁵ and R²⁶ are independently selected from a bond, a C₁-C₈alkylene and a C₁-C₈alkylene containing O in the backbone; and
FG′, L¹ and D are as defined above;
r is an integer selected from 0 to 12;
s is an integer selected from 0 to 6; and
wherein r and s are selected such that the total number of active agents per conjugate is between 1 and 12.

10. The conjugate according to claim 8 or claim 9, wherein Z¹ is a re-bridging moiety of Formula (IIIa), shown below: 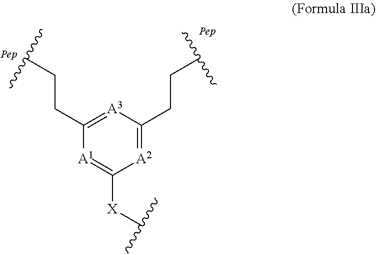
wherein each of A¹, A², A³, X, Pep and 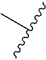
are as defined in claim 6.

11. The conjugate according to any one of claims 1 to 10, wherein the antibody is a monoclonal antibody.

12. The conjugate according to any one of claims 4 to 11, wherein at least two of the re-bridging moieties are separated by between 10 and 60 atoms.

13. The conjugate according to any one of claims 1 to 12, wherein the conjugate is selected from one of the following conjugates: 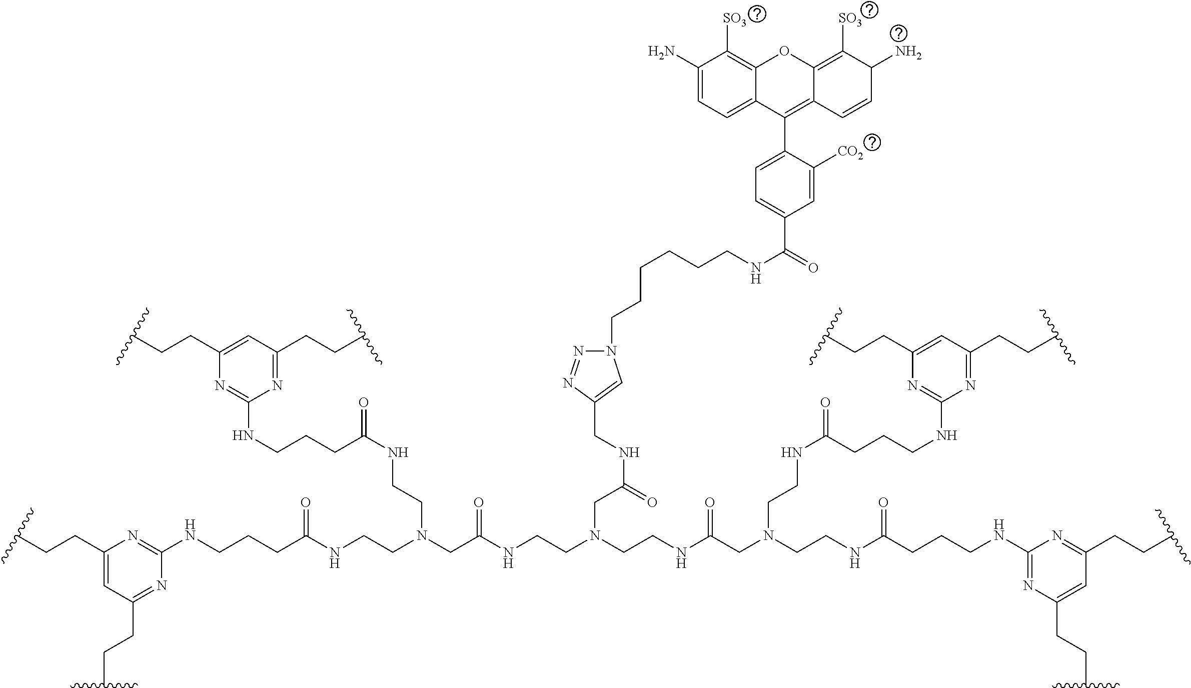 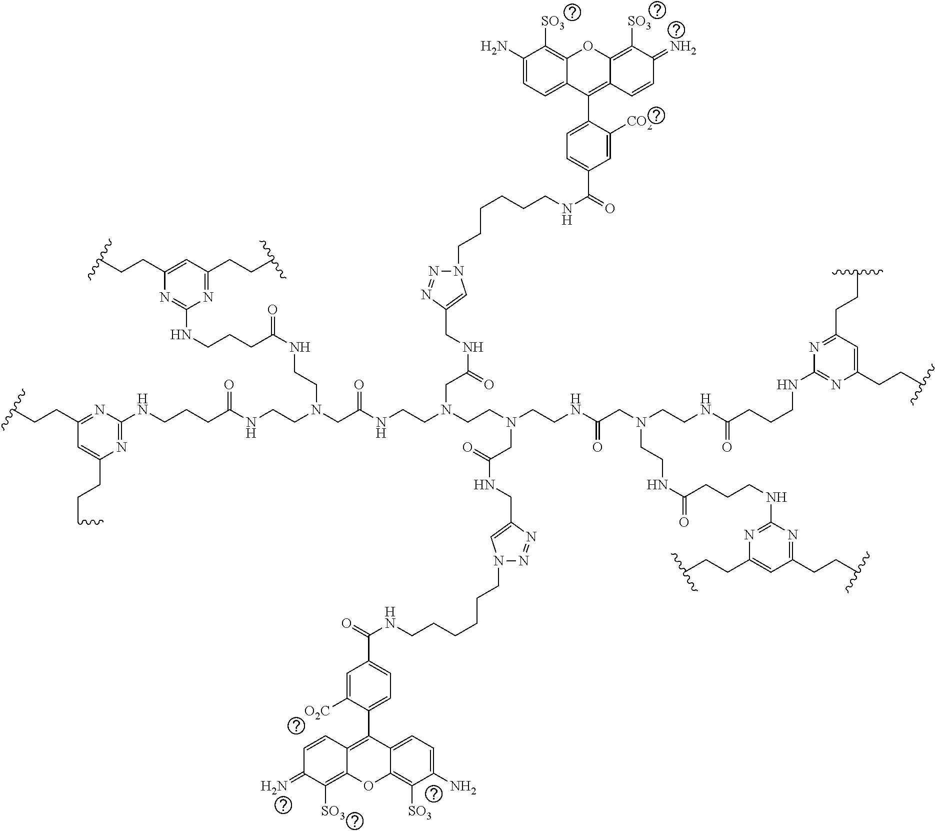 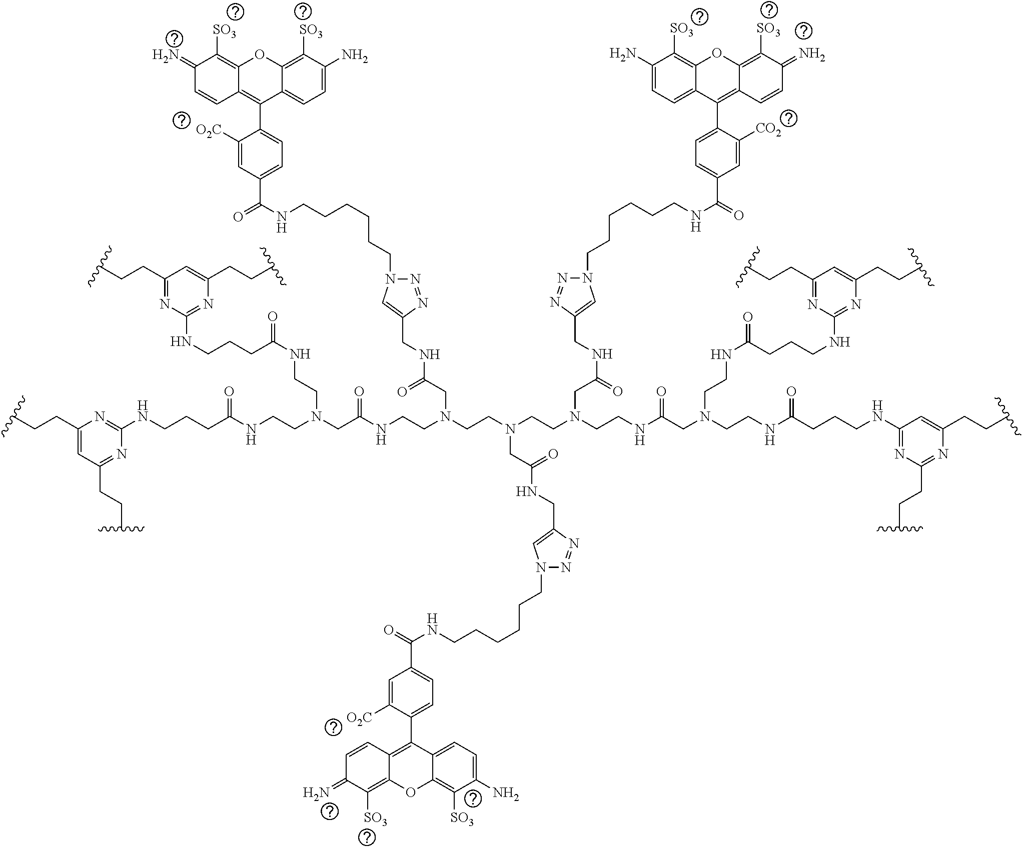 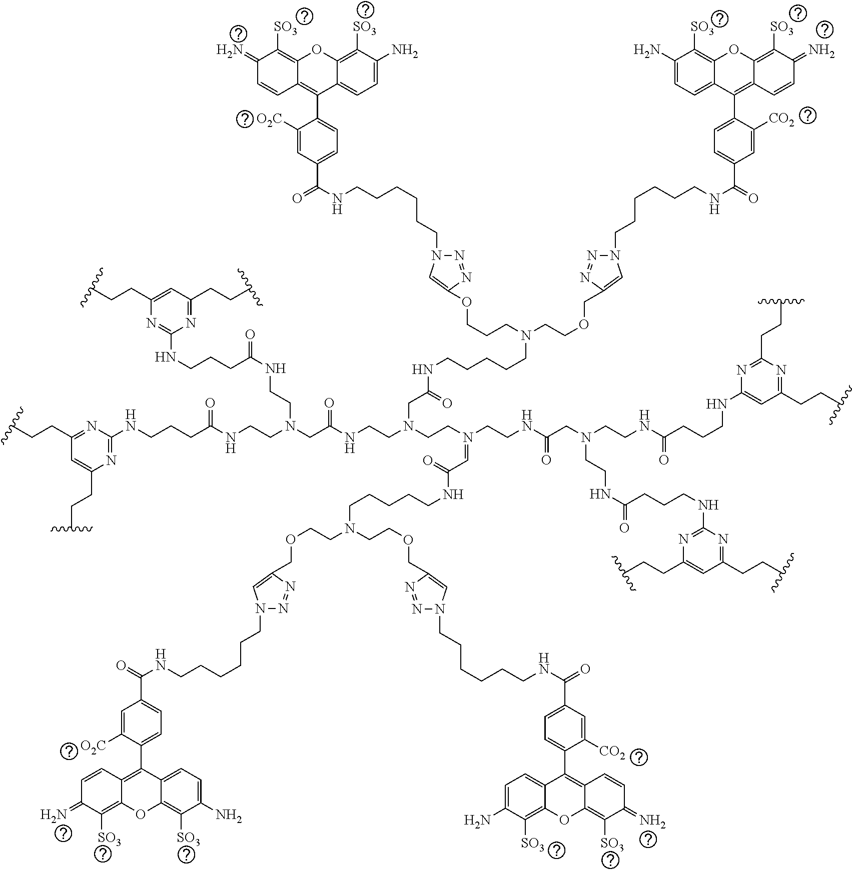 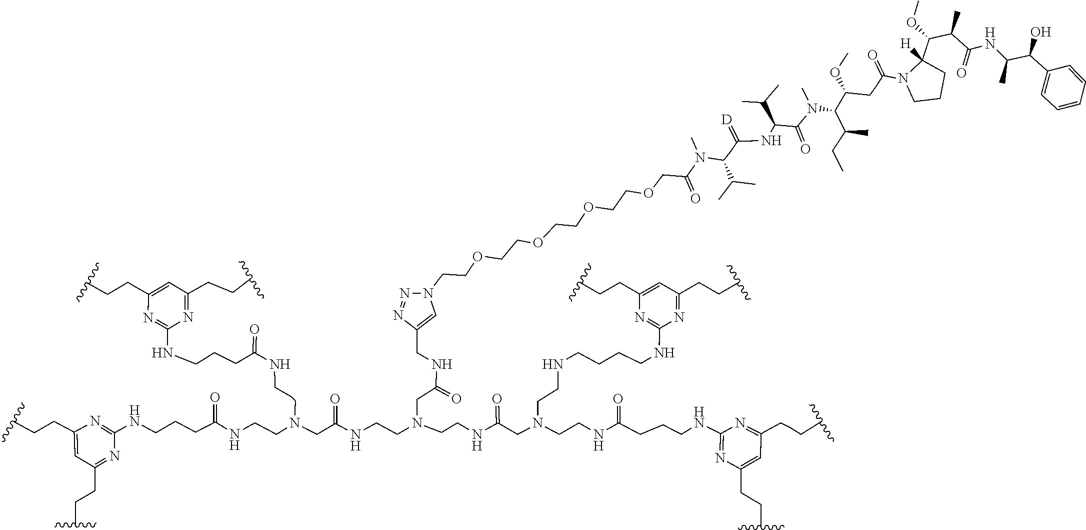 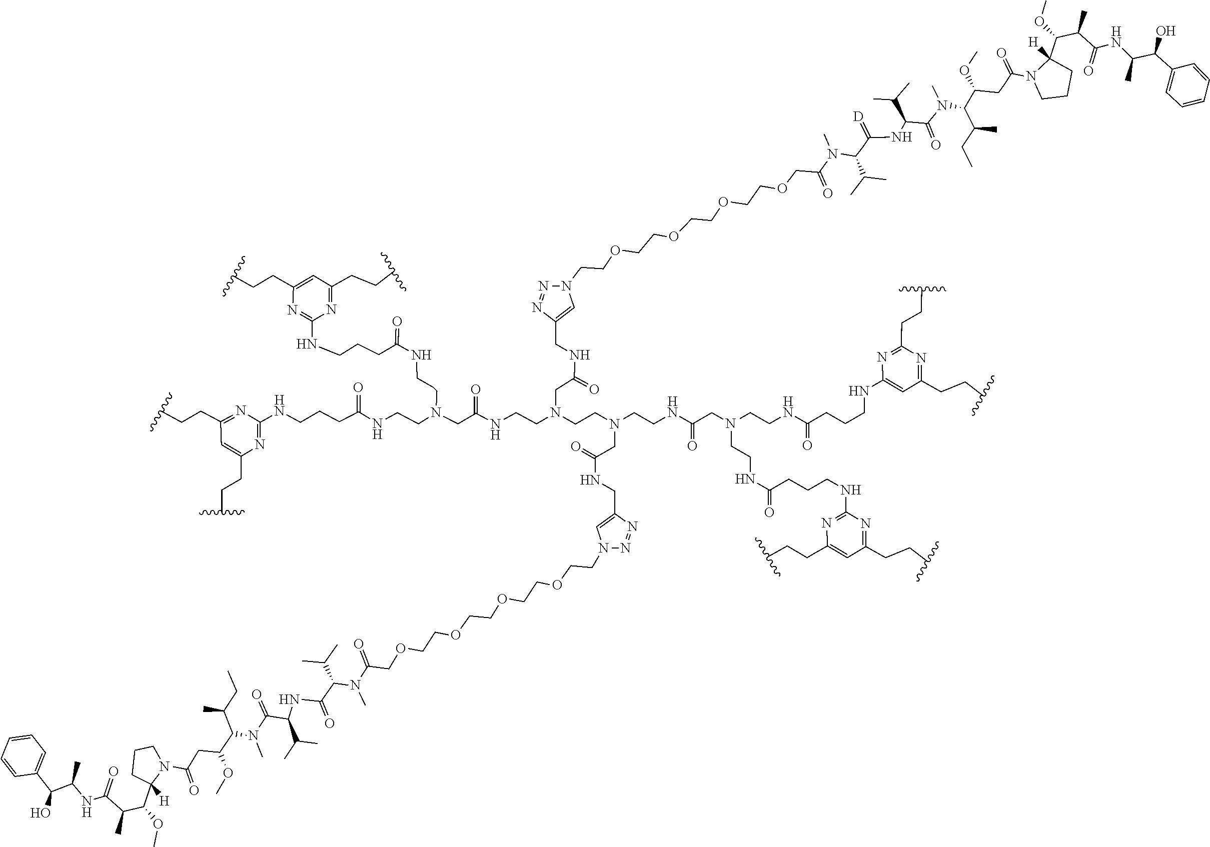 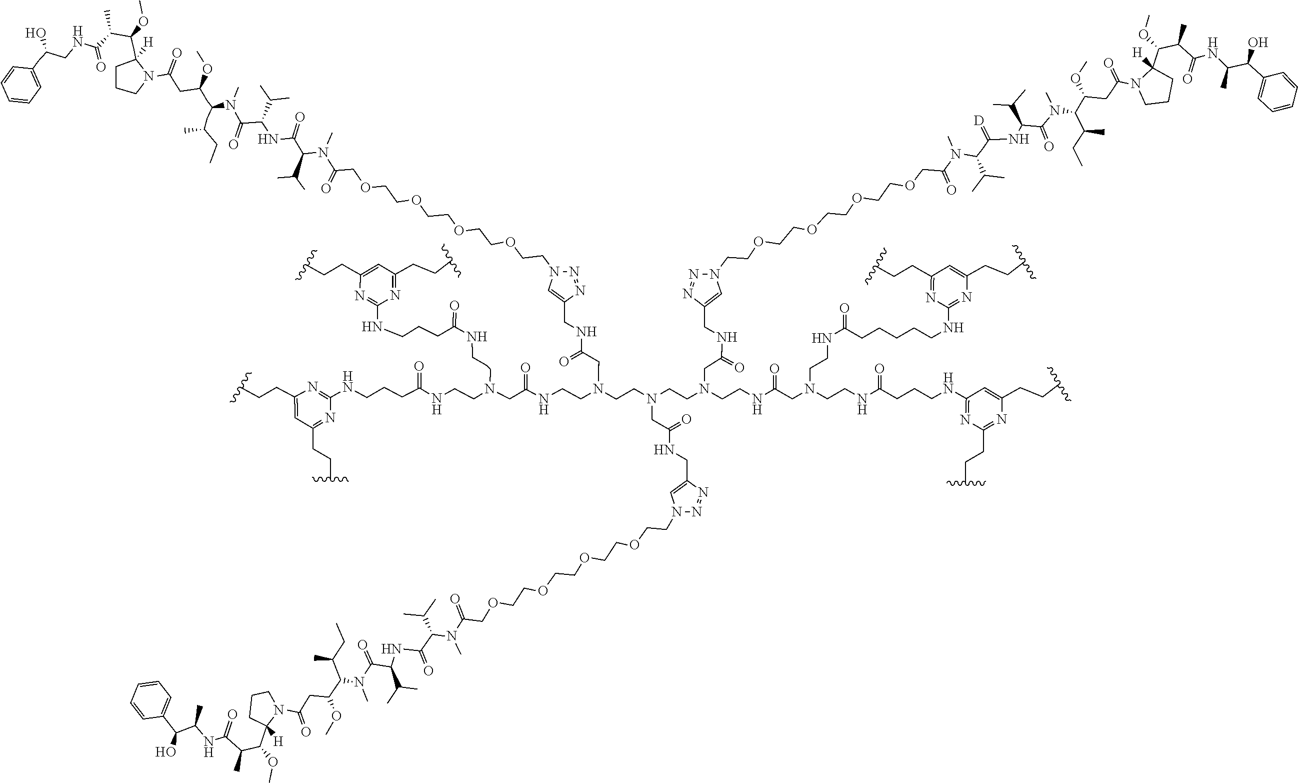 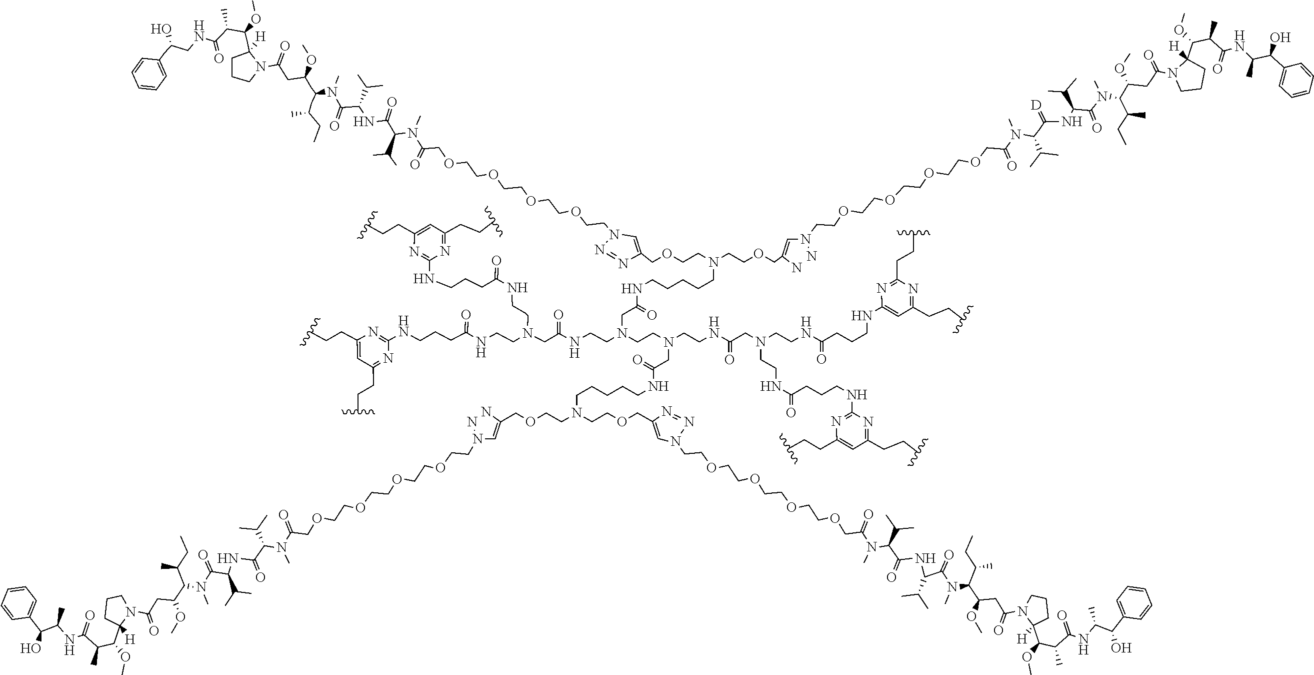 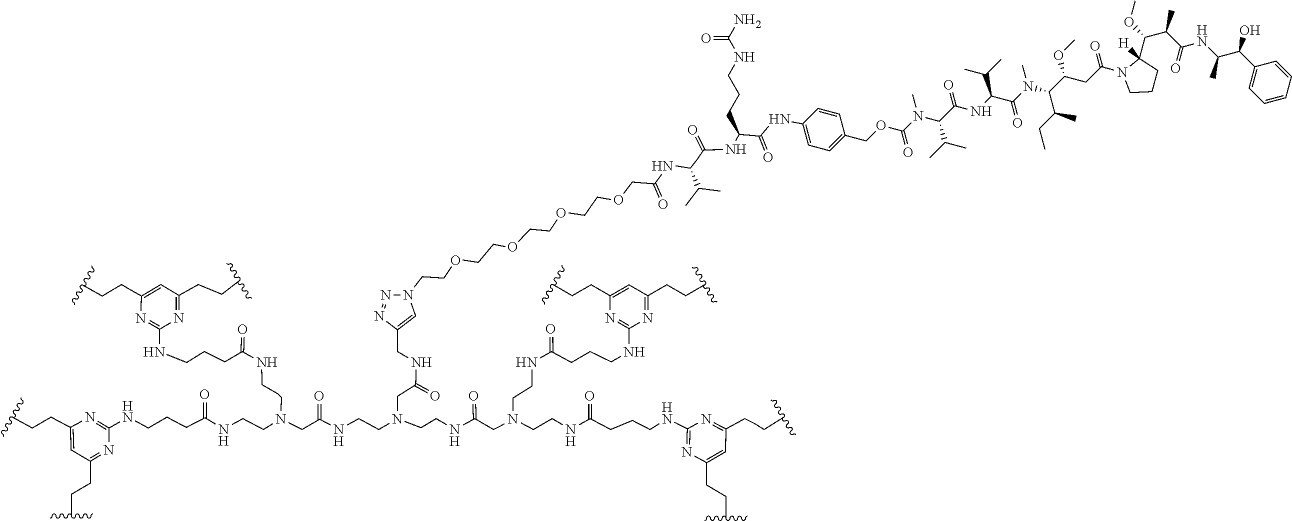 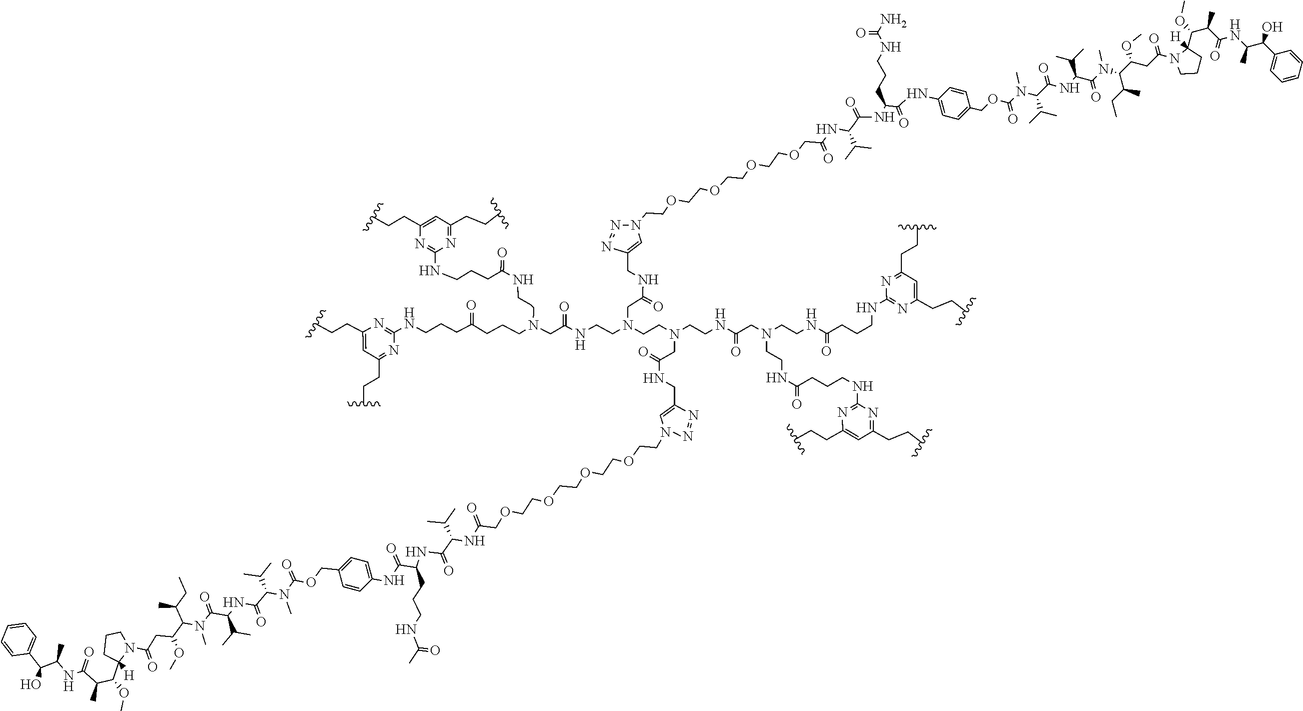 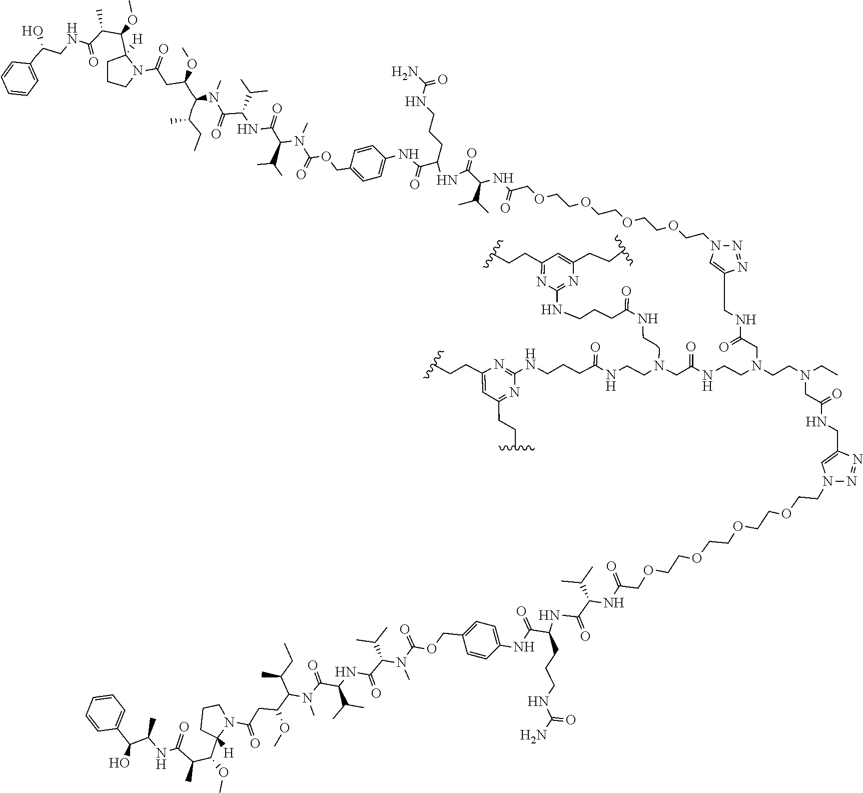 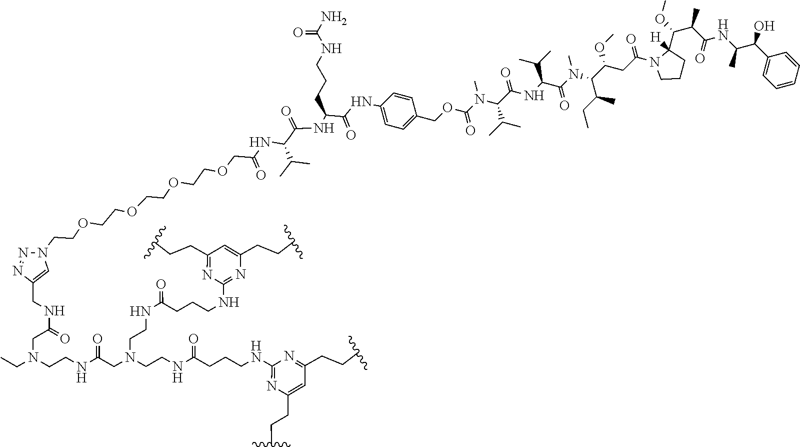 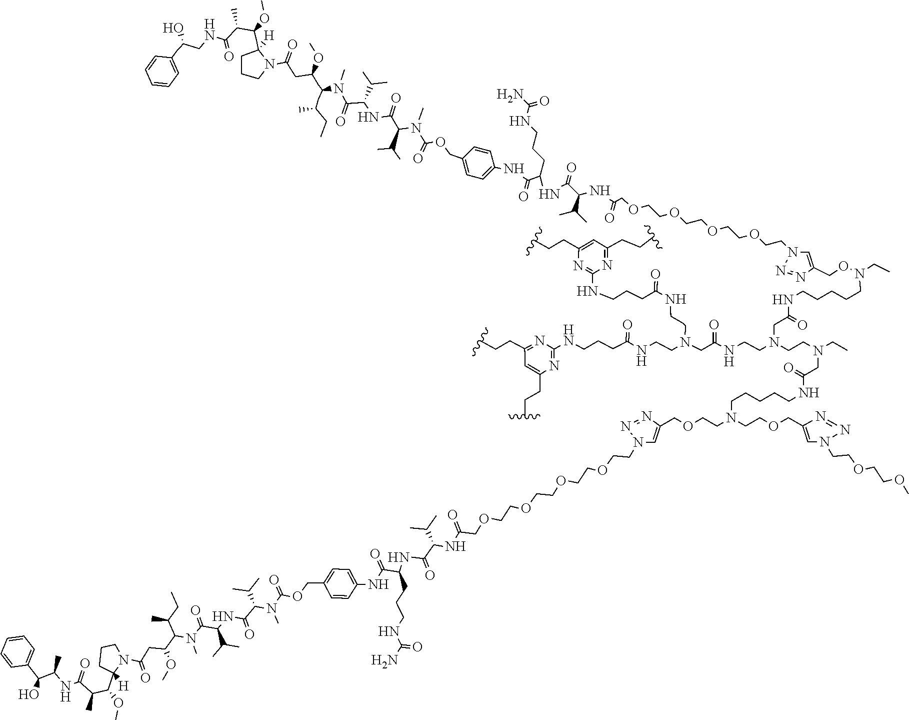 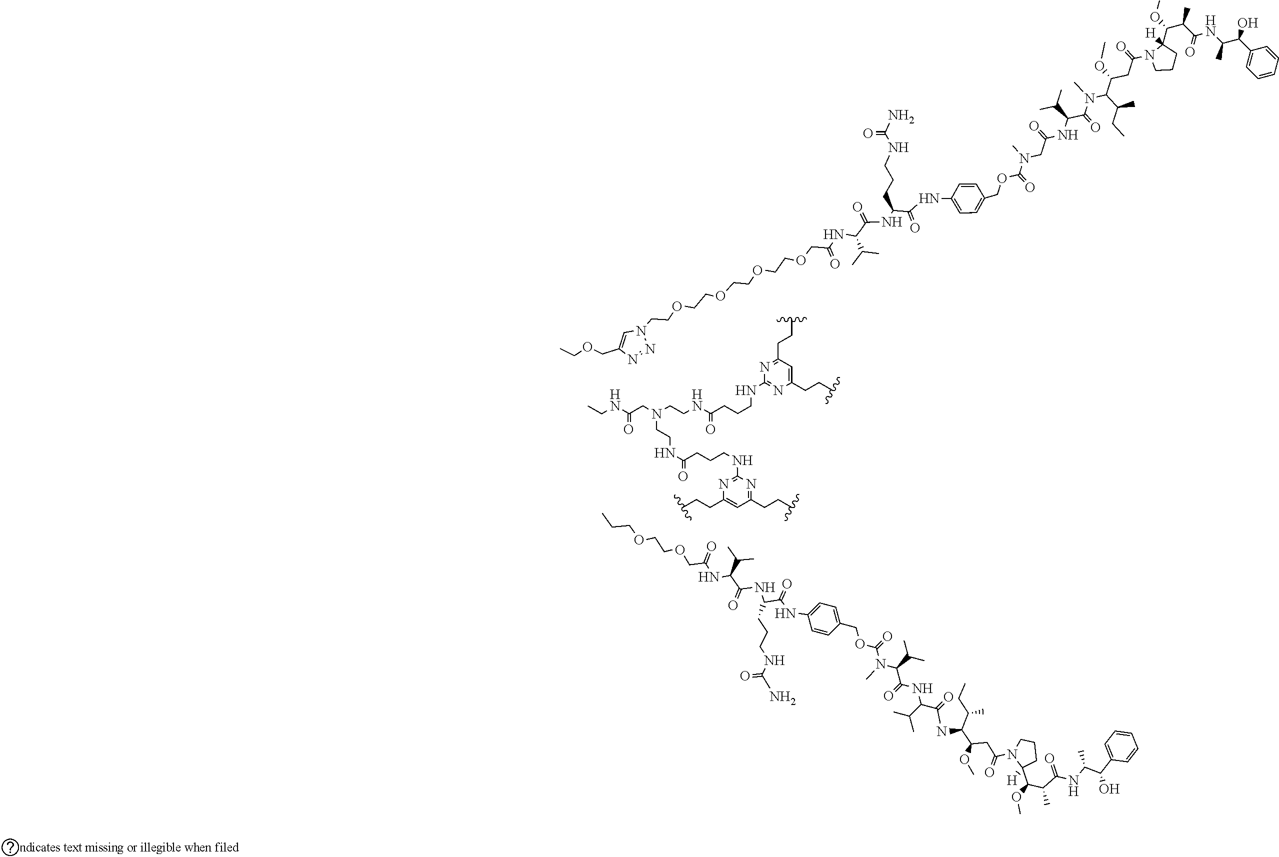
wherein: 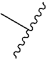
II indicates the position where the antibody is attached; and
the antibody is trastuzumab or brentuximab, suitably trastuzumab.

14. A conjugating reagent of Formula (II), shown below:
(D-L¹-FG′)ₙ-L-(Z)₈ (Formula II)
wherein:
FG′ is a functional linking moiety;
n is an integer selected from 0 to 20;
L and L′ are independently linkers;
Z is a functional group capable of reaction with a sulfur atom from a cysteine of an antibody; and
D is an active agent;
or a pharmaceutically acceptable salt, solvate or hydrate thereof.

15. A conjugating reagent according to claim 14, wherein the conjugating reagent is of Formula (IIa), shown below:
(D-L¹-FG′)ₙ-L-(Z²)₄ (Formula IIa)
wherein:
Z² is a re-bridging linking group comprising two functional groups capable of reaction with a sulfur atom from a cysteine moiety of an antibody; and
L, L′, FG′ and n are as defined in claim 14.

16. A conjugating reagent according to claim 14 or claim 15, wherein the conjugating reagent is selected from one of the following groups: 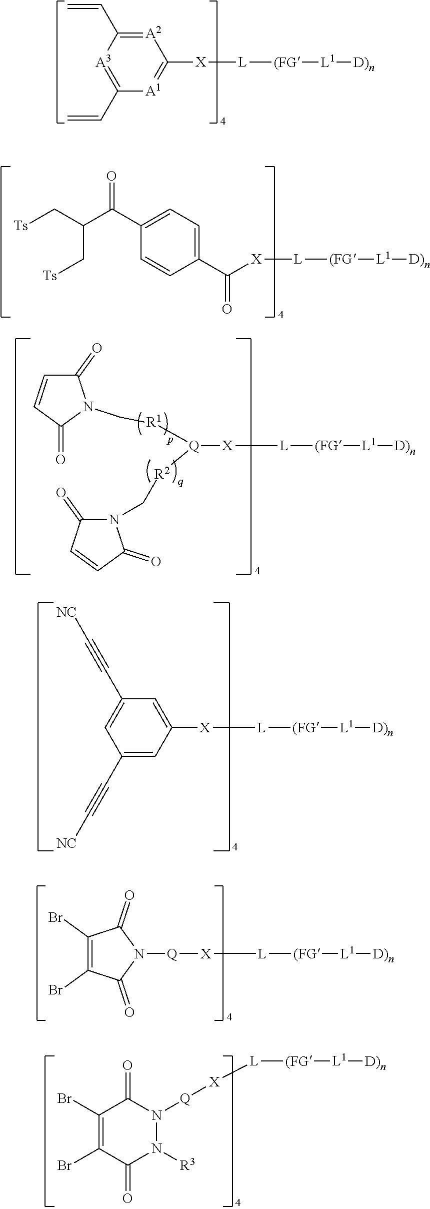 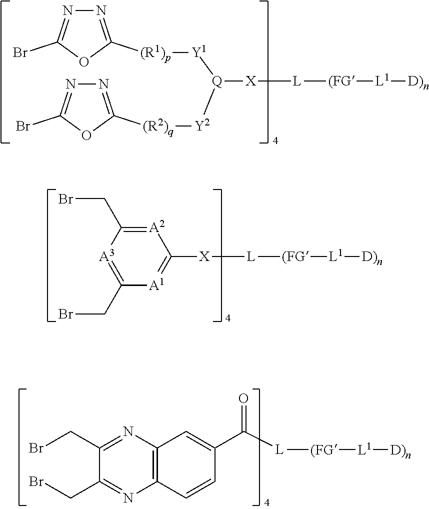
wherein:
A¹, A², A³, X, R¹, R², R³, Y¹, Y², Q, p and 1 are each as defined in claim 6;
Ts is a tosylate;
FG′, L, L¹, n and D are as defined in claim 14.

17. A conjugating reagent according to any one of claims 14 to 16, wherein the conjugating reagent is of Formula (IIc), shown below: 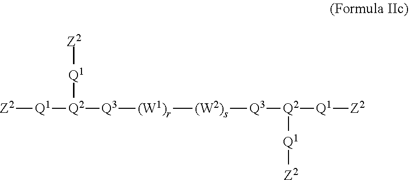
wherein:
Z² is a re-bridging linking group comprising two functional groups capable of reaction with a sulfur atom from a cysteine of an antibody;
Q¹, Q², Q³, W¹, W^{2′}, r and s are as defined in claim 8 or claim 9.

18. A conjugating reagent according to claim 17, wherein Z² is a group of Formula III, shown below: 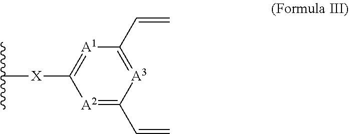
wherein:
two of A¹, A² and A³ are N and the other of A¹, A² and A³ is CH;
X is selected from NR^{N}, O and S, wherein R^{N} is H or C₁₋₂ alkyl; and 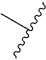
denotes the point of attachment to Q¹.

19. A conjugating reagent according to any one of claims 14 to 18, wherein the conjugating reagent is selected from:

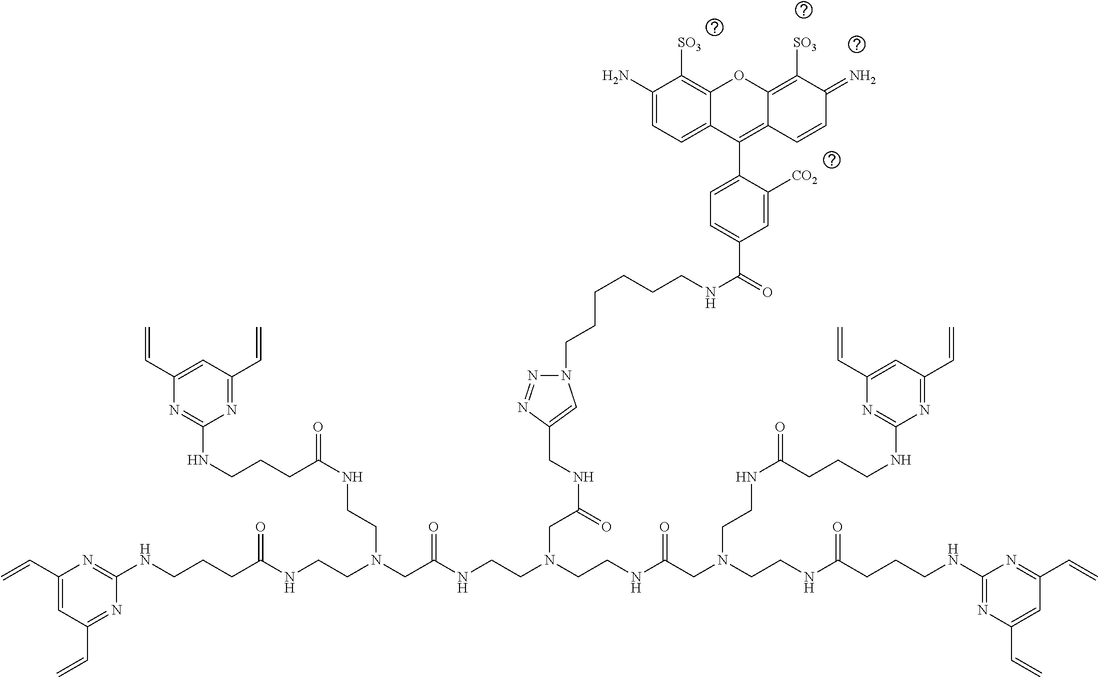

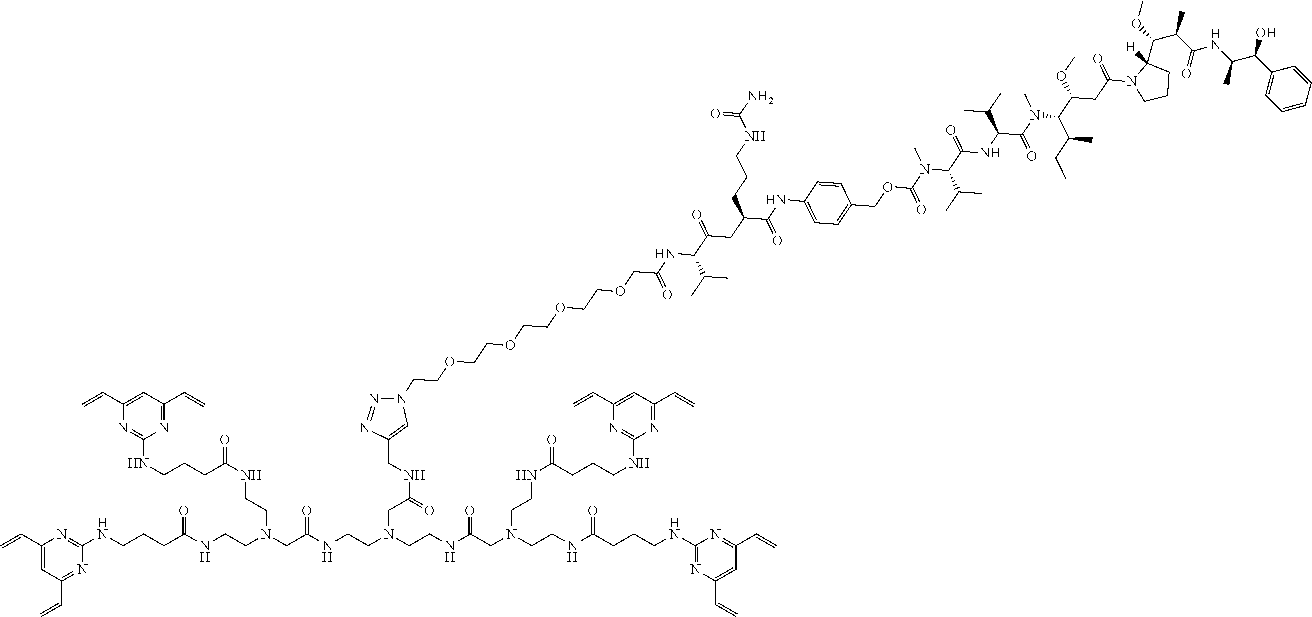 [SMILES: C=Cc1cc(C=C)nc(NCCCC(=O)NCCN(CCNC(=O)CCCNc2nc(C=C)cc(C=C)n2)CC(=O)NCCN(CCNC(=O)CN(CCNC(=O)CCCNc2nc(C=C)cc(C=C)n2)CCNC(=O)CCCNc2nc(C=C)cc(C=C)n2)CC(=O)NCc2cn(CCOCCOCCOCCOCC(=O)N[C@H](C(=O)C[C@@H](CCCNC(N)=O)C(=O)Nc3ccc(COC(=O)N(C)[C@H](C(=O)N[C@H](C(=O)N(C)[C@@H]([C@@H](C)CC)[C@@H](CC(=O)N4CCC[C@H]4[C@H](OC)[C@@H](C)C(=O)N[C@H](C)[C@@H](O)c4ccccc4)OC)C(C)C)C(C)C)cc3)C(C)C)nn2)n1]

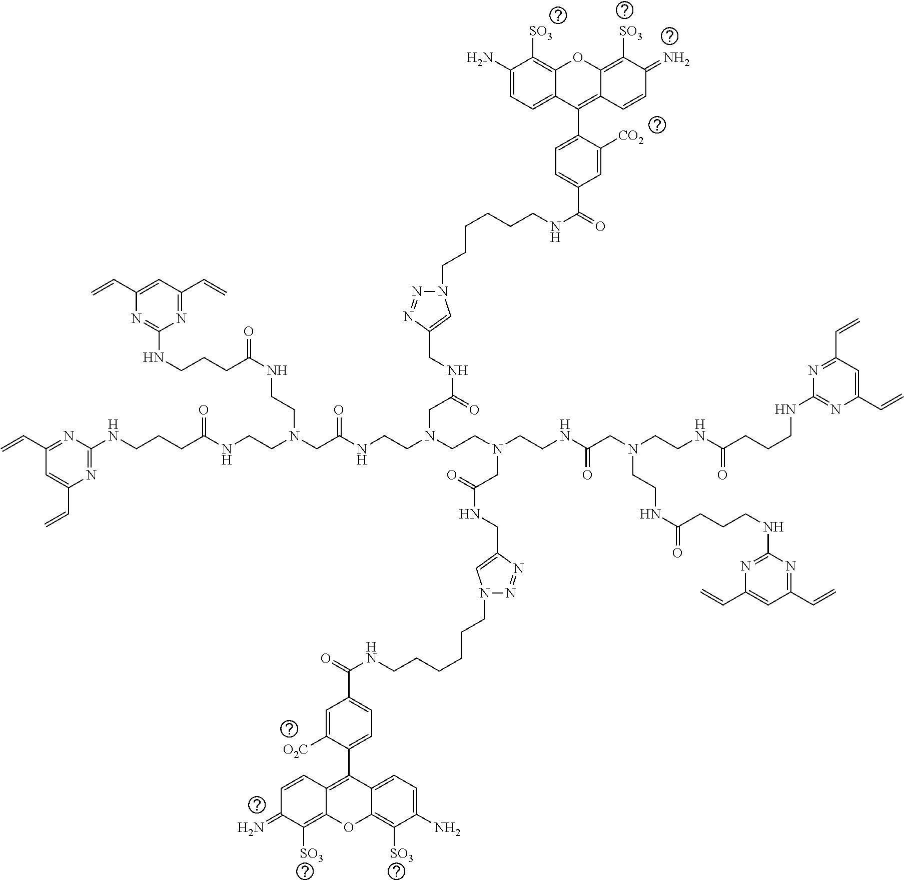

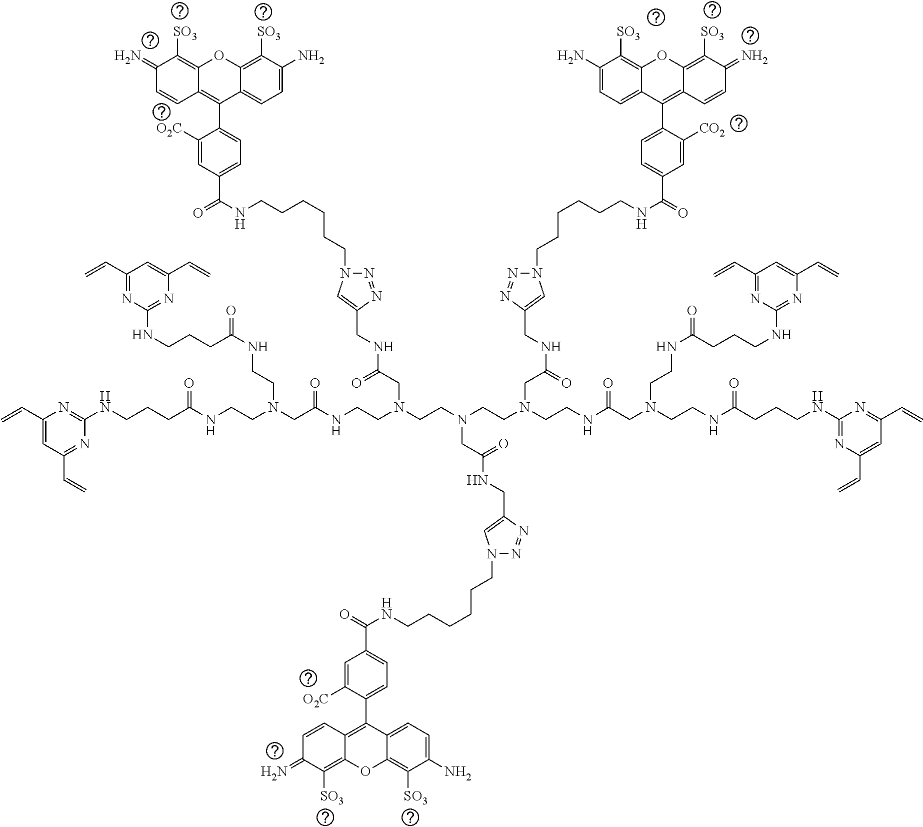

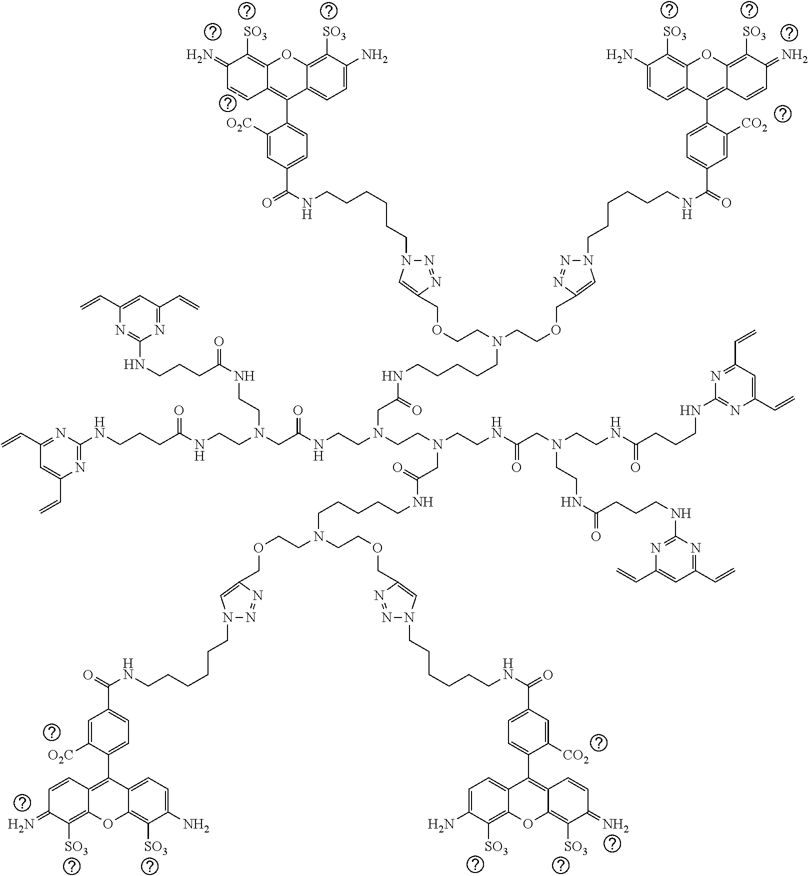

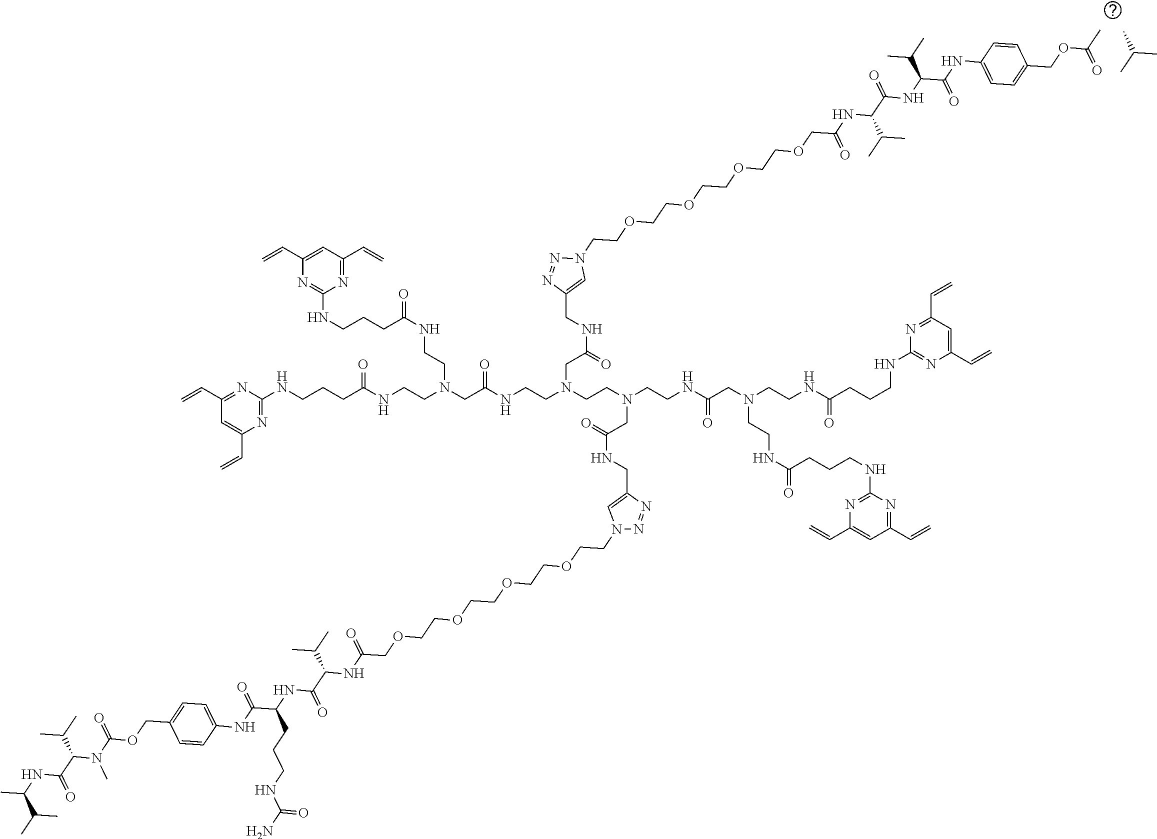

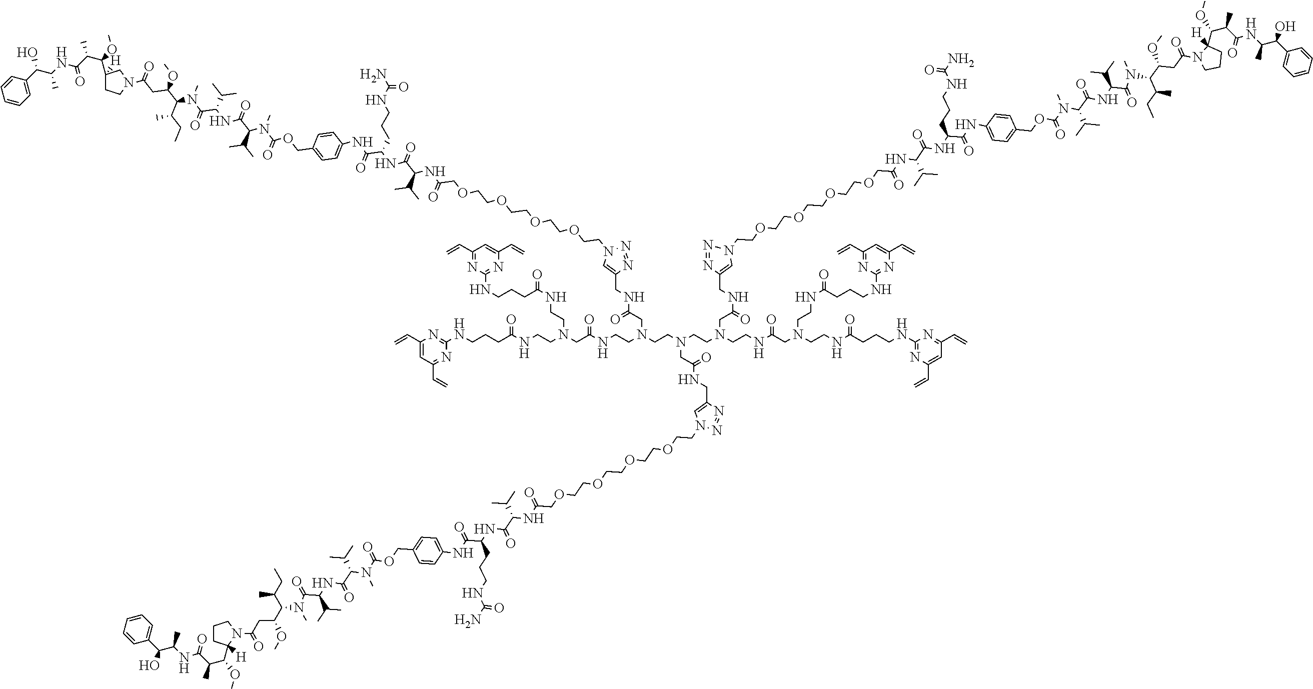

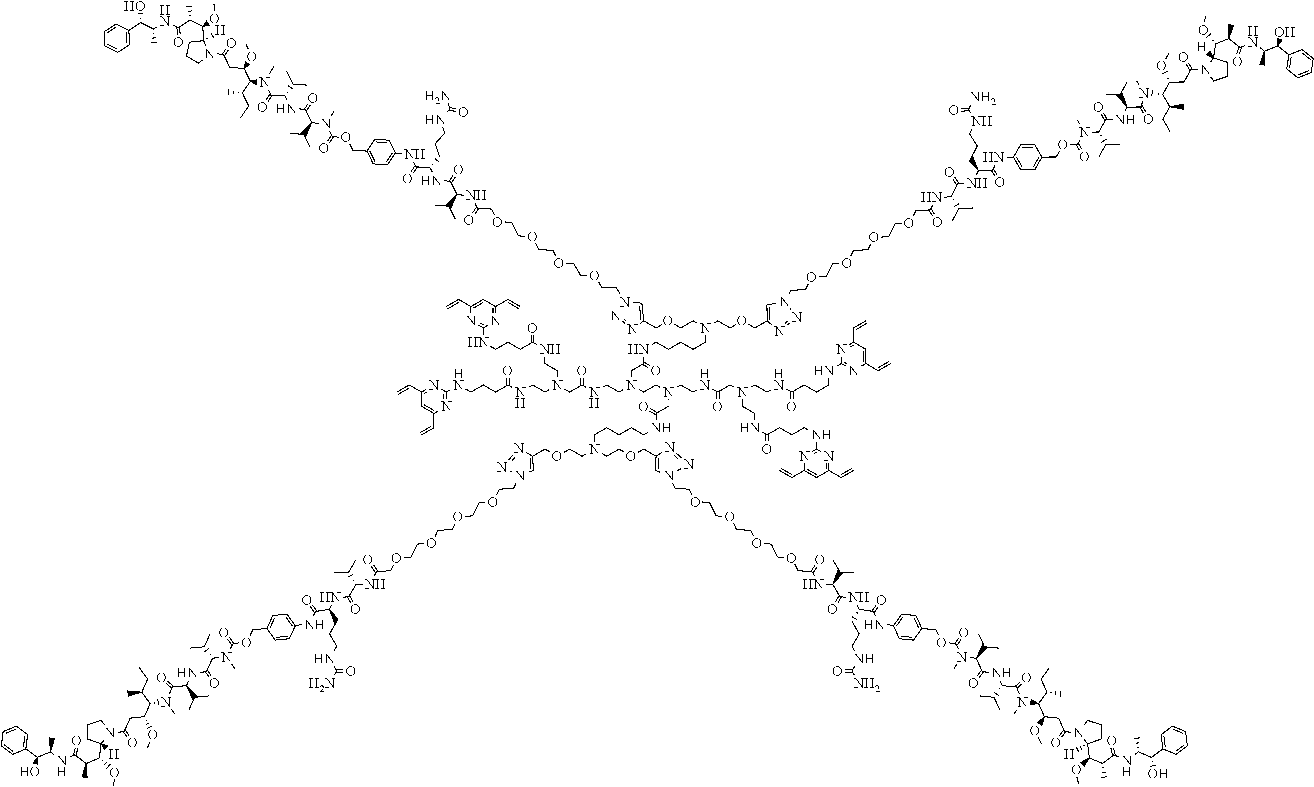

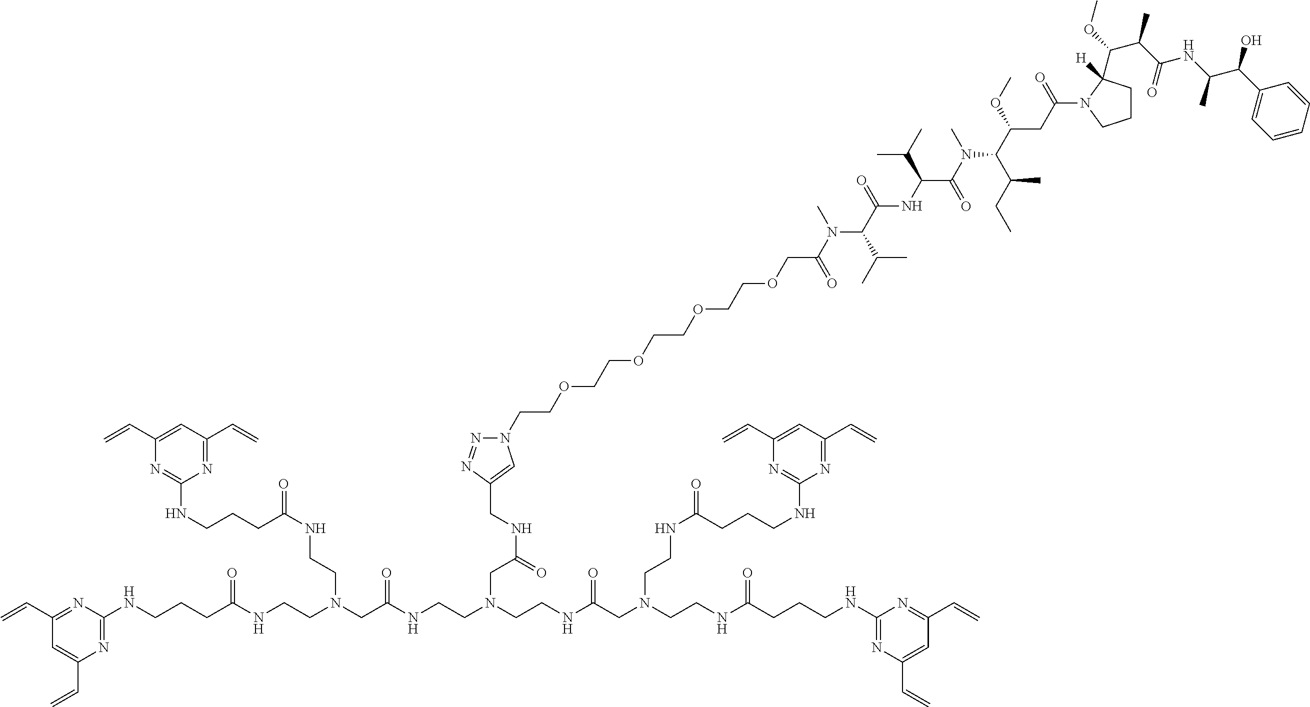 [SMILES: C=Cc1cc(C=C)nc(NCCCC(=O)NCCN(CCNC(=O)CCCNc2nc(C=C)cc(C=C)n2)CC(=O)NCCN(CCNC(=O)CN(CCNC(=O)CCCNc2nc(C=C)cc(C=C)n2)CCNC(=O)CCCNc2nc(C=C)cc(C=C)n2)CC(=O)NCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)n1]

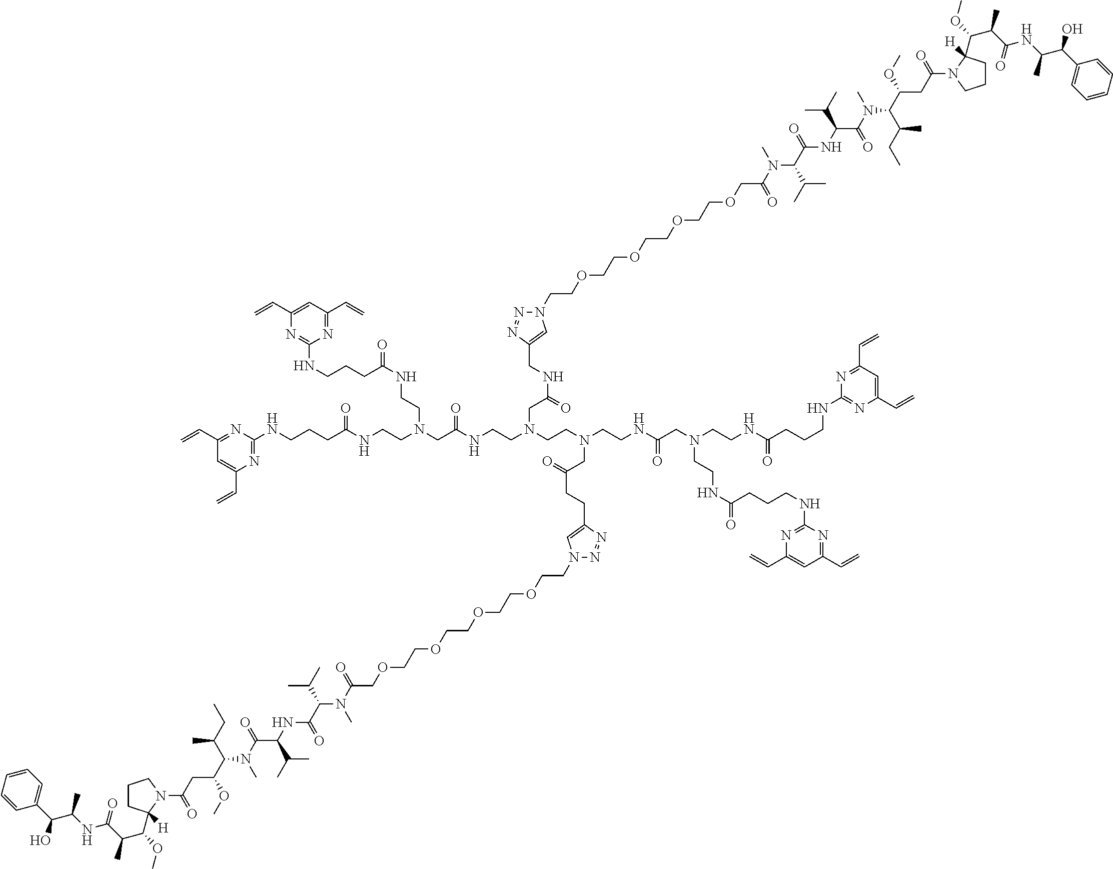 [SMILES: C=Cc1cc(C=C)nc(NCCCC(=O)NCCN(CCNC(=O)CCCNc2nc(C=C)cc(C=C)n2)CC(=O)NCCN(CCN(CCNC(=O)CN(CCNC(=O)CCCNc2nc(C=C)cc(C=C)n2)CCNC(=O)CCCNc2nc(C=C)cc(C=C)n2)CC(=O)NCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)CC(=O)CCc2cn(CCOCCOCCOCCOCC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)nn2)n1]

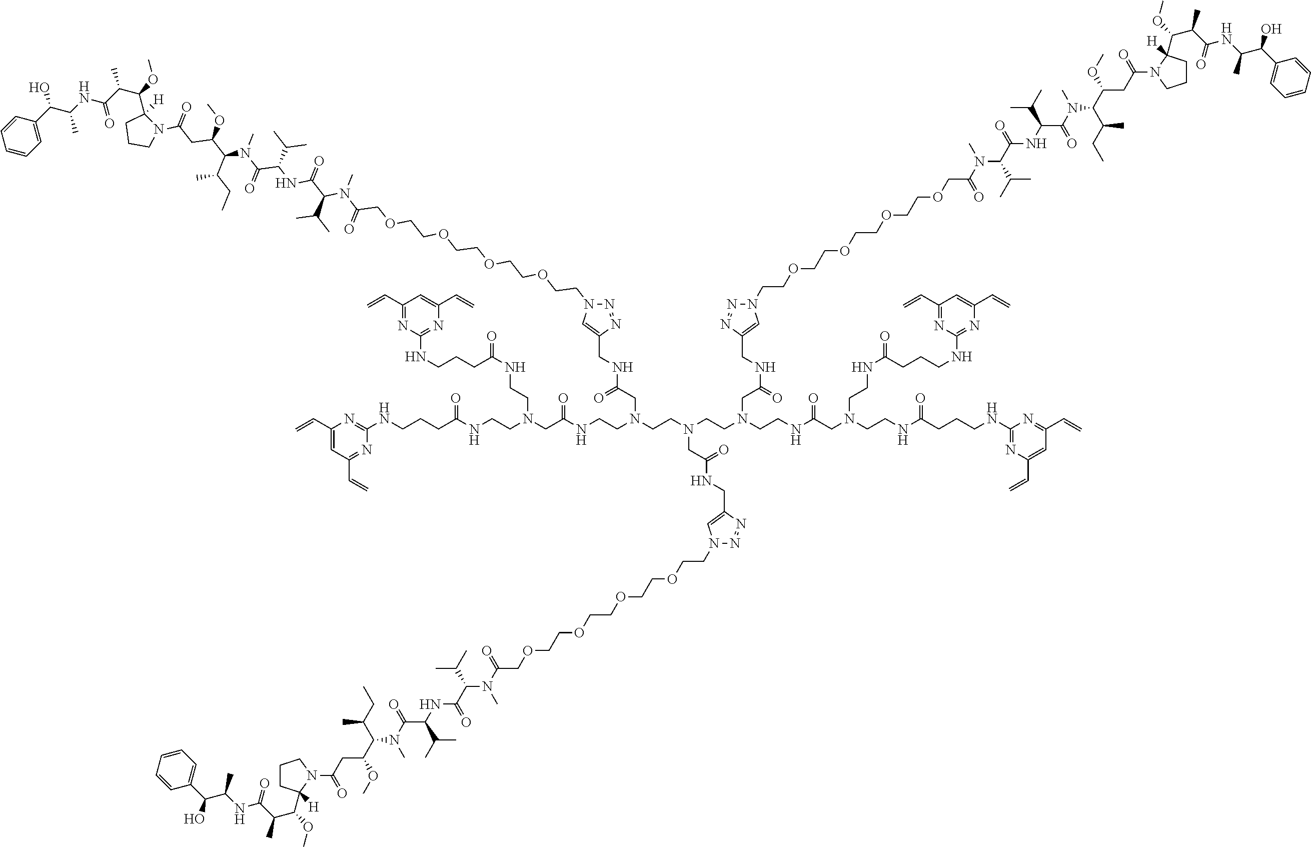

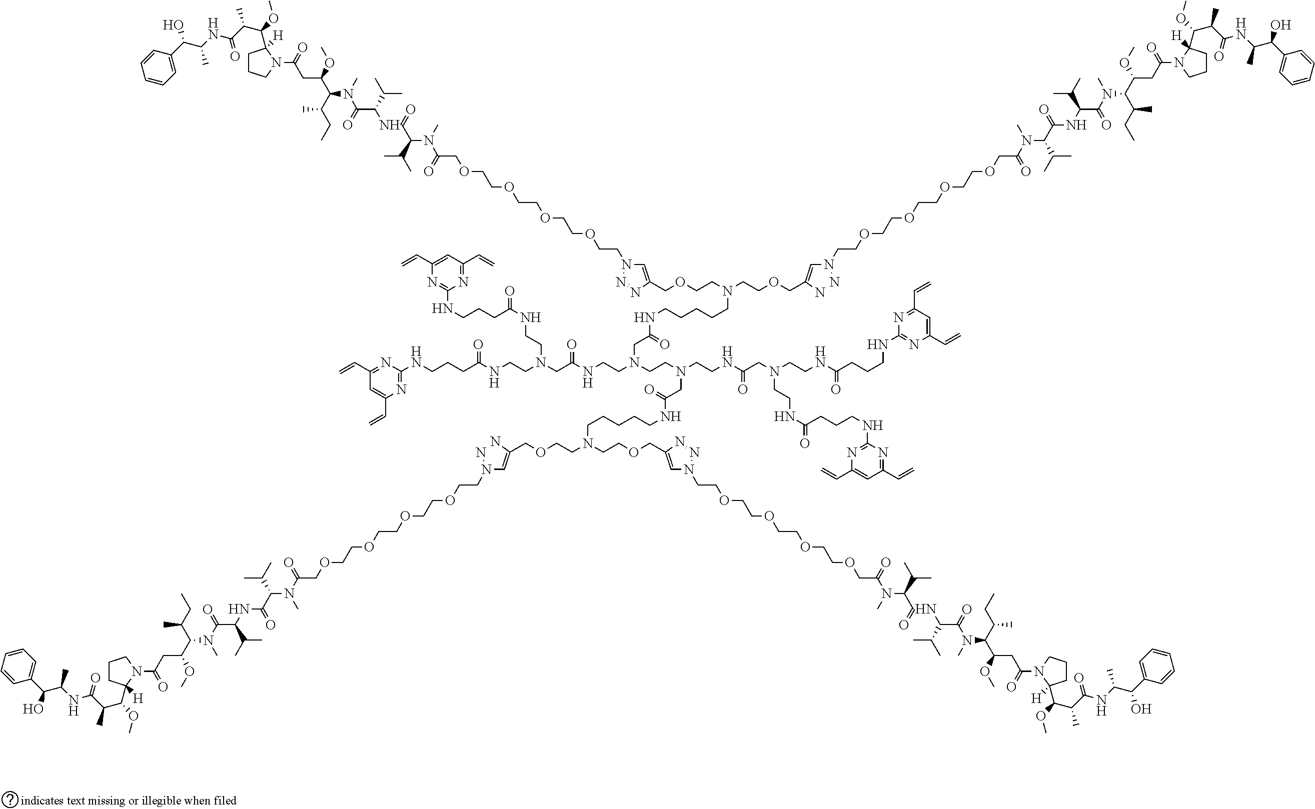

20. A compound of Formula (I), shown below:
(FG)ₙ-L-(Z)₈ (Formula I)
wherein:
FG is a functional group capable reacting with another moiety to form a functional linking moiety;
n is an integer selected from 0 to 20;
L is a linker; and
Z is a functional group capable of reaction with a sulfur atom from a cysteine moiety of an antibody;
or a pharmaceutically acceptable salt, solvate or hydrate thereof.

21. A compound according to claim 20, wherein the compound is of Formula (Ia), shown below:
(FG)ₙ-L-(Z²)₄ (Formula Ia)
wherein:
Z² is a re-bridging linking group comprising two functional groups capable of reaction with a sulfur atom from a cysteine or lysine moiety of an antibody;
L, FG and n are as defined in claim 20 above.

22. A compound according to any one of claims 20 to 21, wherein the compound is selected from one of the following groups: 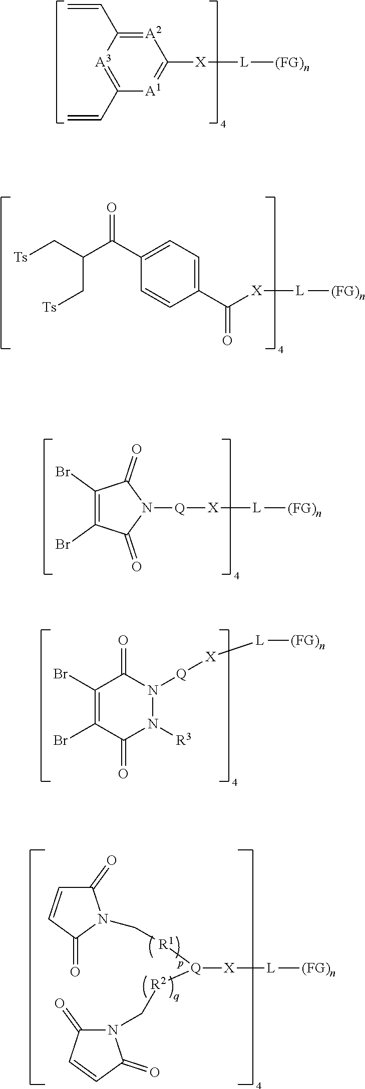 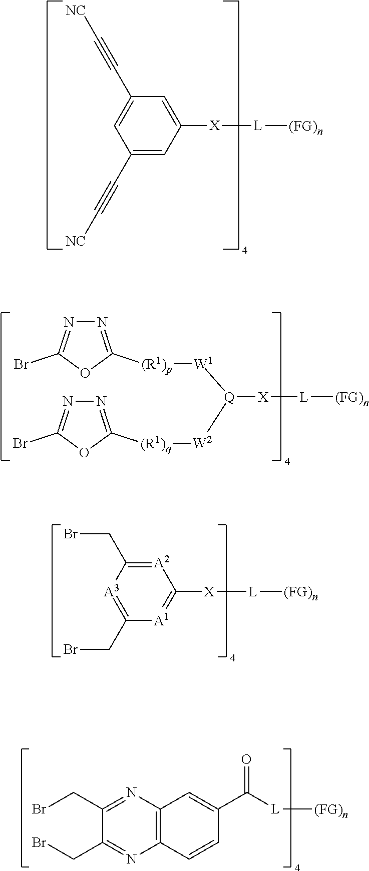
wherein:
A¹, A², A³, X, R¹, R², R³, Y¹, Y², Q, p and 1 are each as defined in claim 6;
Ts is a tosylate;
L and FG are as each defined in claim 20 above.

23. A compound according to any one of claims 20 to 22, wherein the compound is of Formula (Ic), shown below: 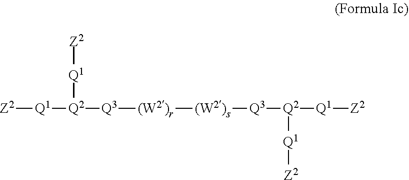
wherein:
Z² is as defined in claim 18 or 21 or Z² is a group of Formula (III) as defined in claim 6;
Q¹, Q², Q³, r and s are as defined in claim 8 or claim 9;
W¹ is a group of Formula IVc₂, Formula IVe₂ or Formula IVc: 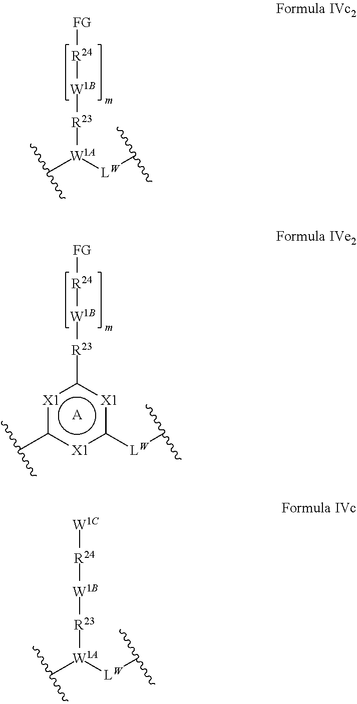
wherein:
W^{1A} is selected from N and CR¹⁴, wherein R¹⁴ is a selected from hydrogen and C₁-C₄alkyl;
L^{W} is absent or selected from a C₁-C₆alkylene and a C₁-C₆alkylene containing amino, O or N in the backbone;
Ring A is a 6-membered aryl, a 6-membered heteroaryl, a 6-membered heterocyclyl or a 6-membered cycloalkyl, wherein X₁ is selected from CH or N;
W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
R¹⁵ and R¹⁶ are independently selected from hydrogen and C₁-C₄alkyl;
R²³ and R²⁴ are independently selected from a bond, a C₁-C₈alkylene and a C₁-C₈alkylene containing O in the backbone;
m is an integer selected from 1 or 2;
W^{1C} is a group of Formula W^{C2}: 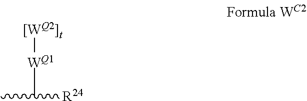
wherein:
W^{Q1} is a group of Formula W^{C3}: 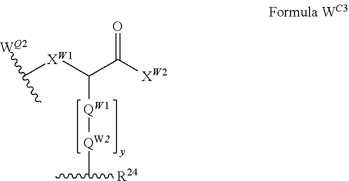
wherein:
Q^{W1} is absent or selected from a C₁-C₁₂alkylene and a C₁-C₁₂alkylene containing O or N in the backbone; and
Q^{W2} is selected from —C(═O)—, —OC(═O)—, —C(═O)O—, —NHC(═O)—, and —C(═O)NH—;
y is an integer selected from 0 or 1;
X^{W1} is selected from O or NH;
X^{W2} is selected from hydrogen, C₁-C₄alkyl, OR^{X1} and
NR^{x1}R^{x2}, wherein R^{x1} and R^{x2} are independently selected from hydrogen and C₁-C₄alkyl; 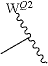
indicates the position where X^{W1} is attached to a group of formula W^{Q2}; 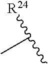
indicates the position where W^{Q1} is attached to a group of formula R²⁴
W^{Q2} is a group of Formula W^{C4B}: 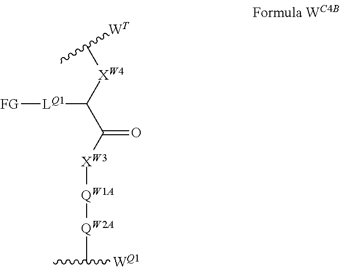
wherein:
Q^{W1A} is absent or selected from C₁-C₁₂alkylene and C₁-C₁₂alkylene containing O or N in the backbone; and
Q^{W2A} is selected from —C(═O)—, —OC(═O)—, —C(═O)O—, —NHC(═O)—, and —C(═O)NH—;
X^{W3} is selected from O or NH; 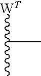
indicates the position where X^{W4} is attached to another group of formula W^{Q2} via the Q^{W2A} substituent group, or 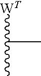
indicates the position where X^{W4} is attached to one of the following groups hydrogen or —C(═O)R^{X3}, wherein R^{x3} is selected from hydrogen and C₁-C₄alkyl; 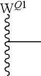
indicates the position where Q^{W2A} is attached to the group of formula W¹ via the X^{W1} group;
X^{W4} is selected from 0 or NH:
L^{Q1} is a linker comprising one or more groups selected from a C₁-C₂₀alkylene, an alkylenediamine moiety, a (poly)ethylene glycol moiety, an amino acid residue and combinations thereof;
t is an integer selected from 1 to 8;
FG is as defined in claim 20;
W^{2′} is a group of Formula IVd: 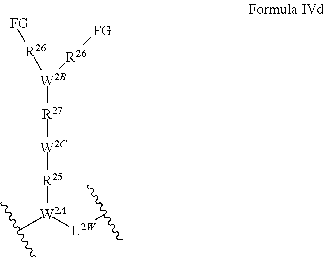
wherein:
W^{2A} is selected from N and CR¹⁷, wherein R¹⁷ is a selected from hydrogen and C₁-C₄alkyl;
W^{2B} is a group selected from N, CR¹⁸, —C(═O)N— and —C(═O)CR¹⁸—;
W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
L^{2W} is absent or selected from a C₁-C₆alkylene and a C₁-C₆alkylene containing amino, O or N in the backbone;
R¹⁷ and R¹⁸ are independently selected from hydrogen and C₁-C₄alkyl;
R²⁵, R²⁶ and R²⁷ are independently selected from a bond, C₁-C₈alkylene and C₁-C₈alkylene containing O in the backbone; and
FG is as in claim 20.

24. A compound according to any one of claims 20 to 23, wherein the compound is of Formula (Ic), shown below: 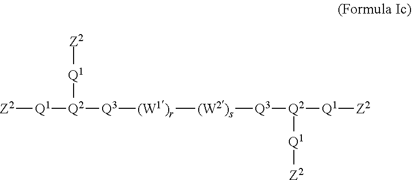
wherein:
Z² is as defined in claim 18 or 21;
Q¹, Q², Q³, r and s are as defined in claim 8 or claim 9;
W^{1′} is a group of Formula IVc₂: 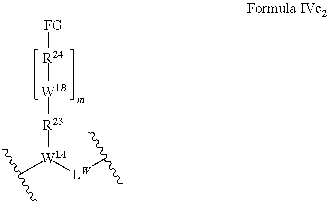
wherein:
W^{1A} is selected from N and CR¹⁴, wherein R¹⁴ is a selected from hydrogen and C₁-C₄alkyl;
L^{W} is absent or selected from a C₁-C₆alkylene and a C₁-C₆alkylene containing O or N in the backbone;
W^{1B} is a group selected from —C(═O)—, —OC(═O)—, —C(═O)O—, —OC(═O)O—, —NR¹⁵C(═O)—, —C(═O)NR¹⁵—, —NR¹⁵C(═O)NR¹⁶—, —C(═NR¹⁵)— and —C(═S)—;
R¹⁵ and R¹⁶ are independently selected from hydrogen and C₁-C₄alkyl;
R²³ and R²⁴ are independently selected from a bond, C₁-C₈alkylene and C₁-C₆alkylene containing O in the backbone;
m is an integer selected from 1 or 2; and
FG is as defined in claim 20;
W^{2′} is a group of Formula IVd: 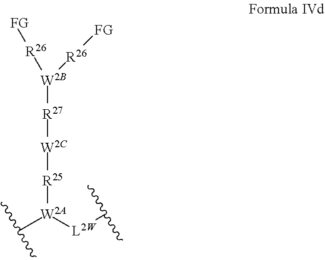
wherein:
W^{2A} is selected from N and CR¹⁷, wherein R¹⁷ is a selected from hydrogen and C₁-C₄alkyl;
W^{2B} is a group selected from N, CR¹³, —C(═O)N— and —C(═O)CR¹⁸—;
W^{2C} is a group selected from —C(═O)—, —OC(═O)—, —C(═O)O—, —OC(═O)O—, —NR¹⁷C(═O)—, —C(═O)NR¹⁷—, —NR¹⁷C(═O)NR¹⁸—, —C(═NR¹⁷)— and —C(═S)—;
L^{2W} is absent or selected from a C₁-C₆alkylene and a C₁-C₆alkylene containing O or N in the backbone;
R¹⁷ and R¹³ are independently selected from hydrogen and C₁-C₄alkyl;
R²⁵, R²⁶ and R²⁷ are independently selected from a bond, C₁-C₈alkylene and C₁-C₈alkylene containing O in the backbone; and
FG is as in claim 20.

25. A compound according to claim 23 or 24, wherein Z² is a group of Formula III, shown below: 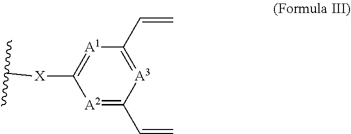
wherein:
two of A¹, A² and A³ are N and the other of A¹, A² and A³ is CH;
X is selected from N, NR^{N}, O and S, wherein R^{N} is H or C₁₋₂alkyl; and 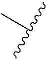
denotes the point of attachment to Q¹.

26. A compound according to any one of claims 20 to 25, wherein FG is selected from an alkene, an alkyne, an azide, a hydroxyl, an amine, a carboxylic acid, an aldehyde, an acyl halide, a tetrazine, an alkoxyamine (e.g. hydroxylamine), a hydrazine, an electron-rich dienophile (e.g. a 1,3-nitrone alkene) and an electron-poor diene (e.g. tetrazine), nitrone, an isocyanate and an isothiocyanate.

27. A compound according to any one of claims 20 to 26, wherein FG is selected from an alkyne or an azide.

28. A compound according to any one of claims 20 to 27, wherein the compound is selected from:

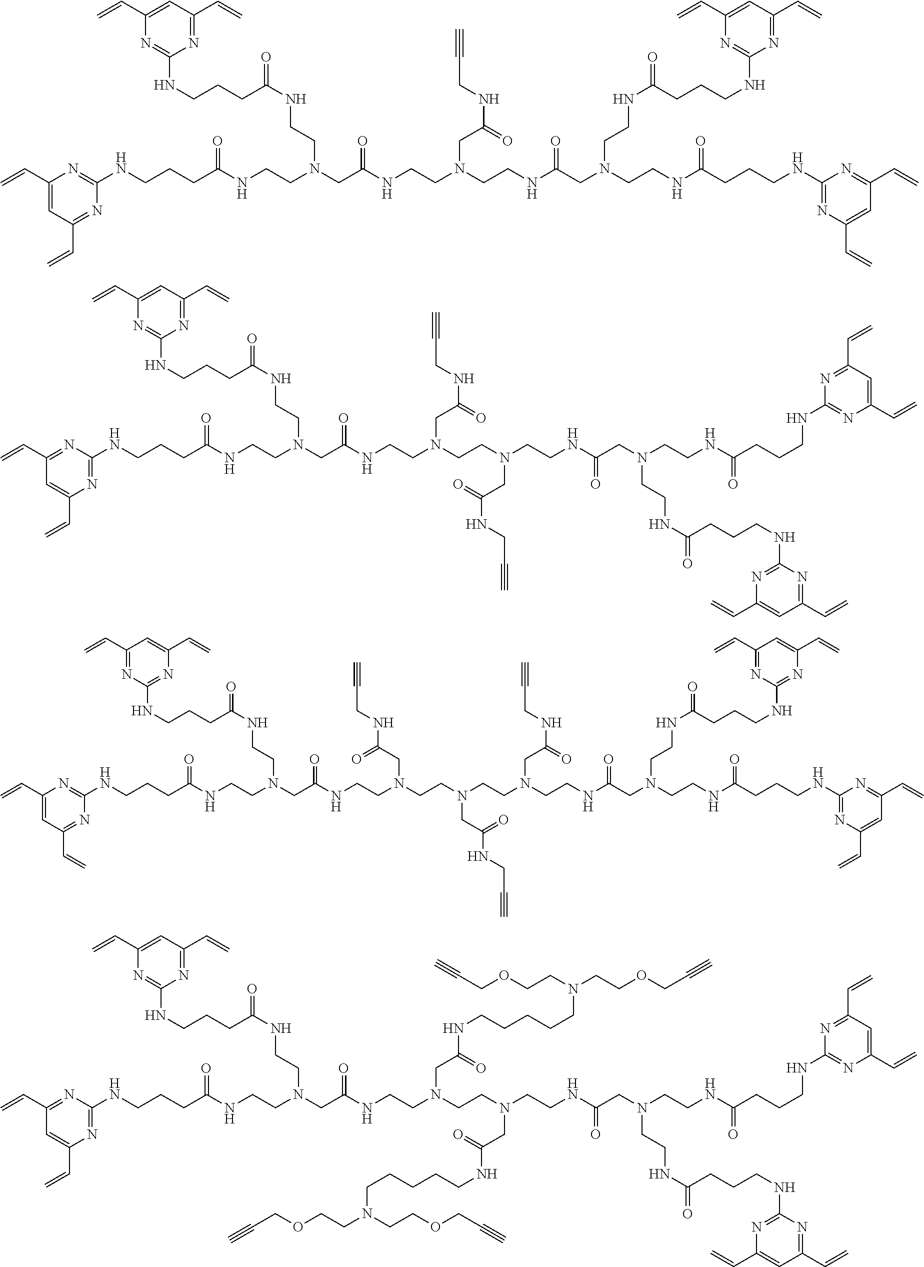

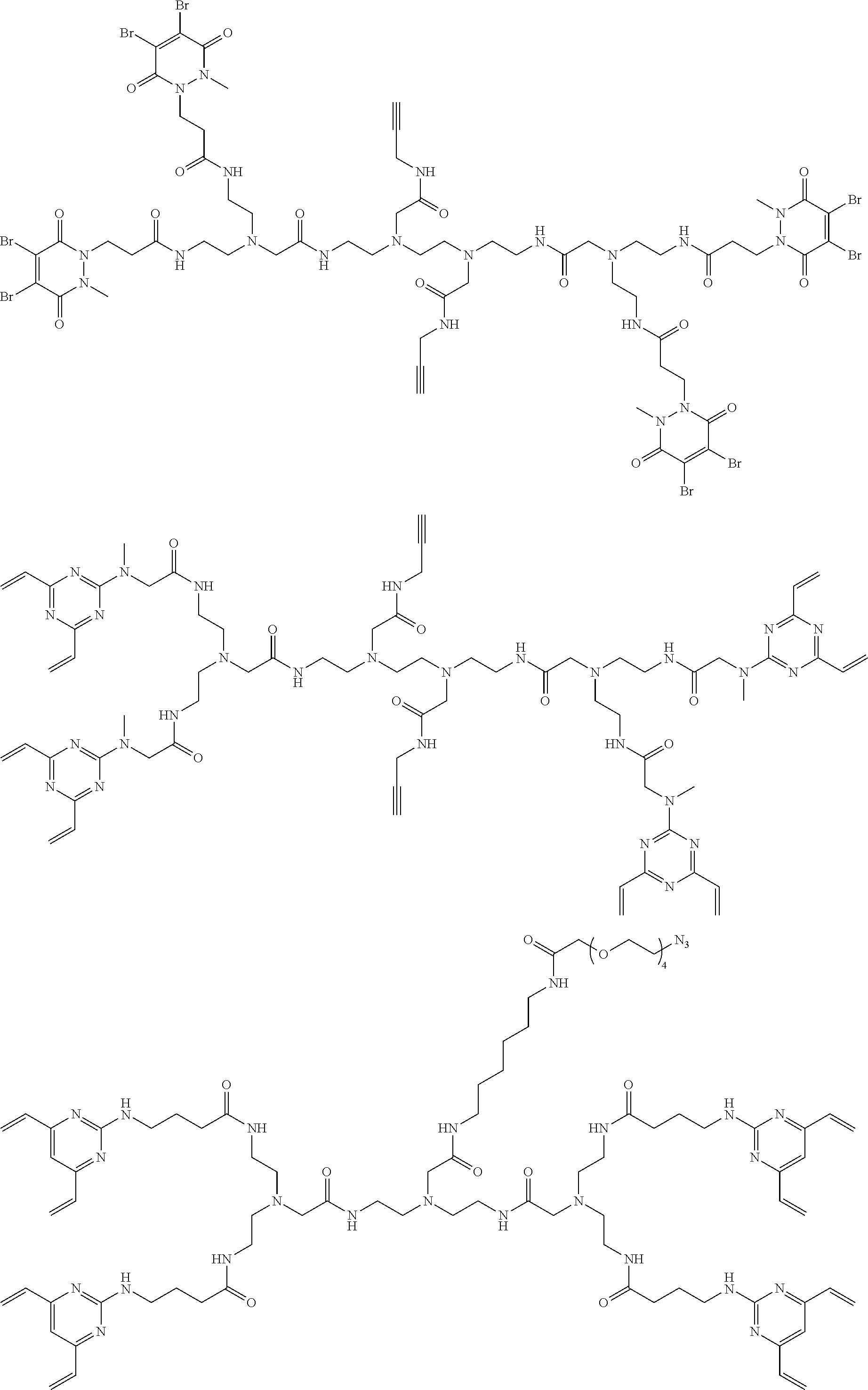

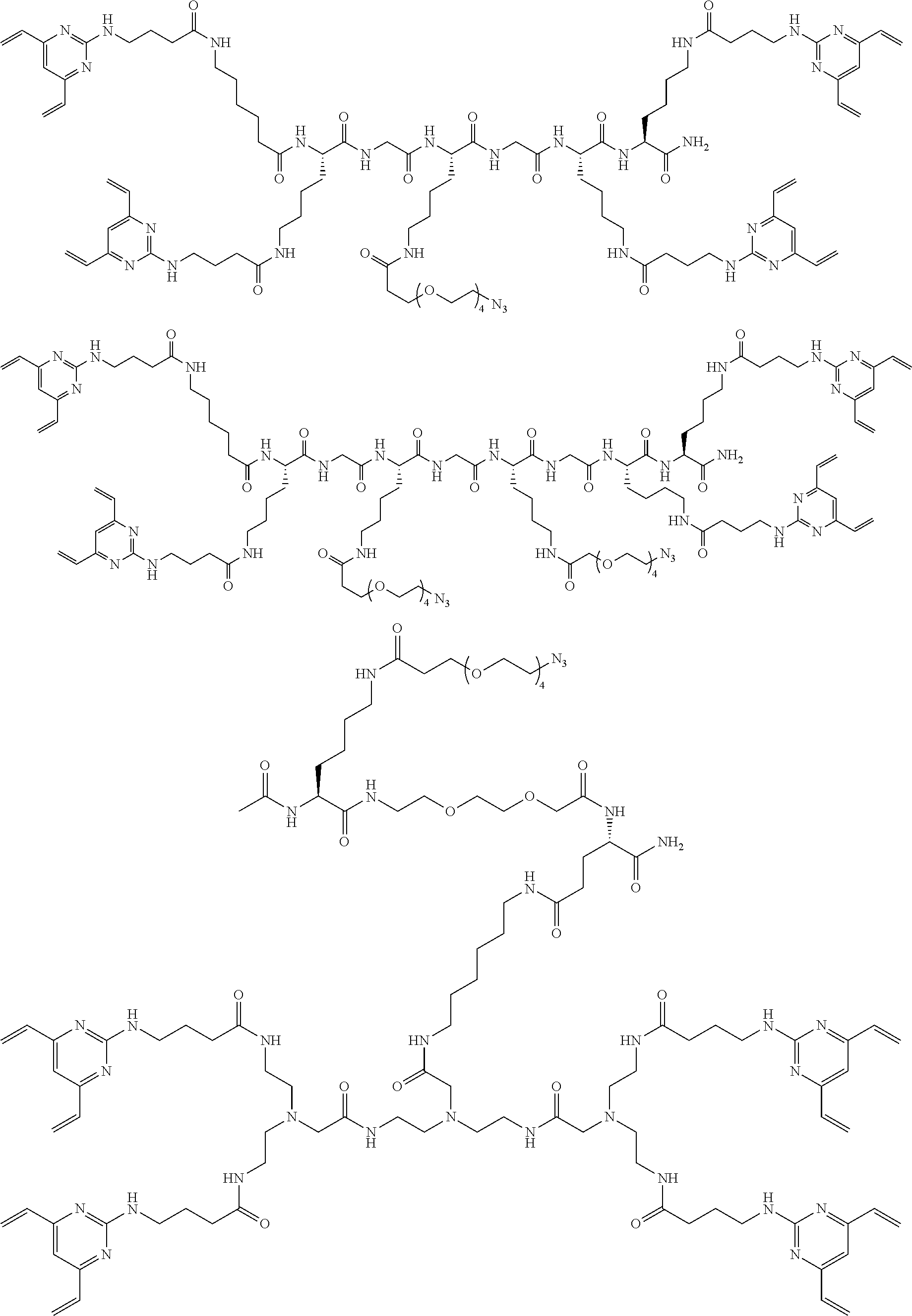

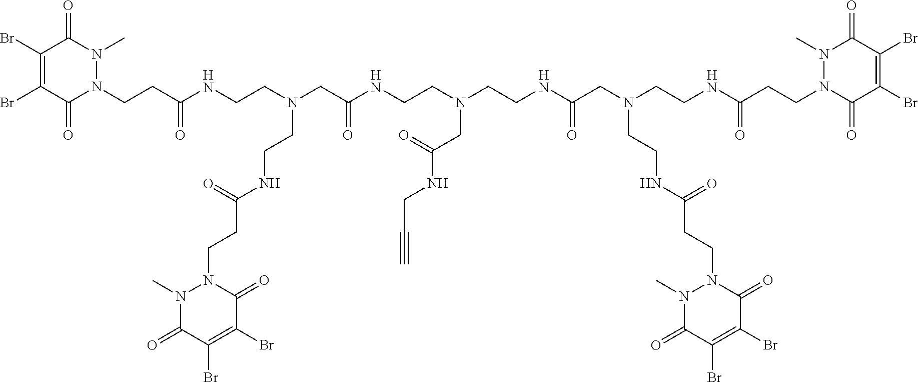 [SMILES: C#CCNC(=O)CN(CCNC(=O)CN(CCNC(=O)CCn1c(=O)c(Br)c(Br)c(=O)n1C)CCNC(=O)CCn1c(=O)c(Br)c(Br)c(=O)n1C)CCNC(=O)CN(CCNC(=O)CCn1c(=O)c(Br)c(Br)c(=O)n1C)CCNC(=O)CCn1c(=O)c(Br)c(Br)c(=O)n1C]

29. A compound according to any one of claims 20 to 27, wherein the compound is selected from:

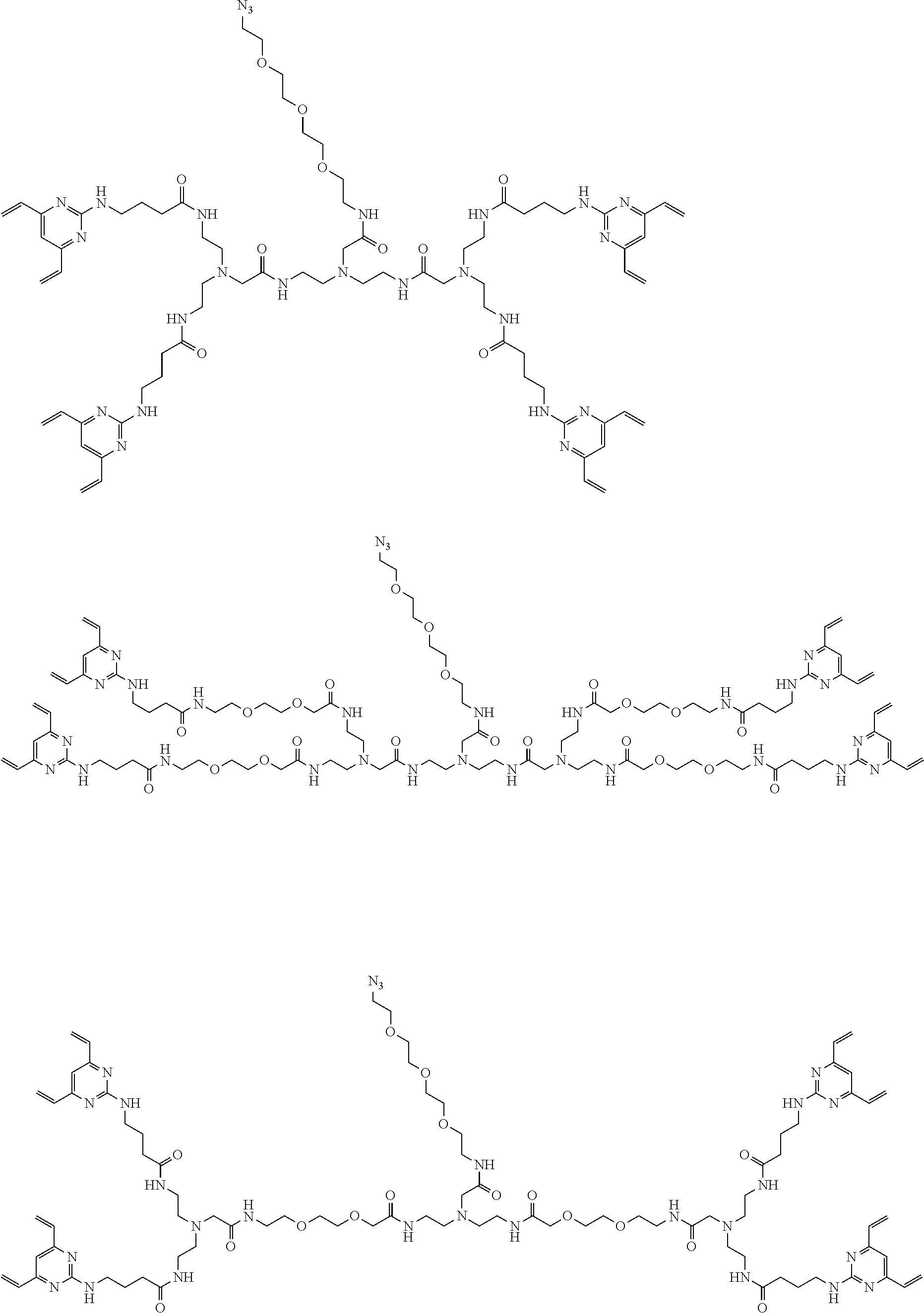

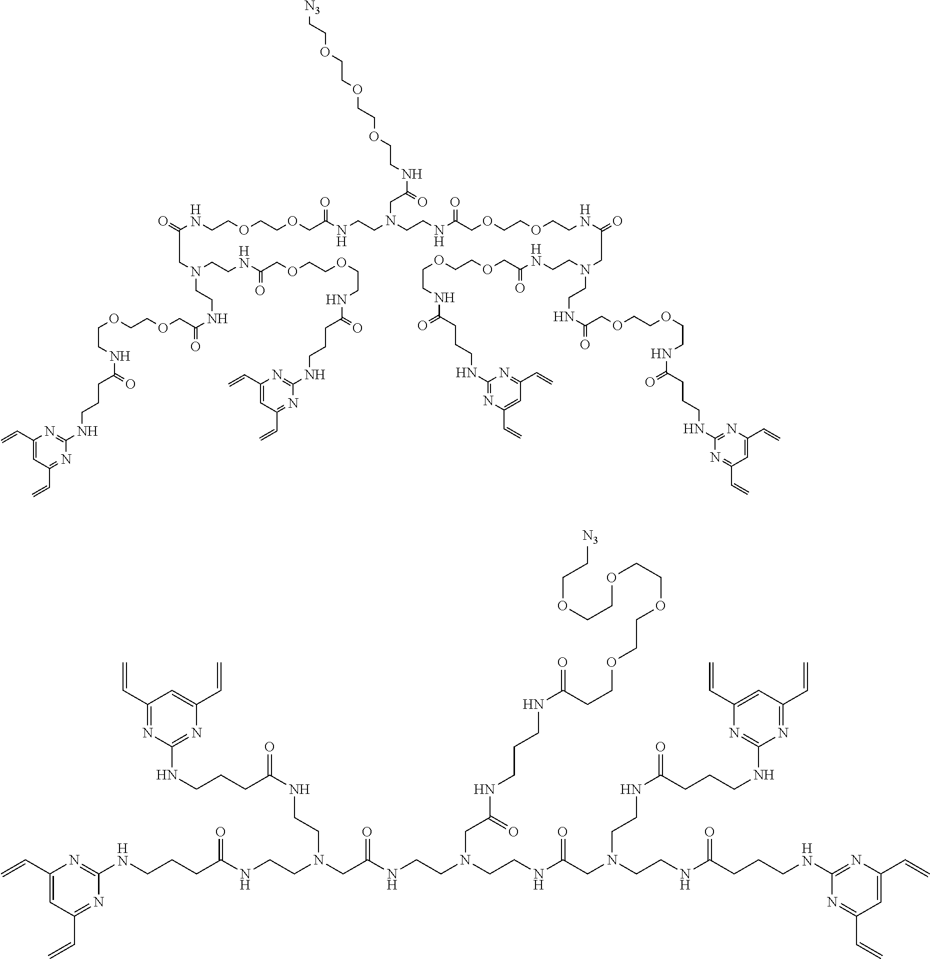

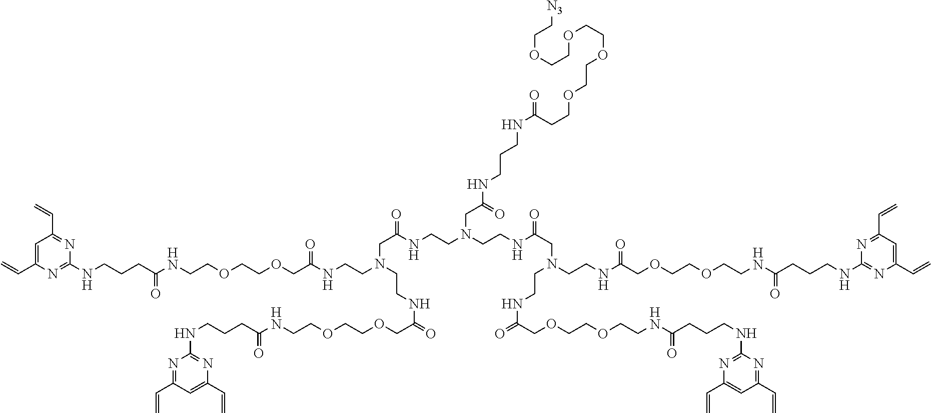

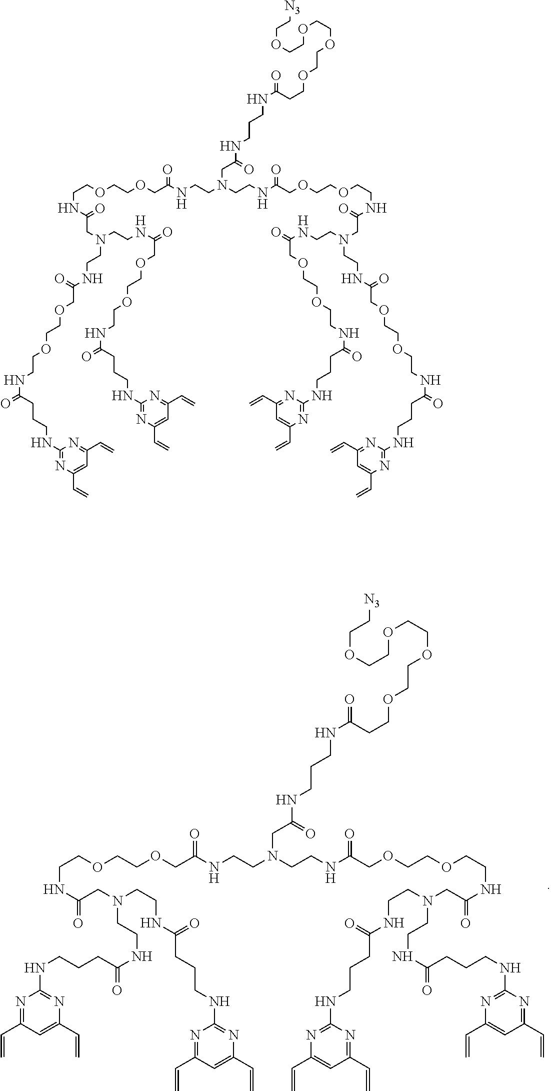

30. A conjugate according to any one of claims 8 to 13, or a conjugating reagent according to any one of claims 15 to 19, wherein FG′ is selected from 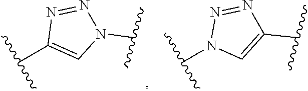 [SMILES: *c1cn(*)nn1 ; *c1cn(*)nn1]
—NH(═O)C— and —C(═O)NH—.

31. A conjugate according to any one of claims 1 to 13, or a conjugating reagent according to any one of claims 14 to 19, wherein the active agent is a cytotoxin, optionally selected from the group comprising auristatins, maytansinoids, tubulysins, calicheamicins, duocarmycins, pyrrolobenzodiazepines, camptothecin analogues and doxorubicin.

32. A conjugate according to any one of claims 1 to 13, or a conjugating reagent according to any one of claims 14 to 19, wherein the conjugate comprises between 1 and 8 active agents.

33. A conjugate according to any one of claims 1 to 7, or a conjugating reagent according to any one of claims 13 to 15, or a compound according to any one of claims 20 to 22, wherein the linker (L) comprises one or more groups selected from an alkylenediamine moiety, a polyethylene glycol moiety and an amino acid residue.

34. A conjugating reagent according to claim 14, or a compound according to claim 20, wherein Z is a Michael Acceptor.

35. A pharmaceutical composition comprising, a conjugate according to any one of claims 1 to 13, and a pharmaceutically acceptable carrier, excipient or diluent.

36. A conjugate according to claims 1 to 13 and 30 to 33, for use in the treatment of a proliferative disease.
